(19) United States
(12) Patent Application Publication
(10) Pub. No.: US 2026/0325681 A1
(43) Pub. Date: Oct. 22, 2026

(54) GENETIC ELEMENTS FOR ADJUSTMENT OF EXPRESSION

(71) Applicant: Remedium Bio, Inc., Needham, MA (US)

(72) Inventors: Alexei Goraltchouk, Cambridge, MA (US); Frank Luppino, Needham, MA (US); Alexey Seregin, Westford, MA (US); Judith Hollander, Needham, MA (US)

(21) Appl. No.: 19/556,455

(22) Filed: Mar. 4, 2026

Related U.S. Application Data
(63) Continuation of application No. PCT/US2024/045412, filed on Sep. 5, 2024.
(60) Provisional application No. 63/536,530, filed on Sep. 5, 2023.

(51) Int. Cl. A61K 48/00

(52) U.S. Cl. CPC A61K 48/0066; A61K 48/0075

(57) ABSTRACT

Most therapeutic treatments require the adjustment of the prescribed dose following initiation of treatment. Dose adjustment is important to ensure an optimal safety and efficacy profile of a treatment. Gene therapy is inherently limited by its inability to adjust the dose following initial administration. This invention describes genetic elements that enable dose adjustment following initial dosing with a gene therapy. Enablement of dose adjustment allows for the ability of single-injection gene therapy to replace multi-injection subcutaneous protein regiments improving pharmacokinetics, safety/efficacy profile, medication adherence, and reducing healthcare costs.

### CROSS-REFERENCE TO RELATED APPLICATIONS

This application is a continuation of international PCT application PCT/US2024/045412, filed Sep. 5, 2024, which claims the benefit under 35 U.S.C. § 119(e) of U.S. Provisional Application No. 63/536,530, filed Sep. 5, 2023, both entitled “GENETIC ELEMENTS FOR ADJUSTMENT OF EXPRESSION,” the disclosure of each of which is hereby incorporated by reference in its entirety.

### REFERENCE TO AN ELECTRONIC SEQUENCE LISTING

The contents of the electronic sequence listing (R087270004US01-SEQ-JAV.xml; Size: 246,329 bytes; and Date of Creation: Mar. 3, 2026) are herein incorporated by reference in their entirety.

### FIELD

The present invention generally relates to human and veterinary therapeutics and more specifically to gene therapy treatments of conditions that may require the adjustment of the dose following initial administration.

### SUMMARY

Effects of pharmacological treatments are not homogeneous nor consistent due to the diversity of human biology. As a result, most treatments could benefit from the adjustment of the dosing regimen following the initial treatment based on the physician assessment of the treatment's effect. Dose adjustment is important to ensure an optimal safety and efficacy profile of a treatment. Should the initial treatment prove insufficiently effective at managing the pathology or symptoms and the side effects optimally tolerable, the treatment dose may be increased. Conversely, should the treatment dose be such that the side effects are excessive or cannot be tolerated by the patient, the treatment dose should be decreased. Similarly, if a patient is cured of a condition, the dose should be reduced to a maintenance level or completely to zero, depending on the pathology. Gene therapy, however, is inherently limited by its inability to adjust the dose following initial administration as a therapeutic modality.

When genetic cargo is delivered by a viral or non-viral vector the genetic cargo either integrates into the host cell genome and resides within the cell permanently and becomes passed on to progeny, or the genetic cargo enters the nucleus and forms an episome expressing for years at a level defined by the promoter, chromatic, epigenetics, or generally the natural intracellular environment and extracellular signals. As an example, AAV-delivered non-integrating gene therapies have demonstrated stable transgene expression over a period of multiple years in preclinical and clinical studies. Similarly, lentivirally delivered therapeutic transgenes integrate permanently into the host cell genome and provide multi-year stable expression of the therapeutic transgene. Currently gene expression of gene therapy treatments cannot be adjusted up or down in a manner that would enable therapeutic treatment of conditions that require dose adjustment with gene therapies. While transcriptional control with inducible promoters is possible, this control is transient and does not provide for stable up or down titration. Similarly, suicide genes can be used to completely turn off gene expression in cells such as CAR-T cells by fully eliminating the cell via induction of apoptosis. The latter approach, however, is incompatible with protein replacement therapies as creating a protein replacement factory in vivo and eliminating the target cells creates significant toxicity in the case of all secretory cell types targeted by previous gene therapies. On top of the unacceptable toxicity to the presently targeted cell types, this approach does not allow for adjustment of the dose as the suicide genes are universally eliminated in a manner that cannot control the resulting level of therapeutic transgene expression.

This invention provides at least in part a combination of target cell types, genetic elements, activators and effectors that enable safe and effective, permanent dose adjustment following initial dosing with a gene therapy. The invention provides at least in part dose adjustment of gene therapy, which allows single-injection gene therapy to replace multi-injection subcutaneous protein regiments while improving pharmacokinetics, safety/efficacy profile, medication adherence, and reducing healthcare costs.

Accordingly, aspects of the present disclosure relate to a system for permanently reducing gene expression from a therapeutic construct or a plethora of constructs within one or more cells comprised of at least: (a) a therapeutic transgene containing nucleic acid construct delivered in a formulation capable of entry into cells and enablement of stable therapeutic transgene expression for a period of at least 4 months, (b) an actuator comprised of one or more chemical or biological molecules, (c) an actuator response element and/or actuator response sequence that enables expression, activation, or stabilizes the effector element or effector sequence, (d) an effector sequence delivered in cis with the therapeutic transgene, (e) an effector element expressed from said effector sequence capable of permanently reducing said therapeutic transgene expression in a manner that elicits minimal bystander effect and may be therefore safe to surrounding tissues, organs, and organism, including in its entirety.

In some embodiments, the system is intended for delivery to subcutaneous tissues. In some embodiments, the system is intended for intraperitoneal delivery. In some embodiments, the system is intended for delivery to adipocytes. In some embodiments, the system is intended for intramuscular delivery. In some embodiments, the system is intended for delivery to the liver. In some embodiments, the system is intended for delivery to the central nervous system. In some embodiments, the system is intended for delivery to the fascia. In some embodiments, the system is intended for delivery to the subarachnoid space. In some embodiments, the system is intended for delivery to the pancreas. In some embodiments, the system is intended for delivery to the epidermis. In some embodiments, the system is intended for ophthalmic delivery. In some embodiments, the system is intended for delivery to the kidneys. In some embodiments, the system is intended for delivery to the bladder. In some embodiments, the system is intended for delivery to the mucous membranes. In some embodiments, the system is intended for delivery to the lung. In some embodiments, the system is intended for delivery to the spleen. In some embodiments, the system is intended for intravascular delivery. In some embodiments, the system is intended for delivery to the heart. In some embodiments, the system is intended to be used in conjunction with therapeutic gene therapy or delivered in conjunction with a therapeutic transgene for the treatment of human, plant, or veterinary diseases or for the augmentation of human, veterinary, or plant function.

In some embodiments, the therapeutic transgene encodes at least a part of a therapeutic antibody. In some embodiments, the therapeutic transgene encodes a protein or enzyme required for protein or enzyme replacement therapy. In some embodiments, the therapeutic transgene encodes a therapeutic peptide. In some embodiments, the therapeutic transgene encodes a protein or fusion protein. In some embodiments, the therapeutic transgene encodes a non-coding RNA. In some embodiments, the therapeutic transgene encodes an aptazyme, aptamer, a ribozyme, or a genetic structure that is capable of enzymatic activity alone or in conjunction with a protein.

In some embodiments, the nucleic acid construct is comprised of double stranded DNA. In some embodiments, the nucleic acid construct is comprised of single stranded DNA. In some embodiments, the nucleic acid construct is comprised of single stranded RNA. In some embodiments, the nucleic acid construct is comprised of double stranded RNA. In some embodiments, the nucleic acid construct is comprised of single stranded or double stranded linear DNA. In some embodiments, the nucleic acid construct is comprised of single stranded or double stranded linear RNA. In some embodiments, the nucleic acid construct is comprised of single stranded or double stranded circular DNA. In some embodiments, the nucleic acid construct is comprised of single stranded or double stranded circular RNA.

In some embodiments, the system includes proteins delivered with the nucleic acid. In some embodiments, the system is delivered as part of a viral or non-viral formulation, in vivo or ex vivo. In some embodiments, the system is delivered by physical methods such as electroporation, nanoparticle bombardment, ultrasound mediated transfection, or chemically mediated transfection.

In some embodiments, the actuator is comprised at least in part from a tetracycline or tetracycline derivative. In some embodiments, the actuator is comprised at least in part from a doxycycline or doxycycline derivative. In some embodiments, the actuator is comprised at least in part from an antibiotic or antimicrobial peptide. In some embodiments, the actuator is comprised at least in part from a rapamycin or derivative thereof. In some embodiments, the actuator is comprised at least in part from a ganciclovir or derivative thereof. In some embodiments, the actuator is comprised at least in part from a ganciclovir, valganciclovir, valaciclovir, or derivative thereof. In some embodiments, the actuator is comprised at least in part from a tamoxifen or derivative thereof. In some embodiments, the actuator is comprised at least in part from an estrogen or derivative thereof. In some embodiments, the actuator is comprised at least in part from an acetaldehyde or derivative thereof. In some embodiments, the actuator is comprised at least in part from an L-arginine or derivative thereof. In some embodiments, the actuator is comprised at least in part from a Biotinyl-AMP or derivative thereof. In some embodiments, the actuator is comprised at least in part from a 2-phenylethyl butyrate or derivative thereof. In some embodiments, the actuator is comprised at least in part from a 6-hydroxy nicotine or derivative thereof. In some embodiments, the actuator is comprised at least in part from an erythromycin or derivative thereof. In some embodiments, the actuator is comprised at least in part from a macrolide or derivative thereof. In some embodiments, the actuator is comprised at least in part from a streptogramin or derivative thereof. In some embodiments, the actuator is comprised at least in part from NADH or derivative thereof. In some embodiments, the actuator is comprised at least in part from an element resulting in the formation of increased energy or heat. In some embodiments, the actuator is comprised at least in part from SCB1 or derivative thereof. In some embodiments, the actuator is comprised at least in part from 3-oxo-C8-HSL or derivative thereof. In some embodiments, the actuator is comprised at least in part from Phloretin or derivative thereof. In some embodiments, the actuator is a small molecule, peptide, protein or nucleic acid that is capable of increasing, decreasing, or modulating local or systemic inflammation, or a part of the inflammatory response. In some embodiments, the actuator is comprised at least in part from theophylline or derivative thereof. In some embodiments, the actuator is comprised at least in part from IPTG or derivative thereof. In some embodiments, the actuator is comprised at least in part from 5-fluorocytosine or derivative thereof. In some embodiments, the actuator is comprised at least in part from propionate or derivative thereof. In some embodiments, the actuator is comprised at least in part from acetate or derivative thereof. In some embodiments, the actuator is comprised at least in part from cumate, cumate ester, or cumic acid, or derivative thereof. In some embodiments, the actuator is comprised at least in part from gluconate or derivative thereof. In some embodiments, the actuator is comprised at least in part from maltose or derivative thereof. In some embodiments, the actuator is comprised at least in part from arabinose or derivative thereof. In some embodiments, the actuator is comprised at least in part from mono- or di-saccharide. In some embodiments, the actuator is comprised at least in part from a trimethoprim or derivative thereof. In some embodiments, the actuator is comprised at least in part from an exogenously administered small molecule, peptide, protein, RNA, DNA, or pharmacological formulation. In some embodiments, at least a part of the actuator can act as at least a part of the effector element. In some embodiments, the actuator is comprised at least in part from an endogenously expressed or presented peptide, hormone, protein, RNA, DNA, enzyme, mineral or chemical or biochemical substance and is capable of interacting with the actuator response element or actuator response sequence.

In some embodiments, the effector element is comprised at least in part from an inducible Caspase 9. In some embodiments, the effector element is comprised at least in part from a natural Caspase 9. In some embodiments, the effector element is comprised at least in part from a Zinc-Finger Nuclease. In some embodiments, the effector element is comprised at least in part from a transcription activator-like effector nucleases (TALEN). In some embodiments, the effector element is comprised at least in part from a meganuclease. In some embodiments, the effector element is comprised at least in part from a deoxycytidine deamination-derived editor. In some embodiments, the effector element is comprised at least in part from a deoxyadenosine deamination-derived base editor. In some embodiments, the effector element is comprised at least in part from a DNA-binding peptide. In some embodiments, the effector element is comprised at least in part from a viral or bacterial DNA-binding protein. In some embodiments, the effector element is comprised at least in part from a natural Caspase 8. In some embodiments, the effector element is comprised at least in part from a natural Caspase or a Caspase pathway protein. In some embodiments, the effector element is a FK506-binding protein 12 (e.g., with F36V mutation) fused with human caspase-8 protein deleted for 1-215 AA and linked to the N-myristoylation signal from Src kinase. In some embodiments, the effector element is comprised in part from an FK506-binding protein 12 (e.g., with F36V mutation) fused with human caspase-9 protein deleted for 1-134 AA. In some embodiments, the effector element is comprised at least in part from at least a part of a viral thymidine kinase. In some embodiments, the effector element is comprised at least in part from at least a part of an Escherichia Coli cytosine deaminase. In some embodiments, the effector element is a protein with catalytic activity capable of chemically modifying a nucleotide, nucleoside, or nucleic acid. In some embodiments, the effector element is comprised at least a part from at least a part of a diphtheria toxin subunit A. In some embodiments, the effector element is comprised at least a part from at least a part of a diphtheria toxin subunit B. In some embodiments, the effector element is a dimerizable or trimerizable toxin, protein, or enzyme. In some embodiments, the effector element is comprised at least in part from, at least a part of the human or simian diphtheria toxin receptor. In some embodiments, the effector element is comprised at least a part from at least a part of a bacterial, viral, or eukaryotic toxin. In some embodiments, the effector element is comprised at least a part from at least a part of a protein capable of inhibiting transcription. In some embodiments, the effector element is comprised at least a part from at least a part of a protein capable of inhibiting translation. In some embodiments, the effector element is comprised at least a part from at least a part of a protein capable of inducing apoptosis. In some embodiments, the effector element is comprised at least a part from at least a part of a protein capable of inducing senescence. In some embodiments, the effector element is comprised at least a part from at least a part of a protein capable of reducing general gene expression. In some embodiments, the effector element is comprised at least a part from at least a part of a nuclease. In some embodiments, the effector element is comprised at least a part from at least a part of a nuclease capable of site specific mutagenesis. In some embodiments, the effector element is comprised at least a part from at least a part of a nuclease and at least a part of a targeting element. In some embodiments, the effector element is comprised at least a part from at least a part of a Cas nuclease and guide RNA targeting the nuclease to the sequence. In some embodiments, the effector element is comprised at least a part from at least a part of a nuclease targeting the sequence. In some embodiments, the effector element is comprised at least a part from at least a part of a nuclease targeting a promoter region of the sequence. In some embodiments, the effector element is comprised at least a part from at least a part of a nuclease targeting a coding region of the sequence. In some embodiments, the effector element is comprised at least a part from at least a part of a nuclease targeting a post translational regulation element region of the sequence. In some embodiments, the effector element is comprised at least a part from at least a part of a recombinase. In some embodiments, the effector element is comprised at least a part from at least a part of a Cre recombinase. In some embodiments, the effector element is comprised at least a part from at least a part of a Krüppel-associated box domain from human gene ZNF10. In some embodiments, the effector element is comprised at least a part from at least a part of a zinc finger protein. In some embodiments, the effector element is comprised at least a part from at least a part of a cyclization recombinase with SV40 nuclear localization signal. In some embodiments, the effector element is comprised at least a part from at least a part of a Cre recombinase with SV40 T-antigen intron inserted in the ORE In some embodiments, the effector element is comprised at least a part from at least a part of a Flpe recombinase. In some embodiments, the effector element is comprised at least a part from at least a part of a Flpo recombinase. In some embodiments, the effector element is comprised at least a part from at least a part of a Dre recombinase. In some embodiments, the effector element is comprised at least a part from at least a part of an SV40 T-antigen sequence. In some embodiments, the effector element is comprised at least a part from at least a part of a piggyBac transposase. In some embodiments, the effector element is comprised at least a part from at least a part of a transposase. In some embodiments, the effector element is comprised at least a part from at least a part of a sleeping beauty transposase. In some embodiments, the effector element is comprised at least a part from at least a part of an argonaut protein. In some embodiments, the effector element is comprised at least a part from at least a part of a DNA methyltransferase. In some embodiments, the effector element is comprised at least a part from at least a part of a protein which makes up or interacts with chromatin remodeling complex. In some embodiments, the effector element is comprised at least a part from at least a part of a protein that makes up or interacts with the DNA methylation pathway. In some embodiments, the effector element is comprised at least a part from at least a part of a base editor. In some embodiments, the effector element is comprised at least a part from at least a part of a prime editor. In some embodiments, the effector element is comprised at least a part from at least a part of a nickase. In some embodiments, the effector element is comprised at least a part from at least a part of a viral, bacterial, or eukaryotic gene silencing protein. In some embodiments, the effector element is comprised at least a part from at least a part of a siRNA. In some embodiments, the effector element is comprised at least a part from at least a part of a miRNA. In some embodiments, the effector element is comprised at least a part from at least a part of FRT. In some embodiments, the effector element is comprised at least a part from at least a part of an integrase. In some embodiments, the effector element is comprised at least a part from at least a part of an RNA capable of reducing gene expression. In some embodiments, the effector element is comprised at least a part from at least a part of a protein enabling the effector degradation in the absence of an inducer. In some embodiments, the effector element is comprised at least a part from at least a part of a dihydrofolate reductase. In some embodiments, the effector element is comprised at least a part from at least a part of RNA, which is at least partially complementary to the genetic construct. In some embodiments, the effector element is comprised at least a part from at least a part of transposon or retrotransposon. In some embodiments, the effector element is comprised at least a part from at least a part of a Streptococcus pyogenes Cas9. In some embodiments, the effector element is comprised at least a part from at least a part of a catalytically inactive Cas9. In some embodiments, the effector element is comprised at least a part from at least a part of a codon optimized Cas9. In some embodiments, the effector element is comprised at least a part from at least a part of Cas9 with a D10A mutation. In some embodiments, the effector element is comprised at least a part from at least a part of a dCas9 fused with KRAB. In some embodiments, the effector element is comprised at least a part from at least a part of dCas9 fused with a carboxy terminal bipartite repressor domain, KRAB-MeCP2 or derivative. In some embodiments, the effector element is comprised at least a part from at least a part of a Campylobacter jujeni Cas9 ortholog. In some embodiments, the effector element is comprised at least a part from at least a part of a CRISPR associated protein or an ortholog. In some embodiments, the effector element is comprised at least a part from at least a part of a Cas12a or Cas12b protein. In some embodiments, the effector element is comprised at least a part from at least a part of Cas13. In some embodiments, the effector element is comprised at least a part from at least a part of Cas12f or Cas13a. In some embodiments, the effector element is comprised at least a part from at least a part of a DNA, DNA capable of expressing, RNA, RNA, protein, or combination thereof capable of effecting DNA sequences in a sequence specific manner to induce double stranded breaks, single stranded breaks, methylation, chromatin modification, or generally epigenetic silencing. In some embodiments, the effector element is comprised at least in part from at least a part of a toxin, toxin derivative, modified toxin, or a homolog, ortholog, or paralog thereof. In some embodiments, the effector element is comprised at least in part from at least a part of a bacterial, fungal, plant, or animal toxin. In some embodiments, the effector element is comprised at least in part from at least a part of a Vibrio cholerae toxin, shiga toxin, Shigella toxin, ricin, saporin, Staphylococcus aureus enterotoxin H, Clostridium perfringens enterotoxin, streptolysin O, ribosome inactivating protein (RIP) enzyme, immunotoxin, Pseudomonas exotoxin A, diphtheria toxin (DT), or DT fragment. In some embodiments, the effector element is comprised at least in part from at least a part of a viral thymidine kinase or derivative thereof. In some embodiments, the effector element is comprised at least in part from at least a part of a bacterial cytosine deaminase or derivative thereof. In some embodiments, the effector element is comprised at least in part from at least a part of a D-amino-acid oxidase or derivative thereof. In some embodiments, the effector element is comprised at least in part from at least a part of a bacterial carboxypeptidase G2 or derivative thereof. In some embodiments, the effector element is comprised at least in part from at least a part of a purine nucleoside phosphorylase or derivative thereof. In some embodiments, the effector element is comprised at least in part from at least a part of a thymidine phosphorylase or derivative thereof. In some embodiments, the effector element is comprised at least in part from at least a part of a xanthine-guanine phosphoribosyl transferase or derivative thereof. In some embodiments, the effector element is comprised at least in part from at least a part of a nitroreductase or derivative thereof. In some embodiments, the effector element is comprised at least in part from at least a part of a penicillin-G amidase or derivative thereof. In some embodiments, the effector element is comprised at least in part from at least a part of a multi-drug activation enzyme or derivative thereof. In some embodiments, the effector element is comprised at least in part from at least a part of a horseradish peroxidase or derivative thereof. In some embodiments, the effector element is comprised at least in part from at least a part of a Beta-galactosidase or derivative thereof. In some embodiments, the effector element is comprised at least in part from at least a part of a deoxyribonucleotide kinase or derivative thereof. In some embodiments, the effector element is comprised at least in part from at least a part of a Beta-glucoronidase or derivative thereof. In some embodiments, the effector element is comprised at least in part from at least a part of a carboxypeptidase A or derivative thereof. In some embodiments, the effector element is comprised at least in part from at least a part of a cytochrome P450 or derivative thereof. In some embodiments, the effector element is comprised at least in part from at least a part of a deoxycytidine kinase or derivative thereof.

In some embodiments, the effector sequence encodes at least the effector element. In some embodiments, the effector sequence encodes at least a part of the consensus Lox sequence or a sequence capable of mediating the effect of a Lox sequence. In some embodiments, the effector sequence encodes at least a part of the consensus FRT sequence or a sequence capable of mediating the effect of an FRT sequence. In some embodiments, the effector sequence encodes at least a part of the consensus Rox sequence or a sequence capable of mediating the effect of a Rox sequence. In some embodiments, the effector sequence is comprised at least in part from GTGTAGTTCGACCATTCGTG (SEQ ID NO: 1). In some embodiments, the effector sequence is comprised at least in part from GTTCAGGATCACGTTACCGC (SEQ ID NO: 2). In some embodiments, the effector sequence is comprised at least in part from GGGCGAGGAGCTGTTCACCG (SEQ ID NO: 3). In some embodiments, the effector sequence is comprised at least in part from GAAGTTCGAGGGCGACACCC (SEQ ID NO: 4). In some embodiments, the effector sequence is comprised at least in part from GCAGATGTAGTGTTTCCACA (SEQ ID NO: 5). In some embodiments, the effector sequence is comprised at least in part from CCTAAGGTTAAGTCGCCCTCG (SEQ ID NO: 6). In some embodiments, the effector sequence is comprised at least in part from CAACAAGATGAAGAGCACCAA (SEQ ID NO: 7). In some embodiments, the effector sequence encodes at least a part of attB, attR, attL, or attP sites. In some embodiments, the effector sequence is comprised at least in part from genetic sequences, which can be targeted by integrases, recombinases, nucleases, nickases, chromatin complexes, or methylases or methyltransferases. In some embodiments, the effector sequence is comprised at least in part from genetic sequences, which can be targeted for silencing RNA or protein binding or can be itself silenced. In some embodiments, the effector sequence is a DNA sequence that can serve as a target of a guide RNA for a CRISPR associated protein. In some embodiments, the effector sequence is a DNA sequence derived from viral or bacterial genomes. In some embodiments, the effector sequence forms a secondary structure in the absence of an inducer molecule. In some embodiments, the effector sequence is an inflammation responsive promoter. In some embodiments, the effector sequence is derived at least in part from a mammalian promoter or enhancer. In some embodiments, the effector sequence forms a secondary structure in the presence of an inducer molecule.

In some embodiments, the actuator response element is a nucleic acid sequence or a protein that interacts with an actuator to enable activity of the effector element or effector sequence. In some embodiments, the actuator response element is comprised at least in part from a tetracycline-controlled transcriptional silencer (tTS). In some embodiments, the actuator response element is comprised at least in part from a Kruppel associated box (KRAB) AB domain of the kid-1 transcriptional repressor. In some embodiments, the actuator response element is composed at least in part of a reverse tetracycline-controlled transactivator. In some embodiments, the actuator response element is comprised at least in part of a viral protein such as VP16 fused with a Tet-Repressor (TetR) or a reverse Tet-Repressor (rTetR). In some embodiments, the actuator response element is comprised of a tTS and rtTA driven by the same promoter. In some embodiments, the actuator response element is comprised of tTS and rtTA driven by different promoters. In some embodiments, the actuator response element is comprised at least in part from a transcriptional repressor. In some embodiments, the actuator response element is comprised at least in part from a transcriptional silencer. In some embodiments, the actuator response element is comprised at least in part from a DNA binding protein. In some embodiments, the actuator response element is comprised at least in part from a promoter binding protein. In some embodiments, the actuator response element is comprised at least in part from a transcription initiation complex binding protein. In some embodiments, the actuator response element is comprised at least in part of a protein responsible for DNA methylation. In some embodiments, the actuator response element is comprised at least in part of a protein responsible for histone modification. In some embodiments, the actuator response element is comprised at least in part of at least a part of a Tet-ON system. In some embodiments, the actuator response element is comprised at least in part of at least a part of a Tet-OFF system.

In some embodiments, the actuator response element is comprised at least in part of at least a part of AlcR, ArgR, BirA, EthR, HdnoR, HucR, MphR(A), PIP, Rex, RheA, ScbR, TraR, TtgR or their analog, homolog, ortholog or protein fragment, protein, or fusion protein of similar functionality. In some embodiments, the actuator response element is comprised at least in part from at least a part of lacUV5, lac, tac, trc, prpB, aceA, aceB, gntP, gntK, CJ10X2, tacM, malE1, git1, BAD, SPL, P4-N14, cspB, aprE, sod, dapA, porB, ilvC, L10, L26, L16, L51, H30, H36 or natural, synthetic or semi-synthetic analogs, homologs, orthologs or protein fragments, proteins, or fusion proteins of similar functionality. In some embodiments, the actuator response element is comprised at least in part from at least a part of ABI1-Gal4DBD, ABA, PVL1-VP16, PHR-p65, CIB1-TetR, or their analog, homolog, ortholog or protein fragment, protein, or fusion protein of similar functionality. In some embodiments, the actuator response element is comprised at least in part from at least a part of a transcriptional activator, enhancer, de-repressor, or protein, RNA, or DNA capable of increasing gene expression directly or indirectly in a targeted or generally cis-acting manner.

Aspects of the present disclosure relate, at least in part, to methods of using any one of the systems described herein. In some embodiments, any one of the systems described herein is used in a method of reducing gene expression in a cell. In some embodiments, the cell is a liver cell, a muscle cell, a central nervous system cell, a pancreatic cell, a skin cell, a kidney cell, a bladder cell, a lung cell, a cardiac cell, an adipocyte, or a spleen cell. In some embodiments, any one of the systems described herein is used in a method of reducing gene expression in an organ. In some embodiments, the organ is liver, pancreas, skin, brain, eye, spleen, kidney, heart, or bladder.

Aspects of the present disclosure relate, at least in part, to methods of delivering any one of the systems described herein to a cell, tissue, or organ of a subject. Aspects of the present disclosure relate, at least in part, to methods of treating a subject in need of treatment. In some embodiments, the subject is a mammalian subject. In some embodiments, the mammalian subject is a human. In some embodiments, the mammalian subject is a rodent. In some embodiments, the rodent is a mouse. In some embodiments, the rodent is a rat. In some embodiments, the mammalian subject is a non-human primate. In some embodiments, the non-human primate is a macaque. In some embodiments, the non-human primate is a chimpanzee. In some embodiments, the non-human primate is a monkey. In some embodiments, the mammalian subject is a canine. In some embodiments, the mammalian subject is a cat.

### BRIEF DESCRIPTION OF THE DRAWINGS

The following drawings form part of the present specification and are included to further demonstrate certain aspects of the present disclosure which may be better understood by reference to one or more of these drawings in combination with the detailed description of specific embodiments presented in this disclosure. The accompanying drawings are not intended to be drawn to scale. The drawings are illustrative only. For purposes of clarity, not every component may be labeled in every drawing. In the drawings:

FIG. 1 shows an npRB2.29v1 plasmid map.

FIG. 2 shows an npRB2.28v1 plasmid map.

FIGS. 3A-3B are bar graphs showing effective expression of reporter genes. FIG. 3A show multiple systems demonstrating effective expression of a reporter gene. FIG. 3B shows relative reduction in expression following equal dose of inducer.

FIG. 4 shows an npRB2.31v1 plasmid map.

FIG. 5 shows an npRB2.33v1 plasmid map.

FIG. 6 is a bar graph showing relative bioluminescence of primary human adipocytes treated with liquid nanoparticles (LNP).

FIG. 7 shows an npRB2.32v1 plasmid map.

FIG. 8 shows an npRB2.30v1 plasmid map.

FIG. 9 is a bar graph showing relative bioluminescence of primary human adipocytes treated with liquid nanoparticles (LNP).

FIG. 10 shows an npRB2.35v1 plasmid map.

FIG. 11 shows an npRB2.34v1T plasmid map.

FIG. 12 is a bar graph showing relative expression in primary human adipocytes treated with liquid nanoparticles (LNP).

FIG. 13 shows an npRB2.22v1 (CBh-IGF2-P2A-Tet3G+TRE3G-GSDMD-NT) plasmid map.

FIG. 14 shows an npRB2.23v1 (CMV-GBA1-P2A-Tet3G+TRE3G-IE2-P2A-tTA) plasmid map.

FIG. 15 shows an npRB2.24v1 (attP-CBh-attB-LMNA-P2A-Tet3G+TRE3G-Bxb1) plasmid map.

FIG. 16 shows an npRB2.25v1 (CBh-CFTR-P2A-iCasp9) plasmid map.

FIG. 17 shows an npRB2.26v1 (CBh-HEXA-P2A-iCasp8) plasmid map.

FIG. 18 shows an npRB2.27v2 (TRE3G-FGF18-P2A-LacI-NLS-KRAB+7×LacOPromoter-tTS) plasmid map.

### DETAILED DESCRIPTION

The present invention generally describes a combination of target cell types and anatomical regions, genetic elements, actuators, effector elements, effector sequences, and actuator response elements that enable safe and effective, permanent dose adjustment following initial dosing with a gene therapy. The invention for the first time enables dose adjustment of gene therapy in a permanent, durable, safe and effective manner. The invention therefore for the first time allows for the use of gene therapy to replace protein replacement, enzyme replacement, or general protein injection treatments with a single injection adjustable gene therapy approach.

In general, the invention describes a genetic construct for the delivery to a broad range of cell types, but most ideally adipocytes or other non-life sustaining or organ sustaining cell type that includes an actuator, effector element, effector sequence, and actuator response element. For the purpose of this invention an actuator can be a small molecule, large molecule, peptide, protein, nucleic acid, physical or chemical stimulus that enables, increases, or activates the expression or activity of an effector element by acting directly on the effector element or on the actuator response element or both. Similarly, for the purpose of this invention, an effector element is a protein, nucleic acid, fusion protein, peptide, enzyme, ribozyme, ribonucleoprotein, sugar, small molecule, or a combination of one or more of the aforementioned elements which can target therapeutic genetic sequences or more specifically effector sequences to enable general dose reduction or specifically reduction in the level of transcription, expression, translation, or functionality of the therapeutic genetic cargo. In line with this, effector sequences are targets, or downstream mediators of activity or recognition sites, or sites of activity of, or are generally associated with the effector element to enable down-titration or dose reduction of the gene therapy treatment. Finally, actuator response elements are proteins, nucleic acid sequences, DNA, promoters, enhancers, insulators, or generally genetic sequences, that once acted upon by the actuator change the level of expression, translation, transcription, or activation of the effector element. In general, said elements or genetic sequences encoding said elements are delivered in cis at the time of treatment or re-treatment, with the exception of the actuator, which is delivered at the time of dose adjustment. The sequences of the actuator, effector element, effector sequence, and/or actuator response element may be optionally codon-optimized for the species of interest.

In one representative example, an actuator such as tetracycline induces expression of a CRISPR-Cas9 protein by activating a Tet-ON operator, which drives expression of a CRISPR-Cas9 protein and guide RNA. The CRISPR-Cas9 protein and guide RNA, which are the effector elements, target the effector sequence which separates a constitutive promoter that drives therapeutic transgene expression. As a result, administration of tetracycline, enables expression of a nuclease that separates the promoter from the coding region of the therapeutic transgene reducing the level of expression of the therapeutic transgene from this construct. Since the effect is sequence specific and the DNA encoding the effector element, effector sequence, actuator response element, therapeutic transgene, and associated promoters are delivered in cis (or all together in one genetic construct), the initial treatment produces expression at a level L, which is reduced to a level less than L after activation of the dose reduction system by administration of an actuator, which can be dosed as a safe exogenous small molecule to reduce the level of therapeutic transgene expression.

Another optional actuator can be derived from a class of Stop-Codon readthrough enhancers. One example of Stop-Codon readthrough enhancers are aminoglycoside antibiotics. The therapeutic transgene can be expressed in cis ending with a stop codon, the read-through of which can be facilitated by the administration of a Stop-Codon readthrough enhancer or a molecule enabling or enhancing Stop-Codon readthrough directly or indirectly. The Stop-Codon, may for example be followed by a self-cleaving peptide and a suicide gene such as for example Diphtheria Toxin or Diphtheria Toxin subunit. Some representative examples of readthrough enhancers include Gentamicin, G418, Paromomycin, Neomycin, Sisomycin, Lividomycin or derivatives or combinations thereof. Some examples of stop codons may include UGA, UAG, UAA, with optional 4^{th} base allowing for suppression of natural read-through and/or enhancement or readthrough following activation by a readthrough enhancer. As an example, Gentamicin can enhance readthrough when an UGA stop codon is used and then followed by a Cytosine (C) or Uracil (U), less preferably Adenine (A) and least preferably Guanine (G). Said system may be used alone or in conjunction with other systems such as for example the use of a base editor to change one of the bases neighboring the stop codon in the stop codon, or in a codon to turn the said codon into a stop codon.

In general, the invention is comprised of a system for permanently or less preferably and optionally transiently reducing gene expression from a therapeutic construct or a plethora of constructs within one or more cells comprised of at least several elements. Said elements can include a therapeutic transgene containing nucleic acid construct delivered in a formulation capable of entry into cells and enablement of stable therapeutic transgene expression for a period of at least 4 months, one or more actuators comprised of one or more chemical or biological molecules that can be administered exogenously to the system, cell, tissue, organ, or organism undergoing treatment with said system, one or more actuator response elements and/or actuator response sequences that enable expression, activation, or stabilize one or more effector element or effector sequence, an effector sequence delivered in cis with the therapeutic transgene, and one or more effector elements expressed from said effector sequence(s) capable of permanently reducing said therapeutic transgene expression in a manner that elicits minimal bystander effect and is therefore safe to surrounding tissues, organs, and organism, including in its entirety.

The system may be part of a larger formulation or one or more formulations and be administered via drug delivery system or delivered in a vial for subsequent administration, or a prefilled syringe or cartridge. Alternatively, the treatment can be administered via an autoinjector or a patch injector, via ultrasonic guidance, or via other guidance techniques to ensure delivery to the correct location. Alternatively, and optionally the treatment can be delivered by a set of needles or via a set or series of injections to more evenly distribute the constructs in the treatment site. The gene therapy treatment can be delivered via a patch, microneedles, a set of microneedles, jet injection, on a carrier, or using other means to alter the integrity of the tissues through which or into which it is being delivered.

In some embodiments of the present invention the target cell type of said system may be one or more of an adipocyte, pre-adipocyte, progenitor capable of differentiating into an adipocyte, stem cell capable of differentiating into an adipocyte, a resident cell type of the subcutaneous tissues, or a transient cell or stem cell, but one that can become a resident cell of the subcutaneous tissues. The system is intended to treat or prevent any human or veterinary disorder, but may be used in some specific embodiments to treat monogenic disorders, enzyme insufficiency disorders, protein insufficiency disorders (as protein replacement therapy), metabolic disorders, autoimmune disorders, oncologies, neurologic disorders, cardiovascular pathologies, musculoskeletal disorders, hematologic disorders, infectious disorders, dermatologic disorders, immune system disorders, pulmonary system disorders, kidney or bladder disorders, or disorders of other organs, tissues, systems, or combinations thereof. In some embodiments of the present invention the system is intended for the delivery of therapeutic or prophylactic antibodies via expression from the genetic construct, therapeutic, preventative, augmentative, or prophylactic proteins, fusion proteins, peptides, immunogens, antigens, enzymes, ribozymes, mRNA, non-coding RNA, miRNA, shRNA, or other RNA, or molecules that can act as vaccines or adjuvants via expression from the genetic construct.

Generally, the system may be delivered via one or more means to the subcutaneous tissues, intraperitoneally, delivered directly to adipose tissues, to white adipose tissues, or targeted or delivered to adipocytes. Alternatively and optionally in conjunction with the aforementioned delivery locations, tissues, and forms, the system can be delivered via intramuscular delivery, dosed intravenously or directly to the liver via an artery or a vein, the system can be used in the central nervous system via direct or indirect delivery, delivered to the fascia, subarachnoid space, the pancreas, epidermis, the eye or generally to parts of the eye as part of ophthalmic or intravitreous delivery, the kidneys, bladder, mucous membranes, lung, spleen, heart, or any other cell, tissue, organ, or system of cells, tissues, organs, or the entire organism.

Said system can be used to treat disease or in conjunction with other therapeutics to manage symptoms or treat a disease or a pathology, or to bestow function upon an organism, organ, or tissue. The system may be used in conjunction with a therapeutic gene to treat human, veterinary, or plant diseases or augment human, veterinary, or plant function.

The system is intended to contain at least one therapeutic genetic element which can be a coding gene or a non-coding gene, or a DNA segment that directly provides a therapeutic element or is transcribed into an RNA that provides a therapeutic element. Said system may therefore encode at least a part of a therapeutic antibody, protein, enzyme, fusion protein, peptide, aptazyme, or transfer RNA (tRNA), ribosomal RNA (rRNA), long non-coding RNA (lnc-RNA or In-RNA or long nc-RNA), short non-coding RNA (short ncRNA or snc-RNA), micro RNA (miRNA), messenger RNA (mRNA), small interfering RNA (siRNA), small nuclear RNA (snRNA), PIWI-interacting RNA (piRNA) or other RNA, protein, or combination thereof. The therapeutic transgene may directly encode an aptazyme or maybe flanked by an aptazyme or multiple aptazymes, ribozymes, or genetic structures capable of enzymatic activity alone or in conjunction with a protein or other nucleic acids.

The system disclosed in this invention contains a nucleic acid, which may be one or more of a double stranded DNA (dsDNA), single stranded DNA (ssDNA), double stranded RNA (dsRNA), or single stranded RNA (ssRNA). Optionally, the construct may contain a DNA-RNA hybrid where one or more strands are RNA and one or more strands are DNA or one or more parts of one or more strands are RNA and one or more parts of one or more strands are DNA. Said DNA or RNA may be linear, circular, supercoiled, partially supercoiled, or not-supercoiled, partially linear, segmented, covalently closed, modified with chemical modifications such as caps and polyA tails, or chemically modified for enhanced stability, decreased immunogenicity, or generally to bestow any other biological function.

The system may be delivered as a protein-nucleic acid complex or may generally contain proteins inside the formulation that contains the nucleic acid or as part of the formulation that contains the nucleic acid. Said proteins can range from DNA binding proteins, to proteins promoting decreased immunogenicity, to proteins promoting nucleic acid transport inside a cell, to proteins promoting protection of the nucleic acid, to proteins promoting binding to cell proteins, membranes, organelles, transmembrane proteins, nuclear proteins, or other cellular structures and may generally serve structural or therapeutic activity or augment structural or therapeutic activity of the system.

The system can be delivered using a viral or non-viral vector containing formulation, or a formulation that contains both viral and non-viral vectors with or without targeting or additional targeting. Said delivery can be performed in vivo or ex vivo, as in for example the delivery to immune system cells for the purposes of production of Chimeric Antigen Receptor T Cells or CAR-T cells. The system can optionally or additionally be delivered by physical or chemical methods including one or more of electroporation, nanoparticle bombardment, ultrasound mediated transfection, or chemically mediated transfection using a transfection reagent in vivo or ex vivo. Generally, the system is intended to be delivered on its own or via a carrier, vehicle, formulation, or as part of greater constructs or superstructures to enable therapeutic effect, targeting, dose adjustment, safety, or durability.

The system entails the use of an actuator the delivery of which is performed at a time when the system requires adjustment of therapeutic effect, titration, or dose adjustment. Said actuator may be an exogenous or endogenous small molecule and may be delivered or the production of said actuator may be elicited by the delivery of another means, physical or chemical. In some embodiments of the present invention said actuator is comprised at least in part from a tetracycline or tetracycline derivative, a doxycycline or doxycycline derivative, an antibiotic or antimicrobial peptide or derivative, a rapamycin or derivative thereof, a ganciclovir or derivative thereof, a valganciclovir or derivative thereof, a valaciclovir or derivative thereof, a tamoxifen or derivative thereof, a hormone or an estrogen or derivative thereof, an acetaldehyde or derivative thereof, an L-arginine or derivative thereof, a Biotinyl-AMP or derivative thereof, caffeine or derivative thereof, a 2-phenylethyl butyrate or derivative thereof, a 6-hydroxy nicotine or derivative thereof, an erythromycin or derivative thereof. In some embodiments the actuator may be comprised at least in part of a macrolide or derivative thereof, a streptogramin or derivative thereof, theophylline or derivative thereof, 5-fluorouracile or derivative thereof, propionate or derivative thereof, 5-fluorocytosine or derivative thereof, Isopropyl β-D-1-thiogalactopyranoside (IPTG) or derivative thereof, acetate or derivative thereof, NADH or derivative thereof, or generally an antibiotic, antimicrobial, antiviral, anti-inflammatory, hormone, or pro-inflammatory agent, or generally an approved therapeutic agent for the treatment of some other disease, symptom, or pathology. In some embodiments of the present invention the actuator may be derived from the application of heat or energy, or may be an element of heat, light, magnetic field, or other energy, or cold or energy loss. In some embodiments the actuator may be comprised at least in part of a natural or synthetic peptide or protein such as SCB1 or derivative thereof or a fusion protein or peptide that is chemically modified. In yet other embodiments of the present invention the actuator may be comprised at least in part from 3-oxo-C8-HSL or derivative thereof, phloretin or derivative thereof, or generally a small molecule, peptide, protein or nucleic acid that is capable of increasing, decreasing, or modulating local or systemic inflammation, or a part of the inflammatory response. In yet other embodiments the actuator is comprised at least in part form a cumate, cumate ester, cumic acid, or derivative thereof, or 2,3-dihydroxy-p-cumate or derivative thereof, p-cumate or derivative thereof, or cis-5,6-dihydroxy-4-isopropylcyclohexa-1,3-dienecarboxylate or derivative thereof. In other embodiments of the present invention the actuator may be comprised at least in part from at least a part of gluconate, maltose, dextrose, idonate, arabinose or derivative thereof, or be generally comprised from a monosaccharide, disaccharide, polysaccharide, or oligosaccharide. In yet other embodiments of the present invention the actuator may be comprised at least in part from at least a part of trimethoprim or derivative thereof. Finally, for the purposes of this invention the actuator can be comprised at least in part from an exogenously administered small molecule, peptide, protein, RNA, DNA, or pharmacological formulation with or without the additional physical activity or action from within or outside of the treated organism. Moreover, an actuator may be comprised from the effector element or serve the purpose of the actuator and effector in the therapeutic formulation and may be comprised at least in part of an endogenously expressed or presented peptide, hormone, protein, RNA, DNA, enzyme, mineral or chemical or biochemical substance and is capable of interacting with the actuator response element or actuator response sequence.

The system disclosed in the present invention utilizes one or more effector elements, which when induced, activated, or produced following action by an actuator cause by direct or indirect action the reduction of the therapeutic dose, therapeutic transgene expression, elimination of the cell carrying the system, or reduction in the production of therapeutic effect. Said effector elements may be comprised at least in part from an inducible Caspase 9, natural Caspase 9, Zinc-Finger Nuclease, transcription activator-like effector nucleases (TALEN), meganuclease or homing endonuclease, deoxycytidine deamination-derived editor, deoxyadenosine deamination-derived base editor, or combination thereof and mediate DNA-binding, cleavage, single stranded breaks, double stranded breaks, or death of the cell or cells carrying the system or therapeutic cargo. Other effectors, which may be used for the purpose of down-titration include Caspase 1, 3, 4, 5, 6, 8, 9, 11, and 12, FK506-binding protein 12 (with F36V mutation) fused with human caspase-8 protein deleted for 1-215 AA and linked to the N-myristoylation signal from Src kinase, FK506-binding protein 12 (with F36V mutation) fused with human caspase-9 protein deleted for 1-134 AA, bacterial or viral thymidine kinase, bacterial or viral cytosine deaminase, or generally any kinase, Caspase, or Caspase pathway protein a fragment or hybrid thereof. In some embodiments of the present invention the effector element may be comprised at last in part from at least a part of a toxin or diphtheria toxin (DT) or a toxin subunit or diphtheria toxin subunit A (DTA) or at least a part of or in combination with a diphtheria toxin subunit B (DTB), homologs, analogs, or fragments thereof or generally dimerizable, trimerizable, or monomeric toxins with or without stabilization or destabilization or binding domains. In general, the effector element may mediate down titration by eliminating the cell carrying the system or block expression, transcription, translation, or general production of the therapeutic protein. Some specific effectors may be comprised at least a part from at least a part of Botulinum toxin, Cholera toxin, Clostridium perfringens toxin, Corynebacterium diphtheriae toxin, Diphtheria toxin, E. coli Shiga toxin, E. coli heat-labile toxin, E. coli heat-stable toxin, Helicobacter pylori VacA toxin, Pertussis toxin, Pseudomonas aeruginosa exotoxin A, Staphylococcus aureus enterotoxin, Streptococcus pyogenes streptolysin O, Tetanus toxin, Vibrio cholerae cytolysin, Adenovirus E1A protein, Ebola virus VP24 protein, Epstein-Barr virus BHRF1 protein, Hepatitis C virus NS5A protein, Human immunodeficiency virus (HIV) Vpr protein, Human T-cell leukemia virus type 1 (HTLV-1) Tax protein, Influenza A virus PB1-F2 protein, Poxvirus F1L protein, Rabies virus P protein, Rotavirus NSP4 protein, Severe acute respiratory syndrome coronavirus 2 (SARS-CoV-2) N protein, Vaccinia virus F1L protein, West Nile virus NS3 protein or related protein. The origin of the effector may be bacterial, viral, archaeal or eukaryotic and may include at least a part of Bax, Bak, Caspases, Apaf-1, Cytochrome c, Fas receptor, Fas ligand, TNF receptor 1, TNF-related apoptosis-inducing ligand, p53, Bad, Bid, Bim, Noxa, CD47 and may function in conjunction with external stimuli, such as for example CAR-T cells targeting CD47 transmembrane proteins. In general, the effector may be comprised at least in part from a toxin, such as a diphtheria toxin or a toxin receptor such as a human or a simian diphtheria toxin receptor with the use of a toxin to mediate effect; said toxin or receptor may be viral, bacterial, archaeal or eukaryotic origin and optionally able to inhibit transcription, translation, cell division, mitosis, respiration, oxidative phosphorylation, glycolysis, promote apoptosis, induce senescence or generally one or more cellular functions required for transgene or gene expression to completely eliminate said function or reduce it ideally permanently, but optionally temporarily. In other embodiments of the present invention the effector element may be a nuclease, with or without targeting, capable of inactivating the therapeutic construct by for example separating the promoter from the therapeutic transgene to prevent therapeutic transgene expression. As such, the effector element may be comprised at least in part of at least a part of a nuclease, nuclease capable of site specific mutagenesis, nuclease and at least a part of a targeting element, a CRISPR-associated protein (Cas) nuclease with or without a single guide RNA targeting the nuclease to at least a part of the transgene delivered as part of the system, nuclease targeting a coding region of the therapeutic transgene, nuclease targeting a post translational regulation element region of the sequence the encodes or follows the therapeutic transgene, or a recombinase, such as a Cre recombinase, or other bacterial, viral, archaeal, or eukaryotic recombinase or related protein. The effector element may be comprised of one or more domains or components or itself be a plethora of elements and may be comprised at least in part of at least a part of a Krüppel-associated box domain from human gene ZNF10, or generally a zinc finger protein (ZFN) or ZFN domain, or a part of a cyclization recombinase with SV40 nuclear localization signal, or a Cre recombinase with SV40 T-antigen or a fragment thereof, or a Flpe recombinase, or a Flpo recombinase, or an SV40 T-antigen sequence or fragment thereof or a piggyBac transposase, or generally a transposases, an argonaut protein, a DNA methyltransferase, or a protein which makes up or interacts with chromatin remodeling complex, makes up or interacts with the DNA methylation pathway. Said effector elements may act directly on the therapeutic transgene delivered as part of the system or on proximal, neighboring, flanking, or distal sequences, or indirectly to reduce gene expression, transcription, translation, or generally destabilize expression, transcription, translation, or mRNA to reduce the dose of the therapeutic expressed permanently or induce expression of another protein that can reduce the expression, transcription, or translation of the therapeutic directly or indirectly.

In some embodiments of the present invention the effector element acts by epigenetic silencing or chromatin remodeling, and in other embodiments of the present invention the effector element acts to cut, edit, deactivate, or create double or single stranded breaks in the therapeutic transgene or other nucleic acids delivered in cis with the system. For the aforementioned purpose or other purposes the effector element may be comprised at least in part from at least a part of a base editor, prime editor, nickase, nuclease, recombinase, FRT, transposon, transposase, retrotransposon, integrase, dihydrofolate reductase, or reverse transcriptase, or generally a protein with the ability to modify DNA, chromatins, or RNA.

In other embodiments of the present invention the effector element may be comprised of a non-coding gene that produces an RNA, such as a non-coding RNA that can interfere with gene expression, transcription, translation to cause permanent epigenetic silencing of the therapeutic construct as in for example X-inactivation, which may or may not be at least partially complementary or antisense to the genetic construct delivered in cis, or to create a downstream secondary effect that induces apoptosis, genetic silencing, or recombination or genetic material to ultimate effect down-titration. The effector element may be comprised of one or more elements such as a protein and an RNA, as an example, it can be comprised at least in part of a Streptococcus pyogenes Cas9 (SpCas9), SpCas9-VQR, SpCas9-VRER, SpCas9-NG, SpCas9-xCas9, SpCas9-Sc++, SpCas9-SpG, SpCas9-SpRY, FnCas9, FnCas9-RHA, SaCas9, SaCas9-KKH, StlCas9, NmCas9, GeoCas9, or catalytically inactive Cas9, Campylobacter jujeni Cas9 ortholog, or a part of a codon optimized Cas9, or a part of Cas9 with a D10A mutation, CRISPR associated protein or an ortholog, or a Cas protein such as dCas9 fused with Krüppel associated box (KRAB), or a KRAB-MeCP2 (methyl-CpG binding protein 2), Cas12a, AsCas12a, AsCas12a-RR, AsCas12a-RVR, FnCas12a, Cas12b, Cas12e, Cas12f, UnlCas12f1, CnCas12f1, Cas12j, Cas13, Cas13a or derivative ortholog, homolog, or paralog thereof.

The effector element may have stabilizing or destabilizing domains, activating or inactivating domains, or dimerizing or trimerizing domains, which may or may not be responsive to an external inducer molecule or an inducer that is inherently present at certain times or in certain events in vivo, as such, the effector element may be comprised at least in part of a destabilization domain or a part of a protein that enables effector degradation in the absence of some exogenous or endogenous inducer-based stimulus.

In other embodiments of the present invention the effector element may be comprised at least a part from at least a part of a DNA, DNA capable of expressing, RNA, RNA, protein, or combination thereof capable of effecting DNA sequences in a sequence specific manner to induce double stranded breaks, single stranded breaks, methylation, chromatin modification, or generally epigenetic silencing. Some examples of said DNA are mobile transposable elements, which in the presence of endogenous or exogenous proteins can move or insert into the system's nucleic acid in such a way as to decrease the expression of the therapeutic transgene from said system. In some embodiments of the present invention, the effector may be comprised of a triplex-forming oligonucleotide, with or without a stabilizer, to inhibit expression of the therapeutic transgene.

In yet other embodiments of the present invention the effector element in the presence of an exogenous inducer or on its own may be capable of reducing transcription, inducing apoptosis, inducing necrosis, inducing pyroptosis, inducing ferroptosis, inducing senescence, reducing gene expression, reducing secretion, or reducing translation of the therapeutic transgene or generally of some of the genes in the cell that hosts the system's nucleic acid and may be comprised from at least a part of at least a part of a toxin, toxin derivative, modified toxin, or a homolog, ortholog, or paralog thereof or a combination thereof. Said toxins may be at least a part of a bacterial, fungal, plant, or animal toxin, or specifically at least a part of a Vibrio cholerae toxin, shiga toxin, Shigella toxin, ricin, saporin, Staphylococcus aureus enterotoxin H, Clostridium perfringens enterotoxin, streptolysin O, ribosome inactivating protein (RIP) enzyme, immunotoxin, Pseudomonas exotoxin A, diphtheria toxin (DT), or DT fragment with or without coupling to activation, inactivation, nuclear localization, or signaling domains. In some specific embodiments of the present invention the effector element may be comprised at least in part from at least a part of a viral thymidine kinase, a cytosine deaminase, D-amino-acid oxidase, carboxypeptidase G2, purine nucleoside phosphorylase, thymidine phosphorylase, xanthine-guanine phosphoribosyl transferase, nitroreductase, penicillin-G amidase, multi-drug activation enzyme, Beta-lactamase, horseradish peroxidase, Beta-galactosidase, deoxyribonucleotide kinase, Beta-glucoronidase, carboxypeptidase A, cytochrome P450, deoxycytidine kinase of bacterial, viral, archaeal or eukaryotic origin or derivative thereof.

In yet other embodiments of the present invention the effector element in the presence of an exogenous inducer or on its own may be capable of inducing programmed cell death, including apoptosis, pyroptosis, ferroptosis, or necrosis and may be comprised at least in part of gasdermin A, gasdermin B, gasdermin C, gasdermin D, or gasdermin E, homologs, analogs, or fragments thereof or generally oligomerizable molecules, nucleic acids, proteins or peptides capable of forming pores in various cellular membranes. In general, the effector may be comprised at least in part of a DNA-binding peptide, viral or bacterial DNA-binding protein, a protein with catalytic activity capable of chemically modifying a nucleotide, nucleoside, or nucleic acid.

One or more effector elements of the system may act on one or more effector sequences of the system, which may themselves encode at least a portion of the effector element. Said effector sequences are intended to mediate the dose down-titration activity of the effector element and may do it directly, indirectly, enhance activity, moderate activity, mediate activity, or amplify the activity of the effector element. Said effector sequences may be comprised at least a part from at least a part of the consensus Lox sequence or a sequence capable of mediating the effect of a Lox sequence, the consensus FRT sequence or a sequence capable of mediating the effect of an FRT sequence, the consensus Rox sequence or a sequence capable of mediating the effect of a Rox sequence, or be any sequence that can be targeted by a DNA-binding enzyme, restriction enzyme, DNase, nickase, nuclease, or any other effector element and may be specifically comprised at least a part from at least a part of GTGTAGTTCGACCATTCGTG (SEQ ID NO: 1), GTTCAGGATCACGTTACCGC (SEQ ID NO: 2), GGGCGAGGAGCTGTTCACCG (SEQ ID NO: 3), GAAGTTCGAGGGCGACACCC (SEQ ID NO: 4), GCAGATGTAGTGTTTCCACA (SEQ ID NO: 5), CCTAAGGTTAAGTCGCCCTCG (SEQ ID NO: 6), CAACAAGATGAAGAGCACCAA (SEQ ID NO: 7) or any other sequences showing a low degree of homology with the genome of the species being targeted for the therapeutic or any other benefit of said system. In other specific embodiments the effector sequence can be comprised from at least a part of a protospacer adjacent motif (PAM) sequence, such as NGG, NGAG, NGCG, NGN, NRN, NGG, YG, NNGRRT, NNNRRT, NNAGAA, NNNNGATT, NNNNCRAA, TTIV, TYCV, TATV, TTTV, TIN, TTCN, TTN, or CCN, or a variant thereof that is recognizable by an enzyme. In some embodiments a PAM sequence may be optimized and lengthened to reduce off-target effects. In other specific embodiments the effector sequence can be comprised from at least a part of the attB, attR, attL, or attP sites or other restriction enzyme coding sites, or generally sites that can be safely targeted by integrases, recombinases, nucleases, nickases, chromatin complexes, or methylases or methyltransferases with minimum deleterious off-target activity. In some specific embodiments of the present invention said effector sequences can be at least in part comprised of at least a part of genetic sequences, which can be targeted for silencing RNA or protein binding or can be itself silenced, or serve as a target of a guide RNA for a CRISPR associated protein, or be derived from viral or bacterial genomes, or sequences that form secondary structures in the absence or presence of an exogenous or endogenous inducer molecule, or sequences that comprise at least a part of an inflammation responsive promoter or enhancer, a mammalian promoter or enhancer, or promoters enhancers or trans-activators capable of forming secondary structures in the presence or absence of inducer molecules.

Moreover, the present invention discloses a system, which may optionally contain an actuator response element, which is a nucleic acid sequence or a protein that interacts with an actuator to enable activity of the effector element or effector sequence, or which is comprised at least in part from a tetracycline-controlled transcriptional silencer (tTS), or which is comprised at least in part from a Kruppel associated box (KRAB) AB domain of the kid-1 transcriptional repressor, or which is composed at least in part of a reverse tetracycline-controlled transactivator, or which is comprised at least in part of a viral protein such as VP16 fused with a Tet-Repressor (TetR) or a reverse Tet-Repressor (rTetR), or which is comprised of a tTS and rtTA driven by the same promoter, or which is comprised of tTS and rtTA driven by different promoters, or which is comprised at least in part from a transcriptional repressor, or which is comprised at least in part from a transcriptional silencer, or which is comprised at least in part from a DNA binding protein, or which is comprised at least in part from a promoter binding protein, or which is comprised at least in part from a transcription initiation complex binding protein, or which is comprised at least in part of a protein responsible for DNA methylation, or which is comprised at least in part of a protein responsible for histone modification, or which is comprised at least in part of at least a part of a Tet-ON system, or which is comprised at least in part of at least a part of a Tet-OFF system, or which is comprised at least in part of at least a part of AlcR, ArgR, BirA, EthR, HdnoR, HSP70B (HSPA7), HucR, LgnR, MphR(A), PIP, Rex, RheA, ScbR, TraR, TtgR or their analog, homolog, ortholog or protein fragment, protein, or fusion protein of similar functionality, or which is comprised at least in part from at least a part of lacUV5, lac, tac, trc, prpB, aceA, aceB, gntP, gntK, CJ10X2, tacM, malE1, git1, BAD, SPL, P4-N14, cspB, aprE, sod, dapA, porB, ilvC, L10, L26, L16, L51, H30, H36 or natural, synthetic or semi-synthetic analogs, homologs, orthologs or protein fragments, proteins, or fusion proteins of similar functionality or combination of one or more of the aforementioned elements.

Moreover, the actuator response element disclosed in this invention may be comprised at least in part from at least a part of ABI1-Gal4DBD, ABA, PVL1-VP16, PHR-p65, CIB1-TetR, or their analog, homolog, ortholog or protein fragment, protein, or fusion protein of similar functionality, or which is comprised at least in part from at least a part of a transcriptional activator, enhancer, de-repressor, or protein, RNA, or DNA capable of increasing gene expression directly or indirectly in a targeted or generally cis-acting manner.

The components described in this invention can optionally work in concert to enable down-titration of the system and therefore a reduction in the level of expression of one or more therapeutic elements, but may also optionally have additional functionality such as transient down-regulation of expression or transient upregulation of expression, or may impact other cellular elements or pathways, or interact with other cellular sequences, proteins, organelles, or function, with at least one of the main goals being changing the level of therapeutic effect, expression, transcription, translation, benefit, or side-effect of the overall organism, system, organ, tissue, or cell effected by the system, or in essence dose reduction, or dose titration, or dose down-titration, or dose up-titration, or some similar action or activity.

### ADDITIONAL EMBODIMENTS

1. A system for permanently reducing gene expression from a therapeutic construct or a plethora of constructs within one or more cells comprised of at least: a. A therapeutic transgene containing nucleic acid construct delivered in a
formulation capable of entry into cells and enablement of stable therapeutic transgene expression for a period of at least 4 months,
b. An actuator comprised of one or more chemical or biological molecules,
c. An actuator response element and/or actuator response sequence that enables expression, activation, or stabilizes the effector element or effector sequence,
d. An effector sequence delivered in cis with the therapeutic transgene,
e. An effector element expressed from said effector sequence capable of permanently reducing said therapeutic transgene expression in a manner that elicits minimal bystander effect and is therefore safe to surrounding tissues, organs, and organism in its entirety.

2. A system in Embodiment 1 intended for delivery to subcutaneous tissues.

3. A system in Embodiment 1 intended for intraperitoneal delivery.

4. A system in Embodiment 1 intended for delivery to adipocytes.

5. A system in Embodiment 1 intended for intramuscular delivery.

6. A system in Embodiment 1 intended for delivery to the liver.

7. A system in Embodiment 1 intended for delivery to the central nervous system.

8. A system in Embodiment 1 intended for delivery to the fascia.

9. A system in Embodiment 1 intended for delivery to the subarachnoid space.

10. A system in Embodiment 1 intended for delivery to the pancreas.

11. A system in Embodiment 1 intended for delivery to the epidermis.

12. A system in Embodiment 1 intended for ophthalmic delivery.

13. A system in Embodiment 1 intended for delivery to the kidneys.

14. A system in Embodiment 1 intended for delivery to the bladder.

15. A system in Embodiment 1 intended for delivery to the mucous membranes.

16. A system in Embodiment 1 intended for delivery to the lung.

17. A system in Embodiment 1 intended for delivery to the spleen.

18. A system in Embodiment 1 intended for intravascular delivery.

19. A system in Embodiment 1 intended for delivery to the heart.

20. A system in Embodiment 1 intended to be used in conjunction with therapeutic gene therapy or delivered in conjunction with a therapeutic transgene for the treatment of human, plant, or veterinary diseases or for the augmentation of human, veterinary, or plant function.

21. A therapeutic transgene in Embodiment 1, which encodes at least a part of a therapeutic antibody.

22. A therapeutic transgene in Embodiment 1, which encodes a protein or enzyme required for protein or enzyme replacement therapy.

23. A therapeutic transgene in Embodiment 1, which encodes a therapeutic peptide.

24. A therapeutic transgene in Embodiment 1, which encodes a protein or fusion protein.

25. A therapeutic transgene in Embodiment 1, which encodes a non-coding RNA.

26. A therapeutic transgene in Embodiment 1, which encodes an aptazyme, aptamer, a ribozyme, or a genetic structure that is capable of enzymatic activity alone or in conjunction with a protein.

27. A nucleic acid construct in Embodiment 1, which is comprised of double stranded DNA.

28. A nucleic acid construct in Embodiment 1, which is comprised of single stranded DNA.

29. A nucleic acid construct in Embodiment 1, which is comprised of single stranded RNA.

30. A nucleic acid construct in Embodiment 1, which is comprised of double stranded RNA.

31. A nucleic acid construct in Embodiment 1, which is comprised of single stranded or double stranded linear DNA.

32. A nucleic acid construct in Embodiment 1, which is comprised of single stranded or double stranded linear RNA.

33. A nucleic acid construct in Embodiment 1, which is comprised of single stranded or double stranded circular DNA.

34. A nucleic acid construct in Embodiment 1, which is comprised of single stranded or double stranded circular RNA.

35. A system in Embodiment 1 which includes proteins delivered with the nucleic acid.

36. A system in Embodiment 1, which is delivered as part of a viral or non-viral formulation, in vivo or ex vivo.

37. A system in Embodiment 1, which is delivered by physical methods such as electroporation, nanoparticle bombardment, ultrasound mediated transfection, or chemically mediated transfection.

38. An actuator in Embodiment 1, which is comprised at least in part from a tetracycline or tetracycline derivative.

39. An actuator in Embodiment 1, which is comprised at least in part from a doxycycline or doxycycline derivative.

40. An actuator in Embodiment 1, which is comprised at least in part from an antibiotic or antimicrobial peptide.

41. An actuator in Embodiment 1, which is comprised at least in part from a rapamycin or derivative thereof.

42. An actuator in Embodiment 1, which is comprised at least in part from a ganciclovir or derivative thereof.

43. An actuator in Embodiment 1, which is comprised at least in part from a ganciclovir, valganciclovir, valaciclovir, or derivative thereof.

44. An actuator in Embodiment 1, which is comprised at least in part from a tamoxifen or derivative thereof.

45. An actuator in Embodiment 1, which is comprised at least in part from an estrogen or derivative thereof.

46. An actuator in Embodiment 1, which is comprised at least in part from an acetaldehyde or derivative thereof.

47. An actuator in Embodiment 1, which is comprised at least in part from an L-arginine or derivative thereof.

48. An actuator in Embodiment 1, which is comprised at least in part from a Biotinyl-AMP or derivative thereof.

49. An actuator in Embodiment 1, which is comprised at least in part from a 2-phenylethyl butyrate or derivative thereof.

50. An actuator in Embodiment 1, which is comprised at least in part from a 6-hydroxy nicotine or derivative thereof.

51. An actuator in Embodiment 1, which is comprised at least in part from an erythromycin or derivative thereof.

52. An actuator in Embodiment 1, which is comprised at least in part from a macrolide or derivative thereof.

53. An actuator in Embodiment 1, which is comprised at least in part from a streptogramin or derivative thereof.

54. An actuator in Embodiment 1, which is comprised at least in part from NADH or derivative thereof.

55. An actuator in Embodiment 1, which is comprised at least in part from an element resulting in the formation of increased energy or heat.

56. An actuator in Embodiment 1, which is comprised at least in part from SCB1 or derivative thereof.

57. An actuator in Embodiment 1, which is comprised at least in part from 3-oxo-C8-HSL or derivative thereof.

58. An actuator in Embodiment 1, which is comprised at least in part from Phloretin or derivative thereof.

59. An actuator in Embodiment 1, which is a small molecule, peptide, protein or nucleic acid that is capable of increasing, decreasing, or modulating local or systemic inflammation, or a part of the inflammatory response.

60. An actuator in Embodiment 1, which is comprised at least in part from theophylline or derivative thereof.

61. An actuator in Embodiment 1, which is comprised at least in part from IPTG or derivative thereof.

62. An actuator in Embodiment 1, which is comprised at least in part from 5-fluorocytosine or derivative thereof.

63. An actuator in Embodiment 1, which is comprised at least in part from propionate or derivative thereof.

64. An actuator in Embodiment 1, which is comprised at least in part from acetate or derivative thereof.

65. An actuator in Embodiment 1, which is comprised at least in part from cumate, cumate ester, or cumic acid, or derivative thereof.

66. An actuator in Embodiment 1, which is comprised at least in part from gluconate or derivative thereof.

67. An actuator in Embodiment 1, which is comprised at least in part from maltose or derivative thereof.

68. An actuator in Embodiment 1, which is comprised at least in part from arabinose or derivative thereof.

69. An actuator in Embodiment 1, which is comprised at least in part from mono- or di-saccharide.

70. An actuator in Embodiment 1, which is comprised at least in part from a trimethoprim or derivative thereof.

71. An actuator in Embodiment 1, which is comprised at least in part from an exogenously administered small molecule, peptide, protein, RNA, DNA, or pharmacological formulation.

72. An actuator in Embodiment 1, at least a part of which can act as at least a part of the effector element in Embodiment 1.

73. An actuator in any Embodiment which is comprised at least in part from an endogenously expressed or presented peptide, hormone, protein, RNA, DNA, enzyme, mineral or chemical or biochemical substance and is capable of interacting with the actuator response element or actuator response sequence.

74. An effector element in Embodiment 1, which is comprised at least in part from an inducible Caspase 9.

75. An effector element in Embodiment 1, which is comprised at least in part from a natural Caspase 9.

76. An effector element in Embodiment 1, which is comprised at least in part from a Zinc-Finger Nuclease.

77. An effector element in Embodiment 1, which is comprised at least in part from a transcription activator-like effector nucleases (TALEN).

78. An effector element in Embodiment 1, which is comprised at least in part from a meganuclease.

79. An effector element in Embodiment 1, which is comprised at least in part from a deoxycytidine deamination-derived editor

80. An effector element in Embodiment 1, which is comprised at least in part from a deoxyadenosine deamination-derived base editor.

81. An effector element in Embodiment 1, which is comprised at least in part from a DNA-binding peptide.

82. An effector element in Embodiment 1, which is comprised at least in part from a viral or bacterial DNA-binding protein.

83. An effector element in Embodiment 1, which is comprised at least in part from a natural Caspase 8.

84. An effector element in Embodiment 1, which is comprised at least in part from a natural Caspase or a Caspase pathway protein.

85. An effector element in Embodiment 1, which is a FK506-binding protein 12 (with F36V mutation) fused with human caspase-8 protein deleted for 1-215 AA and linked to the N-myristoylation signal from Src kinase.

86. An effector element in Embodiment 1, which is comprised in part from an FK506-binding protein 12 (with F36V mutation) fused with human caspase-9 protein deleted for 1-134 AA.

87. An effector element in Embodiment 1, which is comprised at least in part from at least a part of a viral thymidine kinase.

88. An effector element in Embodiment 1, which is comprised at least in part from at least a part of an Escherichia Coli cytosine deaminase.

89. An effector element in Embodiment 1, which is a protein with catalytic activity capable of chemically modifying a nucleotide, nucleoside, or nucleic acid.

90. An effector element in Embodiment 1, which is comprised at least a part from at least a part of a diphtheria toxin subunit A.

91. An effector element in Embodiment 1, which is comprised at least a part from at least a part of a diphtheria toxin subunit B.

92. An effector element in Embodiment 1, which is a dimerizable or trimerizable toxin, protein, or enzyme.

93. An effector element in Embodiment 1, which is comprised at least in part from, at least a part of the human or simian diphtheria toxin receptor.

94. An effector element in Embodiment 1, which is comprised at least a part from at least a part of a bacterial, viral, or eukaryotic toxin.

95. An effector element in Embodiment 1, which is comprised at least a part from at least a part of a protein capable of inhibiting transcription.

96. An effector element in Embodiment 1, which is comprised at least a part from at least a part of a protein capable of inhibiting translation.

97. An effector element in Embodiment 1, which is comprised at least a part from at least a part of a protein capable of inducing apoptosis.

98. An effector element in Embodiment 1, which is comprised at least a part from at least a part of a protein capable of inducing senescence.

99. An effector element in Embodiment 1, which is comprised at least a part from at least a part of a protein capable of reducing general gene expression.

100. An effector element in Embodiment 1, which is comprised at least a part from at least a part of a nuclease.

101. An effector element in Embodiment 1, which is comprised at least a part from at least a part of a nuclease capable of site specific mutagenesis.

102. An effector element in Embodiment 1, which is comprised at least a part from at least a part of a nuclease and at least a part of a targeting element.

103. An effector element in Embodiment 1, which is comprised at least a part from at least a part of a Cas nuclease and guide RNA targeting the nuclease to the sequence in Embodiment 1.

104. An effector element in Embodiment 1, which is comprised at least a part from at least a part of a nuclease targeting the sequence in Embodiment 1.

105. An effector element in Embodiment 1, which is comprised at least a part from at least a part of a nuclease targeting a promoter region of the sequence in Embodiment 1.

106. An effector element in Embodiment 1, which is comprised at least a part from at least a part of a nuclease targeting a coding region of the sequence in Embodiment 1.

107. An effector element in Embodiment 1, which is comprised at least a part from at least a part of a nuclease targeting a post translational regulation element region of the sequence in Embodiment 1.

108. An effector element in Embodiment 1, which is comprised at least a part from at least a part of a recombinase.

109. An effector element in Embodiment 1, which is comprised at least a part from at least a part of a Cre recombinase.

110. An effector element in Embodiment 1, which is comprised at least a part from at least a part of a Krüppel-associated box domain from human gene ZNF10.

111. An effector element in Embodiment 1, which is comprised at least a part from at least a part of a zinc finger protein.

112. An effector element in Embodiment 1, which is comprised at least a part from at least a part of a cyclization recombinase with SV40 nuclear localization signal.

113. An effector element in Embodiment 1, which is comprised at least a part from at least a part of a Cre recombinase with SV40 T-antigen intron inserted in the ORF.

114. An effector element in Embodiment 1, which is comprised at least a part from at least a part of a Flpe recombinase.

115. An effector element in Embodiment 1, which is comprised at least a part from at least a part of a Flpo recombinase.

116. An effector element in Embodiment 1, which is comprised at least a part from at least a part of a Dre recombinase.

117. An effector element in Embodiment 1, which is comprised at least a part from at least a part of an SV40 T-antigen sequence.

118. An effector element in Embodiment 1, which is comprised at least a part from at least a part of a piggyBac transposase.

119. An effector element in Embodiment 1, which is comprised at least a part from at least a part of a transposase.

120. An effector element in Embodiment 1, which is comprised at least a part from at least a part of a sleeping beauty transposase.

121. An effector element in Embodiment 1, which is comprised at least a part from at least a part of an argonaut protein.

122. An effector element in Embodiment 1, which is comprised at least a part from at least a part of a DNA methyltransferase.

123. An effector element in Embodiment 1, which is comprised at least a part from at least a part of a protein which makes up or interacts with chromatin remodeling complex.

124. An effector element in Embodiment 1, which is comprised at least a part from at least a part of a protein that makes up or interacts with the DNA methylation pathway.

125. An effector element in Embodiment 1, which is comprised at least a part from at least a part of a base editor.

126. An effector element in Embodiment 1, which is comprised at least a part from at least a part of a prime editor.

127. An effector element in Embodiment 1, which is comprised at least a part from at least a part of a nickase.

128. An effector element in Embodiment 1, which is comprised at least a part from at least a part of a viral, bacterial, or eukaryotic gene silencing protein.

129. An effector element in Embodiment 1, which is comprised at least a part from at least a part of a siRNA.

130. An effector element in Embodiment 1, which is comprised at least a part from at least a part of a miRNA.

131. An effector element in Embodiment 1, which is comprised at least a part from at least a part of FRT.

132. An effector element in Embodiment 1, which is comprised at least a part from at least a part of an integrase.

133. An effector element in Embodiment 1, which is comprised at least a part from at least a part of an RNA capable of reducing gene expression.

134. An effector element in Embodiment 1, which is comprised at least a part from at least a part of a protein enabling the effector degradation in the absence of an inducer.

135. An effector element in Embodiment 1, which is comprised at least a part from at least a part of a dihydrofolate reductase.

136. An effector element in Embodiment 1, which is comprised at least a part from at least a part of RNA, which is at least partially complementary to the genetic construct in Embodiment 1.

137. An effector element in Embodiment 1, which is comprised at least a part from at least a part of transposon or retrotransposon.

138. An effector element in Embodiment 1, which is comprised at least a part from at least a part of a Streptococcus pyogenes Cas9.

139. An effector element in Embodiment 1, which is comprised at least a part from at least a part of a catalytically inactive Cas9.

140. An effector element in Embodiment 1, which is comprised at least a part from at least a part of a codon optimized Cas9.

141. An effector element in Embodiment 1, which is comprised at least a part from at least a part of Cas9 with a D10A mutation.

142. An effector element in Embodiment 1, which is comprised at least a part from at least a part of a dCas9 fused with KRAB.

143. An effector element in Embodiment 1, which is comprised at least a part from at least a part of dCas9 fused with a carboxy terminal bipartite repressor domain, KRAB-MeCP2 or derivative.

144. An effector element in Embodiment 1, which is comprised at least a part from at least a part of a Campylobacter jujeni Cas9 ortholog.

145. An effector element in Embodiment 1, which is comprised at least a part from at least a part of a CRISPR associated protein or an ortholog.

146. An effector element in Embodiment 1, which is comprised at least a part from at least a part of a Cas12a or Cas12b protein.

147. An effector element in Embodiment 1, which is comprised at least a part from at least a part of Cas13.

148. An effector element in Embodiment 1, which is comprised at least a part from at least a part of Cas12f or Cas13a.

149. An effector element in Embodiment 1, which is comprised at least a part from at least a part of a DNA, DNA capable of expressing, RNA, RNA, protein, or combination thereof capable of effecting DNA sequences in a sequence specific manner to induce double stranded breaks, single stranded breaks, methylation, chromatin modification, or generally epigenetic silencing.

150. An effector element in Embodiment 1, which is comprised at least in part from at least a part of a toxin, toxin derivative, modified toxin, or a homolog, ortholog, or paralog thereof.

151. An effector element in Embodiment 1, which is comprised at least in part from at least a part of a bacterial, fungal, plant, or animal toxin.

152. An effector element in Embodiment 1, which is comprised at least in part from at least a part of a Vibrio cholerae toxin, shiga toxin, Shigella toxin, ricin, saporin, Staphylococcus aureus enterotoxin H, Clostridium perfringens enterotoxin, streptolysin O, ribosome inactivating protein (RIP) enzyme, immunotoxin, Pseudomonas exotoxin A, diphtheria toxin (DT), or DT fragment.

153. An effector element in Embodiment 1, which is comprised at least in part from at least a part of a viral thymidine kinase or derivative thereof.

154. An effector element in Embodiment 1, which is comprised at least in part from at least a part of a bacterial cytosine deaminase or derivative thereof.

155. An effector element in Embodiment 1, which is comprised at least in part from at least a part of a D-amino-acid oxidase or derivative thereof.

156. An effector element in Embodiment 1, which is comprised at least in part from at least a part of a bacterial carboxypeptidase G2 or derivative thereof.

157. An effector element in Embodiment 1, which is comprised at least in part from at least a part of a purine nucleoside phosphorylase or derivative thereof.

158. An effector element in Embodiment 1, which is comprised at least in part from at least a part of a thymidine phosphorylase or derivative thereof.

159. An effector element in Embodiment 1, which is comprised at least in part from at least a part of a xanthine-guanine phosphoribosyl transferase or derivative thereof.

160. An effector element in Embodiment 1, which is comprised at least in part from at least a part of a nitroreductase or derivative thereof.

161. An effector element in Embodiment 1, which is comprised at least in part from at least a part of a penicillin-G amidase or derivative thereof.

162. An effector element in Embodiment 1, which is comprised at least in part from at least a part of a multi-drug activation enzyme or derivative thereof.

163. An effector element in Embodiment 1, which is comprised at least in part from at least a part of a horseradish peroxidase or derivative thereof.

164. An effector element in Embodiment 1, which is comprised at least in part from at least a part of a Beta-galactosidase or derivative thereof.

165. An effector element in Embodiment 1, which is comprised at least in part from at least a part of a deoxyribonucleotide kinase or derivative thereof.

166. An effector element in Embodiment 1, which is comprised at least in part from at least a part of a Beta-glucoronidase or derivative thereof.

167. An effector element in Embodiment 1, which is comprised at least in part from at least a part of a carboxypeptidase A or derivative thereof.

168. An effector element in Embodiment 1, which is comprised at least in part from at least a part of a cytochrome P450 or derivative thereof.

169. An effector element in Embodiment 1, which is comprised at least in part from at least a part of a deoxycytidine kinase or derivative thereof.

170. An effector sequence in Embodiment 1, which encodes at least the effector element.

171. An effector sequence in Embodiment 1, which encodes at least a part of the consensus Lox sequence or a sequence capable of mediating the effect of a Lox sequence.

172. An effector sequence in Embodiment 1, which encodes at least a part of the consensus FRT sequence or a sequence capable of mediating the effect of an FRT sequence.

173. An effector sequence in Embodiment 1, which encodes at least a part of the consensus Rox sequence or a sequence capable of mediating the effect of a Rox sequence.

174. An effector sequence in Embodiment 1, which is comprised at least in part from GTGTAGTTCGACCATTCGTG (SEQ ID NO: 1).

175. An effector sequence in Embodiment 1, which is comprised at least in part from GTTCAGGATCACGTTACCGC (SEQ ID NO: 2).

176. An effector sequence in Embodiment 1, which is comprised at least in part from GGGCGAGGAGCTGTTCACCG (SEQ ID NO: 3).

177. An effector sequence in Embodiment 1, which is comprised at least in part from GAAGTTCGAGGGCGACACCC (SEQ ID NO: 4).

178. An effector sequence in Embodiment 1, which is comprised at least in part from GCAGATGTAGTGTTTCCACA (SEQ ID NO: 5).

179. An effector sequence in Embodiment 1, which is comprised at least in part from CCTAAGGTTAAGTCGCCCTCG (SEQ ID NO: 6).

180. An effector sequence in Embodiment 1, which is comprised at least in part from CAACAAGATGAAGAGCACCAA (SEQ ID NO: 7).

181. An effector sequence in Embodiment 1, which encodes at least a part of attB, attR, attL, or attP sites.

182. An effector sequence in Embodiment 1, which is comprised at least in part from genetic sequences, which can be targeted by integrases, recombinases, nucleases, nickases, chromatin complexes, or methylases or methyltransferases.

183. An effector sequence in Embodiment 1, which is comprised at least in part from genetic sequences, which can be targeted for silencing RNA or protein binding or can be itself silenced.

184. An effector sequence in Embodiment 1, which is a DNA sequence that can serve as a target of a guide RNA for a CRISPR associated protein.

185. An effector sequence in Embodiment 1, which is a DNA sequence derived from viral or bacterial genomes.

186. An effector sequence in Embodiment 1, which forms a secondary structure in the absence of an inducer molecule.

187. An effector sequence in Embodiment 1, which is an inflammation responsive promoter.

188. An effector sequence in Embodiment 1, which is derived at least in part from a mammalian promoter or enhancer.

189. An effector sequence in Embodiment 1, which forms a secondary structure in the presence of an inducer molecule.

190. An actuator response element in Embodiment 1, which is a nucleic acid sequence or a protein that interacts with an actuator to enable activity of the effector element or effector sequence.

191. An actuator response element in Embodiment 1, which is comprised at least in part from a tetracycline-controlled transcriptional silencer (tTS).

192. An actuator response element in Embodiment 1, which is comprised at least in part from a Kruppel associated box (KRAB) AB domain of the kid-1 transcriptional repressor.

193. An actuator response element in Embodiment 1, which is composed at least in part of a reverse tetracycline-controlled transactivator.

194. An actuator response element in Embodiment 1, which is comprised at least in part of a viral protein such as VP16 fused with a Tet-Repressor (TetR) or a reverse Tet-Repressor (rTetR).

195. An actuator response element in Embodiment 1, which is comprised of a tTS and rtTA driven by the same promoter.

196. An actuator response element in Embodiment 1, which is comprised of tTS and rtTA driven by different promoters.

197. An actuator response element in Embodiment 1, which is comprised at least in part from a transcriptional repressor.

198. An actuator response element in Embodiment 1, which is comprised at least in part from a transcriptional silencer.

199. An actuator response element in Embodiment 1, which is comprised at least in part from a DNA binding protein.

200. An actuator response element in Embodiment 1, which is comprised at least in part from a promoter binding protein.

201. An actuator response element in Embodiment 1, which is comprised at least in part from a transcription initiation complex binding protein.

202. An actuator response element in Embodiment 1, which is comprised at least in part of a protein responsible for DNA methylation.

203. An actuator response element in Embodiment 1, which is comprised at least in part of a protein responsible for histone modification.

204. An actuator response element in Embodiment 1, which is comprised at least in part of at least a part of a Tet-ON system.

205. An actuator response element in Embodiment 1, which is comprised at least in part of at least a part of a Tet-OFF system.

206. An actuator response element in Embodiment 1, which is comprised at least in part of at least a part of AlcR, ArgR, BirA, EthR, HdnoR, HucR, MphR(A), PIP, Rex, RheA, ScbR, TraR, TtgR or their analog, homolog, ortholog or protein fragment, protein, or fusion protein of similar functionality.

207. An actuator response element in Embodiment 1, which is comprised at least in part from at least a part of lacUV5, lac, tac, trc, prpB, aceA, aceB, gntP, gntK, CJ10X2, tacM, malE1, git1, BAD, SPL, P4-N14, cspB, aprE, sod, dapA, porB, ilvC, L10, L26, L16, L51, H30, H36 or natural, synthetic or semi-synthetic analogs, homologs, orthologs or protein fragments, proteins, or fusion proteins of similar functionality.

208. An actuator response element in Embodiment 1, which is comprised at least in part from at least a part of ABI1-Gal4DBD, ABA, PVL1-VP16, PHR-p65, CIB1-TetR, or their analog, homolog, ortholog or protein fragment, protein, or fusion protein of similar functionality.

209. An actuator response element in Embodiment 1, which is comprised at least in part from at least a part of a transcriptional activator, enhancer, de-repressor, or protein, RNA, or DNA capable of increasing gene expression directly or indirectly in a targeted or generally cis-acting manner.

It should be understood that within the scope of this invention the formulation, materials, genetic constructs, sequences, biological and chemical compositions, or methods of use may be varied by one skilled in the art, to the extent that the structures described herewithin perform the desired function and remain within the scope of the present invention. Various parts, components or characteristics may be used in combination, with or without modification by someone skilled in the art to achieve the desired functionality of the aforedescribed formulation.

Moreover, all individual features and methods of use described herein, and each and every combination of two or more of such features and methods of use, are included within the scope of the present invention provided that these features and methods of use in such a combination are not mutually inconsistent. It is understood that certain portions or combinations of such portions can be varied by someone trained in the art while still achieving the main goal of the invention.

Finally, it is understood that the specific ranges provided in the current invention are not restrictive and are for example purposes only, values outside of the specified ranges may be used to achieve the goal of the invention without modification to the proposed mechanistic principals.

The phraseology and terminology used in this application is for the purpose of description and should not be regarded as limiting. The use of terms such as “including,” “comprising,” “having,” “containing,” “involving,” and/or variations thereof in this application, is meant to encompass the items listed thereafter and equivalents thereof as well as additional items.

In embodiments of any of the compositions and methods provided herein, “comprising” may be replaced with “consisting essentially of” or “consisting of”. The phrase “consisting essentially of” is used herein to require the specified integer(s) or steps as well as those which do not materially affect the character or function of the claimed invention. As used herein, the term “consisting” is used to indicate the presence of the recited integer (e.g. a feature, element, characteristic, property, method/process step or limitation) or group of integers (e.g. features, elements, characteristics, properties, method/process steps or limitations) alone.

The present invention is further illustrated by the following Examples, which in no way should be construed as further limiting. The entire contents of all of the references (including literature references, issued patents, published patent applications, and co-pending patent application) cited throughout this application are hereby expressly incorporated by reference.

### EXAMPLES

In order that the invention described in the present application may be more fully understood, the following examples are set forth. The examples described in this application are offered to illustrate the systems and methods provided in this disclosure and are not to be construed in any way as limiting their scope.

### Example 1

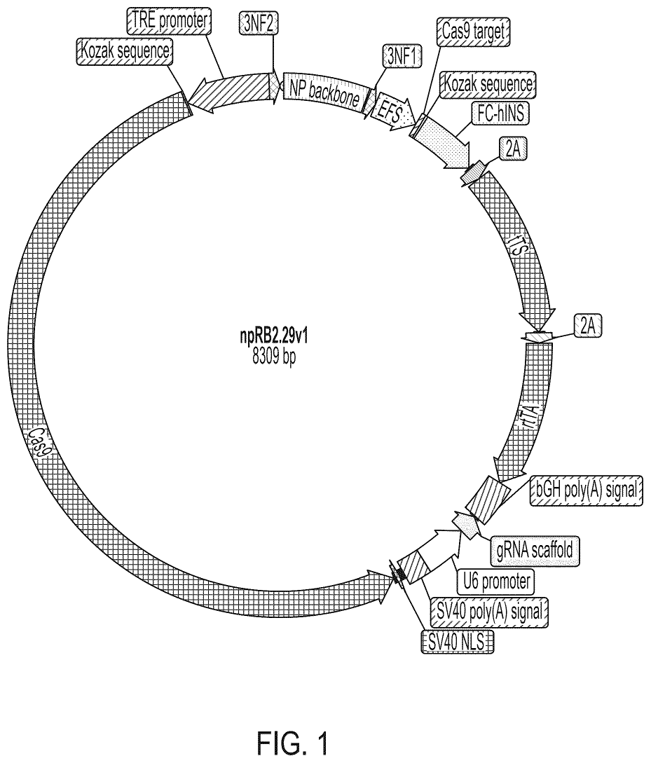

In the first example, a therapeutic genetic construct with dose adjustment functionality is created by placing a furin-cleavable insulin gene under the regulation of an EFS promoter to drive basal insulin expression. To enable dose reduction a tetracycline inducible promoter and tetracycline responsive transcriptional activator or TetOn operator are added to drive expression of a CRISPR Cas9 protein and sgRNA in the presence of the tetracycline actuator. The sgRNA targets the Cas9 protein to cleave a target sequence separating the EFS promoter from the furin-cleavable insulin and eliminating expression of the furin-cleavable insulin therapeutic transgene. The construct self-inactivates and therefore reduces the overall level of therapeutic transgene expression following activation by an exogenously administered actuator—tetracycline. FIG. 1 shows a plasmid map for the genetic construct described in this example. Actuator: tetracycline, atomic formula C22H24N2O8, CAS Number: 60-54-8 or equivalent therapeutic/USP equivalent thereof. CAS RN 64-75-5—. Tetracycline Hydrochloride, Molecular Formula: C22H24N2O8.ClH.

Nanoplasmid backbone
(SEQ ID NO: 8)
CTAGCTGGCTTGTTGTCCACAACCATTAAACCTTAAAAGCTTTAAAAGCCTTATATA
TTCTTTTTTTTCTTATAAAACTTAAAACCTTAGAGGCTATTTAAGTTGCTGATTTATA
TTAATTTTATTGTTCAAACATGAGAGCTTAGTACGTGAAACATGAGAGCTTAGTAC
ATTAGCCATGAGAGCTTAGTACATTAGCCATGAGGGTTTAGTTCATTAAACATGAG
AGCTTAGTACATTAAACATGAGAGCTTAGTACATTAAACATGAGAGCTTAGTACAT
ACTATCAACAGGTTGAACTGCTGATCTGTACAGTAGAATTGGTAAAGAGAGTTGT
GTAAAATATTGAGTTCGCACATCTTGTTGTCTGATTATTGATTTTTGGCGAAACCAT
TTGATCATATGACAAGATGTGTATCTACCTTAACTTAATGATTTTGATAAAAATCATT
AGGTAC
Full sequence (except nanoplasmid backbone)
(SEQ ID NO: 9)
ctggggactttccagcctggggactttccagctgggactttccaggggctccggtgcccgtcagtgggcagagcgcacatcgcccac
agtccccgagaagttggggggaggggtcggcaattgatccggtgcctagagaaggtggcgcggggtaaactgggaaagtgatgtc
gtgtactggctccgcctttttcccgagggtgggggagaaccgtatataagtgcagtagtcgccgtgaacgttctttttcgcaacgggtttg
ccgccagaacacagggttcaggatcacgttaccgcggggccaccatggccctgtggatgagactgctgcctctgctggccctcctgg
ccctgtggggccccgaccccgccgccgcttttgtgaaccagcacctgtgcggaagcgacctggtcgaggccctgtacctggtgtgcg
gcgagcggggcttcttctacacccctagaaccaagcgggaagccgaggatctgcaggtgggccaggtggagctgggcggcggac
caggcgccggctccctgcaacctctggctctggaaggatctagacagaagagaggcatcgtggagcagtgttgtacaagcatctgca
gcctgtatcagctggaaaactactgcaatggatctggcgccacaaacttcagcctgctgaagcaggccggagatgtggaagaaaatc
ctggccccatgtctagattagataaaagtaaagtgattaacagcgcattagagctgcttaatgaggtcggaatcgaaggtttaacaaccc
gtaaactcgcccagaagctaggtgtagagcagcctacattgtattggcacgtgcgcaacaagcagactcttatgaacatgctttcagag
gcaatactggcgaagcatcacacccgttcagcaccgttaccgactgagagttggcagcagtttctccaggaaaatgctctgagtttccg
taaagcattactggtccatcgtgatggagcccgattgcatatagggacctctcctacgcccccccagtttgaacaagcagaggcgcaa
ctacgctgtctatgcgatgcagggttttcggtcgaggaggctcttttcattctgcaatctatcagccattttacgttgggtgcagtattagag
gagcaagcaacaaaccagatagaaaataatcatgtgatagacgctgcaccaccattattacaagaggcatttaatattcaggcgagaa
cctctgctgaaatggccttccatttcgggctgaaatcattaatatttggattttctgcacagttagatgaaaaaaagcatacacccattgag
gatggtaataaaccaaaaaagaagagaaagctagcagtgtcagtgacatttgaagatgtggctgtgctctttactcgggacgagtgga
agaagctggatctgtctcagagaagcctgtaccgtgaggtgatgctggagaattacagcaacctggcctccatggcaggattcctgttt
accaaaccaaaggtgatctccctgttgcagcaaggagaggatccctggggctccggagagggcaggggaagtcttctaacatgcgg
ggacgtggaggaaaatcccggccccatgtctagactggacaagagcaaagtcataaacggcgctctggaattactcaatggagtcgg
tatcgaaggcctgacgacaaggaaactcgctcaaaagctgggagttgagcagcctaccctgtactggcacgtgaagaacaagcggg
ccctgctcgatgccctgccaatcgagatgctggacaggcatcatacccacttctgccccctggaaggcgagtcatggcaagactttctg
cggaacaacgccaagtcattccgctgtgctctcctctcacatcgcgacggggctaaagtgcatctcggcacccgcccaacagagaaa
cagtacgaaaccctggaaaatcagctcgcgttcctgtgtcagcaaggcttctccctggagaacgcactgtacgctctgtccgccgtgg
gccactttacactgggctgcgtattggaggaacaggagcatcaagtagcaaaagaggaaagagagacacctaccaccgattctatgc
ccccacttctgagacaagcaattgagctgttcgaccggcagggagccgaacctgccttccttttcggcctggaactaatcatatgtggc
ctggagaaacagctaaagtgcgaaagcggcgggccggccgacgcccttgacgattttgacttagacatgctcccagccgatgccctt
gacgactttgaccttgatatgctgcctgctgacgctcttgacgattttgaccttgacatgctccccgggtaactgtgccttctagttgccag
ccatctgttgtttgcccctcccccgtgccttccttgaccctggaaggtgccactcccactgtcctttcctaataaaatgaggaaattgcatc
gcattgtctgagtaggtgtcattctattctggggggtggggtggggcaggacagcaagggggaggattgggaagagaatagcaggc
aggctggggaaaaaaagcaccgactcggtgccactttttcaagttgataacggactagccttattttaacttgctatttctagctctaaaac
gcggtaacgtgatcctgaaccgtcctttccacaagatatataaagccaagaaatcgaaatactttcaagttacggtaagcatatgatagtc
cattttaaaacataattttaaaactgcaaactacccaagaaattattactttctacgtcacgtattttgtactaatatctttgtgtttacagtca
aattaattccaattatctctctaacagccttgtatcgtatatgcaaatatgaaggaatcatgggaaataggccctcaacttgtttattgcagctt
ataatggttacaaataaagcaatagcatcacaaatttcacaaataaagcatttttttcactgcattctagttgtggtttgtccaaactcatcaat
gtatcttatcattattaggcgtagtcgggcacgtcgtaggggtatacttttctctttttctttggaggtccggattactttttcttttttgcctg
gccggcctttttcgtggccgccggccttttgtcgcctcccagctgagacaggtcgatccgtgtctcgtacaggccggtgatgctctggtggatc
agggtggcgtccagcacctctttggtgctggtgtacctcttccggtcgatggtggtgtcaaagtacttgaaggcggcaggggctccca
gattggtcagggtaaacaggtggatgatattctcggcctgctctctgatgggcttatcccggtgcttgttgtaggcggacagcactttgtc
cagattagcgtcggccaggatcactctcttggagaactcgctgatctgctcgatgatctcgtccaggtagtgcttgtgctgttccacaaac
agctgtttctgctcattatcctcgggggagcccttcagcttctcatagtggctggccaggtacaggaagttcacatatttggagggcagg
gccagttcgtttcccttctgcagttcgccggcagaggccagcattctcttccggccgttttccagctcgaacagggagtacttaggcagc
ttgatgatcaggtcctttttcacttctttgtagcccttggcttccagaaagtcgatgggattcttctcgaagctgcttctttccatgatggtgat
ccccagcagctctttcacactcttcagtttcttggacttgcccttttccactttggccaccaccagcacagaataggccacggtggggctg
tcgaagccgccgtacttcttagggtcccagtccttctttctggcgatcagcttatcgctgttcctcttgggcaggatagactctttgctgaag
ccgcctgtctgcacctcggtctttttcacgatattcacttggggcatgctcagcactttccgcacggtggcaaaatcccggcccttatccc
acacgatctccccggtttcgccgtttgtctcgatcagaggccgcttccggatctcgccgttggccagggtaatctcggtcttgaaaaagtt
catgatgttgctgtagaagaagtacttggcggtagccttgccgatttcctgctcgctcttggcgatcatcttccgcacgtcgtacaccttgt
agtcgccgtacacgaactcgctttccagcttagggtactttttgatcagggcggttcccacgacggcgttcaggtaggcgtcgtgggcg
tggtggtagttgttgatctcgcgcactttgtaaaactggaaatccttccggaaatcggacaccagcttggacttcagggtgatcactttca
cttcccggatcagcttgtcattctcgtcgtacttagtgttcatccgggagtccaggatctgtgccacgtgctttgtgatctgccgggtttcca
ccagctgtctcttgatgaagccggccttatccagttcgctcaggccgcctctctcggccttggtcagattgtcgaactttctctgggtaatc
agcttggcgttcagcagctgccgccagtagttcttcatcttcttcacgacctcttcggagggcacgttgtcgctcttgccccggttcttgtc
gcttctggtcagcaccttgttgtcgatggagtcgtccttcagaaagctctgaggcacgatatggtccacatcgtagtcggacagccggtt
gatgtccagttcctggtccacgtacatatcccgcccattctgcaggtagtacaggtacagcttctcgttctgcagctgggtgttttccacg
gggtgttctttcaggatctggctgcccagctctttgatgccctcttcgatccgcttcattctctcgcggctgttcttctgtcccttctgggtggt
ctggttctctctggccatttcgatcacgatgttctcgggcttgtgccggcccatcactttcacgagctcgtccaccaccttcactgtctgca
ggatgcccttcttaatggcggggctgccggccagattggcaatgtgctcgtgcaggctatcgccctggccggacacctgggctttctg
gatgtcctctttaaaggtcaggctgtcgtcgtggatcagctgcatgaagtttctgttggcgaagccgtcggacttcaggaaatccaggatt
gtcttgccggactgcttgtcccggatgccgttgatcagcttccggctcagcctgccccagccggtgtatctccgccgcttcagctgcttc
atcactttgtcgtcgaacaggtgggcataggttttcagccgttcctcgatcatctctctgtcctcaaacagtgtcagggtcagcacgatatc
ttccagaatgtcctcgttttcctcattgtccaggaagtccttgtccttgataattttcagcagatcgtggtatgtgcccagggaggcgttgaa
ccgatcttccacgccggagatttccacggagtcgaagcactcgattttcttgaagtagtcctctttcagctgcttcacggtcactttccggtt
ggtcttgaacagcaggtccacgatggcctttttctgctcgccgctcaggaaggcgggctttctcattccctcggtcacgtatttcactttgg
tcagctcgttatacacggtgaagtactcgtacagcaggctgtgcttgggcagcaccttctcgttgggcaggttcttatcgaagttggtcat
ccgctcgatgaagctctgggcggaagcgcccttgtccaccacttcctcgaagttccagggggtgatggtttcctcgctctttctggtcat
ccaggcgaatctgctgtttcccctggccagagggcccacgtagtaggggatgcggaaggtcaggatcttctcgatcttttcccggttgt
ccttcaggaatgggtaaaaatcttcctgccgccgcagaatggcgtgcagctctcccaggtggatctggtgggggatgctgccgttgtc
gaaggtccgctgcttccgcagcaggtcctctctgttcagcttcacgagcagttcctcggtgccgtccatcttttccaggatgggcttgatg
aacttgtagaactcttcctggctggctccgccgtcaatgtagccggcgtagccgttcttgctctggtcgaagaaaatctctttgtacttctc
aggcagctgctgccgcacgagagctttcagcagggtcaggtcctggtggtgctcgtcgtatctcttgatcatagaggcgctcaggggg
gccttggtgatctcggtgttcactctcaggatgtcgctcagcaggatggcgtcggacaggttcttggcggccagaaacaggtcggcgt
actggtcgccgatctgggccagcaggttgtccaggtcgtcgtcgtaggtgtccttgctcagctgcagtttggcatcctcggccaggtcg
aagttgctcttgaagttgggggtcaggcccaggctcagggcaatcaggtttccgaacaggccattcttcttctcgccgggcagctggg
cgatcagattttccagccgtctgctcttgctcagtctggcagacaggatggccttggcgtccacgccgctggcgttgatggggttttcctc
gaacagctggttgtaggtctgcaccagctggatgaacagcttgtccacgtcgctgttgtcggggttcaggtcgccctcgatcaggaagt
ggccccggaacttgatcatgtgggccagggccagatagatcagccgcaggtcggccttgtcggtgctgtccaccagtttctttctcagg
tggtagatggtggggtacttctcgtggtaggccacctcgtccacgatgttgccgaagatggggtgccgctcgtgcttcttatcctcttcca
ccaggaaggactcttccagtctgtggaagaagctgtcgtccaccttggccatctcgttgctgaagatctcttgcagatagcagatccggt
tcttccgtctggtgtatcttcttctggcggttctcttcagccgggtggcctcggctgtttcgccgctgtcgaacagcagggctccgatcag
gttcttcttgatgctgtgccggtcggtgttgcccagcaccttgaatttcttgctgggcaccttgtactcgtcggtgatcacggcccagccca
cagagttggtgccgatgtccaggccgatgctgtacttcttgtcggctgctgggactccgtggataccgaccttccgcttcttctttggggc
catcttatcgtcatcgtctttgtaatcaatatcatgatccttgtagtctccgtcgtggtccttatagtccatggtggccaattcggggccgcg
gaggctggatcggtcccggtgtcttctatggaggtcaaaacagcgtggatggcgtctccaggcgatctgacggttcactaaacgagct
ctgcttatataggtcgagtttaccactccctatcagtgatagagaaaagtgaaagtcgagtttaccactccctatcagtgatagagaaaag
tgaaagtcgagtttaccactccctatcagtgatagagaaaagtgaaagtcgagtttaccactccctatcagtgatagagaaaagtgaaag
tcgagtttaccactccctatcagtgatagagaaaagtgaaagtcgagtttaccactccctatcagtgatagagaaaagtgaaagtcgagt
ttaccactccctatcagtgatagagaaaagtgaaagtcgagctcggtaccctggggactttccagctggggactttccagctgggacttt
ccaggag
3NF1
(SEQ ID NO: 10)
Ctggggactttccagcctggggactttccagctgggactttccagg
3NF2
(SEQ ID NO: 11)
Ctggggactttccagctggggactttccagctgggactttccaggag
Effector element: Cas9 and sgRNA
Cas9
Nucleotide sequence:
(SEQ ID NO: 12)
ATGGACTATAAGGACCACGACGGAGACTACAAGGATCATGATATTGATTACAAAGA
CGATGACGATAAGATGGCCCCAAAGAAGAAGCGGAAGGTCGGTATCCACGGAGT
CCCAGCAGCCGACAAGAAGTACAGCATCGGCCTGGACATCGGCACCAACTCTGTG
GGCTGGGCCGTGATCACCGACGAGTACAAGGTGCCCAGCAAGAAATTCAAGGTG
CTGGGCAACACCGACCGGCACAGCATCAAGAAGAACCTGATCGGAGCCCTGCTG
TTCGACAGCGGCGAAACAGCCGAGGCCACCCGGCTGAAGAGAACCGCCAGAAG
AAGATACACCAGACGGAAGAACCGGATCTGCTATCTGCAAGAGATCTTCAGCAAC
GAGATGGCCAAGGTGGACGACAGCTTCTTCCACAGACTGGAAGAGTCCTTCCTG
GTGGAAGAGGATAAGAAGCACGAGCGGCACCCCATCTTCGGCAACATCGTGGAC
GAGGTGGCCTACCACGAGAAGTACCCCACCATCTACCACCTGAGAAAGAAACTG
GTGGACAGCACCGACAAGGCCGACCTGCGGCTGATCTATCTGGCCCTGGCCCACA
TGATCAAGTTCCGGGGCCACTTCCTGATCGAGGGCGACCTGAACCCCGACAACAG
CGACGTGGACAAGCTGTTCATCCAGCTGGTGCAGACCTACAACCAGCTGTTCGAG
GAAAACCCCATCAACGCCAGCGGCGTGGACGCCAAGGCCATCCTGTCTGCCAGA
CTGAGCAAGAGCAGACGGCTGGAAAATCTGATCGCCCAGCTGCCCGGCGAGAAG
AAGAATGGCCTGTTCGGAAACCTGATTGCCCTGAGCCTGGGCCTGACCCCCAACT
TCAAGAGCAACTTCGACCTGGCCGAGGATGCCAAACTGCAGCTGAGCAAGGACA
CCTACGACGACGACCTGGACAACCTGCTGGCCCAGATCGGCGACCAGTACGCCG
ACCTGTTTCTGGCCGCCAAGAACCTGTCCGACGCCATCCTGCTGAGCGACATCCT
GAGAGTGAACACCGAGATCACCAAGGCCCCCCTGAGCGCCTCTATGATCAAGAGA
TACGACGAGCACCACCAGGACCTGACCCTGCTGAAAGCTCTCGTGCGGCAGCAG
CTGCCTGAGAAGTACAAAGAGATTTTCTTCGACCAGAGCAAGAACGGCTACGCCG
GCTACATTGACGGCGGAGCCAGCCAGGAAGAGTTCTACAAGTTCATCAAGCCCAT
CCTGGAAAAGATGGACGGCACCGAGGAACTGCTCGTGAAGCTGAACAGAGAGG
ACCTGCTGCGGAAGCAGCGGACCTTCGACAACGGCAGCATCCCCCACCAGATCC
ACCTGGGAGAGCTGCACGCCATTCTGCGGCGGCAGGAAGATTTTTACCCATTCCT
GAAGGACAACCGGGAAAAGATCGAGAAGATCCTGACCTTCCGCATCCCCTACTAC
GTGGGCCCTCTGGCCAGGGGAAACAGCAGATTCGCCTGGATGACCAGAAAGAGC
GAGGAAACCATCACCCCCTGGAACTTCGAGGAAGTGGTGGACAAGGGCGCTTCC
GCCCAGAGCTTCATCGAGCGGATGACCAACTTCGATAAGAACCTGCCCAACGAGA
AGGTGCTGCCCAAGCACAGCCTGCTGTACGAGTACTTCACCGTGTATAACGAGCT
GACCAAAGTGAAATACGTGACCGAGGGAATGAGAAAGCCCGCCTTCCTGAGCGG
CGAGCAGAAAAAGGCCATCGTGGACCTGCTGTTCAAGACCAACCGGAAAGTGAC
CGTGAAGCAGCTGAAAGAGGACTACTTCAAGAAAATCGAGTGCTTCGACTCCGT
GGAAATCTCCGGCGTGGAAGATCGGTTCAACGCCTCCCTGGGCACATACCACGAT
CTGCTGAAAATTATCAAGGACAAGGACTTCCTGGACAATGAGGAAAACGAGGAC
ATTCTGGAAGATATCGTGCTGACCCTGACACTGTTTGAGGACAGAGAGATGATCG
AGGAACGGCTGAAAACCTATGCCCACCTGTTCGACGACAAAGTGATGAAGCAGC
TGAAGCGGCGGAGATACACCGGCTGGGGCAGGCTGAGCCGGAAGCTGATCAACG
GCATCCGGGACAAGCAGTCCGGCAAGACAATCCTGGATTTCCTGAAGTCCGACGG
CTTCGCCAACAGAAACTTCATGCAGCTGATCCACGACGACAGCCTGACCTTTAAA
GAGGACATCCAGAAAGCCCAGGTGTCCGGCCAGGGCGATAGCCTGCACGAGCAC
ATTGCCAATCTGGCCGGCAGCCCCGCCATTAAGAAGGGCATCCTGCAGACAGTGA
AGGTGGTGGACGAGCTCGTGAAAGTGATGGGCCGGCACAAGCCCGAGAACATCG
TGATCGAAATGGCCAGAGAGAACCAGACCACCCAGAAGGGACAGAAGAACAGC
CGCGAGAGAATGAAGCGGATCGAAGAGGGCATCAAAGAGCTGGGCAGCCAGATC
CTGAAAGAACACCCCGTGGAAAACACCCAGCTGCAGAACGAGAAGCTGTACCTG
TACTACCTGCAGAATGGGGGGATATGTACGTGGACCAGGAACTGGACATCAACC
GGCTGTCCGACTACGATGTGGACCATATCGTGCCTCAGAGCTTTCTGAAGGACGA
CTCCATCGACAACAAGGTGCTGACCAGAAGCGACAAGAACCGGGGCAAGAGCG
ACAACGTGCCCTCCGAAGAGGTCGTGAAGAAGATGAAGAACTACTGGCGGCAGC
TGCTGAACGCCAAGCTGATTACCCAGAGAAAGTTCGACAATCTGACCAAGGCCGA
GAGAGGCGGCCTGAGCGAACTGGATAAGGCCGGCTTCATCAAGAGACAGCTGGT
GGAAACCCGGCAGATCACAAAGCACGTGGCACAGATCCTGGACTCCCGGATGAA
CACTAAGTACGACGAGAATGACAAGCTGATCCGGGAAGTGAAAGTGATCACCCTG
AAGTCCAAGCTGGTGTCCGATTTCCGGAAGGATTTCCAGTTTTACAAAGTGCGCG
AGATCAACAACTACCACCACGCCCACGACGCCTACCTGAACGCCGTCGTGGGAAC
CGCCCTGATCAAAAAGTACCCTAAGCTGGAAAGCGAGTTCGTGTACGGCGACTAC
AAGGTGTACGACGTGCGGAAGATGATCGCCAAGAGCGAGCAGGAAATCGGCAAG
GCTACCGCCAAGTACTTCTTCTACAGCAACATCATGAACTTTTTCAAGACCGAGAT
TACCCTGGCCAACGGCGAGATCCGGAAGCGGCCTCTGATCGAGACAAACGGCGA
AACCGGGGAGATCGTGTGGGATAAGGGCCGGGATTTTGCCACCGTGCGGAAAGT
GCTGAGCATGCCCCAAGTGAATATCGTGAAAAAGACCGAGGTGCAGACAGGCGG
CTTCAGCAAAGAGTCTATCCTGCCCAAGAGGAACAGCGATAAGCTGATCGCCAGA
AAGAAGGACTGGGACCCTAAGAAGTACGGCGGCTTCGACAGCCCCACCGTGGCC
TATTCTGTGCTGGTGGTGGCCAAAGTGGAAAAGGGCAAGTCCAAGAAACTGAAG
AGTGTGAAAGAGCTGCTGGGGATCACCATCATGGAAAGAAGCAGCTTCGAGAAG
AATCCCATCGACTTTCTGGAAGCCAAGGGCTACAAAGAAGTGAAAAAGGACCTG
ATCATCAAGCTGCCTAAGTACTCCCTGTTCGAGCTGGAAAACGGCCGGAAGAGAA
TGCTGGCCTCTGCCGGCGAACTGCAGAAGGGAAACGAACTGGCCCTGCCCTCCA
AATATGTGAACTTCCTGTACCTGGCCAGCCACTATGAGAAGCTGAAGGGCTCCCCC
GAGGATAATGAGCAGAAACAGCTGTTTGTGGAACAGCACAAGCACTACCTGGAC
GAGATCATCGAGCAGATCAGCGAGTTCTCCAAGAGAGTGATCCTGGCCGACGCTA
ATCTGGACAAAGTGCTGTCCGCCTACAACAAGCACCGGGATAAGCCCATCAGAGA
GCAGGCCGAGAATATCATCCACCTGTTTACCCTGACCAATCTGGGAGCCCCTGCCG
CCTTCAAGTACTTTGACACCACCATCGACCGGAAGAGGTACACCAGCACCAAAGA
GGTGCTGGACGCCACCCTGATCCACCAGAGCATCACCGGCCTGTACGAGACACGG
ATCGACCTGTCTCAGCTGGGAGGCGACAAAAGGCCGGCGGCCACGAAAAAGGCC
GGCCAGGCAAAAAAGAAAAAGTAA
Amino acid sequence:
(SEQ ID NO: 13)
MDYKDHDGDYKDHDIDYKDDDDKMAPKKKRKVGIHGVPAADKKYSIGLDIGTNSV
GWAVITDEYKVPSKKFKVLGNTDRHSIKKNLIGALLFDSGETAEATRLKRTARRRYTR
RKNRICYLQEIFSNEMAKVDDSFFHRLEESFLVEEDKKHERHPIFGNIVDEVAYHEKY
PTIYHLRKKLVDSTDKADLRLIYLALAHMIKFRGHFLIEGDLNPDNSDVDKLFIQLVQ
TYNQLFEENPINASGVDAKAILSARLSKSRRLENLIAQLPGEKKNGLFGNLIALSLGLT
PNFKSNFDLAEDAKLQLSKDTYDDDLDNLLAQIGDQYADLFLAAKNLSDAILLSDIL
RVNTEITKAPLSASMIKRYDEHHQDLTLLKALVRQQLPEKYKEIFFDQSKNGYAGYID
GGASQEEFYKFIKPILEKMDGTEELLVKLNREDLLRKQRTFDNGSIPHQIHLGELHAIL
RRQEDFYPFLKDNREKIEKILTFRIPYYVGPLARGNSRFAWMTRKSEETITPWNFEEV
VDKGASAQSFIERMTNFDKNLPNEKVLPKHSLLYEYFTVYNELTKVKYVTEGMRKP
AFLSGEQKKAIVDLLFKTNRKVTVKQLKEDYFKKIECFDSVEISGVEDRFNASLGTY
HDLLKIIKDKDFLDNEENEDILEDIVLTLTLFEDREMIEERLKTYAHLFDDKVMKQLK
RRRYTGWGRLSRKLINGIRDKQSGKTILDFLKSDGFANRNFMQLIHDDSLTFKEDIQK
AQVSGQGDSLHEHIANLAGSPAIKKGILQTVKVVDELVKVMGRHKPENIVIEMAREN
QTTQKGQKNSRERMKRIEEGIKELGSQILKEHPVENTQLQNEKLYLYYLQNGRDMY
VDQELDINRLSDYDVDHIVPQSFLKDDSIDNKVLTRSDKNRGKSDNVPSEEVVKKMK
NYWRQLLNAKLITQRKFDNLTKAERGGLSELDKAGFIKRQLVETRQITKHVAQILDSR
MNTKYDENDKLIREVKVITLKSKLVSDFRKDFQFYKVREINNYHHAHDAYLNAVVG
TALIKKYPKLESEFVYGDYKVYDVRKMIAKSEQEIGKATAKYFFYSNIMNFFKTEITL
ANGEIRKRPLIETNGETGEIVWDKGRDFATVRKVLSMPQVNIVKKTEVQTGGFSKESI
LPKRNSDKLIARKKDWDPKKYGGFDSPTVAYSVLVVAKVEKGKSKKLKSVKELLGIT
IMERSSFEKNPIDFLEAKGYKEVKKDLIIKLPKYSLFELENGRKRMLASAGELQKGNE
LALPSKYVNFLYLASHYEKLKGSPEDNEQKQLFVEQHKHYLDEIIEQISEFSKRVILAD
ANLDKVLSAYNKHRDKPIREQAENIIHLFTLTNLGAPAAFKYFDTTIDRKRYTSTKEV
LDATLIHQSITGLYETRIDLSQLGGDKRPAATKKAGQAKKKK
sgRNA
(SEQ ID NO: 2)
GTTCAGGATCACGTTACCGC
sgRNA scaffold
(SEQ ID NO: 14)
GTTCAGGATCACGTTACCGCGTTTTAGAGCTAGAAATAGCAAGTTAAAATAAGGCT
AGTCCGTTATCAACTTGAAAAAGTGGCACCGAGTCGGTGC
U6 promoter
(SEQ ID NO: 15)
GAGGGCCTATTTCCCATGATTCCTTCATATTTGCATATACGATACAAGGCTGTTAGA
GAGATAATTGGAATTAATTTGACTGTAAACACAAAGATATTAGTACAAAATACGTG
ACGTAGAAAGTAATAATTTCTTGGGTAGTTTGCAGTTTTAAAATTATGTTTTAAAAT
GGACTATCATATGCTTACCGTAACTTGAAAGTATTTCGATTTCTTGGCTTTATATATC
TTGTGGAAAGGAC
Effector sequence: target of Cas9 corresponding to above sgRNA
(SEQ ID NO: 16)
CAAGTCCATGTGCAATGGCG
Protospacer adjacent motif (PAM sequence)
NGG (N-any nucleotide)
Actuator response element: TetRepressor/Gen 1 TetOn Reverse tetracycline responsive
transcriptional activator M2 (rtTA)
Nucleotide sequence:
(SEQ ID NO: 17)
ATGTCTAGACTGGACAAGAGCAAAGTCATAAACGGCGCTCTGGAATTACTCAATG
GAGTCGGTATCGAAGGCCTGACGACAAGGAAACTCGCTCAAAAGCTGGGAGTTG
AGCAGCCTACCCTGTACTGGCACGTGAAGAACAAGCGGGCCCTGCTCGATGCCCT
GCCAATCGAGATGCTGGACAGGCATCATACCCACTTCTGCCCCCTGGAAGGCGAG
TCATGGCAAGACTTTCTGCGGAACAACGCCAAGTCATTCCGCTGTGCTCTCCTCTC
ACATCGCGACGGGGCTAAAGTGCATCTCGGCACCCGCCCAACAGAGAAACAGTA
CGAAACCCTGGAAAATCAGCTCGCGTTCCTGTGTCAGCAAGGCTTCTCCCTGGAG
AACGCACTGTACGCTCTGTCCGCCGTGGGCCACTTTACACTGGGCTGCGTATTGGA
GGAACAGGAGCATCAAGTAGCAAAAGAGGAAAGAGAGACACCTACCACCGATTC
TATGCCCCCACTTCTGAGACAAGCAATTGAGCTGTTCGACCGGCAGGGAGCCGAA
CCTGCCTTCCTTTTCGGCCTGGAACTAATCATATGTGGCCTGGAGAAACAGCTAAA
GTGCGAAAGCGGCGGGCCGGCCGACGCCCTTGACGATTTTGACTTAGACATGCTC
CCAGCCGATGCCCTTGACGACTTTGACCTTGATATGCTGCCTGCTGACGCTCTTGA
CGATTTTGACCTTGACATGCTCCCCGGG
Amino acid sequence:
(SEQ ID NO: 18)
MSRLDKSKVINGALELLNGVGIEGLTTRKLAQKLGVEQPTLYWHVKNKRALLDALPI
EMLDRHHTHFCPLEGESWQDFLRNNAKSFRCALLSHRDGAKVHLGTRPTEKQYETL
ENQLAFLCQQGFSLENALYALSAVGHFTLGCVLEEQEHQVAKEERETPTTDSMPPLLR
QAIELFDRQGAEPAFLFGLELIICGLEKQLKCESGGPADALDDFDLDMLPADALDDED
LDMLPADALDDFDLDMLPG
Tetracycline transcriptional silencer (tTS):
Nucleotide sequence:
(SEQ ID NO: 19)
ATGTCTAGATTAGATAAAAGTAAAGTGATTAACAGCGCATTAGAGCTGCTTAATGA
GGTCGGAATCGAAGGTTTAACAACCCGTAAACTCGCCCAGAAGCTAGGTGTAGAG
CAGCCTACATTGTATTGGCACGTGCGCAACAAGCAGACTCTTATGAACATGCTTTC
AGAGGCAATACTGGCGAAGCATCACACCCGTTCAGCACCGTTACCGACTGAGAGT
TGGCAGCAGTTTCTCCAGGAAAATGCTCTGAGTTTCCGTAAAGCATTACTGGTCCA
TCGTGATGGAGCCCGATTGCATATAGGGACCTCTCCTACGCCCCCCCAGTTTGAAC
AAGCAGAGGCGCAACTACGCTGTCTATGCGATGCAGGGTTTTCGGTCGAGGAGGC
TCTTTTCATTCTGCAATCTATCAGCCATTTTACGTTGGGTGCAGTATTAGAGGAGCA
AGCAACAAACCAGATAGAAAATAATCATGTGATAGACGCTGCACCACCATTATTAC
AAGAGGCATTTAATATTCAGGCGAGAACCTCTGCTGAAATGGCCTTCCATTTCGGG
CTGAAATCATTAATATTTGGATTTTCTGCACAGTTAGATGAAAAAAAGCATACACC
CATTGAGGATGGTAATAAACCAAAAAAGAAGAGAAAGCTAGCAGTGTCAGTGAC
ATTTGAAGATGTGGCTGTGCTCTTTACTCGGGACGAGTGGAAGAAGCTGGATCTG
TCTCAGAGAAGCCTGTACCGTGAGGTGATGCTGGAGAATTACAGCAACCTGGCCT
CCATGGCAGGATTCCTGTTTACCAAACCAAAGGTGATCTCCCTGTTGCAGCAAGG
AGAGGATCCCTGG
Amino acid sequence:
(SEQ ID NO: 20)
MSRLDKSKVINSALELLNEVGIEGLTTRKLAQKLGVEQPTLYWHVRNKQTLMNMLS
EAILAKHHTRSAPLPTESWQQFLQENALSFRKALLVHRDGARLHIGTSPTPPQFEQAE
AQLRCLCDAGFSVEEALFILQSISHFTLGAVLEEQATNQIENNHVIDAAPPLLQEAFNI
QARTSAEMAFHFGLKSLIFGFSAQLDEKKHTPIEDGNKPKKKRKLAVSVTFEDVAVLF
TRDEWKKLDLSQRSLYREVMLENYSNLASMAGFLFTKPKVISLLQQGEDPW
Therapeutic transgene: furin-cleavable human insulin
Nucleotide sequence:
(SEQ ID NO: 21)
Atggccctgtggatgagactgctgcctctgctggccctcctggccctgtggggccccgaccccgccgccgcttttgtgaaccagcac
ctgtgcggaagcgacctggtcgaggccctgtacctggtgtgcggcgagcggggcttcttctacacccctagaaccaagcgggaagc
cgaggatctgcaggtgggccaggtggagctgggcggcggaccaggcgccggctccctgcaacctctggctctggaaggatctaga
cagaagagaggcatcgtggagcagtgttgtacaagcatctgcagcctgtatcagctggaaaactactgcaat
Amino acid sequence:
(SEQ ID NO: 22)
MALWMRLLPLLALLALWGPDPAAAFVNQHLCGSDLVEALYLVCGERGFFYTPRTKR
EAEDLQVGQVELGGGPGAGSLQPLALEGSRQKRGIVEQCCTSICSLYQLENYCN
Auxiliary Sequences:
Tetracycline-Responsive Element Promoter (TRE promoter)
(SEQ ID NO: 23)
GGTACCGAGCTCGACTTTCACTTTTCTCTATCACTGATAGGGAGTGGTAAACTCGA
CTTTCACTTTTCTCTATCACTGATAGGGAGTGGTAAACTCGACTTTCACTTTTCTCT
ATCACTGATAGGGAGTGGTAAACTCGACTTTCACTTTTCTCTATCACTGATAGGGA
GTGGTAAACTCGACTTTCACTTTTCTCTATCACTGATAGGGAGTGGTAAACTCGAC
TTTCACTTTTCTCTATCACTGATAGGGAGTGGTAAACTCGACTTTCACTTTTCTCTA
TCACTGATAGGGAGTGGTAAACTCGACCTATATAAGCAGAGCTCGTTTAGTGAACC
GTCAGATCGCCTGGAGACGCCATCCACGCTGTTTTGACCTCCATAGAAGACACCG
GGACCGATCCAGCCTCCGCGGCCCCGAATTG
BGH pA
(SEQ ID NO: 24)
CTGTGCCTTCTAGTTGCCAGCCATCTGTTGTTTGCCCCTCCCCCGTGCCTTCCTTGA
CCCTGGAAGGTGCCACTCCCACTGTCCTTTCCTAATAAAATGAGGAAATTGCATCG
CATTGTCTGAGTAGGTGTCATTCTATTCTGGGGGGTGGGGTGGGGCAGGACAGCA
AGGGGGAGGATTGGGAAGACAATAGCAGGCATGCTGGGGATGCGGTGGGCTCTAT
GG
SV40 late pA
(SEQ ID NO: 25)
CAGACATGATAAGATACATTGATGAGTTTGGACAAACCACAACTAGAATGCAGTG
AAAAAAATGCTTTATTTGTGAAATTTGTGATGCTATTGCTTTATTTGTAACCATTATA
AGCTGCAATAAACAAGTTAACAACAACAATTGCATTCATTTTATGTTTCAGGTTCA
GGGGGAGGTGTGGGAGGTTTTTTAAAGCAAGTAAAACCTCTACAAATGTGGTA
Kozak
GCCACC
Ribosome-skipping 2A peptide from Thosea Asigna Virus
Nucleotide sequence:
(SEQ ID NO: 26)
GAGGGCAGGGGAAGTCTTCTAACATGCGGGGACGTGGAGGAAAATCCCGGCCCC
Amino acid sequence:
(SEQ ID NO: 27)
EGRGSLLTCGDVEENPGP
P2A
Nucleotide sequence
(SEQ ID NO: 28)
GCCACAAACTTCAGCCTGCTGAAGCAGGCCGGAGATGTGGAAGAAAATCCTGGC
CCC
Amino acid sequence
(SEQ ID NO: 29)
ATNFSLLKQAGDVEENPGP
EFS promoter
(SEQ ID NO: 30)
GGCTCCGGTGCCCGTCAGTGGGCAGAGCGCACATCGCCCACAGTCCCCGAGAAG
TTGGGGGGAGGGGTCGGCAATTGATCCGGTGCCTAGAGAAGGTGGCGCGGGGTA
AACTGGGAAAGTGATGTCGTGTACTGGCTCCGCCTTTTTCCCGAGGGTGGGGGAG
AACCGTATATAAGTGCAGTAGTCGCCGTGAACGTTCTTTTTCGCAACGGGTTTGCC
GCCAGAACACAGG

### Example 2

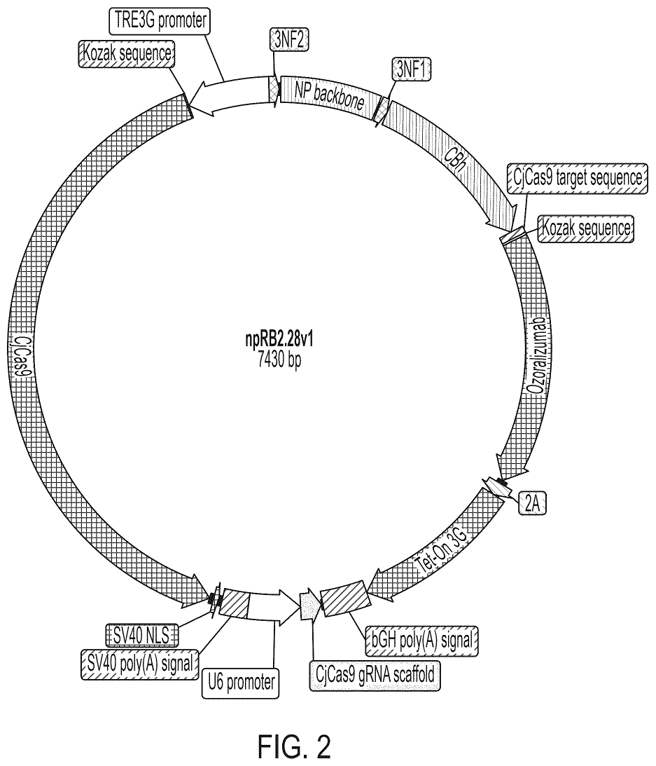

In the present example, a doxycycline-inducible, self-inactivating construct is delivered in cis with a therapeutic transgene encoding a secretable anti-TNF-alpha nanobody (Ozoralizumab). To reduce the level of anti-TNF-alpha nanobody expression, an in-cis delivered Campylobacter jejuni CRISPR Cas9 protein is inducibly expressed under the regulation of a third generation Tetracycline repressor/operator (rtTA/TRE3g). Upon delivery of doxycycline, the Cas9 and sgRNA are expressed from the construct and target a sequence on the construct to separate the promoter from the therapeutic transgene. FIG. 2 shows a plasmid map for the genetic construct described in this example. The sequences and constructs are shown below:

Full sequence (except nanoplasmid backbone)
(SEQ ID NO: 31)
ctggggactttccagcctggggactttccagctgggactttccaggcgttacataacttacggtaaatggcccgcctggctgaccgccc
aacgacccccgcccattgacgtcaatagtaacgccaatagggactttccattgacgtcaatgggtggagtatttacggtaaactgccca
cttggcagtacatcaagtgtatcatatgccaagtacgccccctattgacgtcaatgacggtaaatggcccgcctggcattgtgcccagta
catgaccttatgggactttcctacttggcagtacatctacgtattagtcatcgctattaccatggtcgaggtgagccccacgttctgcttcac
tctccccatctcccccccctccccacccccaattttgtatttatttattttttaattattttgtgcagcgatgggggcgggggggggggggg
ggcgcgcgccaggcggggcggggcggggcgaggggcggggcggggcgaggcggagaggtgcggcggcagccaatcagag
cggcgcgctccgaaagtttccttttatggcgaggcggcggcggcggcggccctataaaaagcgaagcgcgcggcgggcgggagt
cgctgcgcgctgccttcgccccgtgccccgctccgccgccgcctcgcgccgcccgccccggctctgactgaccgcgttactcccac
aggtgagcgggcgggacggcccttctcctccgggctgtaattagctgagcaagaggtaagggtttaagggatggttggttggtgggg
tattaatgtttaattacctggagcacctgcctgaaatcactttttttcaggttggtcgcaacgggtttgccgccagagtgaacacgccacca
tggactggacatggcgggtgttctgcctgctggccgtggcccctggtgctcacagcgaagtgcagctggtggaaagcggcggcggc
ctggtgcagcccggcggaagcctccgcctgagttgcgctgccagcggcttcaccttcagcgactactggatgtactgggtgcggcag
gcccctggcaaaggcctggaatgggtctctgaaatcaacaccaacggccttatcaccaagtaccccgactccgtgaagggccggttc
accatctccagagataatgccaaaaacaccctgtacctgcagatgaacagcctgcggcccgaggacacagccgtgtactactgcgct
agaagcccttctggctttaaccggggccagggcacccttgttacagtgtcaagcggcggaggtggctctggcggtggatctgaggtg
cagctggttgagagcggaggcggactggtgcaacctggcaatagcctgagactgtcttgtgccgcctctggtttcaccttttctagcttc
ggcatgagctgggtcagacaggctcctggcaagggcctggaatgggtgtccagcatcagcggaagcggctccgacaccctgtacg
ccgacagcgtgaaagggagattcacaatcagcagggacaatgccaagaccaccctgtacctgcaaatgaacagcctgagacctgag
gacaccgccgtgtactattgcaccatcggcggaagcctgagcagatccagccagggcaccctggtgacagtgtcttctggtggtggc
ggatcaggaggcgggagcgaggtgcagctggtcgagagcggcggcggactggtgcagccaggcggaagcctgagactgagctg
tgccgctagcggcttcacattcagcgattactggatgtactgggtgcggcaggcccctggaaagggcctcgagtgggtctccgagatc
aacacaaacggcctgatcacaaagtacccagatagcgtgaagggcagatttacaatttctagagacaacgccaagaacaccctgtatc
tgcagatgaacagcctgcggcctgaggataccgccgtgtactactgcgccagaagccccagcggattcaacagaggccagggcac
cctggtgaccgtgtcctccagaaagaggggggatctggcgccacaaacttcagcctgctgaagcaggccggagatgtggaagaa
aatcctggccccatgagcagactggataagtccaaggtgatcaacagcgccctggaactgctcaacggcgtcggcatcgagggact
caccacccggaagctggcccagaaactgggcgtggaacaacctacactgtactggcacgtgaagaacaaaagagccctgctggat
gccctgccaatcgagatgctggaccggcaccatacccacagctgccccctggagggagaaagctggcaggacttcctgagaaaca
acgctaagagctaccggtgcgccctgctgagccaccgggacggcgccaaggtgcacttggggaccagacccacggaaaagcaat
acgagacactggagaaccagctggccttcctgtgccagcagggcttcagcctggagaatgccctctacgccctgtctgccgtgggcc
actttaccctgggatgtgtgctggaagagcaggagcaccaggtggccaaggaagagagggaaacccctaccaccgacagcatgcc
tcctctgctgaagcaagctatcgagctgttcgacagacagggcgccgagcctgctttcctgtttggcctggagctgatcatctgcggctt
ggagaaacagctgaagtgcgagagcggcggccctaccgacgccctggacgatttcgacctggatatgctgcctgccgacgccctg
gacgacttcgacctggatatgctgcctgccgatgctctggacgacttcgacctggacatgcttccaggctaactgtgccttctagttgcc
agccatctgttgtttgcccctcccccgtgccttccttgaccctggaaggtgccactcccactgtcctttcctaataaaatgaggaaattgca
tcgcattgtctgagtaggtgtcattctattctggggggtggggtggggcaggacagcaagggggaggattgggaagagaatagcag
gcaggctggggaaaaaaagcggttttaggggattgtaaccccgcagagtcccgcaaactctttattttagtcccttcagggactaaaact
ctggcggcaaacccgttgcgacgtcctttccacaagatatataaagccaagaaatcgaaatactttcaagttacggtaagcatatgatag
tccattttaaaacataattttaaaactgcaaactacccaagaaattattactttctacgtcacgtattttgtactaatatctttgtgtttacagt
caaattaattccaattatctctctaacagccttgtatcgtatatgcaaatatgaaggaatcatgggaaataggccctcaacttgtttattgcagc
ttataatggttacaaataaagcaatagcatcacaaatttcacaaataaagcatttttttcactgcattctagttgtggtttgtccaaactcatca
atgtatcttatcattattaggcgtagtcgggcacgtcgtaggggtatacttttctcttttttttggaggtccggactttttgaagtcctctctct
gccggaactcggcctttgtcacttctcccagggcggacacgatgtacttctcgaacaccttcagattctggatgccgatgctcttggcgat
cacttctttctcgttggcgttcttgaacaggatcttctggttcttgctcagtgtctcgaacttgttgtcgtgcttagacacgatcagggacacg
gtgctgctggtgaaggcgttgtagtagacgaactcgggttcctgcatgtccttggtctggatcaggatcagggagtccttgtacaggcta
aagcagaactcgtagttctcgtccatcagaatccagtccttgatctcgcccttcttggaccgggccacggccttgttgggcagcaccttc
agggcgaagtccatggtgtagatgggcacggcgtagaacttgttggtctttttgtgcttgaagatgtccacccggaacatgtccccgttc
ttcacgatcttgccgttcacttttctgatcttgcccagctcgagggccttcagcacgccttctttgccgccgtaggactggtagaactcttcc
tcttttctgaaggtttcctcgtgcagggcgccagagggcttcttccgctcgggcttggacacgaagatctcgtcgatcttatccagcacct
tctgccggaagccgctgaagggctcgaagaacttccgcttgttcttgtagtccagctcagagatcttcttggcgtacagctcggcgctgt
tgctttcctgttctttcttgaagtcggagaaggccttcacgatgctgttgttggcgtaggcaatgatcacggcgtcgatggcgtggtgcag
atggttgttccgatccttggcgctgaagccccaggtgtgtctcagggcgctggtcagcatgccgctcttggcttccacgtgcaccttgga
gcctttctgggtatcgttcagcttggtgttctcgtcgtcggacaggggcaggaaatccaggtagtcctttgtgtagttcagcaccagccg
ggcgatataccgggtgtcgttcaggttccggtccttgaagttcttctgctctttgtccttgtaattcttgtccaggattctcttctgtttcttgg
tgggcaggttcttggccagcacctcgattttctgccacttggcgctgtcgttgccgaaggcctcgaagggggtctggttcagtttttcctggt
tctgtttggtgaacaccagcaccttgttcatgtagctgtcgtcgaaggaccggctgtaggggtagatgtggtcgatttccagcatcttctc
gtcctgcagatcgctgatcttgatcttctcgccgctgtaggcgcagaactctttctgttctttgaacagccgcagcttcaggatgttcttgct
gttgatcttcagtcccagcttctcgcattccagctcagcgtccttcttggccttgtagttttcgttctgctctttctcgatcttggcccgctggct
gtggttcttgcccacttcccgggccagctcgatgttgatcttgtgcaccttgccgtatttcttgagcagggcattcagcaccttccggtattc
tttgatggcccgcagcaccacggggttggtcacttcgtccttgtagtaggtttcgttgaaggcgggcaggaagtccttcttgtcctcgttg
atggccaccttcaggttcagctcgttgcaggcctcgtcgtacttcttgccttccagcatcaggggggtgaccagcttcagagccttgaag
ctgatgttcaggtgatccttgaattccagcttgctcaggctgtcgatctggttctgattcaggtcgtatttggccagggccttcttcagcttaa
tctcgtccttgatcagggtgatgtccttggcgatctcattcagatcgtcctggctcaggttgtgctcgcccagggccttgatgaactctttgt
acttcttgaactcgatgaagtaggtgcccttctcgcccttaaactcgtagtcgtcgctcaggcccagcagcttcttggtctgcttgtaggtc
agggtgccgttcttcagcacctcattcagcagggcgttcaggtcgtccttggtgtacaggatgccctcggtgttcttcagattgttcagca
ggttgatgatccgggtcagggccacgaacatgaaggccagggggctgttcttgggggctctcttctcgtcggtgaagaagctgcagtt
gcccacgaggtggctgaagtccttcagggctctcttgtagaaggcgacgctcagcacctcttcctcgaatttcttgctgaagctgaagc
cgaactctctctgcttcttgaagatcagcttcagctcatccttcaggaagctctgggcgatacaccgctcgtagctttctttcttgttccgca
cgttggtgaattctttgctgttctctttgaacttctggaagtactctttgtacaggtactcgcccacggactggtagttggccagcttttcctc
gttctgcttgatggccttcaggatggcgcccttctctttgtcgtcgctgttcttgatgtcgtcgtagcctctccgcttggcaatgtgcaggat
cactctggcgaagtcctgcttgctcagcagctcgttcagggcccggaaccgcagctcgtaggggctgatcaggctgcccttgtaggc
cttggccagggactcgtcgaagctctggtagtcctcgtagttcagcttgaactcgttggcgatcaggtgcttcaggtggttcagccggg
ccttccgtctggccagtctcttccgggcgcttctggccagccttctgggcagggccaggctctcgccggtcttggggttttccaccttgg
tgaagatccgcacgccgcagtccttcagctcgtcgttctcgctgaaggcccagccgatgctgctgatgccgatgtcgaaggccaggat
tctggccatggtggctttacgagggtaggaagtggtacggaaagttggtataagacaaaagtgttgtggaattgctccaggcgatctga
cggttcactaaacgagctctgcttttataggcgcccaccgtacacgcctaaagcttatacgttctctatcactgatagggagtaaactgga
tatacgttctctatcactgatagggagtaaactgtagatacgttctctatcactgatagggagtaaactggtcatacgttctctatcactgata
gggagtaaactccttatacgttctctatcactgatagggagtaaagtctgcatacgttctctatcactgatagggagtaaactcttcatacgt
tctctatcactgatagggagtaaactggggactttccagctggggactttccagctgggactttccaggag
Actuator: doxycycline or Doxycycline Hyclate, CAS RN: 24390-14-5, molecular formula
C12H24N208.1/2C2H60.CIH.1/22H2O.
3NF1
(SEQ ID NO: 10)
Ctggggactttccagcctggggactttccagctgggactttccagg
3NF2
(SEQ ID NO: 11)
Ctggggactttccagctggggactttccagctgggactttccaggag
CBh promoter
(SEQ ID NO: 32)
CGTTACATAACTTACGGTAAATGGCCCGCCTGGCTGACCGCCCAACGACCCCCGC
CCATTGACGTCAATAGTAACGCCAATAGGGACTTTCCATTGACGTCAATGGGTGGA
GTATTTACGGTAAACTGCCCACTTGGCAGTACATCAAGTGTATCATATGCCAAGTAC
GCCCCCTATTGACGTCAATGACGGTAAATGGCCCGCCTGGCATTGTGCCCAGTACA
TGACCTTATGGGACTTTCCTACTTGGCAGTACATCTACGTATTAGTCATCGCTATTAC
CATGGTCGAGGTGAGCCCCACGTTCTGCTTCACTCTCCCCATCTCCCCCCCCTCCC
CACCCCCAATTTTGTATTTATTTATTTTTTAATTATTTTGTGCAGCGATGGGGGCGGG
GGGGGGGGGGGGGCGCGCGCCAGGCGGGGCGGGGCGGGGCGAGGGGCGGGGC
GGGGCGAGGCGGAGAGGTGCGGCGGCAGCCAATCAGAGCGGCGCGCTCCGAAA
GTTTCCTTTTATGGCGAGGCGGCGGCGGCGGCGGCCCTATAAAAAGCGAAGCGCG
CGGCGGGCGGGAGTCGCTGCGCGCTGCCTTCGCCCCGTGCCCCGCTCCGCCGCCG
CCTCGCGCCGCCCGCCCCGGCTCTGACTGACCGCGTTACTCCCACAGGTGAGCGG
GCGGGACGGCCCTTCTCCTCCGGGCTGTAATTAGCTGAGCAAGAGGTAAGGGTTT
AAGGGATGGTTGGTTGGTGGGGTATTAATGTTTAATTACCTGGAGCACCTGCCTGA
AATCACTTTTTTTCAGGTTGG
Ozoralizumab
Nucleotide sequence
(SEQ ID NO: 33)
ATGGACTGGACATGGCGGGTGTTCTGCCTGCTGGCCGTGGCCCCTGGTGCTCACA
GCGAAGTGCAGCTGGTGGAAAGCGGCGGCGGCCTGGTGCAGCCCGGCGGAAGC
CTCCGCCTGAGTTGCGCTGCCAGCGGCTTCACCTTCAGCGACTACTGGATGTACTG
GGTGCGGCAGGCCCCTGGCAAAGGCCTGGAATGGGTCTCTGAAATCAACACCAA
CGGCCTTATCACCAAGTACCCCGACTCCGTGAAGGGCCGGTTCACCATCTCCAGA
GATAATGCCAAAAACACCCTGTACCTGCAGATGAACAGCCTGCGGCCCGAGGACA
CAGCCGTGTACTACTGCGCTAGAAGCCCTTCTGGCTTTAACCGGGGCCAGGGCAC
CCTTGTTACAGTGTCAAGCGGCGGAGGTGGCTCTGGCGGTGGATCTGAGGTGCAG
CTGGTTGAGAGCGGAGGCGGACTGGTGCAACCTGGCAATAGCCTGAGACTGTCTT
GTGCCGCCTCTGGTTTCACCTTTTCTAGCTTCGGCATGAGCTGGGTCAGACAGGCT
CCTGGCAAGGGCCTGGAATGGGTGTCCAGCATCAGCGGAAGCGGCTCCGACACC
CTGTACGCCGACAGCGTGAAAGGGAGATTCACAATCAGCAGGGACAATGCCAAG
ACCACCCTGTACCTGCAAATGAACAGCCTGAGACCTGAGGACACCGCCGTGTACT
ATTGCACCATCGGCGGAAGCCTGAGCAGATCCAGCCAGGGCACCCTGGTGACAGT
GTCTTCTGGTGGTGGCGGATCAGGAGGCGGGAGCGAGGTGCAGCTGGTCGAGAG
CGGCGGCGGACTGGTGCAGCCAGGCGGAAGCCTGAGACTGAGCTGTGCCGCTAG
CGGCTTCACATTCAGCGATTACTGGATGTACTGGGTGCGGCAGGCCCCTGGAAAG
GGCCTCGAGTGGGTCTCCGAGATCAACACAAACGGCCTGATCACAAAGTACCCAG
ATAGCGTGAAGGGCAGATTTACAATTTCTAGAGACAACGCCAAGAACACCCTGTA
TCTGCAGATGAACAGCCTGCGGCCTGAGGATACCGCCGTGTACTACTGCGCCAGA
AGCCCCAGCGGATTCAACAGAGGCCAGGGCACCCTGGTGACCGTGTCCTCC
Amino acid sequence
(SEQ ID NO: 34)
MDWTWRVFCLLAVAPGAHSEVOLVESGGGLVQPGGSLRLSCAASGFTFSDYWMYW
VRQAPGKGLEWVSEINTNGLITKYPDSVKGRFTISRDNAKNTLYLQMNSLRPEDTAV
YYCARSPSGFNRGQGTLVTVSSGGGGSGGGSEVQLVESGGGLVQPGNSLRLSCAASG
FTFSSFGMSWVRQAPGKGLEWVSSISGSGSDTLYADSVKGRFTISRDNAKTTLYLQM
NSLRPEDTAVYYCTIGGSLSRSSQGTLVTVSSGGGGSGGGSEVQLVESGGGLVQPGGS
LRLSCAASGFTFSDYWMYWVRQAPGKGLEWVSEINTNGLITKYPDSVKGRFTISRD
NAKNTLYLQMNSLRPEDTAVYYCARSPSGFNRGQGTLVTVSS
Furin cleavage site
Nucleotide sequence
(SEQ ID NO: 35)
AGAAAGAGGCGG
Amino acid sequene
(SEQ ID NO: 36)
RKRR
P2A
Nucleotide sequence
(SEQ ID NO: 37)
GGATCTGGCGCCACAAACTTCAGCCTGCTGAAGCAGGCCGGAGATGTGGAAGAA
AATCCTGGCCCC
Amino acid sequence
(SEQ ID NO: 38)
GSGATNFSLLKQAGDVEENPGP
Effector element: Cas9
(SEQ ID NO: 39)
ATGGCCAGAATCCTGGCCTTCGACATCGGCATCAGCAGCATCGGCTGGGCCTTCA
GCGAGAACGACGAGCTGAAGGACTGCGGCGTGCGGATCTTCACCAAGGTGGAAA
ACCCCAAGACCGGCGAGAGCCTGGCCCTGCCCAGAAGGCTGGCCAGAAGCGCCC
GGAAGAGACTGGCCAGACGGAAGGCCCGGCTGAACCACCTGAAGCACCTGATCG
CCAACGAGTTCAAGCTGAACTACGAGGACTACCAGAGCTTCGACGAGTCCCTGG
CCAAGGCCTACAAGGGCAGCCTGATCAGCCCCTACGAGCTGCGGTTCCGGGCCCT
GAACGAGCTGCTGAGCAAGCAGGACTTCGCCAGAGTGATCCTGCACATTGCCAA
GCGGAGAGGCTACGACGACATCAAGAACAGCGACGACAAAGAGAAGGGCGCCA
TCCTGAAGGCCATCAAGCAGAACGAGGAAAAGCTGGCCAACTACCAGTCCGTGG
GCGAGTACCTGTACAAAGAGTACTTCCAGAAGTTCAAAGAGAACAGCAAAGAAT
TCACCAACGTGCGGAACAAGAAAGAAAGCTACGAGCGGTGTATCGCCCAGAGCT
TCCTGAAGGATGAGCTGAAGCTGATCTTCAAGAAGCAGAGAGAGTTCGGCTTCA
GCTTCAGCAAGAAATTCGAGGAAGAGGTGCTGAGCGTCGCCTTCTACAAGAGAG
CCCTGAAGGACTTCAGCCACCTCGTGGGCAACTGCAGCTTCTTCACCGACGAGAA
GAGAGCCCCCAAGAACAGCCCCCTGGCCTTCATGTTCGTGGCCCTGACCCGGATC
ATCAACCTGCTGAACAATCTGAAGAACACCGAGGGCATCCTGTACACCAAGGACG
ACCTGAACGCCCTGCTGAATGAGGTGCTGAAGAACGGCACCCTGACCTACAAGC
AGACCAAGAAGCTGCTGGGCCTGAGCGACGACTACGAGTTTAAGGGCGAGAAGG
GCACCTACTTCATCGAGTTCAAGAAGTACAAAGAGTTCATCAAGGCCCTGGGCGA
GCACAACCTGAGCCAGGACGATCTGAATGAGATCGCCAAGGACATCACCCTGATC
AAGGACGAGATTAAGCTGAAGAAGGCCCTGGCCAAATACGACCTGAATCAGAAC
CAGATCGACAGCCTGAGCAAGCTGGAATTCAAGGATCACCTGAACATCAGCTTCA
AGGCTCTGAAGCTGGTCACCCCCCTGATGCTGGAAGGCAAGAAGTACGACGAGG
CCTGCAACGAGCTGAACCTGAAGGTGGCCATCAACGAGGACAAGAAGGACTTCC
TGCCCGCCTTCAACGAAACCTACTACAAGGACGAAGTGACCAACCCCGTGGTGCT
GCGGGCCATCAAAGAATACCGGAAGGTGCTGAATGCCCTGCTCAAGAAATACGGC
AAGGTGCACAAGATCAACATCGAGCTGGCCCGGGAAGTGGGCAAGAACCACAGC
CAGCGGGCCAAGATCGAGAAAGAGCAGAACGAAAACTACAAGGCCAAGAAGGA
CGCTGAGCTGGAATGCGAGAAGCTGGGACTGAAGATCAACAGCAAGAACATCCT
GAAGCTGCGGCTGTTCAAAGAACAGAAAGAGTTCTGCGCCTACAGCGGCGAGAA
GATCAAGATCAGCGATCTGCAGGACGAGAAGATGCTGGAAATCGACCACATCTAC
CCCTACAGCCGGTCCTTCGACGACAGCTACATGAACAAGGTGCTGGTGTTCACCA
AACAGAACCAGGAAAAACTGAACCAGACCCCCTTCGAGGCCTTCGGCAACGACA
GCGCCAAGTGGCAGAAAATCGAGGTGCTGGCCAAGAACCTGCCCACCAAGAAAC
AGAAGAGAATCCTGGACAAGAATTACAAGGACAAAGAGCAGAAGAACTTCAAGG
ACCGGAACCTGAACGACACCCGGTATATCGCCCGGCTGGTGCTGAACTACACAAA
GGACTACCTGGATTTCCTGCCCCTGTCCGACGACGAGAACACCAAGCTGAACGAT
ACCCAGAAAGGCTCCAAGGTGCACGTGGAAGCCAAGAGCGGCATGCTGACCAGC
GCCCTGAGACACACCTGGGGCTTCAGCGCCAAGGATCGGAACAACCATCTGCAC
CACGCCATCGACGCCGTGATCATTGCCTACGCCAACAACAGCATCGTGAAGGCCT
TCTCCGACTTCAAGAAAGAACAGGAAAGCAACAGCGCCGAGCTGTACGCCAAGA
AGATCTCTGAGCTGGACTACAAGAACAAGCGGAAGTTCTTCGAGCCCTTCAGCGG
CTTCCGGCAGAAGGTGCTGGATAAGATCGACGAGATCTTCGTGTCCAAGCCCGAG
CGGAAGAAGCCCTCTGGCGCCCTGCACGAGGAAACCTTCAGAAAAGAGGAAGA
GTTCTACCAGTCCTACGGCGGCAAAGAAGGCGTGCTGAAGGCCCTCGAGCTGGG
CAAGATCAGAAAAGTGAACGGCAAGATCGTGAAGAACGGGGACATGTTCCGGGT
GGACATCTTCAAGCACAAAAAGACCAACAAGTTCTACGCCGTGCCCATCTACACC
ATGGACTTCGCCCTGAAGGTGCTGCCCAACAAGGCCGTGGCCCGGTCCAAGAAG
GGCGAGATCAAGGACTGGATTCTGATGGACGAGAACTACGAGTTCTGCTTTAGCC
TGTACAAGGACTCCCTGATCCTGATCCAGACCAAGGACATGCAGGAACCCGAGTT
CGTCTACTACAACGCCTTCACCAGCAGCACCGTGTCCCTGATCGTGTCTAAGCACG
ACAACAAGTTCGAGACACTGAGCAAGAACCAGAAGATCCTGTTCAAGAACGCCA
ACGAGAAAGAAGTGATCGCCAAGAGCATCGGCATCCAGAATCTGAAGGTGTTCG
AGAAGTACATCGTGTCCGCCCTGGGAGAAGTGACAAAGGCCGAGTTCCGGCAGA
GAGAGGACTTCAAAAAGTCCGGACCTCCAAAGAAAAAGAGAAAAGTATACCCCT
ACGACGTGCCCGACTACGCCTAA
Amino acid sequence
(SEQ ID NO: 40)
MARILAFDIGISSIGWAFSENDELKDCGVRIFTKVENPKTGESLALPRRLARSARKRLA
RRKARLNHLKHLIANEFKLNYEDYQSFDESLAKAYKGSLISPYELRFRALNELLSKQD
FARVILHIAKRRGYDDIKNSDDKEKGAILKAIKQNEEKLANYQSVGEYLYKEYFQKF
KENSKEFTNVRNKKESYERCIAQSFLKDELKLIFKKQREFGFSFSKKFEEEVLSVAFYK
RALKDFSHLVGNCSFFTDEKRAPKNSPLAFMFVALTRIINLLNNLKNTEGILYTKDDL
NALLNEVLKNGTLTYKQTKKLLGLSDDYEFKGEKGTYFIEFKKYKEFIKALGEHNLS
QDDLNEIAKDITLIKDEIKLKKALAKYDLNQNQIDSLSKLEFKDHLNISFKALKLVTPL
MLEGKKYDEACNELNLKVAINEDKKDFLPAFNETYYKDEVTNPVVLRAIKEYRKVL
NALLKKYGKVHKINIELAREVGKNHSQRAKIEKEQNENYKAKKDAELECEKLGLKI
NSKNILKLRLFKEQKEFCAYSGEKIKISDLQDEKMLEIDHIYPYSRSFDDSYMNKVLVF
TKQNQEKLNQTPFEAFGNDSAKWQKIEVLAKNLPTKKQKRILDKNYKDKEQKNFK
DRNLNDTRYIARLVLNYTKDYLDFLPLSDDENTKLNDTQKGSKVHVEAKSGMLTSA
LRHTWGFSAKDRNNHLHHAIDAVIIAYANNSIVKAFSDFKKEQESNSAELYAKKISEL
DYKNKRKFFEPFSGFRQKVLDKIDEIFVSKPERKKPSGALHEETFRKEEEFYQSYGGK
EGVLKALELGKIRKVNGKIVKNGDMFRVDIFKHKKTNKFYAVPIYTMDFALKVLPNK
AVARSKKGEIKDWILMDENYEFCFSLYKDSLILIQTKDMQEPEFVYYNAFTSSTVSLIV
SKHDNKFETLSKNQKILFKNANEKEVIAKSIGIQNLKVFEKYIVSALGEVTKAEFRQR
EDFKKSGPPKKKRKVYPYDVPDYA
Effector sequence: target of cjCas9
(SEQ ID NO: 41)
TCGCAACGGGTTTGCCGCCAGA
PAM
NNNNACAC (N-any nucleotide)
U6 promoter
(SEQ ID NO: 15)
GAGGGCCTATTTCCCATGATTCCTTCATATTTGCATATACGATACAAGGCTGTTAGA
GAGATAATTGGAATTAATTTGACTGTAAACACAAAGATATTAGTACAAAATACGTG
ACGTAGAAAGTAATAATTTCTTGGGTAGTTTGCAGTTTTAAAATTATGTTTTAAAAT
GGACTATCATATGCTTACCGTAACTTGAAAGTATTTCGATTTCTTGGCTTTATATATC
TTGTGGAAAGGAC
gRNA scaffold for CjCas9
(SEQ ID NO: 42)
TCGCAACGGGTTTGCCGCCAGAGTTTTAGTCCCTGAAGGGACTAAAATAAAGAGT
TTGCGGGACTCTGCGGGGTTACAATCCCCTAAAACCGCTTTTTT
Actuator response element: TetRepressor/Gen 3 TetOn, third generation rtTA/TRE3G
Nucleotide sequence
(SEQ ID NO: 43)
ATGAGCAGACTGGATAAGTCCAAGGTGATCAACAGCGCCCTGGAACTGCTCAACG
GCGTCGGCATCGAGGGACTCACCACCCGGAAGCTGGCCCAGAAACTGGGCGTGG
AACAACCTACACTGTACTGGCACGTGAAGAACAAAAGAGCCCTGCTGGATGCCCT
GCCAATCGAGATGCTGGACCGGCACCATACCCACAGCTGCCCCCTGGAGGGAGAA
AGCTGGCAGGACTTCCTGAGAAACAACGCTAAGAGCTACCGGTGCGCCCTGCTG
AGCCACCGGGACGGCGCCAAGGTGCACTTGGGGACCAGACCCACGGAAAAGCA
ATACGAGACACTGGAGAACCAGCTGGCCTTCCTGTGCCAGCAGGGCTTCAGCCTG
GAGAATGCCCTCTACGCCCTGTCTGCCGTGGGCCACTTTACCCTGGGATGTGTGCT
GGAAGAGCAGGAGCACCAGGTGGCCAAGGAAGAGAGGGAAACCCCTACCACCG
ACAGCATGCCTCCTCTGCTGAAGCAAGCTATCGAGCTGTTCGACAGACAGGGCGC
CGAGCCTGCTTTCCTGTTTGGCCTGGAGCTGATCATCTGCGGCTTGGAGAAACAG
CTGAAGTGCGAGAGCGGCGGCCCTACCGACGCCCTGGACGATTTCGACCTGGATA
TGCTGCCTGCCGACGCCCTGGACGACTTCGACCTGGATATGCTGCCTGCCGATGCT
CTGGACGACTTCGACCTGGACATGCTTCCAGGCTAA
Amino acid sequence
(SEQ ID NO: 44)
MSRLDKSKVINSALELLNGVGIEGLTTRKLAQKLGVEQPTLYWHVKNKRALLDALPI
EMLDRHHTHSCPLEGESWQDFLRNNAKSYRCALLSHRDGAKVHLGTRPTEKQYETL
ENQLAFLCQQGFSLENALYALSAVGHFTLGCVLEEQEHQVAKEERETPTTDSMPPLLK
QAIELFDRQGAEPAFLFGLELIICGLEKQLKCESGGPTDALDDFDLDMLPADALDDFD
LDMLPADALDDFDLDMLPG*
TRE3G promoter
(SEQ ID NO: 45)
TTTACTCCCTATCAGTGATAGAGAACGTATGAAGAGTTTACTCCCTATCAGTGATAG
AGAACGTATGCAGACTTTACTCCCTATCAGTGATAGAGAACGTATAAGGAGTTTAC
TCCCTATCAGTGATAGAGAACGTATGACCAGTTTACTCCCTATCAGTGATAGAGAA
CGTATCTACAGTTTACTCCCTATCAGTGATAGAGAACGTATATCCAGTTTACTCCCT
ATCAGTGATAGAGAACGTATAAGCTTTAGGCGTGTACGGTGGGCGCCTATAAAAGC
AGAGCTCGTTTAGTGAACCGTCAGATCGCCTGGAGCAATTCCACAACACTTTTGT
CTTATACCAACTTTCCGTACCACTTCCTACCCTCGTAAA
bGH poly(A) sequence
(SEQ ID NO: 46)
ctgtgccttctagttgccagccatctgttgtttgcccctcccccgtgccttccttgaccctggaaggtgccactcccactgtcctttcctaat
aaaatgaggaaattgcatcgcattgtctgagtaggtgtcattctattctggggggtggggtggggcaggacagcaagggggaggattg
ggaagagaatagcaggcaggctgggga
SV40 poly(A) sequence
(SEQ ID NO: 47)
Taagatacattgatgagtttggacaaaccacaactagaatgcagtgaaaaaaatgctttatttgtgaaatttgtgatgctattgctttatttgt
aaccattataagctgcaataaacaagtt

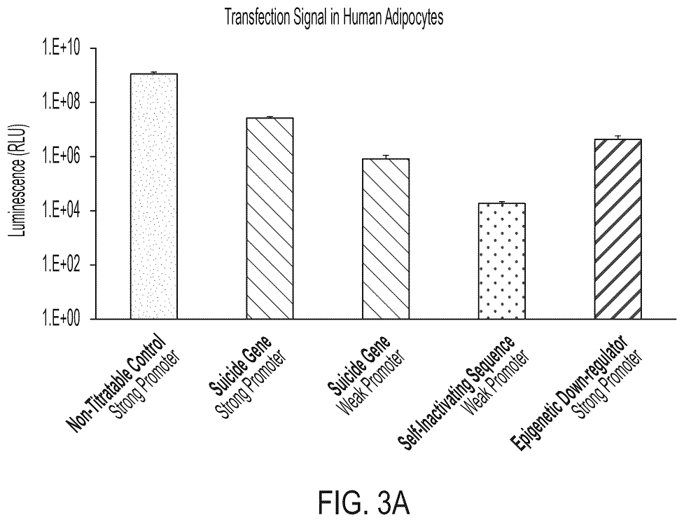

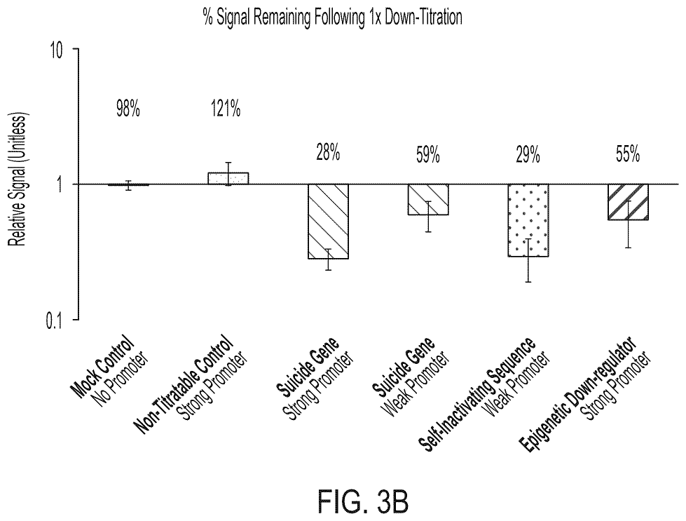

The activity of the suicide gene-based down-titration mechanism is qualified using an nLuc bioluminescent reporter (FIGS. 3A-3B). Activation of the self-silencing signal by delivery of an operator induces a permanent reduction by approximately 2× to 3× in the reporter transgene expression (FIGS. 3A-3B; “Suicide Gene—Strong Promoter” and “Suicide Gene—Weak Promoter”), and is a function of the promoter driving the suicide gene expression. When a strong promoter is used to drive the reporter transgene and the suicide gene post activation, the strong promoter produces a strong initial signal (FIG. 3A; “Suicide Gene—Strong Promoter”) and a more pronounced down-titration (FIG. 3B; “Suicide Gene—Strong Promoter”). Conversely, a low-strength promoter, produces a lower expression signal (FIG. 3A; “Suicide Gene—Weak Promoter”) and a lower level of down-titration following delivery of the operator (FIG. 3B; “Suicide Gene—Weak Promoter”).

### Example 3

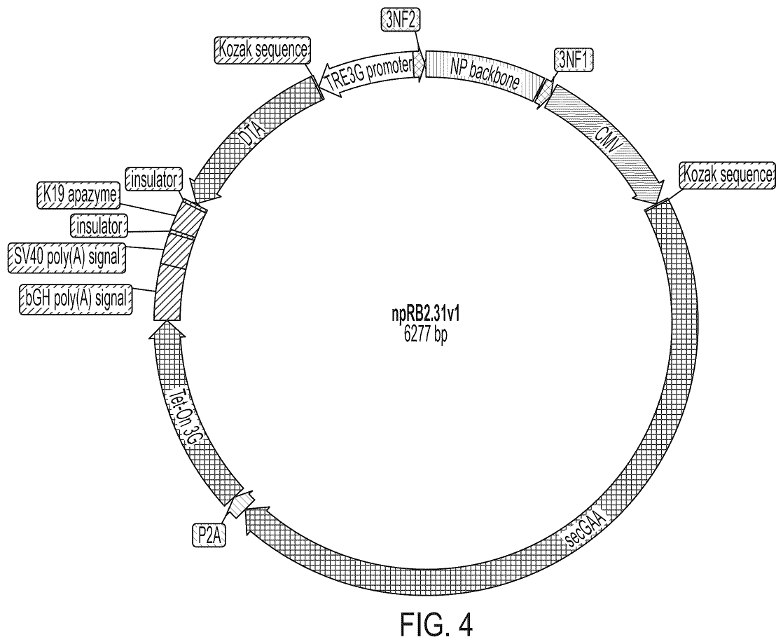

In the present example a secretable alpha-glucosidase is delivered under the regulation of a CMV promoter for the treatment of Pompe disease as an enzyme replacement therapy. To ensure that the enzyme level does not exceed the therapeutic limit, the genetic construct is delivered in cis with a toxin subunit is used to destabilize and inhibit elongation factor 2 (EF-2) thereby preventing elongation and inhibiting protein synthesis. FIG. 4 shows a plasmid map for the genetic construct described in this example.

Full sequence (except nanoplasmid backbone)
(SEQ ID NO: 48)
ctggggactttccagcctggggactttccagctgggactttccaggtagttattaatagtaatcaattacggggtcattagttcatagccca
tatatggagttccgcgttacataacttacggtaaatggcccgcctggctgaccgcccaacgacccccgcccattgacgtcaataatgac
gtatgttcccatagtaacgccaatagggactttccattgacgtcaatgggtggagtatttacggtaaactgcccacttggcagtacatcaa
gtgtatcatatgccaagtacgccccctattgacgtcaatgacggtaaatggcccgcctggcattatgcccagtacatgaccttatgggac
tttcctacttggcagtacatctacgtattagtcatcgctattaccatggtgatgcggttttggcagtacatcaatgggcgtggatagcggttt
gactcacggggatttccaagtctccaccccattgacgtcaatgggagtttgttttggcaccaaaatcaacgggactttccaaaatgtcgta
acaactccgccccattgacgcaaatgggcggtaggcgtgtacggtgggaggtctatataagcagagctggtttagtgaaccgtcagat
cgccaccatggccttcttgtggctgctcagctgctgggccctgctgggcaccacattcggcctgcttgtgcctagagagctgagcggct
ctagcccagtgctggaggaaacccacccagcccaccagcagggagctagccggcctggacccagagacgcccaggcccacccc
ggaagacccagagccgtgcctacccagtgcgacgtgcctcccaacagcagattcgactgcgcccctgataaggccattacccagga
gcagtgtgaagctagaggctgctgctacatccctgccaagcagggcctgcaaggcgcccagatgggccaaccttggtgcttcttccc
accctcttaccctagctacaagctggaaaacctgagctcttctgagatgggctacaccgccaccctgacaagaaccaccccaacattct
tcccaaaagacatcctgaccctgagactggacgtgatgatggaaaccgagaaccggctgcatttcaccattaaggaccccgccaaca
gacggtacgaggtccccctggaaacaccccacgtgcacagccgggccccttcccctctgtacagtgtggaattcagcgaggaaccct
tcggcgtgatcgtgagaaggcagctggatggcagagtgctgctcaacaccacagtggctccattattcttcgccgaccagttcctgca
gctgagcaccagcctgccctcacagtacatcacaggcctcgccgaacacctgtctcctctcatgctgagcaccagctggacaagaatc
acactgtggaaccgggacctggcccccacacctggggctaatctgtacggctcccaccccttctacctggcgctggaagatggcggc
agcgcccacggcgttttcctgctgaacagcaatgccatggacgtggtgctgcagcctagccctgccctgagctggcggagcaccgg
cggaatcctggacgtgtacatcttcctgggacccgagcctaagagcgtggtgcagcaatatctggatgtggtgggctacccctttatgc
ctccttactggggcctgggttttcacctgtgtagatggggctactcatctaccgcgatcaccagacaggtggttgagaatatgacccgg
gcccacttccctctcgacgtgcagtggaacgacctggactacatggacagcagaagagatttcacgttcaacaaggacggcttcagg
gatttccctgctatggtgcaggagctgcaccagggaggcagacggtacatgatgatcgtggatcctgctatcagcagcagcggcccc
gccggcagctatagaccttatgacgagggactgagacggggcgttttcatcacaaacgagacaggacagcctcttatcggcaaggtg
tggcccggctctaccgcctttcctgacttcacaaatcctaccgccctggcctggtgggaagatatggtcgccgagttccacgaccaggt
ccctttcgacgggatgtggatcgacatgaacgagcccagtaacttcattagaggctctgaagatggatgtcctaacaacgagctggag
aaccctccttacgtgcctggcgtggtcggcggaactctgcaggccgctacaatctgtgccagctcccaccagttcctgagcacccact
acaacctgcacaatctgtacggactgacagaggctatcgccagtcatagagccctggtcaaggccagaggcaccagacccttcgtga
tctccagaagcaccttcgccggacacggcagatatgccggccactggacaggcgacgtgtggtcctcttgggagcagctcgcaagc
tctgtgccggaaatcctgcaatttaacttgctgggcgtgcctctggtgggcgctgatgtgtgcggctttctgggcaacacatctgaagaa
ttgtgcgtgcggtggacccagctgggcgctttttacccttttatgagaaatcacaactccctgctgagcctgccacaggagccttacagc
ttcagcgaacctgcccaacaggccatgcggaaggccttaacactgcgatacgcactgctgcctcacctgtacaccctgttccaccagg
ctcatgtggccggcgaaacagtggccagaccactgttcctggagttccccaaggatagctccacatggaccgtggaccaccagctgc
tgtggggcgaggccctgctgatcacccctgtgctgcaggccggcaaagctgaggtgaccggctacttccctctgggcacctggtacg
acctgcagaccgtgcctgttgaggctctgggatctctgccccccccacctgccgcccctcgcgagcctgctatccactctgaaggcca
gtgggtgacactgcctgccccactcgacaccatcaacgtgcacctgagagccggttacatcatccccctgcagggccccggcctgac
caccaccgaaagccggcagcaacctatggccctcgccgtggccctgaccaagggcggcgaggccaggggtgagctattttgggac
gacggcgagagcctggaggtgctggagagaggcgcctacacacaagttatcttcctggctcggaacaacaccatcgtgaacgagct
ggtgcgggtgacaagcgagggcgccggcctccagctgcagaaagtgacggtgctgggcgtggccacagcccctcagcaggtgct
gagcaacggcgtgccagtcagcaatttcacctacagccctgataccaaggttctggacatctgcgtgagcctgctcatgggcgaacag
ttcctggtgtcctggtgcggatctggcgccacaaacttcagcctgctgaagcaggccggagatgtggaagaaaatcctggccccatga
gcagactggataagtccaaggtgatcaacagcgccctggaactgctcaacggcgtcggcatcgagggactcaccacccggaagct
ggcccagaaactgggcgtggaacaacctacactgtactggcacgtgaagaacaaaagagccctgctggatgccctgccaatcgaga
tgctggaccggcaccatacccacagctgccccctggagggagaaagctggcaggacttcctgagaaacaacgctaagagctaccg
gtgcgccctgctgagccaccgggacggcgccaaggtgcacttggggaccagacccacggaaaagcaatacgagacactggagaa
ccagctggccttcctgtgccagcagggcttcagcctggagaatgccctctacgccctgtctgccgtgggccactttaccctgggatgtg
tgctggaagagcaggagcaccaggtggccaaggaagagagggaaacccctaccaccgacagcatgcctcctctgctgaagcaag
ctatcgagctgttcgacagacagggcgccgagcctgctttcctgtttggcctggagctgatcatctgcggcttggagaaacagctgaag
tgcgagagcggcggccctaccgacgccctggacgatttcgacctggatatgctgcctgccgacgccctggacgacttcgacctggat
atgctgcctgccgatgctctggacgacttcgacctggacatgcttccaggctaactgtgccttctagttgccagccatctgttgtttgcccc
tcccccgtgccttccttgaccctggaaggtgccactcccactgtcctttcctaataaaatgaggaaattgcatcgcattgtctgagtaggt
gtcattctattctggggggtggggtggggcaggacagcaagggggaggattgggaagagaatagcaggcaggctggggacaactt
gtttattgcagcttataatggttacaaataaagcaatagcatcacaaatttcacaaataaagcatttttttcactgcattctagttgtggtttgtc
caaactcatcaatgtatcttatttgtttgtttgagcgcgtttcgtcctatttgggactcatcagctggatgtagtaggtggtcgtattcttcacct
ctccagatcgaaatctggtatgttttaccacgaatccaggacgcgcctttgtttgtttgtcattattagagctttaaatctctgtaggtagtttgt
ccaattatgtcacaccacagaagtaaggttccttcacaaagagatcgcctgacacgatttcctgcacaggcttgagccatatactcatac
atcgcatcttggccacgttttccacgggtttcaaaattaatctcaagttctacgcttaacgctttcgcctgttcccagttattaatatattcaac
gctagaactcccctcagcgaagggaaggctgagcactacacgcgaagcaccatcaccgaaccttttgataaactcttccgttccgactt
gctccatcaacggttcagtgagacttaaacctaactctttcttaatagtttcggcattatccacttttagtgcgagaaccttcgtcagtcctgg
atacgtcactttgaccacgcctccagcttttccagagagcgggttttcattatctacagagtatcccgcagcgtcgtatttattgtcggtact
ataaaaccctttccaatcatcgtcataatttccttgtgtaccagattttggcttttgtatacctttttgaatggaatctacataaccaggtttagt
cccgtggtacgaagaaaagttttccatcacaaaagatttagaagaatcaacaacatcatcaggatccatggtggctttacgagggtagga
agtggtacggaaagttggtataagacaaaagtgttgtggaattgctccaggcgatctgacggttcactaaacgagctctgcttttatagg
cgcccaccgtacacgcctaaagcttatacgttctctatcactgatagggagtaaactggatatacgttctctatcactgatagggagtaaa
ctgtagatacgttctctatcactgatagggagtaaactggtcatacgttctctatcactgatagggagtaaactccttatacgttctctatcac
tgatagggagtaaagtctgcatacgttctctatcactgatagggagtaaactcttcatacgttctctatcactgatagggagtaaactggg
gactttccagctggggactttccagctgggactttccaggag
3NF1
(SEQ ID NO: 10)
Ctggggactttccagcctggggactttccagctgggactttccagg
3NF2
(SEQ ID NO: 11)
Ctggggactttccagctggggactttccagctgggactttccaggag
CMV promoter
(SEQ ID NO: 49)
Tagttattaatagtaatcaattacggggtcattagttcatagcccatatatggagttccgcgttacataacttacggtaaatggcccgcctg
gctgaccgcccaacgacccccgcccattgacgtcaataatgacgtatgttcccatagtaacgccaatagggactttccattgacgtcaat
gggtggagtatttacggtaaactgcccacttggcagtacatcaagtgtatcatatgccaagtacgccccctattgacgtcaatgacggta
aatggcccgcctggcattatgcccagtacatgaccttatgggactttcctacttggcagtacatctacgtattagtcatcgctattaccatg
gtgatgcggttttggcagtacatcaatgggcgtggatagcggtttgactcacggggatttccaagtctccaccccattgacgtcaatggg
agtttgttttggcaccaaaatcaacgggactttccaaaatgtcgtaacaactccgccccattgacgcaaatgggcggtaggcgtgtacg
gtgggaggtctatataagcagagctggtttagtgaaccgtcagatc
secGAA (sp7-delta8-hGAA)
Nucleotide sequence
(SEQ ID NO: 50)
atggccttcttgtggctgctcagctgctgggccctgctgggcaccacattcggcctgcttgtgcctagagagctgagcggctctagccc
agtgctggaggaaacccacccagcccaccagcagggagctagccggcctggacccagagacgcccaggcccaccccggaagac
ccagagccgtgcctacccagtgcgacgtgcctcccaacagcagattcgactgcgcccctgataaggccattacccaggagcagtgtg
aagctagaggctgctgctacatccctgccaagcagggcctgcaaggcgcccagatgggccaaccttggtgcttcttcccaccctctta
ccctagctacaagctggaaaacctgagctcttctgagatgggctacaccgccaccctgacaagaaccaccccaacattcttcccaaaa
gacatcctgaccctgagactggacgtgatgatggaaaccgagaaccggctgcatttcaccattaaggaccccgccaacagacggtac
gaggtccccctggaaacaccccacgtgcacagccgggccccttcccctctgtacagtgtggaattcagcgaggaacccttcggcgtg
atcgtgagaaggcagctggatggcagagtgctgctcaacaccacagtggctccattattcttcgccgaccagttcctgcagctgagca
ccagcctgccctcacagtacatcacaggcctcgccgaacacctgtctcctctcatgctgagcaccagctggacaagaatcacactgtg
gaaccgggacctggcccccacacctggggctaattgtacggctcccaccccttctacctggcgctggaagatggcggcagcgccc
acggcgttttcctgctgaacagcaatgccatggacgtggtgctgcagcctagccctgccctgagctggcggagcaccggcggaatcc
tcgacgtgtacatcttcctgggacccgagcctaagagcgtggtgcagcaatatctggatgtggtgggctacccctttatgcctccttact
ggggcctgggttttcacctgtgtagatggggctactcatctaccgcgatcaccagacaggtggttgagaatatgacccgggcccacttc
cctctcgacgtgcagtggaacgacctggactacatggacagcagaagagatttcacgttcaacaaggacggcttcagggatttccctg
ctatggtgcaggagctgcaccagggaggcagacggtacatgatgatcgtggatcctgctatcagcagcagcggccccgccggcag
ctatagaccttatgacgagggactgagacggggcgttttcatcacaaacgagacaggacagcctcttatcggcaaggtgtggcccgg
ctctaccgcctttcctgacttcacaaatcctaccgccctggcctggtgggaagatatggtcgccgagttccacgaccaggtccctttcga
cgggatgtggatcgacatgaacgagcccagtaacttcattagaggctctgaagatggatgtcctaacaacgagctggagaaccctcct
tacgtgcctggcgtggtcggcggaactctgcaggccgctacaatctgtgccagctcccaccagttcctgagcacccactacaacctgc
acaatctgtacggactgacagaggctatcgccagtcatagagccctggtcaaggccagaggcaccagacccttcgtgatctccagaa
gcaccttcgccggacacggcagatatgccggccactggacaggcgacgtgtggtcctcttgggagcagctcgcaagctctgtgccg
gaaatcctgcaatttaacttgctgggcgtgcctctggtgggcgctgatgtgtgggctttctgggcaacacatctgaagaattgtgcgtg
cggtggacccagctgggcgctttttacccttttatgagaaatcacaactccctgctgagcctgccacaggagccttacagcttcagcga
acctgcccaacaggccatgcggaaggccttaacactgcgatacgcactgctgcctcacctgtacaccctgttccaccaggctcatgtg
gccggcgaaacagtggccagaccactgttcctggagttccccaaggatagctccacatggaccgtggaccaccagctgctgtgggg
cgaggccctgctgatcacccctgtgctgcaggccggcaaagctgaggtgaccggctacttccctctgggcacctggtacgacctgca
gaccgtgcctgttgaggctctgggatctctgccccccccacctgccgcccctcgcgagcctgctatccactctgaaggccagtgggtg
acactgcctgccccactcgacaccatcaacgtgcacctgagagccggttacatcatccccctgcagggccccggcctgaccaccacc
gaaagccggcagcaacctatggccctcgccgtggccctgaccaagggcggcgaggccaggggtgagctattttgggacgacggc
gagagcctggaggtgctggagagaggcgcctacacacaagttatcttcctggctcggaacaacaccatcgtgaacgagctggtgcg
ggtgacaagcgagggcgccggcctccagctgcagaaagtgacggtgctgggcgtggccacagcccctcagcaggtgctgagcaa
cggcgtgccagtcagcaatttcacctacagccctgataccaaggttctggacatctgcgtgagcctgctcatgggcgaacagttcctgg
tgtcctggtgctaa
Amino acid sequence
(SEQ ID NO: 51)
MAFLWLLSCWALLGTTFGLLVPRELSGSSPVLEETHPAHQQGASRPGPRDAQAHPGR
PRAVPTQCDVPPNSRFDCAPDKAITQEQCEARGCCYIPAKQGLQGAQMGQPWCFFPP
SYPSYKLENLSSSEMGYTATLTRTTPTFFPKDILTLRLDVMMETENRLHFTIKDPANRR
YEVPLETPHVHSRAPSPLYSVEFSEEPFGVIVRRQLDGRVLLNTTVAPLFFADQFLQLS
TSLPSQYITGLAEHLSPLMLSTSWTRITLWNRDLAPTPGANLYGSHPFYLALEDGGSA
HGVFLLNSNAMDVVLQPSPALSWRSTGGILDVYIFLGPEPKSVVQQYLDVVGYPFMP
PYWGLGFHLCRWGYSSTAITRQVVENMTRAHFPLDVQWNDLDYMDSRRDFTENKD
GFRDFPAMVQELHQGGRRYMMIVDPAISSSGPAGSYRPYDEGLRRGVFITNETGQPLI
GKVWPGSTAFPDFTNPTALAWWEDMVAEFHDQVPFDGMWIDMNEPSNFIRGSEDGC
PNNELENPPYVPGVVGGTLQAATICASSHQFLSTHYNLHNLYGLTEAIASHRALVKAR
GTRPFVISRSTFAGHGRYAGHWTGDVWSSWEQLASSVPEILQFNLLGVPLVGADVCG
FLGNTSEELCVRWTQLGAFYPFMRNHNSLLSLPQEPYSFSEPAQQAMRKALTLRYAL
LPHLYTLFHQAHVAGETVARPLFLEFPKDSSTWTVDHQLLWGEALLITPVLQAGKAE
VTGYFPLGTWYDLQTVPVEALGSLPPPPAAPREPAIHSEGQWVTLPAPLDTINVHLRA
GYIIPLQGPGLTTTESRQQPMALAVALTKGGEARGELFWDDGESLEVLERGAYTQVIF
LARNNTIVNELVRVTSEGAGLQLQKVTVLGVATAPQQVLSNGVPVSNFTYSPDTKVL
DICVSLLMGEQFLVSWC
P2A
Nucleotide sequence
(SEQ ID NO: 37)
GGATCTGGCGCCACAAACTTCAGCCTGCTGAAGCAGGCCGGAGATGTGGAAGAA
AATCCTGGCCCC
Amino acid sequence
(SEQ ID NO: 29)
ATNFSLLKQAGDVEENPGP
Tet-on 3G
Nucleotide sequence
(SEQ ID NO: 43)
Atgagcagactggataagtccaaggtgatcaacagcgccctggaactgctcaacggcgtcggcatcgagggactcaccacccgga
agctggcccagaaactgggcgtggaacaacctacactgtactggcacgtgaagaacaaaagagccctgctggatgccctgccaatc
gagatgctggaccggcaccatacccacagctgccccctggagggagaaagctggcaggacttcctgagaaacaacgctaagagct
accggtgcgccctgctgagccaccgggacggcgccaaggtgcacttggggaccagacccacggaaaagcaatacgagacactgg
agaaccagctggccttcctgtgccagcagggcttcagcctggagaatgccctctacgccctgtctgccgtgggccactttaccctggg
atgtgtgctggaagagcaggagcaccaggtggccaaggaagagagggaaacccctaccaccgacagcatgcctcctctgctgaag
caagctatcgagctgttcgacagacagggcgccgagcctgctttcctgtttggcctggagctgatcatctgcggcttggagaaacagct
gaagtgcgagagcggcggccctaccgacgccctggacgatttcgacctggatatgctgcctgccgacgccctggacgacttcgacc
tggatatgctgcctgccgatgctctggacgacttcgacctggacatgcttccaggctaa
Amino acid sequence
(SEQ ID NO: 44)
MSRLDKSKVINSALELLNGVGIEGLTTRKLAQKLGVEQPTLYWHVKNKRALLDALPI
EMLDRHHTHSCPLEGESWQDFLRNNAKSYRCALLSHRDGAKVHLGTRPTEKQYETL
ENQLAFLCQQGFSLENALYALSAVGHFTLGCVLEEQEHQVAKEERETPTTDSMPPLLK
QAIELFDRQGAEPAFLFGLELIICGLEKQLKCESGGPTDALDDFDLDMLPADALDDFD
LDMLPADALDDFDLDMLPG
TRE3G promoter
(SEQ ID NO: 45)
Tttactccctatcagtgatagagaacgtatgaagagtttactccctatcagtgatagagaacgtatgcagactttactccctatcagtgata
gagaacgtataaggagtttactccctatcagtgatagagaacgtatgaccagtttactccctatcagtgatagagaacgtatctacagttta
ctccctatcagtgatagagaacgtatatccagtttactccctatcagtgatagagaacgtataagctttaggcgtgtacggtgggcgccta
taaaagcagagctcgtttagtgaaccgtcagatcgcctggagcaattccacaacacttttgtcttataccaactttccgtaccacttcctac
cctcgtaaa
Diptheria toxin subunit A (DTA):
Nucleotide sequence
(SEQ ID NO: 52)
ATGGATCCTGATGATGTTGTTGATTCTTCTAAATCTTTTGTGATGGAAAACTTTTCT
TCGTACCACGGGACTAAACCTGGTTATGTAGATTCCATTCAAAAAGGTATACAAAA
GCCAAAATCTGGTACACAAGGAAATTATGACGATGATTGGAAAGGGTTTTATAGTA
CCGACAATAAATACGACGCTGCGGGATACTCTGTAGATAATGAAAACCCGCTCTCT
GGAAAAGCTGGAGGCGTGGTCAAAGTGACGTATCCAGGACTGACGAAGGTTCTC
GCACTAAAAGTGGATAATGCCGAAACTATTAAGAAAGAGTTAGGTTTAAGTCTCA
CTGAACCGTTGATGGAGCAAGTCGGAACGGAAGAGTTTATCAAAAGGTTCGGTG
ATGGTGCTTCGCGTGTAGTGCTCAGCCTTCCCTTCGCTGAGGGGAGTTCTAGCGTT
GAATATATTAATAACTGGGAACAGGCGAAAGCGTTAAGCGTAGAACTTGAGATTAA
TTTTGAAACCCGTGGAAAACGTGGCCAAGATGCGATGTATGAGTATATGGCTCAAG
CCTGTGCAGGAAATCGTGTCAGGCGATCTCTTTGTGAAGGAACCTTACTTCTGTGG
TGTGACATAATTGGACAAACTACCTACAGAGATTTAAAGCTCTAA
Amino acid sequence
(SEQ ID NO: 53)
MDPDDVVDSSKSFVMENFSSYHGTKPGYVDSIQKGIQKPKSGTQGNYDDDWKGFYS
TDNKYDAAGYSVDNENPLSGKAGGVVKVTYPGLTKVLALKVDNAETIKKELGLSLT
EPLMEQVGTEEFIKRFGDGASRVVLSLPFAEGSSSVEYINNWEQAKALSVELEINFET
RGKRGQDAMYEYMAQACAGNRVRRSLCEGTLLLWCDIIGQTTYRDLKL
K19 aptazyme
(SEQ ID NO: 54)
Ggcgcgtcctggattcgtggtaaaacataccagatttcgatctggagaggtgaagaatacgaccacctactacatccagctgatgagt
cccaaataggacgaaacgcgct
Insulator sequence
(SEQ ID NO: 55)
Caaacaaacaaa
bGH poly(A) sequence
(SEQ ID NO: 46)
ctgtgccttctagttgccagccatctgttgtttgcccctcccccgtgccttccttgaccctggaaggtgccactcccactgtcctttcctaat
aaaatgaggaaattgcatcgcattgtctgagtaggtgtcattctattctggggggtggggtggggcaggacagcaagggggaggattg
ggaagagaatagcaggcaggctgggga
SV40 poly(A) sequence
(SEQ ID NO: 47)
Taagatacattgatgagtttggacaaaccacaactagaatgcagtgaaaaaaatgctttatttgtgaaatttgtgatgctattgctttatttgt
aaccattataagctgcaataaacaagtt

### Example 4

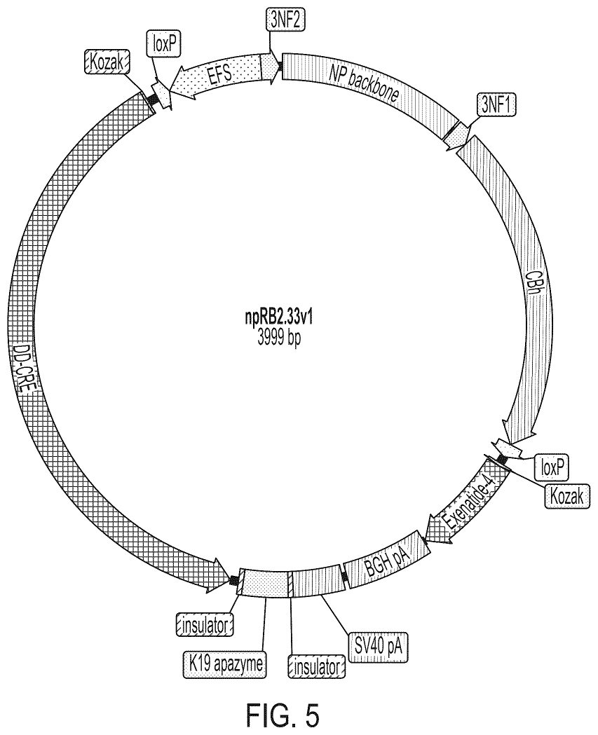

In the present example a GLP-1 Receptor Antagonist Analog Exenatide is delivered under the regulation of the CBh promoter. In cis with the Exenatide sequence cDNA, which is coupled with a secretory peptide, the construct contains a K19 aptazyme and a destabilized Cre recombinase. Upon administration of Tetracycline the K19 aptazyme stabilizes the Cre transcript, which coupled with Trimethoprim activates recombinase activity, which targets a sequence within the promoter region of the construct to eliminate expression of the therapeutic transgene (Exanatide). FIG. 5 shows a plasmid map for the genetic construct described in this example. The construct components, actuators, effectors, and overall sequences are shown below:

Actuator: Tetracycline (for K19 aptazyme) and Trimethoprim CAS RN 738-70-5, molecular formula C14H18N4O3 (for destabilized Cre).

Full sequence (except nanoplasmid backbone)
(SEQ ID NO: 56)
ctggggactttccagcctggggactttccagctgggactttccaggcgttacataacttacggtaaatggcccgcctggctgaccgccc
aacgacccccgcccattgacgtcaatagtaacgccaatagggactttccattgacgtcaatgggtggagtatttacggtaaactgccca
cttggcagtacatcaagtgtatcatatgccaagtacgccccctattgacgtcaatgacggtaaatggcccgcctggcattgtgcccagta
catgaccttatgggactttcctacttggcagtacatctacgtattagtcatcgctattaccatggtcgaggtgagccccacgttctgcttcac
tctccccatctcccccccctccccacccccaattttgtatttatttattttttaattattttgtgcagcgatggggcgggggggggggggg
ggcgcgcgccaggcggggcggggcggggcgaggggcggggcggggcgaggcggagaggtgcggcggcagccaatcagag
cggcgcgctccgaaagtttccttttatggcgaggcggcggcggcggcggccctataaaaagcgaagcgcgcggcgggcgggagt
cgctgcgcgctgccttcgccccgtgccccgctccgccgccgcctcgcgccgcccgccccggctctgactgaccgcgttactcccac
aggtgagcgggcgggacggcccttctcctccgggctgtaattagctgagcaagaggtaagggtttaagggatggttggttggtgggg
tattaatgtttaattacctggagcacctgcctgaaatcactttttttcaggttggataacttcgtatagcatacattatacgaagttatgttgga
cgcgtcgctagcgccaccatggccacagacagcagaacaagctggctgctcaccgtgtccctgctgtgcctgctgtggccccaggag
gccagcgccttccccgctatgcctctgagcagcctgttcagcaacgccgtgctgagagccagaggcaagcggcacggcgagggca
ccttcacctctgatctgtccaagcagatggaagaggaagctgtgcggctgtttatcgagtggctgaaaaacggcggaccttctagcgg
cgcccctccacctagctaatgactgtgccttctagttgccagccatctgttgtttgcccctcccccgtgccttccttgaccctggaaggtgc
cactcccactgtcctttcctaataaaatgaggaaattgcatcgcattgtctgagtaggtgtcattctattctggggggtggggtggggcag
gacagcaagggggaggattgggaagagaatagcaggcaggctggggagggccgccgatcgaacttgtttattgcagcttataatgg
ttacaaataaagcaatagcatcacaaatttcacaaataaagcatttttttcactgcattctagttgtggtttgtccaaactcatcaatgtatctt
atttgtttgtttgagcgcgtttcgtcctatttgggactcatcagctggatgtagtaggtggtcgtattcttcacctctccagatcgaaatctggt
atgttttaccacgaatccaggacgcgcctttgtttgtttggactagaagcggccgcctactaatcgccatcttccagcaggcgcaccattgc
ccctgtttcactatccaggttacggatatagttcatgacaatatttacattggtccagccaccagcttgcatgatctccggtattgaaactcc
agcgcgggccatatctcgcgcggctccgacacgggcactgtgtccagaccaggccaggtatctctgaccagagtcatccttagcgcc
gtaaatcaatcgatgagttgcttcaaaaatcccttccagggcgcgagttgatagctggctggtggcagatggcgcggcaacaccattttt
tctgacccggcaaaacaggtagttattcggatcatcagctacaccagagacggaaatccatcgctcgaccagtttagttacccccaggc
taagtgccttctctacacctgcggtgctaaccagcgttttcgttctgccaatatggattaacattctcccaccgtcagtacgtgagatatcttt
aaccctgatcctggcaatttcggctatacgtaacagggtgttataagcaatccccagaaatgccagattacgtatatcctggcagcgatc
gctattttccatgagtgaacgaacctggtcgaaatcagtgcgttcgaacgctagagcctgttttgcacgttcaccggcatcaacgttttctt
ttcggatccgccgcataaccagtgaaacagcattgctgtcacttggtcgtggcagcccggaccgacgatgaagcatgtttagctggcc
caaatgttgctggatagtttttactgccagaccgcgcgcctgaagatatagaagataatcgcgaacatcttcaggttctgcgggaaacca
tttccggttattcaacttgcaccatgccgcccacgaccggcaaacggacagaagcattttccaggtatgctcagaaaacgcctggcgat
ccctgaacatgtccatcaggttcttgcgaacctcatcactcgttgcatcgaccggtaatgcaggcaaattttggtgtacggtcagtaaatt
ggacaccttcctcttcttcttaggcgcgcctctcctttccaggatctcaaagcaataactatgagaattctgagcatcggcgtcgtgaaact
cggagaacacgctttcccaatcgtctggctcataatcaggaaaatgggtgtccccttccacctcagcgtcaatgtgagtcaggtacagtt
tctgggccttaggcaggaactgctcgatcactctccctccgccgatgaccatgatctcgggcacatctccgcaagcggcaatggcttc
gtccacacttttcacccatgtcaccctgtcgtcggtagagggctgggagctcagaatgatgttcttgcgtccaggcaggggccggcca
atggactcccaagtgtgtctgcccatgatcactggcttgttcagggtgttcctcttgaaccaagccagatcggcgggcagattccatggc
atagcgttttccatcccgatcacgtagtccacggccagagcggcaatcagagagatcatggtggcagcctgcttttttgtacaaacttga
taacttcgtatagcatacattatacgaagttatcctgtgttctggcggcaaacccgttgcgaaaaagaacgttcacggcgactactgcact
tatatacggttctcccccaccctcgggaaaaaggcggagccagtacacgacatcactttcccagtttaccccgcgccaccttctctagg
caccggatcaattgccgacccctccccccaacttctcggggactgtgggcgatgtgcgctctgcccactgacgggcaccggagccct
ggggactttccagctggggactttccagctgggactttccaggag
3NF1
(SEQ ID NO: 10)
Ctggggactttccagcctggggactttccagctgggactttccagg
3NF2
(SEQ ID NO: 11)
Ctggggactttccagctggggactttccagctgggactttccaggag
loxP site
(SEQ ID NO: 57)
ataacttcgtatagcatacattatacgaagttat
CBh promoter
(SEQ ID NO: 32)
Cgttacataacttacggtaaatggcccgcctggctgaccgcccaacgacccccgcccattgacgtcaatagtaacgccaatagggac
tttccattgacgtcaatgggtggagtatttacggtaaactgcccacttggcagtacatcaagtgtatcatatgccaagtacgccccctattg
acgtcaatgacggtaaatggcccgcctggcattgtgcccagtacatgaccttatgggactttcctacttggcagtacatctacgtattagt
catcgctattaccatggtcgaggtgagccccacgttctgcttcactctccccatctcccccccctccccacccccaattttgtatttatttatt
ttttaattattttgtgcagcgatgggggcggggggggggggggggcgcgcgccaggcggggcggggcggggcgaggggcggg
gcggggcgaggcggagaggtgcggcggcagccaatcagagcggcgcgctccgaaagtttccttttatggcgaggcggcggcggc
ggcggccctataaaaagcgaagcgcgcggcgggcgggagtcgctgcgcgctgccttcgccccgtgccccgctccgccgccgcct
cgcgccgcccgccccggctctgactgaccgcgttactcccacaggtgagcgggcgggacggcccttctcctccgggctgtaattag
ctgagcaagaggtaagggtttaagggatggttggttggtggggtattaatgtttaattacctggagcacctgcctgaaatcactttttttca
ggttgg
Exenatide-4
Nucleotide sequence
(SEQ ID NO: 58)
Atggccacagacagcagaacaagctggctgctcaccgtgtccctgctgtgcctgctgtggccccaggaggccagcgccttccccgc
tatgcctctgagcagcctgttcagcaacgccgtgctgagagccagaggcaagcggcacggcgagggcaccttcacctctgatctgtc
caagcagatggaagaggaagctgtgcggctgtttatcgagtggctgaaaaacggcggaccttctagcggcgcccctccacctagcta
a
Amino acid sequence
(SEQ ID NO: 59)
MATDSRTSWLLTVSLLCLLWPQEASAFPAMPLSSLFSNAVLRARGKRHGEGTFTSDLS
KQMEEEAVRLFIEWLKNGGPSSGAPPPS
EFS promoter
(SEQ ID NO: 30)
Ggctccggtgcccgtcagtgggcagagcgcacatcgcccacagtccccgagaagttggggggaggggtcggcaattgatccggt
gcctagagaaggtggcgcggggtaaactgggaaagtgatgtcgtgtactggctccgcctttttcccgagggtgggggagaaccgtat
ataagtgcagtagtcgccgtgaacgttctttttcgcaacgggtttgccgccagaacacagg
Destabilized Cre Recombinase (DD-CRE):
Nucleotide sequence
(SEQ ID NO: 60)
atgatctctctgattgccgctctggccgtggactacgtgatcgggatggaaaacgctatgccatggaatctgcccgccgatctggcttg
gttcaagaggaacaccctgaacaagccagtgatcatgggcagacacacttgggagtccattggccggcccctgcctggacgcaaga
acatcattctgagctcccagccctctaccgacgacagggtgacatgggtgaaaagtgtggacgaagccattgccgcttgcggagatgt
gcccgagatcatggtcatcggcggagggagagtgatcgagcagttcctgcctaaggcccagaaactgtacctgactcacattgacgc
tgaggtggaaggggacacccattttcctgattatgagccagacgattgggaaagcgtgttctccgagtttcacgacgccgatgctcaga
attctcatagttattgctttgagatcctggaaaggagaggcgcgcctaagaagaagaggaaggtgtccaatttactgaccgtacaccaa
aatttgcctgcattaccggtcgatgcaacgagtgatgaggttcgcaagaacctgatggacatgttcagggatcgccaggcgttttctga
gcatacctggaaaatgcttctgtccgtttgccggtcgtgggcggcatggtgcaagttgaataaccggaaatggtttcccgcagaacctg
aagatgttcgcgattatcttctatatcttcaggcgcgcggtctggcagtaaaaactatccagcaacatttgggccagctaaacatgcttcat
cgtcggtccgggctgccacgaccaagtgacagcaatgctgtttcactggttatgcggcggatccgaaaagaaaacgttgatgccggt
gaacgtgcaaaacaggctctagcgttcgaacgcactgatttcgaccaggttcgttcactcatggaaaatagcgatcgctgccaggatat
acgtaatctggcatttctggggattgcttataacaccctgttacgtatagccgaaattgccaggatcagggttaaagatatctcacgtactg
acggtgggagaatgttaatccatattggcagaacgaaaacgctggttagcaccgcaggtgtagagaaggcacttagcctgggggtaa
ctaaactggtcgagcgatggatttccgtctctggtgtagctgatgatccgaataactacctgttttgccgggtcagaaaaaatggtgttgc
cgcgccatctgccaccagccagctatcaactcgcgccctggaagggatttttgaagcaactcatcgattgatttacggcgctaaggatg
actctggtcagagatacctggcctggtctggacacagtgcccgtgtcggagccgcgcgagatatggcccgcgctggagtttcaatac
cggagatcatgcaagctggtggctggaccaatgtaaatattgtcatgaactatatccgtaacctggatagtgaaacaggggcaatggtg
cgcctgctggaagatggcgattag
Amino acid sequence
(SEQ ID NO: 61)
MISLIAALAVDYVIGMENAMPWNLPADLAWFKRNTLNKPVIMGRHTWESIGRPLPGR
KNIILSSQPSTDDRVTWVKSVDEAIAACGDVPEIMVIGGGRVIEQFLPKAQKLYLTHID
AEVEGDTHFPDYEPDDWESVFSEFHDADAQNSHSYCFEILERRGAPKKKRKVSNLLT
VHQNLPALPVDATSDEVRKNLMDMFRDRQAFSEHTWKMLLSVCRSWAAWCKLNN
RKWFPAEPEDVRDYLLYLQARGLAVKTIQQHLGQLNMLHRRSGLPRPSDSNAVSLV
MRRIRKENVDAGERAKQALAFERTDFDQVRSLMENSDRCQDIRNLAFLGIAYNTLLR
IAEIARIRVKDISRTDGGRMLIHIGRTKTLVSTAGVEKALSLGVTKLVERWISVSGVAD
DPNNYLFCRVRKNGVAAPSATSQLSTRALEGIFEATHRLIYGAKDDSGQRYLAWSGH
SARVGAARDMARAGVSIPEIMQAGGWTNVNIVMNYIRNLDSETGAMVRLLEDGD
K19 aptazyme
(SEQ ID NO: 54)
Ggcgcgtcctggattcgtggtaaaacataccagatttcgatctggagaggtgaagaatacgaccacctactacatccagctgatgagt
cccaaataggacgaaacgcgct
Insulator sequence
(SEQ ID NO: 55)
Caaacaaacaaa
bGH poly(A) sequence
(SEQ ID NO: 46)
ctgtgccttctagttgccagccatctgttgtttgcccctcccccgtgccttccttgaccctggaaggtgccactcccactgtcctttcctaat
aaaatgaggaaattgcatcgcattgtctgagtaggtgtcattctattctggggggtggggtggggcaggacagcaagggggaggattg
ggaagagaatagcaggcaggctgggga
SV40 poly(A) sequence
(SEQ ID NO: 47)
Taagatacattgatgagtttggacaaaccacaactagaatgcagtgaaaaaaatgctttatttgtgaaatttgtgatgctattgctttatttgt
aaccattataagctgcaataaacaagtt

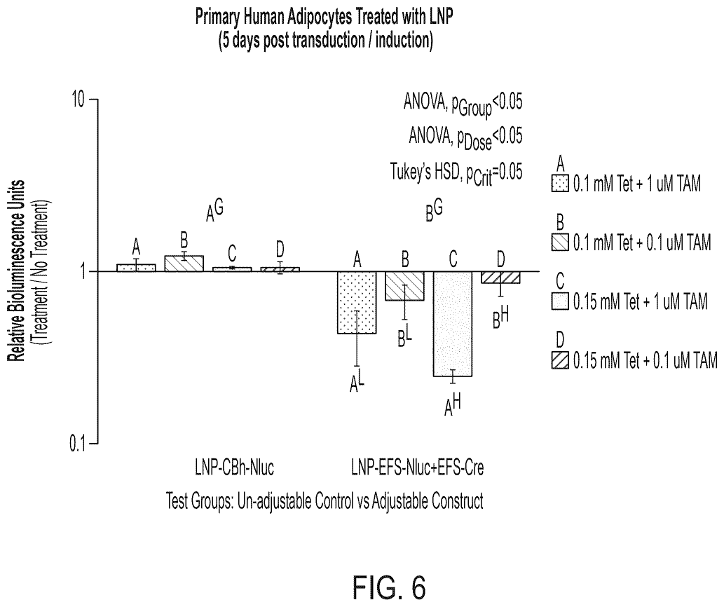

The activity of the self-inactivating Cre-based down-titration mechanism is qualified using an nLuc bioluminescent reporter (FIG. 6). Activation of the self-silencing signal by delivery of an operator induces a permanent reduction by approximately 3× in the reporter transgene expression (FIG. 6; “A”).

### Example 5

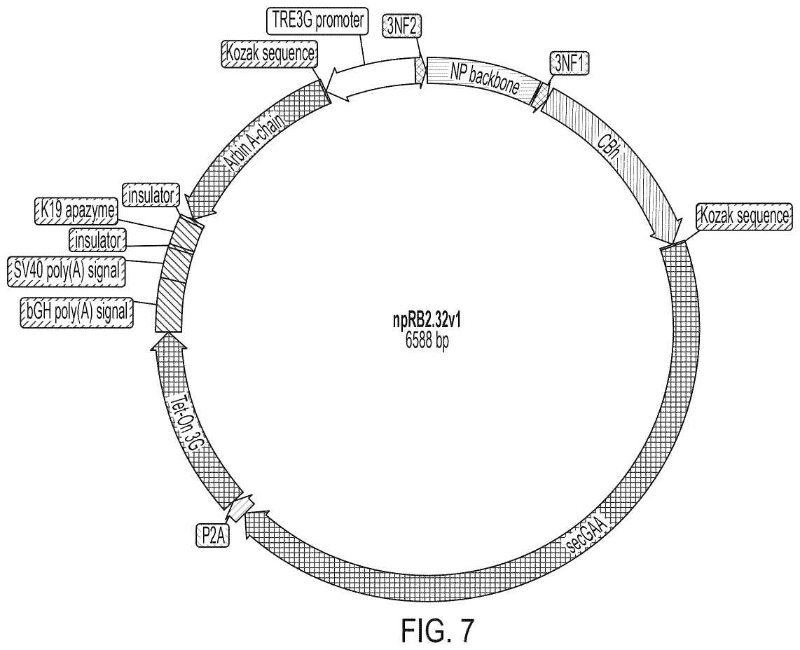

In the present example an enzyme replacement therapy is delivered under the regulation of a constitutive promoter. The down-titration is mediated by a Arbus precatorius L, plant-derived Ribosome Inhibiting Protein (RIP) Abrin-A under the regulation of a 3^{rd} generation TetOn promoter/operator and a K19 aptazyme. The administration of tetracycline or doxycycline activates the TetOn operator/promoter, which drives expression of the RIP protein, which inhibits ribosomes and reduces the overall gene expression from the therapeutic construct. FIG. 7 shows a plasmid map for the genetic construct described in this example.

Full sequence (except nanoplasmid backbone)
(SEQ ID NO: 62)
ctggggactttccagcctggggactttccagctgggactttccaggcgttacataacttacggtaaatggcccgcctggctgaccgccc
aacgacccccgcccattgacgtcaatagtaacgccaatagggactttccattgacgtcaatgggtggagtatttacggtaaactgccca
cttggcagtacatcaagtgtatcatatgccaagtacgccccctattgacgtcaatgacggtaaatggcccgcctggcattgtgcccagta
catgaccttatgggactttcctacttggcagtacatctacgtattagtcatcgctattaccatggtcgaggtgagccccacgttctgcttcac
tctccccatctcccccccctccccacccccaattttgtatttatttattttttaattattttgtgcagcgatgggggcggggggggggggg
ggcgcgcgccaggcggggcggggcggggcgaggggcggggcggggcgaggcggagaggtgcggcagccaatcagag
cggcgcgctccgaaagtttccttttatggcgaggcggcggcggcggcggccctataaaaagcgaagcgcgcggcggggggagt
cgctgcgcgctgccttcgccccgtgccccgctccgccgccgcctcgcgccgcccgccccggctctgactgaccgcgttactcccac
aggtgagcggggggacggcccttctcctccgggctgtaattagctgagcaagaggtaagggtttaagggatggttggttggtgggg
tattaatgtttaattacctggagcacctgcctgaaatcactttttttcaggttgggccaccatggccttcttgtggctgctcagctgctgggc
cctgctgggcaccacattcggcctgcttgtgcctagagagctgagcggctctagcccagtgctggaggaaacccacccagcccacc
agcagggagctagccggcctggacccagagacgcccaggcccaccccggaagacccagagccgtgcctacccagtgcgacgtg
cctcccaacagcagattcgactgcgcccctgataaggccattacccaggagcagtgtgaagctagaggctgctgctacatccctgcca
agcagggcctgcaaggcgcccagatgggccaaccttggtgcttcttcccaccctcttaccctagctacaagctggaaaacctgagctc
ttctgagatgggctacaccgccaccctgacaagaaccaccccaacattcttcccaaaagacatcctgaccctgagactggacgtgatg
atggaaaccgagaaccggctgcatttcaccattaaggaccccgccaacagacggtacgaggtccccctggaaacaccccacgtgca
cagccgggccccttcccctctgtacagtgtggaattcagcgaggaacccttcggcgtgatcgtgagaaggcagctggatggcagagt
gctgctcaacaccacagtggctccattattcttcgccgaccagttcctgcagctgagcaccagcctgccctcacagtacatcacaggcc
tcgccgaacacctgtctcctctcatgctgagcaccagctggacaagaatcacactgtggaaccgggacctggcccccacacctgggg
ctaatctgtacggctcccaccccttctacctggcgctggaagatggcggcagcgcccacggcgttttcctgctgaacagcaatgccat
ggacgtggtgctgcagcctagccctgccctgagctggcggagcaccggcggaatcctggacgtgtacatcttcctgggacccgagc
ctaagagcgtggtgcagcaatatctggatgtggtgggctacccctttatgcctccttactggggcctgggttttcacctgtgtagatgggg
ctactcatctaccgcgatcaccagacaggtggttgagaatatgacccgggcccacttccctctcgacgtgcagtggaacgacctggac
tacatggacagcagaagagatttcacgttcaacaaggacggcttcagggatttccctgctatggtgcaggagctgcaccagggaggc
agacggtacatgatgatcgtggatcctgctatcagcagcagcggccccgccggcagctatagaccttatgacgagggactgagacg
gggcgttttcatcacaaacgagacaggacagcctcttatcggcaaggtgtggcccggctctaccgcctttcctgacttcacaaatcctac
cgccctggcctggtgggaagatatggtcgccgagttccacgaccaggtccctttcgacgggatgtggatcgacatgaacgagcccag
taacttcattagaggctctgaagatggatgtcctaacaacgagctggagaaccctccttacgtgcctggcgtggtcggcggaactctgc
aggccgctacaatctgtgccagctcccaccagttcctgagcacccactacaacctgcacaatctgtacggactgacagaggctatcgc
cagtcatagagccctggtcaaggccagaggcaccagacccttcgtgatctccagaagcaccttcgccggacacggcagatatgccg
gccactggacaggcgacgtgtggtcctcttgggagcagctcgcaagctctgtgccggaaatcctgcaatttaacttgctgggcgtgcct
ctggtgggcgctgatgtgtgcggctttctgggcaacacatctgaagaattgtgcgtgcggtggacccagctgggcgctttttaccctttta
tgagaaatcacaactccctgctgagcctgccacaggagccttacagcttcagcgaacctgcccaacaggccatgcggaaggccttaa
cactgcgatacgcactgctgcctcacctgtacaccctgttccaccaggctcatgtggccggcgaaacagtggccagaccactgttcct
ggagttccccaaggatagctccacatggaccgtggaccaccagctgctgtggggcgaggccctgctgatcacccctgtgctgcagg
ccggcaaagctgaggtgaccggctacttccctctgggcacctggtacgacctgcagaccgtgcctgttgaggctctgggatctctgcc
ccccccacctgccgcccctcgcgagcctgctatccactctgaaggccagtgggtgacactgcctgccccactcgacaccatcaacgt
gcacctgagagccggttacatcatccccctgcagggccccggcctgaccaccaccgaaagccggcagcaacctatggccctcgcc
gtggccctgaccaagggcggcgaggccaggggtgagctattttgggacgacggcgagagcctggaggtgctggagagaggcgc
ctacacacaagttatcttcctggctcggaacaacaccatcgtgaacgagctggtgcgggtgacaagcgagggcgccggcctccagct
gcagaaagtgacggtgctgggcgtggccacagcccctcagcaggtgctgagcaacggcgtgccagtcagcaatttcacctacagcc
ctgataccaaggttctggacatctgcgtgagcctgctcatgggcgaacagttcctggtgtcctggtgcggatctggcgccacaaacttc
agcctgctgaagcaggccggagatgtggaagaaaatcctggccccatgagcagactggataagtccaaggtgatcaacagcgccct
ggaactgctcaacggcgtcggcatcgagggactcaccacccggaagctggcccagaaactgggcgtggaacaacctacactgtact
ggcacgtgaagaacaaaagagccctgctggatgccctgccaatcgagatgctggaccggcaccatacccacagctgccccctgga
gggagaaagctggcaggacttcctgagaaacaacgctaagagctaccggtgcgccctgctgagccaccgggacggcgccaaggt
gcacttggggaccagacccacggaaaagcaatacgagacactggagaaccagctggccttcctgtgccagcagggcttcagcctg
gagaatgccctctacgccctgtctgccgtgggccactttaccctgggatgtgtgctggaagagcaggagcaccaggtggccaaggaa
gagagggaaacccctaccaccgacagcatgcctcctctgctgaagcaagctatcgagctgttcgacagacagggcgccgagcctgc
tttcctgtttggcctggagctgatcatctgcggcttggagaaacagctgaagtgcgagagcggcggccctaccgacgccctggacgat
ttcgacctggatatgctgcctgccgacgccctggacgacttcgacctggatatgctgcctgccgatgctctggacgacttcgacctgga
catgcttccaggctaactgtgccttctagttgccagccatctgttgtttgcccctcccccgtgccttccttgaccctggaaggtgccactcc
cactgtcctttcctaataaaatgaggaaattgcatcgcattgtctgagtaggtgtcattctattctggggggtggggtggggcaggacag
caagggggaggattgggaagagaatagcaggcaggctggggacaacttgtttattgcagcttataatggttacaaataaagcaatagc
atcacaaatttcacaaataaagcatttttttcactgcattctagttgtggtttgtccaaactcatcaatgtatcttatttgtttgtttgagcgcg
tttcgtcctatttgggactcatcagctggatgtagtaggtggtcgtattcttcacctctccagatcgaaatctggtatgttttaccacgaatcca
ggacgcgcctttgtttgtttgtcattattaattgggggggttgcacacaaacagcatcagggccagcacggccacggtaggatggctcag
gctgtcgacgataacaggctcgttgcggatgttggtcagggtcacctggtttgggaatgtatcttgcacgctctcctgcacgcctctgga
caggttgtcccagttgttttccaggctgatcatagcagcgtcaggctgaaaggcggtgccggtctggatggacacccgcactctattag
agatgtaccggaatctggcggcttcagccaccatttggataatcacgatcagtgttctggccttttcctcgttgtcatttccgccgctccgg
aagaaggagattccgtgtgtaagagcttgcaggcccagggggatctgctgtctgctctggtgggcccatctttccaggtcgccgtatgt
gccgtagaagggcagagagtgctgatcggtgccggtgaacaggtagtcagaggcgctgctaggggcgtcccgcaggaagtaaga
ctgggtgccggctctgtaggccaccacataggcgtttgtcacgtcaatgcccacctcgatgctctctgtatcgctgttgctcagctcgac
ggtgatgtatctgtttctctcctgcagggttgttggatcaggcagcacagggatgtcgtggatcagtccacctctgagccgttccctcag
ggcctcgatgaactgcttgtagctctggctggtggcgccctcggtgctgaatttgataggtctatcttccatggtggctttacgagggtag
gaagtggtacggaaagttggtataagacaaaagtgttgtggaattgctccaggcgatctgacggttcactaaacgagctctgcttttata
ggcgcccaccgtacacgcctaaagcttatacgttctctatcactgatagggagtaaactggatatacgttctctatcactgatagggagta
aactgtagatacgttctctatcactgatagggagtaaactggtcatacgttctctatcactgatagggagtaaactccttatacgttctctatc
actgatagggagtaaagtctgcatacgttctctatcactgatagggagtaaactcttcatacgttctctatcactgatagggagtaaactgg
ggactttccagctggggactttccagctgggactttccaggag
3NF1
(SEQ ID NO: 10)
Ctggggactttccagcctggggactttccagctgggactttccagg
3NF2
(SEQ ID NO: 11)
Ctggggactttccagctggggactttccagctgggactttccaggag
CBh promoter
(SEQ ID NO: 32)
Cgttacataacttacggtaaatggcccgcctggctgaccgcccaacgacccccgcccattgacgtcaatagtaacgccaatagggac
tttccattgacgtcaatgggtggagtatttacggtaaactgcccacttggcagtacatcaagtgtatcatatgccaagtacgccccctattg
acgtcaatgacggtaaatggcccgcctggcattgtgcccagtacatgaccttatgggactttcctacttggcagtacatctacgtattagt
catcgctattaccatggtcgaggtgagccccacgttctgcttcactctccccatctcccccccctccccacccccaattttgtatttatttatt
ttttaattattttgtgcagcgatgggggcggggggggggggggggcgcgcgccaggcggggcggggcggggcgaggggcggg
gcggggcgaggcggagaggtgcggcggcagccaatcagagcggcgcgctccgaaagtttccttttatggcgaggcggcggcggc
ggcggccctataaaaagcgaagcgcgcggcgggcgggagtcgctgcgcgctgccttcgccccgtgccccgctccgccgccgcct
cgcgccgcccgccccggctctgactgaccgcgttactcccacaggtgagcgggcgggacggcccttctcctccgggctgtaattag
ctgagcaagaggtaagggtttaagggatggttggttggtggggtattaatgtttaattacctggagcacctgcctgaaatcactttttttca
ggttgg
secGAA (sp7-delta8-hGAA)
Nucleotide sequence
(SEQ ID NO: 50)
atggccttcttgtggctgctcagctgctgggccctgctgggcaccacattcggcctgcttgtgcctagagagctgagcggctctagccc
agtgctggaggaaacccacccagcccaccagcagggagctagccggcctggacccagagacgcccaggcccaccccggaagac
ccagagccgtgcctacccagtgcgacgtgcctcccaacagcagattcgactgcgcccctgataaggccattacccaggagcagtgtg
aagctagaggctgctgctacatccctgccaagcagggcctgcaaggcgcccagatgggccaaccttggtgcttcttcccaccctctta
ccctagctacaagctggaaaacctgagctcttctgagatgggctacaccgccaccctgacaagaaccaccccaacattcttcccaaaa
gacatcctgaccctgagactggacgtgatgatggaaaccgagaaccggctgcatttcaccattaaggaccccgccaacagacggtac
gaggtccccctggaaacaccccacgtgcacagccgggccccttcccctctgtacagtgtggaattcagcgaggaacccttcggcgtg
atcgtgagaaggcagctggatggcagagtgctgctcaacaccacagtggctccattattcttcgccgaccagttcctgcagctgagca
ccagcctgccctcacagtacatcacaggcctcgccgaacacctgtctcctctcatgctgagcaccagctggacaagaatcacactgtg
gaaccgggacctggcccccacacctggggctaatctgtacggctcccaccccttctacctggcgctggaagatggcggcagcgccc
acggcgttttcctgctgaacagcaatgccatggacgtggtgctgcagcctagccctgccctgagctggcggagcaccggcggaatcc
tggacgtgtacatcttcctgggacccgagcctaagagcgtggtgcagcaatatctggatgtggtgggctacccctttatgcctccttact
ggggcctgggttttcacctgtgtagatggggctactcatctaccgcgatcaccagacaggtggttgagaatatgacccgggcccacttc
cctctcgacgtgcagtggaacgacctggactacatggacagcagaagagatttcacgttcaacaaggacggcttcagggatttccctg
ctatggtgcaggagctcaccagggaggcagacggtacatgatgatcgtggatcctgctatcagcagcagcggccccgccggccag
ctatagaccttatgacgagggactgagacggggcgttttcatcacaaacgagacaggacagcctcttatcggcaaggtgtggcccgg
ctctaccgcctttcctgacttcacaaatcctaccgccctggcctggtgggaagatatggtcgccgagttccacgaccaggtccctttcga
cgggatgtggatcgacatgaacgagcccagtaacttcattagaggctctgaagatggatgtcctaacaacgagctggagaaccctcct
tacgtgcctggcgtggtcggcggaactctgcaggccgctacaatctgtgccagctcccaccagttcctgagcacccactacaacctgc
acaatctgtacggactgacagaggctatcgccagtcatagagccctggtcaaggccagaggcaccagacccttcgtgatctccagaa
gcaccttcgccggacacggcagatatgccggccactggacaggcgacgtgtggtcctcttgggagcagctcgcaagctctgtgccg
gaaatcctgcaatttaacttgctgggcgtgcctctggtggggctgatgtgtgcggctttctgggcaacacatctgaagaattgtgcgtg
cggtggacccagctgggcgctttttacccttttatgagaaatcacaactccctgctgagcctgccacaggagccttacagcttcagcga
acctgcccaacaggccatgcggaaggccttaacactgcgatacgcactgctgcctcacctgtacaccctgttccaccaggctcatgtg
gccggcgaaacagtggccagaccactgttcctggagttccccaaggatagctccacatggaccgtggaccaccagctgctgtgggg
cgaggccctgctgatcacccctgtgctgcaggccggcaaagctgaggtgaccggctacttccctctgggcacctggtacgacctgca
gaccgtgcctgttgaggctctgggatctctgccccccccacctgccgcccctcgcgagcctgctatccactctgaaggccagtgggtg
acactgcctgccccactcgacaccatcaacgtgcacctgagagccggttacatcatccccctgcagggccccggcctgaccaccacc
gaaagccggcagcaacctatggccctcgccgtggccctgaccaagggggcgaggccaggggtgagctattttgggacgacggc
gagagcctggaggtgctggagagaggcgcctacacacaagttatcttcctggctcggaacaacaccatcgtgaacgagctggtgcg
ggtgacaagcgagggcgccggcctccagctgcagaaagtgacggtgctgggcgtggccacagcccctcagcaggtgctgagcaa
cggcgtgccagtcagcaatttcacctacagccctgataccaaggttctggacatctgcgtgagcctgctcatgggcgaacagttcctgg
tgtcctggtgctaa
Amino acid sequence
(SEQ ID NO: 51)
MAFLWLLSCWALLGTTFGLLVPRELSGSSPVLEETHPAHQQGASRPGPRDAQAHPGR
PRAVPTQCDVPPNSRFDCAPDKAITQEQCEARGCCYIPAKQGLQGAQMGQPWCFFPP
SYPSYKLENLSSSEMGYTATLTRTTPTFFPKDILTLRLDVMMETENRLHFTIKDPANRR
YEVPLETPHVHSRAPSPLYSVEFSEEPFGVIVRRQLDGRVLLNTTVAPLFFADQFLQLS
TSLPSQYITGLAEHLSPLMLSTSWTRITLWNRDLAPTPGANLYGSHPFYLALEDGGSA
HGVFLLNSNAMDVVLQPSPALSWRSTGGILDVYIFLGPEPKSVVQQYLDVVGYPFMP
PYWGLGFHLCRWGYSSTAITRQVVENMTRAHFPLDVQWNDLDYMDSRRDFTFNKD
GFRDFPAMVQELHQGGRRYMMIVDPAISSSGPAGSYRPYDEGLRRGVFITNETGQPLI
GKVWPGSTAFPDFTNPTALAWWEDMVAEFHDQVPFDGMWIDMNEPSNFIRGSEDGC
PNNELENPPYVPGVVGGTLQAATICASSHQFLSTHYNLHNLYGLTEAIASHRALVKAR
GTRPFVISRSTFAGHGRYAGHWTGDVWSSWEQLASSVPEILQFNLLGVPLVGADVCG
FLGNTSEELCVRWTQLGAFYPFMRNHNSLLSLPQEPYSFSEPAQQAMRKALTLRYAL
LPHLYTLFHQAHVAGETVARPLFLEFPKDSSTWTVDHQLLWGEALLITPVLQAGKAE
VTGYFPLGTWYDLQTVPVEALGSLPPPPAAPREPAIHSEGQWVTLPAPLDTINVHLRA
GYIIPLQGPGLTTTESRQQPMALAVALTKGGEARGELFWDDGESLEVLERGAYTQVIF
LARNNTIVNELVRVTSEGAGLQLQKVTVLGVATAPQQVLSNGVPVSNFTYSPDTKVL
DICVSLLMGEQFLVSWC
P2A
(SEQ ID NO: 37)
Nucleotide sequence
GGATCTGGCGCCACAAACTTCAGCCTGCTGAAGCAGGCCGGAGATGTGGAAGAA
AATCCTGGCCCC
Amino acid sequence
(SEQ ID NO: 29)
ATNFSLLKQAGDVEENPGP
Tet-on 3G
Nucleotide sequence
(SEQ ID NO: 43)
Atgagcagactggataagtccaaggtgatcaacagcgccctggaactgctcaacggcgtcggcatcgagggactcaccacccgga
agctggcccagaaactgggcgtggaacaacctacactgtactggcacgtgaagaacaaaagagccctgctggatgccctgccaatc
gagatgctggaccggcaccatacccacagctgccccctggagggagaaagctggcaggacttcctgagaaacaacgctaagagct
accggtgcgccctgctgagccaccgggacggcgccaaggtgcacttggggaccagacccacggaaaagcaatacgagacactgg
agaaccagctggccttcctgtgccagcagggcttcagcctggagaatgccctctacgccctgtctgccgtgggccactttaccctggg
atgtgtgctggaagagcaggagcaccaggtggccaaggaagagagggaaacccctaccaccgacagcatgcctcctctgctgaag
caagctatcgagctgttcgacagacagggcgccgagcctgctttcctgtttggcctggagctgatcatctgcggcttggagaaacagct
gaagtgcgagagcggcggccctaccgacgccctggacgatttcgacctggatatgctgcctgccgacgccctggacgacttcgacc
tggatatgctgcctgccgatgctctggacgacttcgacctggacatgcttccaggctaa
Amino acid sequence
(SEQ ID NO: 44)
MSRLDKSKVINSALELLNGVGIEGLTTRKLAQKLGVEQPTLYWHVKNKRALLDALPI
EMLDRHHTHSCPLEGESWQDFLRNNAKSYRCALLSHRDGAKVHLGTRPTEKQYETL
ENQLAFLCQQGFSLENALYALSAVGHFTLGCVLEEQEHQVAKEERETPTTDSMPPLLK
QAIELFDRQGAEPAFLFGLELIICGLEKQLKCESGGPTDALDDFDLDMLPADALDDED
LDMLPADALDDFDLDMLPG
TRE3G promoter
(SEQ ID NO: 45)
Tttactccctatcagtgatagagaacgtatgaagagtttactccctatcagtgatagagaacgtatgcagactttactccctatcagtgata
gagaacgtataaggagtttactccctatcagtgatagagaacgtatgaccagtttactccctatcagtgatagagaacgtatctacagttta
ctccctatcagtgatagagaacgtatatccagtttactccctatcagtgatagagaacgtataagctttaggcgtgtacggtgggcgccta
taaaagcagagctcgtttagtgaaccgtcagatcgcctggagcaattccacaacacttttgtcttataccaactttccgtaccacttcctac
cctcgtaaa
Arbin A-chain
Nucleotide sequence
(SEQ ID NO: 63)
Atggaagatagacctatcaaattcagcaccgagggcgccaccagccagagctacaagcagttcatcgaggccctgagggaacggc
tcagaggtggactgatccacgacatccctgtgctgcctgatccaacaaccctgcaggagagaaacagatacatcaccgtcgagctga
gcaacagcgatacagagagcatcgaggtgggcattgacgtgacaaacgcctatgtggtggcctacagagccggcacccagtcttact
tcctgcgggacgcccctagcagcgcctctgactacctgttcaccggcaccgatcagcactctctgcccttctacggcacatacggcga
cctggaaagatgggcccaccagagcagacagcagatccccctgggcctgcaagctcttacacacggaatctccttcttccggagcgg
cggaaatgacaacgaggaaaaggccagaacactgatcgtgattatccaaatggtggctgaagccgccagattccggtacatctctaat
agagtgcgggtgtccatccagaccggcaccgcctttcagcctgacgctgctatgatcagcctggaaaacaactgggacaacctgtcc
agaggcgtgcaggagagcgtgcaagatacattcccaaaccaggtgaccctgaccaacatccgcaacgagcctgttatcgtcgacag
cctgagccatcctaccgtggccgtgctggccctgatgctgtttgtgtgcaacccccccaattaa
Amino acid sequence
(SEQ ID NO: 64)
MEDRPIKFSTEGATSQSYKQFIEALRERLRGGLIHDIPVLPDPTTLQERNRYITVELSNS
DTESIEVGIDVTNAYVVAYRAGTQSYFLRDAPSSASDYLFTGTDQHSLPFYGTYGDLE
RWAHQSRQQIPLGLQALTHGISFFRSGGNDNEEKARTLIVIIQMVAEAARFRYISNRVR
VSIQTGTAFQPDAAMISLENNWDNLSRGVQESVQDTFPNQVTLTNIRNEPVIVDSLSH
PTVAVLALMLFVCNPPN
K19 aptazyme
(SEQ ID NO: 54)
Ggcgcgtcctggattcgtggtaaaacataccagatttcgatctggagaggtgaagaatacgaccacctactacatccagctgatgagt
cccaaataggacgaaacgcgct
Insulator sequence
(SEQ ID NO: 55)
Caaacaaacaaa
bGH poly(A) sequence
(SEQ ID NO: 46)
ctgtgccttctagttgccagccatctgttgtttgcccctcccccgtgccttccttgaccctggaaggtgccactcccactgtcctttcctaat
aaaatgaggaaattgcatcgcattgtctgagtaggtgtcattctattctggggggtggggtggggcaggacagcaagggggaggattg
ggaagagaatagcaggcaggctgggga
SV40 poly(A) sequence
(SEQ ID NO: 47)
Taagatacattgatgagtttggacaaaccacaactagaatgcagtgaaaaaaatgctttatttgtgaaatttgtgatgctattgctttatttgt
aaccattataagctgcaataaacaagtt

### Example 6

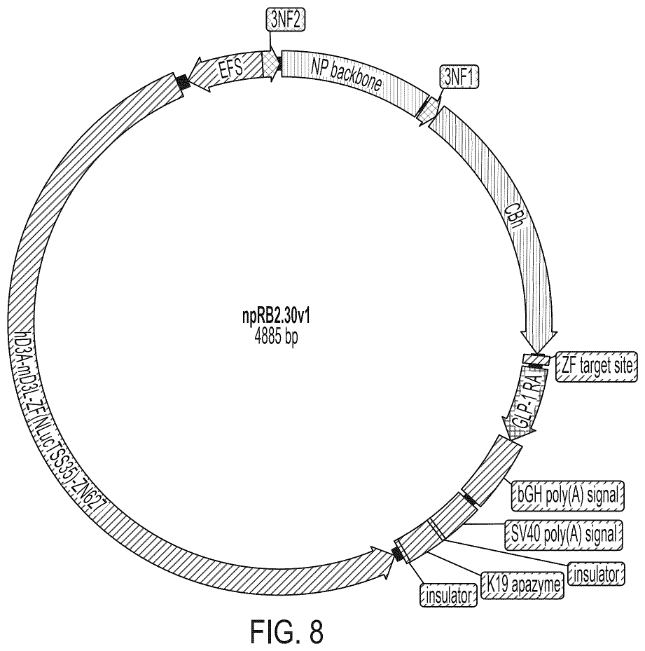

In the present example a Glucagon Like Peptide 1 Receptor Agonist (GLP-1RA) is delivered for the management of type 2 diabetes. The GLP-1RA peptide is delivered with a secretion signal under the regulation of a constitutive promoter. Dose-down titration is enabled by self-directed, small-molecule induced epigenetic silencing of the promoter region of the construct. FIG. 8 shows a plasmid map for the genetic construct described in this example.

Full sequence (except nanoplasmid backbone)
(SEQ ID NO: 65)
ctggggactttccagcctggggactttccagctgggactttccaggcgttacataacttacggtaaatggcccgcctggctgaccgccc
aacgacccccgcccattgacgtcaatagtaacgccaatagggactttccattgacgtcaatgggtggagtatttacggtaaactgccca
cttggcagtacatcaagtgtatcatatgccaagtacgccccctattgacgtcaatgacggtaaatggcccgcctggcattgtgcccagta
catgaccttatgggactttcctacttggcagtacatctacgtattagtcatcgctattaccatggtcgaggtgagccccacgttctgcttcac
tctccccatctcccccccctccccacccccaattttgtatttatttattttttaattattttgtgcagcgatgggggcgggggggggggggg
ggcgcgcgccaggcggggcggggcggggcgaggggcggggcggggcgaggcggagaggtgcggcggcagccaatcagag
cggcgcgctccgaaagtttccttttatggcgaggcggcggcggcggcggccctataaaaagcgaagcgcgcggcgggcgggagt
cgctgcgcgctgccttcgccccgtgccccgctccgccgccgcctcgcgccgcccgccccggctctgactgaccgcgttactcccac
aggtgagcgggcgggacggcccttctcctccgggctgtaattagctgagcaagaggtaagggtttaagggatggttggttggtgggg
tattaatgtttaattacctggagcacctgcctgaaatcactttttttcaggttgggttggacgcgtctggcgacagacagccggcgctagc
gccaccatgaaaatcatcctgtggctgtgcgtgttcggcctctttctggccaccctgttccccatcagctggcagatgcctgtggaatctg
gcctgagctctgaggatagcgccagcagcgagagcttcgccaagagaatcaagcggcacggcgagggaacattcaccagcgacg
tgtcctcctacctggaaggccaggccgctcaggagttcatcgcctggctggtggacggcagaggctaatgactgtgccttctagttgcc
agccatctgttgtttgcccctcccccgtgccttccttgaccctggaaggtgccactcccactgtcctttcctaataaaatgaggaaattgca
tcgcattgtctgagtaggtgtcattctattctggggggtggggtggggcaggacagcaagggggaggattgggaagagaatagcag
gcaggctggggagggccgccgatcgaacttgtttattgcagcttataatggttacaaataaagcaatagcatcacaaatttcacaaataa
agcatttttttcactgcattctagttgtggtttgtccaaactcatcaatgtatcttatttgtttgtttgagcgcgtttcgtcctatttgggactca
tcagctggatgtagtaggtggtcgtattcttcacctctccagatcgaaatctggtatgttttaccacgaatccaggacgcgcctttgtttgtttg
gactagaagcggccgcctattattcctcgccctggcctccctccttggactcgcacagtctctctgggatgtggctgatgttccgtctcgg
gatcttgaaaggatcctcgatgttctgatcctcccactgtttgccgacgctagccaggtttctgaaggtttctctcatcacgtctctgtacag
gttcttttggctagggtcgagcagagcccactcttccagagtgaaattcacagccacgtcctcgaaggccacgctgtcgcttcctccgc
cgccagagccgccgccaccgctgctggtcttcttgcctgtgtgtgtgcgctggtgtgtggtgaggtggtcgcttcttgaaaagctttttcc
acactcaggacacttatagggcttctctccggtgtgtgttctctggtgctctgtcagatgaccgctctgggaaaagctcttgccgcattctg
ggcacttataaggcttttcgccggtgtgggtcctttggtgctcggtcaggttatcggctctggagaagctcttgccgcattcgggacactt
ataaggtttctcgcctgtgtgggtccgctggtgtcttgtcaggtcggcaggagagctgaaggattttccgcactcagggcacttgtaagg
tttttcgcctgtatgggttctctggtgccgggccagatctctgcaatcggagaagctcttgccacattcaggacatttgtagggtttctcac
cggtatgggtccgctggtgccgcaccaggtggccagggtcgctaaaagatttgccgcactctgggcacttgtagggcttctcgccag
gctccaggctgccgcctccgccgctcccgcctccgccgctcaggggcaggctattttgggagaagtatttgaagtactctctaagtgg
cagcaggcagtttttcaccagcaggtcgaccttaggggcatccagcttggatctgcttctcacctgagcttgcaggtactcttcttccttag
gtgtcagaggggcgtgcttggacttcaggccagggatattgctccacacccgcatggcattctggtagtcgcggccccggacatcctg
cagggtcacggcctcggtctgcaggaatcttgtggttgtctcctggtcgtcctcggtcagcagcaggttgtccatgaagatccagaaga
aaggtctctggctttcctgccgaggcagggcgtactgcaggatacggtggaattgaaacatgtaccagccggggcaccggtcgcag
ctgctgcccaggggctgggtgctgccgtacaccagatcgaaaggtccccatttctcgacatctctccgcaccacgtttgtcacatcttcc
acgtacttcagggtgccgccgccagacccgcttccagattccagaaatcccaggctcttcagcaccttgtcgatgtttctgaacaggctc
agcactctcacaggctgcctcttccaagccgacactgtcttgtatatttccataggtcccatgtggctgcctctcaggctgagaggcacc
agtccgctgctgaaggaggggccgcgggagttggcgttagaatttccggagctcacgcaggcaaagtactccttcaggggggcgaa
caggtgtctgatcacaggcacgctccaggatctgcccagcagccgctgtctggccagtctgctcatgttagaaacgtcggtgtagtgc
acggggaagccaaaaactctttccatctcggtgcaccacaggatgtcctccttctcgttcataaacacagggaagtgctgatctttgccc
tgcttgatgctgttgctcctggtggtgatggttctcaccttactgaacttggcgattctgccgtgttccagacactcctgcagttccagcttgt
cattcacggtagaggcgagaggtctgttcatgcctggcaggttgccccagaagtaccgggcccggtgggggcagacacttccttag
catcaatcatcacagggttagactccaggaatctgctgatgtcccgtttgtcgctcacgcccatggccaccacgttctcgaacagccag
aaaaaaggtctgtcatcgccttccttgggacgggcatcgtgcagcagccggtagaactcgaagaacagtctgcctgtgccctcgtaca
ggccctttctagcggggttcacgatgctcaggtcgttgcaggggcttccgccgataaccaggtcgaaggggccccactcctggatatg
cttctgggtcacagaccggacgtcgcccacgtacatgatcttgccttggtgccgcaccatgcccacggtgatagaatcttcgcacactt
cactggcgatgtatctgtccacctggatgcccaggtccttcagcaccagcaggccggtggcgatgccgtcaaagaggctcagcaccc
ggattggcttccgcttctcggctggcacaggagggtacaccttaggtgggtcgaactcctggtcgtggttgacctttctcttttttttaggg
gccatggtggcagcctgcttttttgtacaaacttgcctgtgttctggcggcaaacccgttgcgaaaaagaacgttcacggcgactactgc
acttatatacggttctcccccaccctcgggaaaaaggcggagccagtacacgacatcactttcccagtttaccccgcgccaccttctcta
ggcaccggatcaattgccgacccctccccccaacttctcggggactgtgggcgatgtgcgctctgcccactgacgggcaccggagc
cctggggactttccagctggggactttccagctgggactttccaggag
3NF1
(SEQ ID NO: 10)
Ctggggactttccagcctggggactttccagctgggactttccagg
3NF2
(SEQ ID NO: 11)
Ctggggactttccagctggggactttccagctgggactttccaggag
CBh promoter
(SEQ ID NO: 32)
Cgttacataacttacggtaaatggcccgcctggctgaccgcccaacgacccccgcccattgacgtcaatagtaacgccaatagggac
tttccattgacgtcaatgggtggagtatttacggtaaactgcccacttggcagtacatcaagtgtatcatatgccaagtacgccccctattg
acgtcaatgacggtaaatggcccgcctggcattgtgcccagtacatgaccttatgggactttcctacttggcagtacatctacgtattagt
catcgctattaccatggtcgaggtgagccccacgttctgcttcactctccccatctcccccccctccccacccccaattttgtatttatttatt
ttttaattattttgtgcagcgatgggggcggggggggggggggggcgcgcgccaggcggggcggggcggggcgaggggcggg
gcggggcgaggcggagaggtgcggcggcagccaatcagagcggcgcgctccgaaagtttccttttatggcgaggcggcggcggc
ggcggccctataaaaagcgaagcgcgcggcgggcgggagtcgctgcgcgctgccttcgccccgtgccccgctccgccgccgcct
cgcgccgcccgccccggctctgactgaccgcgttactcccacaggtgagcgggcgggacggcccttctcctccgggctgtaattag
ctgagcaagaggtaagggtttaagggatggttggttggtggggtattaatgtttaattacctggagcacctgcctgaaatcactttttttca
ggttgg
Zink finger target site
(SEQ ID NO: 66)
TGGCGACAGACAGCCGGC
GLP-1 RA
Nucleotide sequence
(SEQ ID NO: 67)
ATGAAAATCATCCTGTGGCTGTGCGTGTTCGGCCTCTTTCTGGCCACCCTGTTCCC
CATCAGCTGGCAGATGCCTGTGGAATCTGGCCTGAGCTCTGAGGATAGCGCCAGC
AGCGAGAGCTTCGCCAAGAGAATCAAGCGGCACGGCGAGGGAACATTCACCAGC
GACGTGTCCTCCTACCTGGAAGGCCAGGCCGCTCAGGAGTTCATCGCCTGGCTGG
TGGACGGCAGAGGCtaa
Amino acid sequence
(SEQ ID NO: 68)
MKIILWLCVFGLFLATLFPISWQMPVESGLSSEDSASSESFAKRIKRHGEGTFTSDVSS
YLEGQAAQEFIAWLVDGRG
bGH poly(A) sequence
(SEQ ID NO: 46)
ctgtgccttctagttgccagccatctgttgtttgcccctcccccgtgccttccttgaccctggaaggtgccactcccactgtcctttcctaat
aaaatgaggaaattgcatcgcattgtctgagtaggtgtcattctattctggggggtggggtggggcaggacagcaagggggaggattg
ggaagagaatagcaggcaggctgggga
SV40 poly(A) sequence
(SEQ ID NO: 47)
Taagatacattgatgagtttggacaaaccacaactagaatgcagtgaaaaaaatgctttatttgtgaaatttgtgatgctattgctttatttgt
aaccattataagctgcaataaacaagtt
K19 aptazyme
Ggcgcgtcctggattcgtggtaaaacataccagatttcgatctggagaggtgaagaatacgaccacctactacatccagctgatgagt
(SEQ ID NO: 54)
cccaaataggacgaaacgcgct
Insulator sequence
(SEQ ID NO: 55)
Caaacaaacaaa
Epigenetics effector (hD3A-mD3L-ZF-ZN627)
Nucleotide sequence
(SEQ ID NO: 69)
atggcccctaaaaaaaagagaaaggtcaaccacgaccaggagttcgacccacctaaggtgtaccctcctgtgccagccgagaagcg
gaagccaatccgggtgctgagcctctttgacggcatcgccaccggcctgctggtgctgaaggacctgggcatccaggtggacagata
catcgccagtgaagtgtgcgaagattctatcaccgtgggcatggtgcggcaccaaggcaagatcatgtacgtgggcgacgtccggtc
tgtgacccagaagcatatccaggagtggggccccttcgacctggttatcggcggaagcccctgcaacgacctgagcatcgtgaaccc
cgctagaaagggcctgtacgagggcacaggcagactgttcttcgagttctaccggctgctgcacgatgcccgtcccaaggaaggcg
atgacagaccttttttctggctgttcgagaacgtggtggccatgggcgtgagcgacaaacgggacatcagcagattcctggagtctaac
cctgtgatgattgatgctaaggaagtgtctgccgcccaccgggcccggtacttctggggcaacctgccaggcatgaacagacctctcg
cctctaccgtgaatgacaagctggaactgcaggagtgtctggaacacggcagaatcgccaagttcagtaaggtgagaaccatcacca
ccaggagcaacagcatcaagcagggcaaagatcagcacttccctgtgtttatgaacgagaaggaggacatcctgtggtgcaccgag
atggaaagagtttttggcttccccgtgcactacaccgacgtttctaacatgagcagactggccagacagcggctgctgggcagatcctg
gagcgtgcctgtgatcagacacctgttcgcccccctgaaggagtactttgcctgcgtgagctccggaaattctaacgccaactcccgc
ggcccctccttcagcagcggactggtgcctctcagcctgagaggcagccacatgggacctatggaaatatacaagacagtgtcggct
tggaagaggcagcctgtgagagtgctgagcctgttcagaaacatcgacaaggtgctgaagagcctgggatttctggaatctggaagc
gggtctggcggcggcaccctgaagtacgtggaagatgtgacaaacgtggtgcggagagatgtcgagaaatggggacctttcgatct
ggtgtacggcagcacccagcccctgggcagcagctgcgaccggtgccccggctggtacatgtttcaattccaccgtatcctgcagta
cgccctgcctcggcaggaaagccagagacctttcttctggatcttcatggacaacctgctgctgaccgaggacgaccaggagacaac
cacaagattcctgcagaccgaggccgtgaccctgcaggatgtccggggccgcgactaccagaatgccatgcgggtgtggagcaata
tccctggcctgaagtccaagcacgcccctctgacacctaaggaagaagagtacctgcaagctcaggtgagaagcagatccaagctg
gatgcccctaaggtcgacctgctggtgaaaaactgcctgctgccacttagagagtacttcaaatacttctcccaaaatagcctgcccctg
agcggcggaggcgggagcggcggaggcggcagcctggagcctggcgagaagccctacaagtgcccagagtgcggcaaatctttt
agcgaccctggccacctggtgcggcaccagcggacccataccggtgagaaaccctacaaatgtcctgaatgtggcaagagcttctcc
gattgcagagatctggcccggcaccagagaacccatacaggcgaaaaaccttacaagtgccctgagtgcggaaaatccttcagctct
cctgccgacctgacaagacaccagcggacccacacaggcgagaaaccttataagtgtcccgaatgcggcaagagcttctccagagc
cgataacctgaccgagcaccaaaggacccacaccggcgaaaagccttataagtgcccagaatgcggcaagagcttttcccagagcg
gtcatctgacagagcaccagagaacacacaccggagagaagccctataagtgtcctgagtgtggaaaaagcttttcaagaagcgacc
acctcaccacacaccagcgcacacacacaggcaagaagaccagcagcggtggcggcggctctggcggcggaggaagcgacag
cgtggccttcgaggacgtggctgtgaatttcactctggaagagtgggctctgctcgaccctagccaaaagaacctgtacagagacgtg
atgagagaaaccttcagaaacctggctagcgtcggcaaacagtgggaggatcagaacatcgaggatcctttcaagatcccgagacg
gaacatcagccacatcccagagagactgtgcgagtccaaggagggaggccagggcgaggaataa
Amino acid sequence
(SEQ ID NO: 70)
MAPKKKRKVNHDQEFDPPKVYPPVPAEKRKPIRVLSLFDGIATGLLVLKDLGIQVDRY
IASEVCEDSITVGMVRHQGKIMYVGDVRSVTQKHIQEWGPFDLVIGGSPCNDLSIVNP
ARKGLYEGTGRLFFEFYRLLHDARPKEGDDRPFFWLFENVVAMGVSDKRDISRFLES
NPVMIDAKEVSAAHRARYFWGNLPGMNRPLASTVNDKLELQECLEHGRIAKFSKVR
TITTRSNSIKQGKDQHFPVFMNEKEDILWCTEMERVFGFPVHYTDVSNMSRLARQRL
LGRSWSVPVIRHLFAPLKEYFACVSSGNSNANSRGPSFSSGLVPLSLRGSHMGPMEIY
KTVSAWKRQPVRVLSLFRNIDKVLKSLGFLESGSGSGGGTLKYVEDVTNVVRRDVE
KWGPFDLVYGSTQPLGSSCDRCPGWYMFQFHRILQYALPRQESQRPFFWIFMDNLLL
TEDDQETTTRFLQTEAVTLQDVRGRDYQNAMRVWSNIPGLKSKHAPLTPKEEEYLQ
AQVRSRSKLDAPKVDLLVKNCLLPLREYFKYFSQNSLPLSGGGGSGGGGSLEPGEKP
YKCPECGKSFSDPGHLVRHQRTHTGEKPYKCPECGKSFSDCRDLARHQRTHTGEKPY
KCPECGKSFSSPADLTRHQRTHTGEKPYKCPECGKSFSRADNLTEHQRTHTGEKPYKC
PECGKSFSQSGHLTEHQRTHTGEKPYKCPECGKSFSRSDHLTTHQRTHTGKKTSSGGG
GSGGGGSDSVAFEDVAVNFTLEEWALLDPSQKNLYRDVMRETFRNLASVGKQWEDQ
NIEDPFKIPRRNISHIPERLCESKEGGQGEE
EFS promoter
(SEQ ID NO: 30)
GGCTCCGGTGCCCGTCAGTGGGCAGAGCGCACATCGCCCACAGTCCCCGAGAAG
TTGGGGGGAGGGGTCGGCAATTGATCCGGTGCCTAGAGAAGGTGGCGCGGGGTA
AACTGGGAAAGTGATGTCGTGTACTGGCTCCGCCTTTTTCCCGAGGGTGGGGGAG
AACCGTATATAAGTGCAGTAGTCGCCGTGAACGTTCTTTTTCGCAACGGGTTTGCC
GCCAGAACACAGG

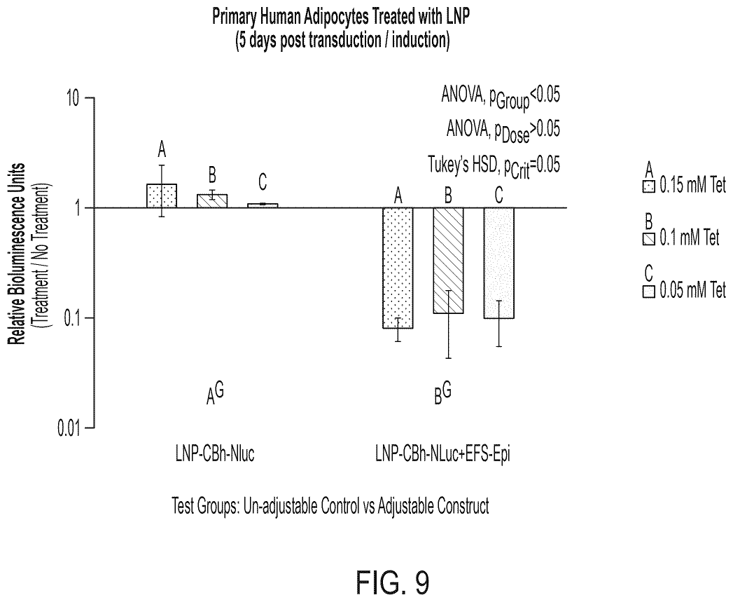

The activity of the self-silencing epigenetic regulator is qualified using an nLuc bioluminescent reporter (FIG. 9). Activation of the self-silencing signal by delivery of an operator induces a reduction by approximately 2× in the reporter transgene expression.

### Example 7

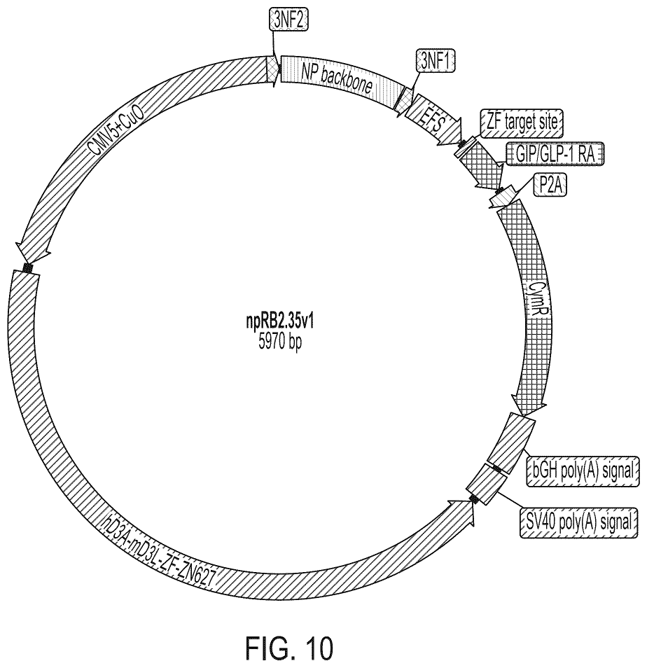

In the present example a Glucagon Like Peptide 1 and Gastric Inhibitor Peptide dual receptor agonist (GLP/GIP) is delivered for the treatment of Type 2 Diabetes or Obesity under the regulation of an EFS promoter. To enable dose down-titration should the therapeutic reach optimal therapeutic effect or to reduce the dose should the therapeutic exceed a tolerability limit, a small molecule actuator (Cumate) is used to activate an in-cis delivered epigenetic effector element to silence expression of the therapeutic transgene. FIG. 10 shows a plasmid map for the genetic construct described in this example. The specific elements of the constructs and the full construct design are shown below.

Actuator: water insoluble cumate, 4-Isopropylbenzoic acid (Sigma 268402) or USP-grade equivalent thereof.

Full sequence (except nanoplasmid backbone)
(SEQ ID NO: 71)
ctggggactttccagcctggggactttccagctgggactttccaggggctccggtgcccgtcagtgggcagagcgcacatcgcccac
agtccccgagaagttggggggaggggtcggcaattgatccggtgcctagagaaggtggcgcggggtaaactgggaaagtgatgtc
gtgtactggctccgcctttttcccgagggtgggggagaaccgtatataagtgcagtagtcgccgtgaacgttctttttcgcaacgggtttg
ccgccagaacacagggttggacgcgtctggcgacagacagccggcgctagcgccaccatgcactggggcaccctgtgcggcttcc
tgtggctgtggccctacctgttctacgtgcaggcttatggcgagggcacattcaccagcgactacagcaagtacctggataagatcgcc
cagaaagcctttgtgcagtggctgatcgccggaggaccttccagcggcgcccctcctccatctggatctggcgccacaaacttcagcc
tgctgaagcaggccggagatgtggaagaaaatcctggccccatggcttacccatacgatgttccagattacgctagtttggtgaggca
gcagagaccgatgacatccactttgcctttctctccacaggtgtccactcccaggtccaagtttggaagatctaccatgccaaagagac
ccagacccgtgatcatgagtccaaagagaagaacacaggcagagcgcgcaatggagacccagggcaagttgattgcagcggccct
gggggttttacgggaaaaaggttacgcgggattccggatcgcagatgtgcccggtgctgcaggtgtctcgagaggagcgcagagcc
atcatttcccgacaaagcttgagcttctgcttgccacttttgaatggctttacgaacagatcaccgaacgcagtcgggctcgattagcgaa
attgaagccagaggatgacgtcatccagcaaatgctggacgacgccgccgaatttttcctcgacgatgacttctctatcagccttgatttg
attgtggctgccgaccgggatccagcgttacgcgagggtattcagcgcacggtagagaggaatcggtttgtcgtcgaggatatgtggc
ttggtgttctggtgagccgtggtctttcgcgtgatgatgcagaagatatcctttggttgatattcaattcggtgcgtgggcttgctgttcgta
gcctatggcagaaggacaaagaacgctttgagcgtgtcaggaactcgacactcgaaattgcgcgagagcggtacgcgaaattcaag
cgcgcggccaaaccctctggccggtctctctctgttcgataataactgtgccttctagttgccagccatctgttgtttgcccctcccccgtg
ccttccttgaccctggaaggtgccactcccactgtcctttcctaataaaatgaggaaattgcatcgcattgtctgagtaggtgtcattctatt
ctggggggtggggtggggcaggacagcaagggggaggattgggaagagaatagcaggcaggctggggagggccgccgatcga
acttgtttattgcagcttataatggttacaaataaagcaatagcatcacaaatttcacaaataaagcatttttttcactgcattctagttgtggtt
tgtccaaactcatcaatgtatcttagactagaagcggccgcctattattcctcgccctggcctccctccttggactcgcacagtctctctgg
gatgtggctgatgttccgtctcgggatcttgaaaggatcctcgatgttctgatcctcccactgtttgccgacgctagccaggtttctgaag
gtttctctcatcacgtctctgtacaggttcttttggctagggtcgagcagagcccactcttccagagtgaaattcacagccacgtcctcga
aggccacgctgtcgcttcctccgccgccagagccgccgccaccgctgctggtcttcttgcctgtgtgtgtgcgctggtgtgtggtgagg
tggtcgcttcttgaaaagctttttccacactcaggacacttatagggcttctctccggtgtgtgttctctggtgctctgtcagatgaccgctct
gggaaaagctcttgccgcattctgggcacttataaggcttttcgccggtgtgggtcctttggtgctcggtcaggttatcggctctggagaa
gctcttgccgcattcgggacacttataaggtttctcgcctgtgtgggtccgctggtgtcttgtcaggtcggcaggagagctgaaggatttt
ccgcactcagggcacttgtaaggtttttcgcctgtatgggttctctggtgccgggccagatctctgcaatcggagaagctcttgccacatt
caggacatttgtagggtttctcaccggtatgggtccgctggtgccgcaccaggtggccagggtcgctaaaagatttgccgcactctgg
gcacttgtagggcttctcgccaggctccaggctgccgcctccgccgctcccgcctccgccgctcaggggcaggctattttgggagaa
gtatttgaagtactctctaagtggcagcaggcagtttttcaccagcaggtcgaccttaggggcatccagcttggatctgcttctcacctga
gcttgcaggtactcttcttccttaggtgtcagaggggcgtgcttggacttcaggccagggatattgctccacacccgcatggcattctggt
agtcgcggccccggacatcctgcagggtcacggcctcggtctgcaggaatcttgtggttgtctcctggtcgtcctcggtcagcagcag
gttgtccatgaagatccagaagaaaggtctctggctttcctgccgaggcagggcgtactgcaggatacggtggaattgaaacatgtac
cagccggggcaccggtcgcagctgctgcccaggggctgggtgctgccgtacaccagatcgaaaggtccccatttctcgacatctctc
cgcaccacgtttgtcacatcttccacgtacttcagggtgccgccgccagacccgcttccagattccagaaatcccaggctcttcagcac
cttgtcgatgtttctgaacaggctcagcactctcacaggctgcctcttccaagccgacactgtcttgtatatttccataggtcccatgtggct
gcctctcaggctgagaggcaccagtccgctgctgaaggaggggccgcgggagttggcgttagaatttccggagctcacgcaggca
aagtactccttcaggggggcgaacaggtgtctgatcacaggcacgctccaggatctgcccagcagccgctgtctggccagtctgctc
atgttagaaacgtcggtgtagtgcacggggaagccaaaaactctttccatctcggtgcaccacaggatgtcctccttctcgttcataaac
acagggaagtgctgatctttgccctgcttgatgctgttgctcctggtggtgatggttctcaccttactgaacttggcgattctgccgtgttcc
agacactcctgcagttccagcttgtcattcacggtagaggcgagaggtctgttcatgcctggcaggttgccccagaagtaccgggccc
ggtgggcggcagacacttccttagcatcaatcatcacagggttagactccaggaatctgctgatgtcccgtttgtcgctcacgcccatg
gccaccacgttctcgaacagccagaaaaaaggtctgtcatcgccttccttgggacgggcatcgtgcagcagccggtagaactcgaag
aacagtctgcctgtgccctcgtacaggccctttctagcggggttcacgatgctcaggtcgttgcaggggcttccgccgataaccaggtc
gaaggggccccactcctggatatgcttctgggtcacagaccggacgtcgcccacgtacatgatcttgccttggtgccgcaccatgccc
acggtgatagaatcttcgcacacttcactggcgatgtatctgtccacctggatgcccaggtccttcagcaccagcaggccggtggcgat
gccgtcaaagaggctcagcacccggattggcttccgcttctcggctggcacaggagggtacaccttaggtgggtcgaactcctggtc
gtggttgacctttctcttttttttaggggccatggtggcagcctgcttttttgtacaaacttggaattcggtaccggatccagtcgactagacc
aaacttggacctgggagtggacacctgtggagagaaaggcaaagtggatgtcattgtcactcaagtgtatggccagatcgggccagg
tgaatatcaaatcctcctcgtttttggaaactgacaatcttagcgcagaagtaatgcccgcttttgagagggagtactcaccccaacagct
ggatctcaagcctgccacacctcacctcgaccatccgccgtctcaagaccgcctactttaattacatcatcagcagcacctccgccaga
aacaaccccgaccgccacccgctgccgcccgccacggtgctcagcctaccttgcgactgtgactggttagacgcctttctcgagagg
ttttccgatccggtcgatgcggactcgctcaggtccctcggtggcggagtaccgttcggaggccgacgggtttccgatccaagagtac
tggaaagaccgcgaagagtttgtcctcaaccgcgagcccaacagctggccctcgcagacagcgatgcggaagagagtgaccgcg
gaggctggatcggtcccggtgtcttctatggaggtcaaaacagcgtggatggcgtctccaggcgatatgaggcgcgcccggttcact
aaacaccggtaacaaacagacaatctggtctgtttgattataccggttgcttatataggcctcccaccgtacacgcctacctcgacccgg
caagcttgcccatttgcgtcaacggggcgggggtattacgacattttggaaagtcccgttgattttggtgccaaaacaaactcccattga
cgtcaatggggtggagacttggaaatccccgtgagtcaaaccgctatccacgcccattggtgtactgccaaaaccgcatcaccatggt
aatagcgatgactaatacgtagatgtactgccaagtaggaaagtcccgtaaggtcatgtactgggcataatgccaggcgggccatttac
cgtcattgacgtcaatagggggcggacttggcatatgatacacttgatgtactgccaagtgggcagtttaccgtaaatactccacccatt
gacgtcaatggaaagtccctattggcgttactatgggaacatacgtcattattgacgtcaatgggcgggggtcgttgggcggtcagcca
ggcgggccatttaccgtaagttatgtaacgctggggactttccagctggggactttccagctgggactttccaggag
Effector element: Our standard epigenetic silencer system
hD3A-mD3L-ZF-ZN627
Nucleotide sequence
(SEQ ID NO: 69)
atggcccctaaaaaaaagagaaaggtcaaccacgaccaggagttcgacccacctaaggtgtaccctcctgtgccagccgagaagcg
gaagccaatccgggtgctgagcctctttgacggcatcgccaccggcctgctggtgctgaaggacctgggcatccaggtggacagata
catcgccagtgaagtgtgcgaagattctatcaccgtgggcatggtgcggcaccaaggcaagatcatgtacgtgggcgacgtccggtc
tgtgacccagaagcatatccaggagtggggccccttcgacctggttatcggcggaagcccctgcaacgacctgagcatcgtgaaccc
cgctagaaagggcctgtacgagggcacaggcagactgttcttcgagttctaccggctgctgcacgatgcccgtcccaaggaaggcg
atgacagaccttttttctggctgttcgagaacgtggtggccatgggcgtgagcgacaaacgggacatcagcagattcctggagtctaac
cctgtgatgattgatgctaaggaagtgtctgccgcccaccgggcccggtacttctggggcaacctgccaggcatgaacagacctctcg
cctctaccgtgaatgacaagctggaactgcaggagtgtctggaacacggcagaatcgccaagttcagtaaggtgagaaccatcacca
ccaggagcaacagcatcaagcagggcaaagatcagcacttccctgtgtttatgaacgagaaggaggacatcctgtggtgcaccgag
atggaaagagtttttggcttccccgtgcactacaccgacgtttctaacatgagcagactggccagacagcggctgctgggcagatcctg
gagcgtgcctgtgatcagacacctgttcgcccccctgaaggagtactttgcctgcgtgagctccggaaattctaacgccaactcccgc
ggcccctccttcagcagcggactggtgcctctcagcctgagaggcagccacatgggacctatggaaatatacaagacagtgtcggct
tggaagaggcagcctgtgagagtgctgagcctgttcagaaacatcgacaaggtgctgaagagcctgggatttctggaatctggaagc
gggtctggcggcggcaccctgaagtacgtggaagatgtgacaaacgtggtgcggagagatgtcgagaaatggggacctttcgatct
ggtgtacggcagcacccagcccctgggcagcagctgcgaccggtgccccggctggtacatgtttcaattccaccgtatcctgcagta
cgccctgcctcggcaggaaagccagagacctttcttctggatcttcatggacaacctgctgctgaccgaggacgaccaggagacaac
cacaagattcctgcagaccgaggccgtgaccctgcaggatgtccggggccgcgactaccagaatgccatgcgggtgtggagcaata
tccctggcctgaagtccaagcacgcccctctgacacctaaggaagaagagtacctgcaagctcaggtgagaagcagatccaagctg
gatgcccctaaggtcgacctgctggtgaaaaactgcctgctgccacttagagagtacttcaaatacttctcccaaaatagcctgcccctg
agcggcggaggcgggagcggcggaggcggcagcctggagcctggcgagaagccctacaagtgcccagagtgcggcaaatctttt
agcgaccctggccacctggtgcggcaccagcggacccataccggtgagaaaccctacaaatgtcctgaatgtggcaagagcttctcc
gattgcagagatctggcccggcaccagagaacccatacaggcgaaaaaccttacaagtgccctgagtgcggaaaatccttcagctct
cctgccgacctgacaagacaccagcggacccacacaggcgagaaaccttataagtgtcccgaatgcggcaagagcttctccagagc
cgataacctgaccgagcaccaaaggacccacaccggcgaaaagccttataagtgcccagaatgcggcaagagcttttcccagagcg
gtcatctgacagagcaccagagaacacacaccggagagaagccctataagtgtcctgagtgtggaaaaagcttttcaagaagcgacc
acctcaccacacaccagcgcacacacacaggcaagaagaccagcagcggtggcggcggctctggcggcggaggaagcgacag
cgtggccttcgaggacgtggctgtgaatttcactctggaagagtgggctctgctcgaccctagccaaaagaacctgtacagagacgtg
atgagagaaaccttcagaaacctggctagcgtcggcaaacagtgggaggatcagaacatcgaggatcctttcaagatcccgagacg
gaacatcagccacatcccagagagactgtgcgagtccaaggagggaggccagggcgaggaataa
Amino acid sequence
(SEQ ID NO: 70)
MAPKKKRKVNHDQEFDPPKVYPPVPAEKRKPIRVLSLFDGIATGLLVLKDLGIQVDRY
IASEVCEDSITVGMVRHQGKIMYVGDVRSVTQKHIQEWGPFDLVIGGSPCNDLSIVNP
ARKGLYEGTGRLFFEFYRLLHDARPKEGDDRPFFWLFENVVAMGVSDKRDISRFLES
NPVMIDAKEVSAAHRARYFWGNLPGMNRPLASTVNDKLELQECLEHGRIAKFSKVR
TITTRSNSIKQGKDQHFPVFMNEKEDILWCTEMERVFGFPVHYTDVSNMSRLARQRL
LGRSWSVPVIRHLFAPLKEYFACVSSGNSNANSRGPSFSSGLVPLSLRGSHMGPMEIY
KTVSAWKRQPVRVLSLFRNIDKVLKSLGFLESGSGSGGGTLKYVEDVTNVVRRDVE
KWGPFDLVYGSTQPLGSSCDRCPGWYMFQFHRILQYALPRQESQRPFFWIFMDNLLL
TEDDQETTTRFLQTEAVTLQDVRGRDYQNAMRVWSNIPGLKSKHAPLTPKEEEYLQ
AQVRSRSKLDAPKVDLLVKNCLLPLREYFKYFSQNSLPLSGGGGSGGGGSLEPGEKP
YKCPECGKSFSDPGHLVRHQRTHTGEKPYKCPECGKSFSDCRDLARHQRTHTGEKPY
KCPECGKSFSSPADLTRHQRTHTGEKPYKCPECGKSFSRADNLTEHQRTHTGEKPYKC
PECGKSFSQSGHLTEHQRTHTGEKPYKCPECGKSFSRSDHLTTHQRTHTGKKTSSGGG
GSGGGGSDSVAFEDVAVNFTLEEWALLDPSQKNLYRDVMRETFRNLASVGKQWEDQ
NIEDPFKIPRRNISHIPERLCESKEGGQGEE
Effector sequence: target DNA sequence of our standard epigenetic silencer system.
ZF target sequence
(SEQ ID NO: 66)
TGGCGACAGACAGCCGGC
Actuator response element: Cumate inducible promoter, repressor, and promoter sequence
Cumate-inducible promoter (CMV5 + CuO)
(SEQ ID NO: 72)
cgttacataacttacggtaaatggcccgcctggctgaccgcccaacgacccccgcccattgacgtcaataatgacgtatgttcccatag
taacgccaatagggactttccattgacgtcaatgggtggagtatttacggtaaactgcccacttggcagtacatcaagtgtatcatatgcc
aagtccgccccctattgacgtcaatgacggtaaatggcccgcctggcattatgcccagtacatgaccttacgggactttcctacttggca
gtacatctacgtattagtcatcgctattaccatggtgatgcggttttggcagtacaccaatgggcgtggatagcggtttgactcacgggga
tttccaagtctccaccccattgacgtcaatgggagtttgttttggcaccaaaatcaacgggactttccaaaatgtcgtaatacccccgccc
cgttgacgcaaatgggcaagcttgccgggtcgaggtaggcgtgtacggtgggaggcctatataagcaaccggtataatcaaacagac
cagattgtctgtttgttaccggtgtttagtgaaccgggcgcgcctcatatcgcctggagacgccatccacgctgttttgacctccatagaa
gacaccgggaccgatccagcctccgcggtcactctcttccgcatcgctgtctgcgagggccagctgttgggctcgcggttgaggaca
aactcttcgcggtctttccagtactcttggatcggaaacccgtcggcctccgaacggtactccgccaccgagggacctgagcgagtcc
gcatcgaccggatcggaaaacctctcgagaaaggcgtctaaccagtcacagtcgcaaggtaggctgagcaccgtggcgggcggca
gcgggtggcggtcggggttgtttctggcggaggtgctgctgatgatgtaattaaagtaggcggtcttgagacggcggatggtcgaggt
gaggtgtggcaggcttgagatccagctgttggggtgagtactccctctcaaaagcgggcattacttctgcgctaagattgtcagtttcca
aaaacgaggaggatttgatattcacctggcccgatctggccatacacttgagtgacaatgacatccactttgcctttctctccacaggtgt
ccactcccaggtccaagtttggtctagtcgactggatccggtaccgaattc
CuO sequence
(SEQ ID NO: 73)
Atcaaacagaccagattgtctgtttgt
CymR repressor
Nucleotide sequence
(SEQ ID NO: 74)
Atggcttacccatacgatgttccagattacgctagtttggtgaggcagcagagaccgatgacatccactttgcctttctctccacaggtgt
ccactcccaggtccaagtttggaagatctaccatgccaaagagacccagacccgtgatcatgagtccaaagagaagaacacaggca
gagcgcgcaatggagacccagggcaagttgattgcagcggccctgggggttttacgggaaaaaggttacgcgggattccggatcgc
agatgtgcccggtgctgcaggtgtctcgagaggagcgcagagccatcatttcccgacaaagcttgagcttctgcttgccacttttgaatg
gctttacgaacagatcaccgaacgcagtcgggctcgattagcgaaattgaagccagaggatgacgtcatccagcaaatgctggacga
cgccgccgaatttttcctcgacgatgacttctctatcagccttgatttgattgtggctgccgaccgggatccagcgttacgcgagggtatt
cagcgcacggtagagaggaatcggtttgtcgtcgaggatatgtggcttggtgttctggtgagccgtggtctttcgcgtgatgatgcaga
agatatcctttggttgatattcaattcggtgcgtgggcttgctgttcgtagcctatggcagaaggacaaagaacgctttgagcgtgtcagg
aactcgacactcgaaattgcgcgagagcggtacgcgaaattcaagcgcgcggccaaaccctctggccggtctctctctgttcgataa
Amino acid sequence
(SEQ ID NO: 75)
MAYPYDVPDYASLVRQQRPMTSTLPFSPQVSTPRSKFGRSTMPKRPRPVIMSPKRRTQ
AERAMETQGKLIAAALGVLREKGYAGFRIADVPGAAGVSRGAQSHHFPTKLELLLAT
FEWLYEQITERSRARLAKLKPEDDVIQQMLDDAAEFFLDDDFSISLDLIVAADRDPAL
REGIQRTVERNRFVVEDMWLGVLVSRGLSRDDAEDILWLIFNSVRGLAVRSLWQKD
KERFERVRNSTLEIARERYAKFKRAAKPSGRSLSVR
Therapeutic transgene for GIP/GLP dual receptor agonist:
GIP/GLP-1 RA
Nucleotide sequence
(SEQ ID NO: 76)
Atgcactggggcaccctgtgcggcttcctgtggctgtggccctacctgttctacgtgcaggcttatggcgagggcacattcaccagcg
actacagcaagtacctggataagatcgcccagaaagcctttgtgcagtggctgatcgccggaggaccttccagcggcgcccctcctc
catct
Amino acid sequence
(SEQ ID NO: 77)
MHWGTLCGFLWLWPYLFYVQAYGEGTFTSDYSKYLDKIAQKAFVQWLIAGGPSSG
APPPS
3NF1
(SEQ ID NO: 10)
Ctggggactttccagcctggggactttccagctgggactttccagg
3NF2
(SEQ ID NO: 11)
Ctggggactttccagctggggactttccagctgggactttccaggag
EFS promoter
(SEQ ID NO: 30)
Ggctccggtgcccgtcagtgggcagagcgcacatcgcccacagtccccgagaagttggggggaggggtcggcaattgatccggt
gcctagagaaggtggcgcggggtaaactgggaaagtgatgtcgtgtactggctccgcctttttcccgagggtgggggagaaccgtat
ataagtgcagtagtcgccgtgaacgttctttttcgcaacgggtttgccgccagaacacagg
P2A
Nucleotide sequence
(SEQ ID NO: 37)
GGATCTGGCGCCACAAACTTCAGCCTGCTGAAGCAGGCCGGAGATGTGGAAGAA
AATCCTGGCCCC
Amino acid sequence
(SEQ ID NO: 29)
ATNFSLLKQAGDVEENPGP
bGH poly(A) sequence
(SEQ ID NO: 46)
ctgtgccttctagttgccagccatctgttgtttgcccctcccccgtgccttccttgaccctggaaggtgccactcccactgtcctttcctaat
aaaatgaggaaattgcatcgcattgtctgagtaggtgtcattctattctggggggtggggtggggcaggacagcaagggggaggattg
ggaagagaatagcaggcaggctgggga
SV40 poly(A) sequence
(SEQ ID NO: 47)
Taagatacattgatgagtttggacaaaccacaactagaatgcagtgaaaaaaatgctttatttgtgaaatttgtgatgctattgctttatttgt
aaccattataagctgcaataaacaagtt

### Example 8

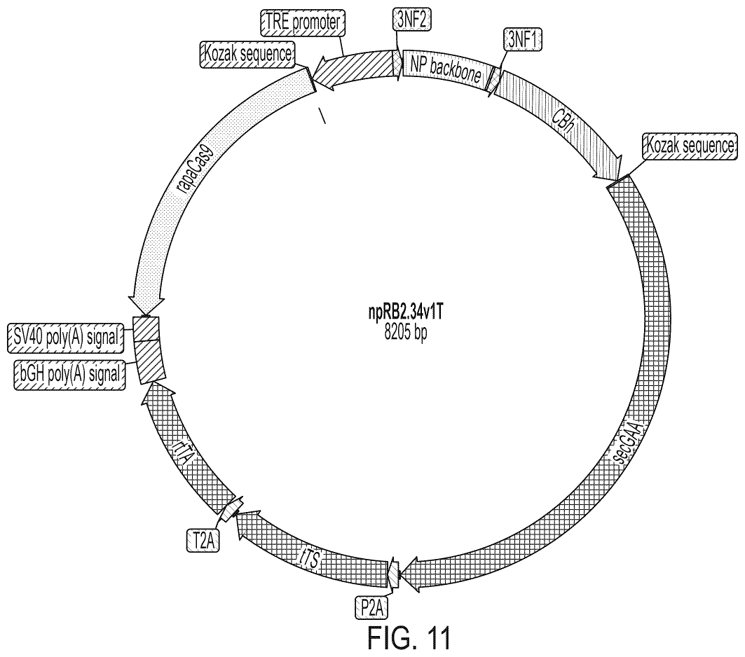

In the present example, a system containing a dual actuator regulated effector element is intended to deliver biologically active recombinant acid alpha-glucosidase for the treatment of Glycogen storage disease type II (Pompe disease). The first actuator is a tetracycline or tetracycline derivative, which acts upon the TetOn inducible promoter and in conjunction with the TetOn operator activates expression of rapamycin inducible-Caspase9. The second actuator is Rapamycin or rapamycin analog or derivative, which activates inducible Caspase9 to promote the initiation of apoptosis in the cells carrying the therapeutic construct. FIG. 11 shows a plasmid map for the genetic construct described in this example.

Full sequence (except nanoplasmid backbone)
(SEQ ID NO: 78)
ctggggactttccagcctggggactttccagctgggactttccaggcgttacataacttacggtaaatggcccgcctggctgaccgccc
aacgacccccgcccattgacgtcaatagtaacgccaatagggactttccattgacgtcaatgggggagtatttacggtaaactgccca
cttggcagtacatcaagtgtatcatatgccaagtacgccccctattgacgtcaatgacggtaaatggcccgcctggcattgtgcccagta
catgaccttatgggactttcctacttggcagtacatctacgtattagtcatcgctattaccatggtcgaggtgagccccacgttctgcttcac
tctccccatctcccccccctccccacccccaattttgtatttatttattttttaattattttgtgcagcgatgggggcgggggggggggggg
ggcgcgcgccaggcggggcggggcggggcgaggggcggggcggggcgaggcggagaggtgcggcggcagccaatcagag
cggcgcgctccgaaagtttccttttatggcgaggcggcggcggcggcggccctataaaaagcgaagcgcgcggcgggcgggagt
cgctgcgcgctgccttcgccccgtgccccgctccgccgccgcctcgcgccgcccgccccggctctgactgaccgcgttactcccac
aggtgagcggggggacggcccttctcctccgggctgtaattagctgagcaagaggtaagggtttaagggatggttggttggtgggg
tattaatgtttaattacctggagcacctgcctgaaatcactttttttcaggttgggccaccatggccttcttgtggctgctcagctgctgggc
cctgctgggcaccacattcggcctgcttgtgcctagagagctgagcggctctagcccagtgctggaggaaacccacccagcccacc
agcagggagctagccggcctggacccagagacgcccaggcccaccccggaagacccagagccgtgcctacccagtgcgacgtg
cctcccaacagcagattcgactgcgcccctgataaggccattacccaggagcagtgtgaagctagaggctgctgctacatccctgcca
agcagggcctgcaaggcgcccagatgggccaaccttggtgcttcttcccaccctcttaccctagctacaagctggaaaacctgagctc
ttctgagatgggctacaccgccaccctgacaagaaccaccccaacattcttcccaaaagacatcctgaccctgagactggacgtgatg
atggaaaccgagaaccggctgcatttcaccattaaggaccccgccaacagacggtacgaggtccccctggaaacaccccacgtgca
cagccgggccccttcccctctgtacagtgtggaattcagcgaggaacccttcggcgtgatcgtgagaaggcagctggatggcagagt
gctgctcaacaccacagtggctccattattcttcgccgaccagttcctgcagctgagcaccagcctgccctcacagtacatcacaggcc
tcgccgaacacctgtctcctctcatgctgagcaccagctggacaagaatcacactgtggaaccgggacctggcccccacacctgggg
ctaatctgtacggctcccaccccttctacctggcgctggaagatggggcagcgcccacggcgttttcctgctgaacagcaatgccat
ggacgtggtgctgcagcctagccctgccctgagctggcggagcaccggcggaatcctggacgtgtacatcttcctgggacccgagc
ctaagagcgtggtgcagcaatatctggatgtggtgggctacccctttatgcctccttactggggcctgggttttcacctgtgtagatgggg
ctactcatctaccgcgatcaccagacaggggttgagaatatgacccgggcccacttccctctcgacgtgcagtggaacgacctggac
tacatggacagcagaagagatttcacgttcaacaaggacggcttcagggatttccctgctatggtgcaggagctgcaccagggaggc
agacggtacatgatgatcgtggatcctgctatcagcagcagcggccccgccggcagctatagaccttatgacgagggactgagacg
gggcgttttcatcacaaacgagacaggacagcctcttatcggcaaggtgtggcccggctctaccgcctttcctgacttcacaaatcctac
cgccctggcctggtgggaagatatggtcgccgagttccacgaccaggtccctttcgacgggatgtggatcgacatgaacgagcccag
taacttcattagaggctctgaagatggatgtcctaacaacgagctggagaaccctccttacgtgcctggcgtggtcggcggaactctgc
aggccgctacaatctgtgccagctcccaccagttcctgagcacccactacaacctgcacaatctgtacggactgacagaggctatcgc
cagtcatagagccctggtcaaggccagaggcaccagacccttcgtgatctccagaagcaccttcgccggacacggcagatatgccg
gccactggacaggcgacgtgtggtcctcttgggagcagctcgcaagctctgtgccggaaatcctgcaatttaacttgctgggcgtgcct
ctggtgggcgctgatgtgtgcggctttctgggcaacacatctgaagaattgtgcgtgcggtggacccagctgggcgctttttaccctttta
tgagaaatcacaactccctgctgagcctgccacaggagccttacagcttcagcgaacctgcccaacaggccatgcggaaggccttaa
cactgcgatacgcactgctgcctcacctgtacaccctgttccaccaggctcatgtggccggcgaaacagtggccagaccactgttcct
ggagttccccaaggatagctccacatggaccgtggaccaccagctgctgtggggcgaggccctgctgatcacccctgtgctgcagg
ccggcaaagctgaggtgaccggctacttccctctgggcacctggtacgacctgcagaccgtgcctgttgaggctctgggatctctgcc
ccccccacctgccgcccctcgcgagcctgctatccactctgaaggccagtgggtgacactgcctgccccactcgacaccatcaacgt
gcacctgagagccggttacatcatccccctgcagggccccggcctgaccaccaccgaaagccggcagcaacctatggccctcgcc
gtggccctgaccaagggcggcgaggccaggggtgagctattttgggacgacggcgagagcctggaggtgctggagagaggcgc
ctacacacaagttatcttcctggctcggaacaacaccatcgtgaacgagctggtgcgggtgacaagcgagggcgccggcctccagct
gcagaaagtgacggtgctgggcgtggccacagcccctcagcaggtgctgagcaacggcgtgccagtcagcaatttcacctacagcc
ctgataccaaggttctggacatctgcgtgagcctgctcatgggcgaacagttcctggtgtcctggtgcggatctggcgccacaaacttc
agcctgctgaagcaggccggagatgtggaagaaaatcctggccccatgtctagattagataaaagtaaagtgattaacagcgcattag
agctgcttaatgaggtcggaatcgaaggtttaacaacccgtaaactcgcccagaagctaggtgtagagcagcctacattgtattggcac
gtgcgcaacaagcagactcttatgaacatgctttcagaggcaatactggcgaagcatcacacccgttcagcaccgttaccgactgaga
gttggcagcagtttctccaggaaaatgctctgagtttccgtaaagcattactggtccatcgtgatggagcccgattgcatatagggacctc
tcctacgcccccccagtttgaacaagcagaggcgcaactacgctgtctatgcgatgcagggttttcggtcgaggaggctcttttcattct
gcaatctatcagccattttacgttgggtgcagtattagaggagcaagcaacaaaccagatagaaaataatcatgtgatagacgctgcac
caccattattacaagaggcatttaatattcaggcgagaacctctgctgaaatggccttccatttcgggctgaaatcattaatatttggattttc
tgcacagttagatgaaaaaaagcatacacccattgaggatggtaataaaccaaaaaagaagagaaagctagcagtgtcagtgacattt
gaagatgtggctgtgctctttactcgggacgagtggaagaagctggatctgtctcagagaagcctgtaccgtgaggtgatgctggaga
attacagcaacctggcctccatggcaggattcctgtttaccaaaccaaaggtgatctccctgttgcagcaaggagaggatccctggg
ctccggagagggcaggggaagtcttctaacatgcggggacgtggaggaaaatcccggccccatgtctagactggacaagagcaaa
gtcataaacggcgctctggaattactcaatggagtcggtatcgaaggcctgacgacaaggaaactcgctcaaaagctgggagttgag
cagcctaccctgtactggcacgtgaagaacaagcgggccctgctcgatgccctgccaatcgagatgctggacaggcatcatacccac
ttctgccccctggaaggcgagtcatggcaagactttctgcggaacaacgccaagtcattccgctgtgctctcctctcacatcgcgacgg
ggctaaagtgcatctcggcacccgcccaacagagaaacagtacgaaaccctggaaaatcagctcgcgttcctgtgtcagcaaggctt
ctccctggagaacgcactgtacgctctgtccgccgtgggccactttacactgggctgcgtattggaggaacaggagcatcaagtagca
aaagaggaaagagagacacctaccaccgattctatgcccccacttctgagacaagcaattgagctgttcgaccggcagggagccga
acctgccttccttttcggcctggaactaatcatatgtggcctggagaaacagctaaagtgcgaaagcggcgggccggccgacgccctt
gacgattttgacttagacatgctcccagccgatgcccttgacgactttgaccttgatatgctgcctgctgacgctcttgacgattttgacctt
gacatgctccccgggtaactgtgccttctagttgccagccatctgttgtttgcccctcccccgtgccttccttgaccctggaaggtgccac
tcccactgtcctttcctaataaaatgaggaaattgcatcgcattgtctgagtaggtgtcattctattctggggggtggggtggggcaggac
agcaagggggaggattgggaagagaatagcaggcaggctggggaaacttgtttattgcagcttataatggttacaaataaagcaatag
catcacaaatttcacaaataaagcatttttttcactgcattctagttgtggtttgtccaaactcatcaatgtatcttatcattattagctggcgc
tggtcttgaaaaacagcttctttctcaagaagttgaagcagccaggcatctgcttgtagatgcccttaacggacacggcattagccaccc
gcagcagcagagattgaaggtcctcgctgtgggcccactgctcaaagatatcgtccagtgtctccacgtaccaggagccgctcttagg
gtcccgccagctcacgaatcctggaaaggtgctatatgacacgaagatatcgctaggtgtaggcaggctggagatggcatccagctg
gtcaaaagttctcaggccctcctgaaaaggggggcgtctggctcaggattagagcctggagactcgtcctcggggcttgtgctggcc
acctcgaagccgtggtccttctgttcgccgccacaagcctggatgaagaacagcttgggcttgccgcccaggctggggcaggatgtg
ccgttgaagatgttcacgatcttttccacggacacagggcaaccgtcggtgccgtacactgctccagggaactgcaggtggctagcct
gacagccatggctcaggatgaccaccacgcagcagtccagggcgccgtggtcctgctgggccagttcgagcagggccagcaccat
cttcttggcggtgagatctcctttcacctcgaccatgaagtgcagagagctgaatcttctccgcagcttctcgcagtcgatgttgctgccg
gttctggtgcgcagtccgctctctctgcagaagttcacgttgttgatgatcaggcagtggccacaaggctccatgctcagaatgtaagcc
agatcggcgttgcctctgaggctttccagggcgcccacgtcgccgaagccgtccacgccggaaccgcgccgccgctgccaccgc
ctccagagccgccgcctccgctctccagcttcagcagctccacatcaaagaccagggggcgtggggggggatgatgccagggtgt
ccggtagcgccgtaggcgtagtcaggagaaattgtcagtttggcccgctggcccacgctcatttgggccacgccttcctcccagcctct
gatcacctcctgtttgcccagcatgaacttgaaaggcttgttccgatctctgctgctgtcgaatttctttccatcttccagcattccggtgtag
tgcaccacacaggtctgtcctcttttggggaatgttctgccatcgccaggagagatggtttccacctgaactccttccaggcttcctcctc
cgctgtattccagcttgctgatccgtctgaacacgtggtagtacaggtcccaggcttgcagcaggtccttcacattcccgcttttcatgtac
ttccggcaccactcctgggcttccatcagatcgcggccataggcctggttgaagcttgtttccttcagggtctgagggcctctttccatca
tggcgtgcaggggttccaggacctcgaacatgcccttcacgttccgctcgccgaagtacagtctactagcctcttccaggccctcgtgc
cacatctcatgccacaggattctagaggccatggtggccaattcggggccgcggaggctggatcggtcccggtgtcttctatggaggt
caaaacagcgtggatggcgtctccaggcgatctgacggttcactaaacgagctctgcttatataggtcgagtttaccactccctatcagt
gatagagaaaagtgaaagtcgagtttaccactccctatcagtgatagagaaaagtgaaagtcgagtttaccactccctatcagtgataga
gaaaagtgaaagtcgagtttaccactccctatcagtgatagagaaaagtgaaagtcgagtttaccactccctatcagtgatagagaaaa
gtgaaagtcgagtttaccactccctatcagtgatagagaaaagtgaaagtcgagtttaccactccctatcagtgatagagaaaagtgaaa
gtcgagctcggtaccctggggactttccagctggggactttccagctgggactttccaggag
3NF1
(SEQ ID NO: 10)
Ctggggactttccagcctggggactttccagctgggactttccagg
3NF2
(SEQ ID NO: 11)
Ctggggactttccagctggggactttccagctgggactttccaggag
CBh promoter
(SEQ ID NO: 32)
Cgttacataacttacggtaaatggcccgcctggctgaccgcccaacgacccccgcccattgacgtcaatagtaacgccaatagggac
tttccattgacgtcaatgggtggagtatttacggtaaactgcccacttggcagtacatcaagtgtatcatatgccaagtacgccccctattg
acgtcaatgacggtaaatggcccgcctggcattgtgcccagtacatgaccttatgggactttcctacttggcagtacatctacgtattagt
catcgctattaccatggtcgaggtgagccccacgttctgcttcactctccccatctcccccccctccccacccccaattttgtatttatttatt
ttttaattattttgtgcagcgatgggggcggggggggggggggggcgcgcgccaggcggggcggggcggggcgaggggcggg
gcggggcgaggcggagaggtgcggcggcagccaatcagagcggcgcgctccgaaagtttccttttatggcgaggcggcggcggc
ggcggccctataaaaagcgaagcgcgcggcgggcgggagtcgctgcgcgctgccttcgccccgtgccccgctccgccgccgcct
cgcgccgcccgccccggctctgactgaccgcgttactcccacaggtgagcgggcgggacggcccttctcctccgggctgtaattag
ctgagcaagaggtaagggtttaagggatggttggttggtggggtattaatgtttaattacctggagcacctgcctgaaatcactttttttca
ggttgg
secGAA (sp7-delta8-hGAA)
Nucleotide sequence
(SEQ ID NO: 50)
atggccttcttgtggctgctcagctgctgggccctgctgggcaccacattcggcctgcttgtgcctagagagctgagcggctctagccc
agtgctggaggaaacccacccagcccaccagcagggagctagccggcctggacccagagacgcccaggcccaccccggaagac
ccagagccgtgcctacccagtgcgacgtgcctcccaacagcagattcgactgcgcccctgataaggccattacccaggagcagtgtg
aagctagaggctgctgctacatccctgccaagcagggcctgcaaggcgcccagatgggccaaccttggtgcttcttcccaccctctta
ccctagctacaagctggaaaacctgagctcttctgagatgggctacaccgccaccctgacaagaaccaccccaacattcttcccaaaa
gacatcctgaccctgagactggacgtgatgatggaaaccgagaaccggctgcatttcaccattaaggaccccgccaacagacggtac
gaggtccccctggaaacaccccacgtgcacagccgggccccttcccctctgtacagtgtggaattcagcgaggaacccttcggcgtg
atcgtgagaaggcagctggatggcagagtgctgctcaacaccacagtggctccattattcttcgccgaccagttcctgcagctgagca
ccagcctgccctcacagtacatcacaggcctcgccgaacacctgtctcctctcatgctgagcaccagctggacaagaatcacactgtg
gaaccgggacctggcccccacacctggggctaatctgtacggctcccaccccttctacctggcgctggaagatggggcagcgccc
acggcgttttcctgctgaacagcaatgccatggacgtggtgctgcagcctagccctgccctgagctggcggagcaccggcggaatcc
tggacgtgtacatcttcctgggacccgagcctaagagcgtggtgcagcaatatctggatgtggtgggctacccctttatgcctccttact
ggggcctgggttttcacctgtgtagatggggctactcatctaccgcgatcaccagacaggtggttgagaatatgacccgggcccacttc
cctctcgacgtgcagtggaacgacctggactacatggacagcagaagagatttcacgttcaacaaggacggcttcagggatttccctg
ctatggtgcaggagctgcaccagggaggcagacggtacatgatgatcgtggatcctgctatcagcagcagcggccccgccggcag
ctatagaccttatgacgagggactgagacggggcgttttcatcacaaacgagacaggacagcctcttatcggcaaggtgtggcccgg
ctctaccgcctttcctgacttcacaaatcctaccgccctggcctggtgggaagatatggtcgccgagttccacgaccaggtccctttcga
cgggatgtggatcgacatgaacgagcccagtaacttcattagaggctctgaagatggatgtcctaacaacgagctggagaaccctcct
tacgtgcctggcgtggtcggcggaactctgcaggccgctacaatctgtgccagctcccaccagttcctgagcacccactacaacctgc
acaatctgtacggactgacagaggctatcgccagtcatagagccctggtcaaggccagaggcaccagacccttcgtgatctccagaa
gcaccttcgccggacacggcagatatgccggccactggacaggcgacgtgtggtcctcttgggagcagctcgcaagctctgtgccg
gaaatcctgcaatttaacttgctgggcgtgcctctggtgggcgctgatgtgtgcggctttctgggcaacacatctgaagaattgtgcgtg
cggtggacccagctgggcgctttttacccttttatgagaaatcacaactccctgctgagcctgccacaggagccttacagcttcagcga
acctgcccaacaggccatgcggaaggccttaacactgcgatacgcactgctgcctcacctgtacaccctgttccaccaggctcatgtg
gccggcgaaacagtggccagaccactgttcctggagttccccaaggatagctccacatggaccgtggaccaccagctgctgtgggg
cgaggccctgctgatcacccctgtgctgcaggccggcaaagctgaggtgaccggctacttccctctgggcacctggtacgacctgca
gaccgtgcctgttgaggctctgggatctctgccccccccacctgccgcccctcgcgagcctgctatccactctgaaggccagtgggtg
acactgcctgccccactcgacaccatcaacgtgcacctgagagccggttacatcatccccctgcagggccccggcctgaccaccacc
gaaagccggcagcaacctatggccctcgccgtggccctgaccaagggcggcgaggccaggggtgagctattttgggacgacggc
gagagcctggaggtgctggagagaggcgcctacacacaagttatcttcctggctcggaacaacaccatcgtgaacgagctggtgcg
ggtgacaagcgagggcgccggcctccagctgcagaaagtgacggtgctgggcgtggccacagcccctcagcaggtgctgagcaa
cggcgtgccagtcagcaatttcacctacagccctgataccaaggttctggacatctgcgtgagcctgctcatgggcgaacagttcctgg
tgtcctggtgctaa
Amino acid sequence
(SEQ ID NO: 51)
MAFLWLLSCWALLGTTFGLLVPRELSGSSPVLEETHPAHQQGASRPGPRDAQAHPGR
PRAVPTQCDVPPNSRFDCAPDKAITQEQCEARGCCYIPAKQGLQGAQMGQPWCFFPP
SYPSYKLENLSSSEMGYTATLTRTTPTFFPKDILTLRLDVMMETENRLHFTIKDPANRR
YEVPLETPHVHSRAPSPLYSVEFSEEPFGVIVRRQLDGRVLLNTTVAPLFFADQFLQLS
TSLPSQYITGLAEHLSPLMLSTSWTRITLWNRDLAPTPGANLYGSHPFYLALEDGGSA
HGVFLLNSNAMDVVLQPSPALSWRSTGGILDVYIFLGPEPKSVVQQYLDVVGYPFMP
PYWGLGFHLCRWGYSSTAITRQVVENMTRAHFPLDVQWNDLDYMDSRRDFTENKD
GFRDFPAMVQELHQGGRRYMMIVDPAISSSGPAGSYRPYDEGLRRGVFITNETGQPLI
GKVWPGSTAFPDFTNPTALAWWEDMVAEFHDQVPFDGMWIDMNEPSNFIRGSEDGC
PNNELENPPYVPGVVGGTLQAATICASSHQFLSTHYNLHNLYGLTEAIASHRALVKAR
GTRPFVISRSTFAGHGRYAGHWTGDVWSSWEQLASSVPEILQFNLLGVPLVGADVCG
FLGNTSEELCVRWTQLGAFYPFMRNHNSLLSLPQEPYSFSEPAQQAMRKALTLRYAL
LPHLYTLFHQAHVAGETVARPLFLEFPKDSSTWTVDHQLLWGEALLITPVLQAGKAE
VTGYFPLGTWYDLQTVPVEALGSLPPPPAAPREPAIHSEGQWVTLPAPLDTINVHLRA
GYIIPLQGPGLTTTESRQQPMALAVALTKGGEARGELFWDDGESLEVLERGAYTQVIF
LARNNTIVNELVRVTSEGAGLQLQKVTVLGVATAPQQVLSNGVPVSNFTYSPDTKVL
DICVSLLMGEQFLVSWC
P2A
Nucleotide sequence
(SEQ ID NO: 37)
GGATCTGGCGCCACAAACTTCAGCCTGCTGAAGCAGGCCGGAGATGTGGAAGAA
AATCCTGGCCCC
Amino acid sequence
(SEQ ID NO: 29)
ATNFSLLKQAGDVEENPGP
tTS
Nucleotide sequence
(SEQ ID NO: 19)
Atgtctagattagataaaagtaaagtgattaacagcgcattagagctgcttaatgaggtcggaatcgaaggtttaacaacccgtaaactc
gcccagaagctaggtgtagagcagcctacattgtattggcacgtgcgcaacaagcagactcttatgaacatgctttcagaggcaatact
ggcgaagcatcacacccgttcagcaccgttaccgactgagagttggcagcagtttctccaggaaaatgctctgagtttccgtaaagcat
tactggtccatcgtgatggagcccgattgcatatagggacctctcctacgcccccccagtttgaacaagcagaggcgcaactacgctgt
ctatgcgatgcagggttttcggtcgaggaggctcttttcattctgcaatctatcagccattttacgttgggtgcagtattagaggagcaagc
aacaaaccagatagaaaataatcatgtgatagacgctgcaccaccattattacaagaggcatttaatattcaggcgagaacctctgctga
aatggccttccatttcgggctgaaatcattaatatttggattttctgcacagttagatgaaaaaaagcatacacccattgaggatggtaata
aaccaaaaaagaagagaaagctagcagtgtcagtgacatttgaagatgtggctgtgctctttactcgggacgagtggaagaagctgga
tctgtctcagagaagcctgtaccgtgaggtgatgctggagaattacagcaacctggcctccatggcaggattcctgtttaccaaaccaa
aggtgatctccctgttgcagcaaggagaggatccctgg
(SEQ ID NO: 20)
MSRLDKSKVINSALELLNEVGIEGLTTRKLAQKLGVEQPTLYWHVRNKQTLMNMLS
EAILAKHHTRSAPLPTESWQQFLQENALSFRKALLVHRDGARLHIGTSPTPPQFEQAE
AQLRCLCDAGFSVEEALFILQSISHFTLGAVLEEQATNQIENNHVIDAAPPLLQEAFNI
QARTSAEMAFHFGLKSLIFGFSAQLDEKKHTPIEDGNKPKKKRKLAVSVTFEDVAVLF
TRDEWKKLDLSQRSLYREVMLENYSNLASMAGFLFTKPKVISLLQQGEDPW
T2A
Nucleotide sequence
(SEQ ID NO: 26)
GAGGGCAGGGGAAGTCTTCTAACATGCGGGGACGTGGAGGAAAATCCCGGCCCC
Amino acid sequence
(SEQ ID NO: 27)
EGRGSLLTCGDVEENPGP
rtTA
Nucleotide sequence
(SEQ ID NO: 17)
Atgtctagactggacaagagcaaagtcataaacggcgctctggaattactcaatggagtcggtatcgaaggcctgacgacaaggaaa
ctcgctcaaaagctgggagttgagcagcctaccctgtactggcacgtgaagaacaagcgggccctgctcgatgccctgccaatcgag
atgctggacaggcatcatacccacttctgccccctggaaggcgagtcatggcaagactttctgcggaacaacgccaagtcattccgct
gtgctctcctctcacatcgcgacggggctaaagtgcatctcggcacccgcccaacagagaaacagtacgaaaccctggaaaatcagc
tcgcgttcctgtgtcagcaaggcttctccctggagaacgcactgtacgctctgtccgccgtgggccactttacactgggctgcgtattgg
aggaacaggagcatcaagtagcaaaagaggaaagagagacacctaccaccgattctatgcccccacttctgagacaagcaattgag
ctgttcgaccggcagggagccgaacctgccttccttttcggcctggaactaatcatatgtggcctggagaaacagctaaagtgcgaaa
gcgggggccggccgacgcccttgacgattttgacttagacatgctcccagccgatgcccttgacgactttgaccttgatatgctgcct
gctgacgctcttgacgattttgaccttgacatgctccccggg
Amino acid sequence
(SEQ ID NO: 18)
MSRLDKSKVINGALELLNGVGIEGLTTRKLAQKLGVEQPTLYWHVKNKRALLDALPI
EMLDRHHTHFCPLEGESWQDFLRNNAKSFRCALLSHRDGAKVHLGTRPTEKQYETL
ENQLAFLCQQGFSLENALYALSAVGHFTLGCVLEEQEHQVAKEERETPTTDSMPPLLR
QAIELFDROGAEPAFLFGLELIICGLEKQLKCESGGPADALDDFDLDMLPADALDDFD
LDMLPADALDDFDLDMLPG
TRE promoter
(SEQ ID NO: 23)
Ggtaccgagctcgactttcacttttctctatcactgatagggagtggtaaactcgactttcacttttctctatcactgatagggagtggtaaa
ctcgactttcacttttctctatcactgatagggagtggtaaactcgactttcacttttctctatcactgatagggagtggtaaactcgactttca
cttttctctatcactgatagggagtggtaaactcgactttcacttttctctatcactgatagggagtggtaaactcgactttcacttttctctat
cactgatagggagtggtaaactcgacctatataagcagagctcgtttagtgaaccgtcagatcgcctggagacgccatccacgctgtttt
gacctccatagaagacaccgggaccgatccagcctccgcggccccgaattg
RapaCasp9
Nucleotide sequence
(SEQ ID NO: 79)
atggcctctagaatcctgtggcatgagatgtggcacgagggcctggaagaggctagtagactgtacttcggcgagcggaacgtgaag
ggcatgttcgaggtcctggaacccctgcacgccatgatggaaagaggccctcagaccctgaaggaaacaagcttcaaccaggcctat
ggccgcgatctgatggaagcccaggagtggtgccggaagtacatgaaaagcgggaatgtgaaggacctgctgcaagcctgggacc
tgtactaccacgtgttcagacggatcagcaagctggaatacagcggaggaggaagcctggaaggagttcaggtggaaaccatctctc
ctggcgatggcagaacattccccaaaagaggacagacctgtgtggtgcactacaccggaatgctggaagatggaaagaaattcgac
agcagcagagatcggaacaagcctttcaagttcatgctgggcaaacaggaggtgatcagaggctgggaggaaggcgtggcccaaa
tgagcgtgggccaggggccaaactgacaatttctcctgactacgcctacggcgctaccggacaccctggcatcatccccccccacg
ccaccctggtctttgatgtggagctgctgaagctggagagcggaggcggcggctctggaggcggtggcagcggcggcggcggttc
cggcgtggacggcttcggcgacgtgggcgccctggaaagcctcagaggcaacgccgatctggcttacattctgagcatggagcctt
gtggccactgcctgatcatcaacaacgtgaacttctgcagagagagcggactgcgcaccagaaccggcagcaacatcgactgcgag
aagctgcggagaagattcagctctctgcacttcatggtcgaggtgaaaggagatctcaccgccaagaagatggtgctggccctgctcg
aactggcccagcaggaccacggcgccctggactgctgcgtggtggtcatcctgagccatggctgtcaggctagccacctgcagttcc
ctggagcagtgtacggcaccgacggttgccctgtgtccgtggaaaagatcgtgaacatcttcaacggcacatcctgccccagcctgg
gcggcaagcccaagctgttcttcatccaggcttgtggggcgaacagaaggaccacggcttcgaggtggccagcacaagccccga
ggacgagtctccaggctctaatcctgagccagacgccaccccttttcaggagggcctgagaacttttgaccagctggatgccatctcca
gcctgcctacacctagcgatatcttcgtgtcatatagcacctttccaggattcgtgagctggcgggaccctaagagcggctcctggtac
gtggagacactggacgatatctttgagcagtgggcccacagcgaggaccttcaatctctgctgctgcgggtggctaatgccgtgtccg
ttaagggcatctacaagcagatgcctggctgcttcaacttcttgagaaagaagctgtttttcaagaccagcgccagctaa
Amino acid sequence
(SEQ ID NO: 80)
MASRILWHEMWHEGLEEASRLYFGERNVKGMFEVLEPLHAMMERGPQTLKETSFN
QAYGRDLMEAQEWCRKYMKSGNVKDLLQAWDLYYHVFRRISKLEYSGGGSLEGV
QVETISPGDGRTFPKRGQTCVVHYTGMLEDGKKFDSSRDRNKPFKFMLGKQEVIRG
WEEGVAQMSVGQRAKLTISPDYAYGATGHPGIIPPHATLVFDVELLKLESGGGGSGGG
GSGGGGSGVDGFGDVGALESLRGNADLAYILSMEPCGHCLIINNVNFCRESGLRTRT
GSNIDCEKLRRRFSSLHFMVEVKGDLTAKKMVLALLELAQQDHGALDCCVVVILSH
GCQASHLQFPGAVYGTDGCPVSVEKIVNIFNGTSCPSLGGKPKLFFIQACGGEQKDHG
FEVASTSPEDESPGSNPEPDATPFQEGLRTFDQLDAISSLPTPSDIFVSYSTFPGFVSWR
DPKSGSWYVETLDDIFEQWAHSEDLQSLLLRVANAVSVKGIYKQMPGCFNFLRKKLF
FKTSAS
bGH poly(A) sequence
(SEQ ID NO: 46)
ctgtgccttctagttgccagccatctgttgtttgcccctcccccgtgccttccttgaccctggaaggtgccactcccactgtcctttcctaat
aaaatgaggaaattgcatcgcattgtctgagtaggtgtcattctattctggggggtggggtggggcaggacagcaagggggaggattg
ggaagagaatagcaggcaggctgggga
SV40 poly(A) sequence
(SEQ ID NO: 47)
Taagatacattgatgagtttggacaaaccacaactagaatgcagtgaaaaaaatgctttatttgtgaaatttgtgatgctattgctttatttgt
aaccattataagctgcaataaacaagtt

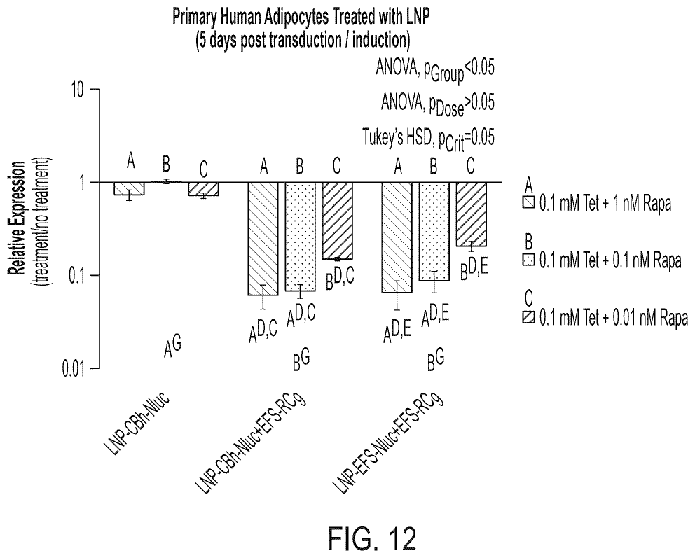

The system is tested with a reporter gene with three concentrations of Rapamycin (ranging from 1 nM to 0.01 nM) and a constant (0.1 nM concentration of Tetracycline and facilitates reduction in expression of nLuc (as measured by the strength of the bioluminescence signal) (FIG. 12). Two promoters are used to drive initial transgene expression, a stronger CBh promoter and a weaker EFS promoter. Constructs with no down-titration genetic element (LNP-CBh-nLuc) are used as a reference control.

No change in expression relative to the “no treatment” control is observed for the LNP-CBh-nLuc constructs. On the other hand, the iCaspase9-containing constructs reduce gene expression in a Rapamycin dose-dependent manner following concurrent activation by tetracycline. During treatment, appropriate rapamycin and tetracycline concentrations can be obtained following administration of the actuator small molecules locally or systemically following treatment with the therapeutic construct.

### Example 9

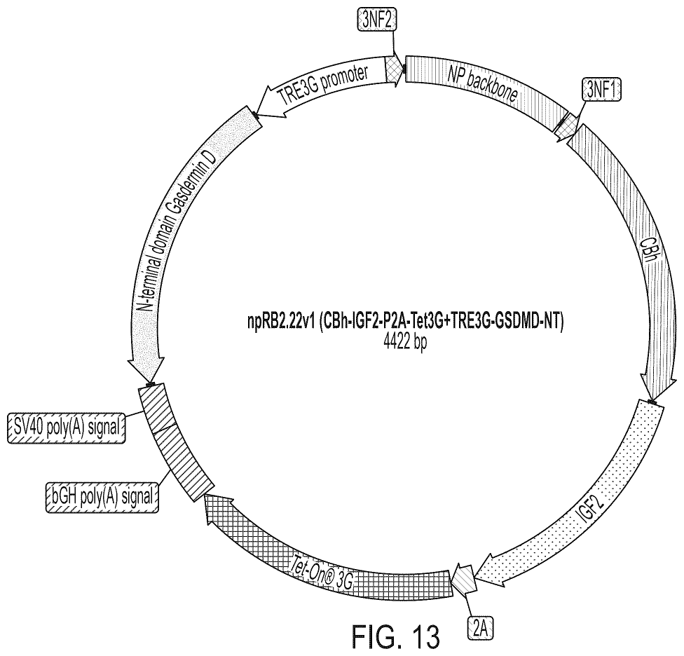

In the present example a single actuator-regulated effector element is intended to deliver biologically active recombinant insulin-like growth factor 2 for the treatment of Silver-Russell Syndrome. The actuator is tetracycline or a tetracycline derivative, which acts upon a 3^{rd} generation TetOn inducible promoter and in conjunction with the TetOn operator activates expression of the N-terminal domain of gasdermin D. Gasdermin D oligomerizes to form pores in the cell membrane and initiates pyroptosis. FIG. 13 shows a plasmid map for the genetic construct described in this example.

Full sequence (except nanoplasmid backbone)
(SEQ ID NO: 81)
CTGGGGACTTTCCAGCCTGGGGACTTTCCAGCTGGGACTTTCCAGGCGTT
ACATAACTTACGGTAAATGGCCCGCCTGGCTGACCGCCCAACGACCCCCG
CCCATTGACGTCAATAGTAACGCCAATAGGGACTTTCCATTGACGTCAAT
GGGTGGAGTATTTACGGTAAACTGCCCACTTGGCAGTACATCAAGTGTAT
CATATGCCAAGTACGCCCCCTATTGACGTCAATGACGGTAAATGGCCCGC
CTGGCATTGTGCCCAGTACATGACCTTATGGGACTTTCCTACTTGGCAGT
ACATCTACGTATTAGTCATCGCTATTACCATGGTCGAGGTGAGCCCCACG
TTCTGCTTCACTCTCCCCATCTCCCCCCCCTCCCCACCCCCAATTTTGTA
TTTATTTATTTTTTAATTATTTTGTGCAGCGATGGGGGCGGGGGGGGGGG
GGGGGCGCGCGCCAGGCGGGGCGGGGCGGGGCGAGGGGCGGGGGGGGCGA
GGCGGAGAGGTGCGGCGGCAGCCAATCAGAGCGGCGCGCTCCGAAAGTTT
CCTTTTATGGCGAGGCGGCGGCGGCGGCGGCCCTATAAAAAGCGAAGCGC
GCGGCGGGCGGGAGTCGCTGCGCGCTGCCTTCGCCCCGTGCCCCGCTCCG
CCGCCGCCTCGCGCCGCCCGCCCCGGCTCTGACTGACCGCGTTACTCCCA
CAGGTGAGCGGGCGGGACGGCCCTTCTCCTCCGGGCTGTAATTAGCTGAG
CAAGAGGTAAGGGTTTAAGGGATGGTTGGTTGGTGGGGTATTAATGTTTA
ATTACCTGGAGCACCTGCCTGAAATCACTTTTTTTCAGGTTGGGCCACCA
TGGTTTCCCCAGACCCCCAAATTATCGTGGTGGCCCCCGAGACCGAACTC
GCGTCTATGCAAGTCCAACGCACTGAGGACGGGGTAACCATTATCCAGAT
ATTTTGGGTGGGCCGCAAAGGCGAGCTACTTAGACGCACCCCGGTGAGCT
CGGCCATGCAGACACCAATGGGAATCCCAATGGGGAAGTCGATGCTGGTG
CTTCTCACCTTCTTGGCCTTCGCCTCGTGCTGCATTGCTGCTTACCGCCC
CAGTGAGACCCTGTGCGGCGGGGAGCTGGTGGACACCCTCCAGTTCGTCT
GTGGGGACCGCGGCTTCTACTTCAGCAGGCCCGCAAGCCGTGTGAGCCGT
CGCAGCCGTGGCATCGTTGAGGAGTGCTGTTTCCGCAGCTGTGACCTGGC
CCTCCTGGAGACGTACTGTGCTACCCCCGCCAAGTCCGAGAGGGACGTGT
CGACCCCTCCGACCGTGCTTCCGGACAACTTCCCCAGATACCCCGTGGGC
AAGTTCTTCCAATATGACACCTGGAAGCAGTCCACCCAGCGCCTGCGCAG
GGGCCTGCCTGCCCTCCTGCGTGCCCGCCGGGGTCACGTGCTCGCCAAGG
AGCTCGAGGCGTTCAGGGAGGCCAAACGTCACCGTCCCCTGATTGCTCTA
CCCACCCAAGACCCCGCCCACGGGGGCGCCCCCCCAGAGATGGCCAGCAA
TCGGAAGGGATCTGGCGCCACAAACTTCAGCCTGCTGAAGCAGGCCGGAG
ATGTGGAAGAAAATCCTGGCCCCATGAGCAGACTGGATAAGTCCAAGGTG
ATCAACAGCGCCCTGGAACTGCTCAACGGCGTCGGCATCGAGGGACTCAC
CACCCGGAAGCTGGCCCAGAAACTGGGCGTGGAACAACCTACACTGTACT
GGCACGTGAAGAACAAAAGAGCCCTGCTGGATGCCCTGCCAATCGAGATG
CTGGACCGGCACCATACCCACAGCTGCCCCCTGGAGGGAGAAAGCTGGCA
GGACTTCCTGAGAAACAACGCTAAGAGCTACCGGTGCGCCCTGCTGAGCC
ACCGGGACGGCGCCAAGGTGCACTTGGGGACCAGACCCACGGAAAAGCAA
TACGAGACACTGGAGAACCAGCTGGCCTTCCTGTGCCAGCAGGGCTTCAG
CCTGGAGAATGCCCTCTACGCCCTGTCTGCCGTGGGCCACTTTACCCTGG
GATGTGTGCTGGAAGAGCAGGAGCACCAGGTGGCCAAGGAAGAGAGGGAA
ACCCCTACCACCGACAGCATGCCTCCTCTGCTGAAGCAAGCTATCGAGCT
GTTCGACAGACAGGGCGCCGAGCCTGCTTTCCTGTTTGGCCTGGAGCTGA
TCATCTGCGGCTTGGAGAAACAGCTGAAGTGCGAGAGCGGCGGCCCTACC
GACGCCCTGGACGATTTCGACCTGGATATGCTGCCTGCCGACGCCCTGGA
CGACTTCGACCTGGATATGCTGCCTGCCGATGCTCTGGACGACTTCGACC
TGGACATGCTTCCAGGCTAACTGTGCCTTCTAGTTGCCAGCCATCTGTTG
TTTGCCCCTCCCCCGTGCCTTCCTTGACCCTGGAAGGTGCCACTCCCACT
GTCCTTTCCTAATAAAATGAGGAAATTGCATCGCATTGTCTGAGTAGGTG
TCATTCTATTCTGGGGGGTGGGGTGGGGCAGGACAGCAAGGGGGAGGATT
GGGAAGAGAATAGCAGGCAGGCTGGGGAAACTTGTTTATTGCAGCTTATA
ATGGTTACAAATAAAGCAATAGCATCACAAATTTCACAAATAAAGCATTT
TTTTCACTGCATTCTAGTTGTGGTTTGTCCAAACTCATCAATGTATCTTA
TCATTAATCTGTCAGGAAGTTGTGGAGGCACCTCATCATGGAGAGGCCAG
AGGGCAGCTGTGGCCAGGCGCCTTCGCTCGTGGAACGCTTGTGGCCTGTC
GCGGGTGGCTGGAAGGTCCTCTGCTTCTTATCCGGGAAGAGAAGGACGTC
CAAGTCAGAGTCAATAACCAGCTGGGCCACCCGGAATGCGAGGGTGCTGC
CTGAGGGGATGGTGACCGTCTTCTTCTGGCTCAGATGGCCCTGGCCCTCA
CCCTGCAAGCACGTGGCTCCGGGCAGGGAAAACCGGCCCGAGCCCTCCCG
CTTGTGGGTGCGCGTGACTTCCACCTCCTTCTGTGTCTGCAGCACCTCAG
TCACCACGTACACGTTGTCCCCGCGGCTGCGCAGCTGCTGCAGGACTTTG
TGTTCTGGCTGCCGCAGGTGCCTCTCATGGAGCAGAGTCTGCCAGGTGTT
AGGGTCCACACTCAGCGAGTACACATTCATTGAGGTGCTGGAGCTGTCAG
ACACCGCGGCCCCGCCTGCGATCTTTGCCTGTCCTGGGGCTGCCAGCTCC
ACGCTGCCCTGTATCTGCCCATCCATGGCATCGTAGAAGTGGAAGCTCCT
GCCACGCTGCACGTCTGGTTCCGCGGCATCCGGCTCCAGGATGTCCTTGA
TAGACAGGTTGACACACTTATAACGGGGTTTCCAGAACCATGAGCTTGAG
GGCTTCCTAACCACCAGGCAGTAGGGCTGGAAGCCAGTGGAGCTCTGCAG
GCTGGTCACAGGGATGAACTCCCCACCATGGTCCAGCTCCTGGACCACTC
TCCGGACTACCCGCTCAAAGGCCGACCCCATGGTGGCTTTACGAGGGTAG
GAAGTGGTACGGAAAGTTGGTATAAGACAAAAGTGTTGTGGAATTGCTCC
AGGCGATCTGACGGTTCACTAAACGAGCTCTGCTTTTATAGGCGCCCACC
GTACACGCCTAAAGCTTATACGTTCTCTATCACTGATAGGGAGTAAACTG
GATATACGTTCTCTATCACTGATAGGGAGTAAACTGTAGATACGTTCTCT
ATCACTGATAGGGAGTAAACTGGTCATACGTTCTCTATCACTGATAGGGA
GTAAACTCCTTATACGTTCTCTATCACTGATAGGGAGTAAAGTCTGCATA
CGTTCTCTATCACTGATAGGGAGTAAACTCTTCATACGTTCTCTATCACT
GATAGGGAGTAAACTGGGGACTTTCCAGCTGGGGACTTTCCAGCTGGGAC
TTTCCAGGAG
Transgene: insulin-like growth factor 2 (IGF2)
Nucleotide sequence
(SEQ ID NO: 82)
ATGGTTTCCCCAGACCCCCAAATTATCGTGGTGGCCCCCGAGACCGAACT
CGCGTCTATGCAAGTCCAACGCACTGAGGACGGGGTAACCATTATCCAGA
TATTTTGGGTGGGCCGCAAAGGCGAGCTACTTAGACGCACCCCGGTGAGC
TCGGCCATGCAGACACCAATGGGAATCCCAATGGGGAAGTCGATGCTGGT
GCTTCTCACCTTCTTGGCCTTCGCCTCGTGCTGCATTGCTGCTTACCGCC
CCAGTGAGACCCTGTGCGGCGGGGAGCTGGTGGACACCCTCCAGTTCGTC
TGTGGGGACCGCGGCTTCTACTTCAGCAGGCCCGCAAGCCGTGTGAGCCG
TCGCAGCCGTGGCATCGTTGAGGAGTGCTGTTTCCGCAGCTGTGACCTGG
CCCTCCTGGAGACGTACTGTGCTACCCCCGCCAAGTCCGAGAGGGACGTG
TCGACCCCTCCGACCGTGCTTCCGGACAACTTCCCCAGATACCCCGTGGG
CAAGTTCTTCCAATATGACACCTGGAAGCAGTCCACCCAGCGCCTGCGCA
GGGGCCTGCCTGCCCTCCTGCGTGCCCGCCGGGGTCACGTGCTCGCCAAG
GAGCTCGAGGCGTTCAGGGAGGCCAAACGTCACCGTCCCCTGATTGCTCT
ACCCACCCAAGACCCCGCCCACGGGGGCGCCCCCCCAGAGATGGCCAGCA
ATCGGAAG
Amino acid sequence:
(SEQ ID NO: 83)
MVSPDPQIIVVAPETELASMQVQRTEDGVTIIQIFWVGRKGELLRRTPVS
SAMQTPMGIPMGKSMLVLLTFLAFASCCIAAYRPSETLCGGELVDTLQFV
CGDRGFYFSRPASRVSRRSRGIVEECCFRSCDLALLETYCATPAKSERDV
STPPTVLPDNFPRYPVGKFFQYDTWKQSTQRLRRGLPALLRARRGHVLAK
ELEAFREAKRHRPLIALPTQDPAHGGAPPEMASNRK
Actuator: Tetracycline or derivative
Actuator response element: TetRepressor/
Gen 3 TetOn, third generation rtTA/TRE3G
TRE3G Promoter
(SEQ ID NO: 45)
TTTACTCCCTATCAGTGATAGAGAACGTATGAAGAGTTTACTCCCTATCA
GTGATAGAGAACGTATGCAGACTTTACTCCCTATCAGTGATAGAGAACGT
ATAAGGAGTTTACTCCCTATCAGTGATAGAGAACGTATGACCAGTTTACT
CCCTATCAGTGATAGAGAACGTATCTACAGTTTACTCCCTATCAGTGATA
GAGAACGTATATCCAGTTTACTCCCTATCAGTGATAGAGAACGTATAAGC
TTTAGGCGTGTACGGTGGGCGCCTATAAAAGCAGAGCTCGTTTAGTGAAC
CGTCAGATCGCCTGGAGCAATTCCACAACACTTTTGTCTTATACCAACTT
TCCGTACCACTTCCTACCCTCGTAAA
TetOn 3G
Nucleotide sequence
(SEQ ID NO: 43)
ATGAGCAGACTGGATAAGTCCAAGGTGATCAACAGCGCCCTGGAACTGCT
CAACGGCGTCGGCATCGAGGGACTCACCACCCGGAAGCTGGCCCAGAAAC
TGGGCGTGGAACAACCTACACTGTACTGGCACGTGAAGAACAAAAGAGCC
CTGCTGGATGCCCTGCCAATCGAGATGCTGGACCGGCACCATACCCACAG
CTGCCCCCTGGAGGGAGAAAGCTGGCAGGACTTCCTGAGAAACAACGCTA
AGAGCTACCGGTGCGCCCTGCTGAGCCACCGGGACGGCGCCAAGGTGCAC
TTGGGGACCAGACCCACGGAAAAGCAATACGAGACACTGGAGAACCAGCT
GGCCTTCCTGTGCCAGCAGGGCTTCAGCCTGGAGAATGCCCTCTACGCCC
TGTCTGCCGTGGGCCACTTTACCCTGGGATGTGTGCTGGAAGAGCAGGAG
CACCAGGTGGCCAAGGAAGAGAGGGAAACCCCTACCACCGACAGCATGCC
TCCTCTGCTGAAGCAAGCTATCGAGCTGTTCGACAGACAGGGCGCCGAGC
CTGCTTTCCTGTTTGGCCTGGAGCTGATCATCTGCGGCTTGGAGAAACAG
CTGAAGTGCGAGAGCGGCGGCCCTACCGACGCCCTGGACGATTTCGACCT
GGATATGCTGCCTGCCGACGCCCTGGACGACTTCGACCTGGATATGCTGC
CTGCCGATGCTCTGGACGACTTCGACCTGGACATGCTTCCAGGCTAA
Amino acid sequence
(SEQ ID NO: 44)
MSRLDKSKVINSALELLNGVGIEGLTTRKLAQKLGVEQPTLYWHVKNKRA
LLDALPIEMLDRHHTHSCPLEGESWQDFLRNNAKSYRCALLSHRDGAKVH
LGTRPTEKQYETLENQLAFLCQQGFSLENALYALSAVGHFTLGCVLEEQE
HQVAKEERETPTTDSMPPLLKQAIELFDRQGAEPAFLFGLELIICGLEKQ
LKCESGGPTDALDDFDLDMLPADALDDFDLDMLPADALDDFDLDMLPG
Effector element: N-terminal domain of
gasdermin D Nucleotide sequence
(SEQ ID NO: 84)
ATGGGGTCGGCCTTTGAGCGGGTAGTCCGGAGAGTGGTCCAGGAGCTGGA
CCATGGTGGGGAGTTCATCCCTGTGACCAGCCTGCAGAGCTCCACTGGCT
TCCAGCCCTACTGCCTGGTGGTTAGGAAGCCCTCAAGCTCATGGTTCTGG
AAACCCCGTTATAAGTGTGTCAACCTGTCTATCAAGGACATCCTGGAGCC
GGATGCCGCGGAACCAGACGTGCAGCGTGGCAGGAGCTTCCACTTCTACG
ATGCCATGGATGGGCAGATACAGGGCAGCGTGGAGCTGGCAGCCCCAGGA
CAGGCAAAGATCGCAGGCGGGGCCGCGGTGTCTGACAGCTCCAGCACCTC
AATGAATGTGTACTCGCTGAGTGTGGACCCTAACACCTGGCAGACTCTGC
TCCATGAGAGGCACCTGCGGCAGCCAGAACACAAAGTCCTGCAGCAGCTG
CGCAGCCGCGGGGACAACGTGTACGTGGTGACTGAGGTGCTGCAGACACA
GAAGGAGGTGGAAGTCACGCGCACCCACAAGCGGGAGGGCTCGGGCCGGT
TTTCCCTGCCCGGAGCCACGTGCTTGCAGGGTGAGGGCCAGGGCCATCTG
AGCCAGAAGAAGACGGTCACCATCCCCTCAGGCAGCACCCTCGCATTCCG
GGTGGCCCAGCTGGTTATTGACTCTGACTTGGACGTCCTTCTCTTCCCGG
ATAAGAAGCAGAGGACCTTCCAGCCACCCGCGACAGGCCACAAGCGTTCC
ACGAGCGAAGGCGCCTGGCCACAGCTGCCCTCTGGCCTCTCCATGATGAG
GTGCCTCCACAACTTCCTGACAGAT
Amino acid sequence
(SEQ ID NO: 85)
MGSAFERVVRRVVQELDHGGEFIPVTSLQSSTGFQPYCLVVRKPSSSWFW
KPRYKCVNLSIKDILEPDAAEPDVQRGRSFHFYDAMDGQIQGSVELAAPG
QAKIAGGAAVSDSSSTSMNVYSLSVDPNTWQTLLHERHLRQPEHKVLQQL
RSRGDNVYVVTEVLQTQKEVEVTRTHKREGSGRESLPGATCLQGEGQGHL
SQKKTVTIPSGSTLAFRVAQLVIDSDLDVLLFPDKKQRTFQPPATGHKRS
TSEGAWPQLPSGLSMMRCLHNFLTD
Auxiliary sequences:
CBh promoter
(SEQ ID NO: 32)
CGTTACATAACTTACGGTAAATGGCCCGCCTGGCTGACCGCCCAACGACC
CCCGCCCATTGACGTCAATAGTAACGCCAATAGGGACTTTCCATTGACGT
CAATGGGTGGAGTATTTACGGTAAACTGCCCACTTGGCAGTACATCAAGT
GTATCATATGCCAAGTACGCCCCCTATTGACGTCAATGACGGTAAATGGC
CCGCCTGGCATTGTGCCCAGTACATGACCTTATGGGACTTTCCTACTTGG
CAGTACATCTACGTATTAGTCATCGCTATTACCATGGTCGAGGTGAGCCC
CACGTTCTGCTTCACTCTCCCCATCTCCCCCCCCTCCCCACCCCCAATTT
TGTATTTATTTATTTTTTAATTATTTTGTGCAGCGATGGGGGCGGGGGGG
GGGGGGGGGCGCGCGCCAGGCGGGGCGGGGCGGGGCGAGGGGCGGGGCGG
GGCGAGGCGGAGAGGTGCGGCGGCAGCCAATCAGAGCGGCGCGCTCCGAA
AGTTTCCTTTTATGGCGAGGCGGCGGCGGCGGCGGCCCTATAAAAAGCGA
AGCGCGCGGCGGGCGGGAGTCGCTGCGCGCTGCCTTCGCCCCGTGCCCCG
CTCCGCCGCCGCCTCGCGCCGCCCGCCCCGGCTCTGACTGACCGCGTTAC
TCCCACAGGTGAGCGGGCGGGACGGCCCTTCTCCTCCGGGCTGTAATTAG
CTGAGCAAGAGGTAAGGGTTTAAGGGATGGTTGGTTGGTGGGGTATTAAT
GTTTAATTACCTGGAGCACCTGCCTGAAATCACTTTTTTTCAGGTTGG
Self-cleaving 2A peptide from porcine
teschovirus-1 Nucleotide sequence
(SEQ ID NO: 37)
GGATCTGGCGCCACAAACTTCAGCCTGCTGAAGCAGGCCGGAGATGTGGA
AGAAAATCCTGGCCCC
Amino acid sequence
(SEQ ID NO: 38)
GSGATNFSLLKQAGDVEENPGP
Kozak
GCCACC
bGH polyA
(SEQ ID NO: 46)
CTGTGCCTTCTAGTTGCCAGCCATCTGTTGTTTGCCCCTCCCCCGTGCCT
TCCTTGACCCTGGAAGGTGCCACTCCCACTGTCCTTTCCTAATAAAATGA
GGAAATTGCATCGCATTGTCTGAGTAGGTGTCATTCTATTCTGGGGGGTG
GGGGGGGCAGGACAGCAAGGGGGAGGATTGGGAAGAGAATAGCAGGCAGG
CTGGGGA
SV40 polyA
(SEQ ID NO: 47)
TAAGATACATTGATGAGTTTGGACAAACCACAACTAGAATGCAGTGAAAA
AAATGCTTTATTTGTGAAATTTGTGATGCTATTGCTTTATTTGTAACCAT
TATAAGCTGCAATAAACAAGTT

### Example 10

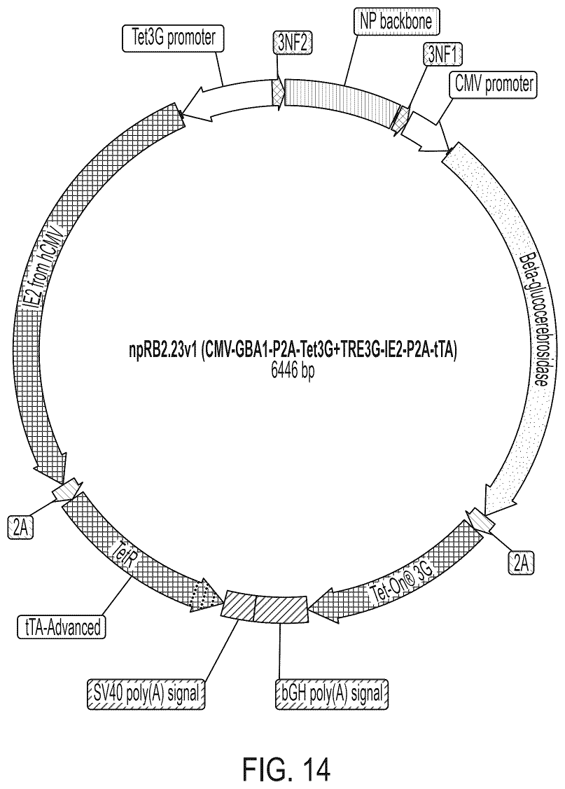

In the present example a single actuator-regulated effector element is intended to deliver biologically active beta-glucocerebrosidase for the treatment of Gaucher's disease. Beta-glucocerebrosidase is expressed under the CMV promoter. The actuator is tetracycline or a tetracycline derivative, which acts upon a 3^{rd} generation TetOn inducible promoter. In conjunction with the TetOn operator, this activates expression of a viral DNA-binding protein (IE2 from human cytomegalovirus) that binds to the CMV promoter and inhibits expression of beta-glucocerebrosidase. Tetracycline and the TetOn operator additionally activate expression of a transcriptional transactivator (tTA) that continuously activates the TetOn operator after the withdrawal of tetracycline. FIG. 14 shows a plasmid map for the genetic construct described in this example.

Full sequence (except nanoplasmid backbone)
(SEQ ID NO: 86)
CTGGGGACTTTCCAGCCTGGGGACTTTCCAGCTGGGACTTTCCAGGTGAT
GCGGTTTTGGCAGTACACCAATGGGCGTGGATAGCGGTTTGACTCACGGG
GATTTCCAAGTCTCCACCCCATTGACGTCAATGGGAGTTTGTTTTGGCAC
CAAAATCAACGGGACTTTCCAAAATGTCGTAATAACCCCGCCCCGTTGAC
GCAAATGGGCGGTAGGCGTGTACGGTGGGAGGTCTATATAAGCAGAGCCA
CCATGGAGTTTTCAAGTCCTTCCAGAGAGGAATGTCCCAAGCCTTTGAGT
AGGGTAAGCATCATGGCTGGCAGCCTCACAGGATTGCTTCTACTTCAGGC
AGTGTCGTGGGCATCAGGTGCCCGCCCCTGCATCCCTAAAAGCTTCGGCT
ACAGCTCGGTGGTGTGTGTCTGCAATGCCACATACTGTGACTCCTTTGAC
CCCCCGACCTTTCCTGCCCTTGGTACCTTCAGCCGCTATGAGAGTACACG
CAGTGGGCGACGGATGGAGCTGAGTATGGGGCCCATCCAGGCTAATCACA
CGGGCACAGGCCTGCTACTGACCCTGCAGCCAGAACAGAAGTTCCAGAAA
GTGAAGGGATTTGGAGGGGCCATGACAGATGCTGCTGCTCTCAACATCCT
TGCCCTGTCACCCCCTGCCCAAAATTTGCTACTTAAATCGTACTTCTCTG
AAGAAGGAATCGGATATAACATCATCCGGGTACCCATGGCCAGCTGTGAC
TTCTCCATCCGCACCTACACCTATGCAGACACCCCTGATGATTTCCAGTT
GCACAACTTCAGCCTCCCAGAGGAAGATACCAAGCTCAAGATACCCCTGA
TTCACCGAGCCCTGCAGTTGGCCCAGCGTCCCGTTTCACTCCTTGCCAGC
CCCTGGACATCACCCACTTGGCTCAAGACCAATGGAGCGGTGAATGGGAA
GGGGTCACTCAAGGGACAGCCCGGAGACATCTACCACCAGACCTGGGCCA
GATACTTTGTGAAGTTCCTGGATGCCTATGCTGAGCACAAGTTACAGTTC
TGGGCAGTGACAGCTGAAAATGAGCCTTCTGCTGGGCTGTTGAGTGGATA
CCCCTTCCAGTGCCTGGGCTTCACCCCTGAACATCAGCGAGACTTCATTG
CCCGTGACCTAGGTCCTACCCTCGCCAACAGTACTCACCACAATGTCCGC
CTACTCATGCTGGATGACCAACGCTTGCTGCTGCCCCACTGGGCAAAGGT
GGTACTGACAGACCCAGAAGCAGCTAAATATGTTCATGGCATTGCTGTAC
ATTGGTACCTGGACTTTCTGGCTCCAGCCAAAGCCACCCTAGGGGAGACA
CACCGCCTGTTCCCCAACACCATGCTCTTTGCCTCAGAGGCCTGTGTGGG
CTCCAAGTTCTGGGAGCAGAGTGTGCGGCTAGGCTCCTGGGATCGAGGGA
TGCAGTACAGCCACAGCATCATCACGAACCTCCTGTACCATGTGGTCGGC
TGGACCGACTGGAACCTTGCCCTGAACCCCGAAGGAGGACCCAATTGGGT
GCGTAACTTTGTCGACAGTCCCATCATTGTAGACATCACCAAGGACACGT
TTTACAAACAGCCCATGTTCTACCACCTTGGCCACTTCAGCAAGTTCATT
CCTGAGGGCTCCCAGAGAGTGGGGCTGGTTGCCAGTCAGAAGAACGACCT
GGACGCAGTGGCACTGATGCATCCCGATGGCTCTGCTGTTGTGGTCGTGC
TAAACCGCTCCTCTAAGGATGTGCCTCTTACCATCAAGGATCCTGCTGTG
GGCTTCCTGGAGACAATCTCACCTGGCTACTCCATTCACACCTACCTGTG
GCGTCGCCAGGGCTCTGGAGCCACAAACTTCAGCCTGCTGAAGCAGGCCG
GAGATGTGGAAGAAAATCCTGGCCCCATGAGCAGACTGGATAAGTCCAAG
GTGATCAACAGCGCCCTGGAACTGCTCAACGGCGTCGGCATCGAGGGACT
CACCACCCGGAAGCTGGCCCAGAAACTGGGCGTGGAACAACCTACACTGT
ACTGGCACGTGAAGAACAAAAGAGCCCTGCTGGATGCCCTGCCAATCGAG
ATGCTGGACCGGCACCATACCCACAGCTGCCCCCTGGAGGGAGAAAGCTG
GCAGGACTTCCTGAGAAACAACGCTAAGAGCTACCGGTGCGCCCTGCTGA
GCCACCGGGACGGCGCCAAGGTGCACTTGGGGACCAGACCCACGGAAAAG
CAATACGAGACACTGGAGAACCAGCTGGCCTTCCTGTGCCAGCAGGGCTT
CAGCCTGGAGAATGCCCTCTACGCCCTGTCTGCCGTGGGCCACTTTACCC
TGGGATGTGTGCTGGAAGAGCAGGAGCACCAGGTGGCCAAGGAAGAGAGG
GAAACCCCTACCACCGACAGCATGCCTCCTCTGCTGAAGCAAGCTATCGA
GCTGTTCGACAGACAGGGCGCCGAGCCTGCTTTCCTGTTTGGCCTGGAGC
TGATCATCTGCGGCTTGGAGAAACAGCTGAAGTGCGAGAGCGGCGGCCCT
ACCGACGCCCTGGACGATTTCGACCTGGATATGCTGCCTGCCGACGCCCT
GGACGACTTCGACCTGGATATGCTGCCTGCCGATGCTCTGGACGACTTCG
ACCTGGACATGCTTCCAGGCTAACTGTGCCTTCTAGTTGCCAGCCATCTG
TTGTTTGCCCCTCCCCCGTGCCTTCCTTGACCCTGGAAGGTGCCACTCCC
ACTGTCCTTTCCTAATAAAATGAGGAAATTGCATCGCATTGTCTGAGTAG
GTGTCATTCTATTCTGGGGGGTGGGGTGGGGCAGGACAGCAAGGGGGAGG
ATTGGGAAGAGAATAGCAGGCAGGCTGGGGAAACTTGTTTATTGCAGCTT
ATAATGGTTACAAATAAAGCAATAGCATCACAAATTTCACAAATAAAGCA
TTTTTTTCACTGCATTCTAGTTGTGGTTTGTCCAAACTCATCAATGTATC
TTATTACCCGGGGAGCATGTCAAGGTCCAAATCGTCAAGAGCGTCAGCAG
GCAGCATATCAAGGTCAAAGTCGTCAAGGGCATCGGCTGGGAGCATGTCT
AAGTCAAAATCGTCAAGGGCGTCGGCCGGCCCGCCGCTTTCGCACTTTAG
CTGTTTCTCCAGGCCACATATGATTAGTTCCAGGCCGAAAAGGAAGGCAG
GTTCGGCTCCCTGATGGTCGAACAGCTCAATTGCTTGTCTCAGAAGTGGG
GGCATAGAATCGGTGGTAGGTGTCTCTCTTTCCTCTTTTGCTACTTGATG
CTCCTGATCCTCCAATACGCAGCCCAGTGTAAAGTGGCCCACGGCGGACA
GAGCGTACAGTGCGTTCTCCAGGGAGAAGCCTTGCTGACACAGGAACGCG
AGCTGATTTTCCAGGGTTTCGTACTGTTTCTCTGTTGGGCGGGTGCCGAG
ATGCACTTTAGCCCCGTCGCGATGTGAGAGGAGAGCACAGCGGAATGACT
TGGCGTTGTTCCGCAGAAAGTCTTGCCATGACTCGCCTTCCAGGGGGCAG
AAGTGGGTATGATGCCTGTCCAGCATCTCGATTGCCAGGGCATCGAGCAG
GGCCCGCTTGTTCTTCACGTGCCAGTACAGGGTAGGCTGCTCAACTCCCA
GCTTTTGAGCGAGTTTCCTTGTCGTCAGGCCTTCGATACCGACTTCATTG
AGTAATTCCAGAGCAGAGTTTATGACTTTGCTCTTGTCCAGTCTAGACAT
GGGGCCAGGATTTTCTTCCACATCTCCGGCCTGCTTCAGCAGGCTGAAGT
TTGTGGCTCCAGAGCCCTGAGATTTGTTCCTCAGGTCCTGGATGGCTGCC
TCGATGGCCAGGCTCAGGGTGTCCAGGTCTTCGGGAGGGGTCTCGGTGGG
CTTCTCAAACTGCCCCACGGCGTAGGCCTTCGCGGCCGTCTCGTAGATAG
GCAGCATGAACCCACCCTGGTTGGTGGAGAAGATGCGCACCATGACCTGT
TTGGGAAACTTTTGCATCAGGGGCAGGCACAGGTTGAGAGCGCCCAACAG
GTCCACGGGGGTGGCAGCGTGGATAATCATGTTGCGGTAATCAGAAGAGC
GGGGGCACAATTGGTGGGTGTGCAGTTCTTTGAGGTTCCACACGGCCTTG
ACACCGTCGTTACAAGCATCGGCCGTGCGCTGCGCCACATCAGGAGGGTG
TGTCACAGGCATAGTGTGCTCCATGAGGAAGGGAGTGGAGAGGGCCAGGT
TGCACATGCTACCCAGGCGACACCGTACCTGATCCACCTCATTCTTCACT
TCATGATTGCGGGTGTAGATGATCTGAATGCCCTTGTTGTTCACCTGCAT
GGTTTTGCAAACTTTGATGGCCTCTTCTAACACTTGGTGCATACTAGGGA
TCATGAATGGCAGATTCTTGTACTCAAGAGAACGATTGGTGTGACGGAAC
ATGCGGCTCACCTCGTCAATCTTGACGCGACCCCGCCGAGTCTGCACGTT
GGGTGTGCAGAAGGGGGTGTTCTTATCTTTCATGATATTGCGCACCTTCT
CGTTGTCCAACTCGGAGATGCGTTTGCTCTTCTTCTTGCGAGGTCCGGTG
CTCGCCCCGCCGCTGCTCTGATGGCCGCAGCTCAGCAGAGAGGAGGAGGC
CGCGCCACCAAAACCGCCGCGCCCATGGTGGCTCGAGGTCACGGATGCTC
CTCCGCCACTGCTGCATTTCATCTCCTCGGACTCACTCTCCGAGTCCGAA
GCCGAACTGCAGGAGGAGGAAGACGAAGAGGAACTATCTTCATCGGGCCG
GCCCAAAGGATCGGGAAGAGGAGGGTGGTTCATCTGGGAGAGCGGGTGCG
TGGGAGAGGTCACTCGCGGCGTGCCGCTGCCGGTGGAAGGGGAAGACGCG
GTAGCACCGCGGGTCTCGACTTCTTCACCCTGTTCTTCCTCGCTATCAGA
GATCACGATACAGCCGGCGGTATCGATAATCTTGTTGCGGTACTGGATGG
TAAAGTCGGGCTCGGGCTTGATGTCTTCCTGTTTGATGAGGGGCAGCATG
ATAGGCGCGGGAGGCACGGGCGGTTTAATAATCACCTTGAAAGGACGCGT
GGTTTTGCGCGGTTTCTTACGCGGGCTGAGCTCGGGAGTAGCGGATGCCC
CGGGGAGAGGAGTGTTAGTAACCGCGACGCTGGTGGGGGTCGGCTTGTTA
AGAGGGGCGCTGCTAACGCTGCAAGAGTGGGTTGTCAGCGTGGGGCCGGT
GCTACTGGAATCGATACCGGCATGATTGACAGCCTGGGCGAGGATGTCAC
CGAGTTCTGTCAGGACATCTTTTTCGGGGTTCTCGTTGCAATCCTCGGTC
ACTTGTTCAAAGGTTTTGAGGGATTCTTCGGCCAATTCTGGGAACAGCGG
GTCTCCCAGGCTCAGCTGACTGTTAACCTCCTTCCTTAACATAGTCTGCA
GGAACGTCGTGGCCTTGGTCACGGGTGTCTCGGGCCGTGGCACCTTGGAG
GAAGGGCCCTCGTCAGGATTATCAGGGTCCATCTTTCTCTTGGCAGAGGA
CTCCATGGTGGCTTTACGAGGGTAGGAAGTGGTACGGAAAGTTGGTATAA
GACAAAAGTGTTGTGGAATTGCTCCAGGCGATCTGACGGTTCACTAAACG
AGCTCTGCTTTTATAGGCGCCCACCGTACACGCCTAAAGCTTATACGTTC
TCTATCACTGATAGGGAGTAAACTGGATATACGTTCTCTATCACTGATAG
GGAGTAAACTGTAGATACGTTCTCTATCACTGATAGGGAGTAAACTGGTC
ATACGTTCTCTATCACTGATAGGGAGTAAACTCCTTATACGTTCTCTATC
ACTGATAGGGAGTAAAGTCTGCATACGTTCTCTATCACTGATAGGGAGTA
AACTCTTCATACGTTCTCTATCACTGATAGGGAGTAAACTGGGGACTTTC
CAGCTGGGGACTTTCCAGCTGGGACTTTCCAGGAGA
Transgene: beta-glucocerebrosidase
Nucleotide sequence
(SEQ ID NO: 87)
ATGGAGTTTTCAAGTCCTTCCAGAGAGGAATGTCCCAAGCCTTTGAGTAG
GGTAAGCATCATGGCTGGCAGCCTCACAGGATTGCTTCTACTTCAGGCAG
TGTCGTGGGCATCAGGTGCCCGCCCCTGCATCCCTAAAAGCTTCGGCTAC
AGCTCGGTGGTGTGTGTCTGCAATGCCACATACTGTGACTCCTTTGACCC
CCCGACCTTTCCTGCCCTTGGTACCTTCAGCCGCTATGAGAGTACACGCA
GTGGGCGACGGATGGAGCTGAGTATGGGGCCCATCCAGGCTAATCACACG
GGCACAGGCCTGCTACTGACCCTGCAGCCAGAACAGAAGTTCCAGAAAGT
GAAGGGATTTGGAGGGGCCATGACAGATGCTGCTGCTCTCAACATCCTTG
CCCTGTCACCCCCTGCCCAAAATTTGCTACTTAAATCGTACTTCTCTGAA
GAAGGAATCGGATATAACATCATCCGGGTACCCATGGCCAGCTGTGACTT
CTCCATCCGCACCTACACCTATGCAGACACCCCTGATGATTTCCAGTTGC
ACAACTTCAGCCTCCCAGAGGAAGATACCAAGCTCAAGATACCCCTGATT
CACCGAGCCCTGCAGTTGGCCCAGCGTCCCGTTTCACTCCTTGCCAGCCC
CTGGACATCACCCACTTGGCTCAAGACCAATGGAGCGGTGAATGGGAAGG
GGTCACTCAAGGGACAGCCCGGAGACATCTACCACCAGACCTGGGCCAGA
TACTTTGTGAAGTTCCTGGATGCCTATGCTGAGCACAAGTTACAGTTCTG
GGCAGTGACAGCTGAAAATGAGCCTTCTGCTGGGCTGTTGAGTGGATACC
CCTTCCAGTGCCTGGGCTTCACCCCTGAACATCAGCGAGACTTCATTGCC
CGTGACCTAGGTCCTACCCTCGCCAACAGTACTCACCACAATGTCCGCCT
ACTCATGCTGGATGACCAACGCTTGCTGCTGCCCCACTGGGCAAAGGTGG
TACTGACAGACCCAGAAGCAGCTAAATATGTTCATGGCATTGCTGTACAT
TGGTACCTGGACTTTCTGGCTCCAGCCAAAGCCACCCTAGGGGAGACACA
CCGCCTGTTCCCCAACACCATGCTCTTTGCCTCAGAGGCCTGTGTGGGCT
CCAAGTTCTGGGAGCAGAGTGTGCGGCTAGGCTCCTGGGATCGAGGGATG
CAGTACAGCCACAGCATCATCACGAACCTCCTGTACCATGTGGTCGGCTG
GACCGACTGGAACCTTGCCCTGAACCCCGAAGGAGGACCCAATTGGGTGC
GTAACTTTGTCGACAGTCCCATCATTGTAGACATCACCAAGGACACGTTT
TACAAACAGCCCATGTTCTACCACCTTGGCCACTTCAGCAAGTTCATTCC
TGAGGGCTCCCAGAGAGTGGGGCTGGTTGCCAGTCAGAAGAACGACCTGG
ACGCAGTGGCACTGATGCATCCCGATGGCTCTGCTGTTGTGGTCGTGCTA
AACCGCTCCTCTAAGGATGTGCCTCTTACCATCAAGGATCCTGCTGTGGG
CTTCCTGGAGACAATCTCACCTGGCTACTCCATTCACACCTACCTGTGGC
GTCGCCAG
Amino acid sequence
(SEQ ID NO: 88)
MEFSSPSREECPKPLSRVSIMAGSLTGLLLLQAVSWASGARPCIPKSFGY
SSVVCVCNATYCDSFDPPTFPALGTFSRYESTRSGRRMELSMGPIQANHT
GTGLLLTLQPEQKFQKVKGFGGAMTDAAALNILALSPPAQNLLLKSYFSE
EGIGYNIIRVPMASCDFSIRTYTYADTPDDFQLHNFSLPEEDTKLKIPLI
HRALQLAQRPVSLLASPWTSPTWLKTNGAVNGKGSLKGQPGDIYHQTWAR
YFVKFLDAYAEHKLQFWAVTAENEPSAGLLSGYPFQCLGFTPEHQRDFIA
RDLGPTLANSTHHNVRLLMLDDQRLLLPHWAKVVLTDPEAAKYVHGIAVH
WYLDFLAPAKATLGETHRLFPNTMLFASEACVGSKFWEQSVRLGSWDRGM
QYSHSIITNLLYHVVGWTDWNLALNPEGGPNWVRNFVDSPIIVDITKDTF
YKQPMFYHLGHFSKFIPEGSQRVGLVASQKNDLDAVALMHPDGSAVVVVL
NRSSKDVPLTIKDPAVGFLETISPGYSIHTYLWRRQ
Actuator: Tetracycline or derivative,
advanced tTA
Advanced tTA
Nucleotide sequence
(SEQ ID NO: 89)
ATGTCTAGACTGGACAAGAGCAAAGTCATAAACTCTGCTCTGGAATTACT
CAATGAAGTCGGTATCGAAGGCCTGACGACAAGGAAACTCGCTCAAAAGC
TGGGAGTTGAGCAGCCTACCCTGTACTGGCACGTGAAGAACAAGCGGGCC
CTGCTCGATGCCCTGGCAATCGAGATGCTGGACAGGCATCATACCCACTT
CTGCCCCCTGGAAGGCGAGTCATGGCAAGACTTTCTGCGGAACAACGCCA
AGTCATTCCGCTGTGCTCTCCTCTCACATCGCGACGGGGCTAAAGTGCAT
CTCGGCACCCGCCCAACAGAGAAACAGTACGAAACCCTGGAAAATCAGCT
CGCGTTCCTGTGTCAGCAAGGCTTCTCCCTGGAGAACGCACTGTACGCTC
TGTCCGCCGTGGGCCACTTTACACTGGGCTGCGTATTGGAGGATCAGGAG
CATCAAGTAGCAAAAGAGGAAAGAGAGACACCTACCACCGATTCTATGCC
CCCACTTCTGAGACAAGCAATTGAGCTGTTCGACCATCAGGGAGCCGAAC
CTGCCTTCCTTTTCGGCCTGGAACTAATCATATGTGGCCTGGAGAAACAG
CTAAAGTGCGAAAGCGGCGGGCCGGCCGACGCCCTTGACGATTTTGACTT
AGACATGCTCCCAGCCGATGCCCTTGACGACTTTGACCTTGATATGCTGC
CTGCTGACGCTCTTGACGATTTGGACCTTGACATGCTCCCCGGGTAA
Amino acid sequence
(SEQ ID NO: 90)
MSRLDKSKVINSALELLNEVGIEGLTTRKLAQKLGVEQPTLYWHVKNKRA
LLDALAIEMLDRHHTHFCPLEGESWQDFLRNNAKSFRCALLSHRDGAKVH
LGTRPTEKQYETLENQLAFLCQQGFSLENALYALSAVGHFTLGCVLEDQE
HQVAKEERETPTTDSMPPLLRQAIELFDHQGAEPAFLFGLELIICGLEKQ
LKCESGGPADALDDFDLDMLPADALDDFDLDMLPADALDDLDLDMLPG
Actuator response element: TetRepressor/
Gen 3 TetOn, third generation rtTA/TRE3G
TRE3G
(SEQ ID NO: 45)
TTTACTCCCTATCAGTGATAGAGAACGTATGAAGAGTTTACTCCCTATCA
GTGATAGAGAACGTATGCAGACTTTACTCCCTATCAGTGATAGAGAACGT
ATAAGGAGTTTACTCCCTATCAGTGATAGAGAACGTATGACCAGTTTACT
CCCTATCAGTGATAGAGAACGTATCTACAGTTTACTCCCTATCAGTGATA
GAGAACGTATATCCAGTTTACTCCCTATCAGTGATAGAGAACGTATAAGC
TTTAGGCGTGTACGGTGGGCGCCTATAAAAGCAGAGCTCGTTTAGTGAAC
CGTCAGATCGCCTGGAGCAATTCCACAACACTTTTGTCTTATACCAACTT
TCCGTACCACTTCCTACCCTCGTAAA
TetOn3G
Nucleotide sequence
(SEQ ID NO: 43)
ATGAGCAGACTGGATAAGTCCAAGGTGATCAACAGCGCCCTGGAACTGCT
CAACGGCGTCGGCATCGAGGGACTCACCACCCGGAAGCTGGCCCAGAAAC
TGGGCGTGGAACAACCTACACTGTACTGGCACGTGAAGAACAAAAGAGCC
CTGCTGGATGCCCTGCCAATCGAGATGCTGGACCGGCACCATACCCACAG
CTGCCCCCTGGAGGGAGAAAGCTGGCAGGACTTCCTGAGAAACAACGCTA
AGAGCTACCGGTGCGCCCTGCTGAGCCACCGGGACGGCGCCAAGGTGCAC
TTGGGGACCAGACCCACGGAAAAGCAATACGAGACACTGGAGAACCAGCT
GGCCTTCCTGTGCCAGCAGGGCTTCAGCCTGGAGAATGCCCTCTACGCCC
TGTCTGCCGTGGGCCACTTTACCCTGGGATGTGTGCTGGAAGAGCAGGAG
CACCAGGTGGCCAAGGAAGAGAGGGAAACCCCTACCACCGACAGCATGCC
TCCTCTGCTGAAGCAAGCTATCGAGCTGTTCGACAGACAGGGCGCCGAGC
CTGCTTTCCTGTTTGGCCTGGAGCTGATCATCTGCGGCTTGGAGAAACAG
CTGAAGTGCGAGAGCGGCGGCCCTACCGACGCCCTGGACGATTTCGACCT
GGATATGCTGCCTGCCGACGCCCTGGACGACTTCGACCTGGATATGCTGC
CTGCCGATGCTCTGGACGACTTCGACCTGGACATGCTTCCAGGCTAA
Amino acid sequence
(SEQ ID NO: 44)
MSRLDKSKVINSALELLNGVGIEGLTTRKLAQKLGVEQPTLYWHVKNKRA
LLDALPIEMLDRHHTHSCPLEGESWQDFLRNNAKSYRCALLSHRDGAKVH
LGTRPTEKQYETLENQLAFLCQQGFSLENALYALSAVGHFTLGCVLEEQE
HQVAKEERETPTTDSMPPLLKQAIELFDRQGAEPAFLFGLELIICGLEKQ
LKCESGGPTDALDDFDLDMLPADALDDFDLDMLPADALDDFDLDMLPG
Effector element: IE2 from human
cytomegalovirus
Nucleotide sequence
(SEQ ID NO: 91)
ATGGAGTCCTCTGCCAAGAGAAAGATGGACCCTGATAATCCTGACGAGGG
CCCTTCCTCCAAGGTGCCACGGCCCGAGACACCCGTGACCAAGGCCACGA
CGTTCCTGCAGACTATGTTAAGGAAGGAGGTTAACAGTCAGCTGAGCCTG
GGAGACCCGCTGTTCCCAGAATTGGCCGAAGAATCCCTCAAAACCTTTGA
ACAAGTGACCGAGGATTGCAACGAGAACCCCGAAAAAGATGTCCTGACAG
AACTCGGTGACATCCTCGCCCAGGCTGTCAATCATGCCGGTATCGATTCC
AGTAGCACCGGCCCCACGCTGACAACCCACTCTTGCAGCGTTAGCAGCGC
CCCTCTTAACAAGCCGACCCCCACCAGCGTCGCGGTTACTAACACTCCTC
TCCCCGGGGCATCCGCTACTCCCGAGCTCAGCCCGCGTAAGAAACCGCGC
AAAACCACGCGTCCTTTCAAGGTGATTATTAAACCGCCCGTGCCTCCCGC
GCCTATCATGCTGCCCCTCATCAAACAGGAAGACATCAAGCCCGAGCCCG
ACTTTACCATCCAGTACCGCAACAAGATTATCGATACCGCCGGCTGTATC
GTGATCTCTGATAGCGAGGAAGAACAGGGTGAAGAAGTCGAGACCCGCGG
TGCTACCGCGTCTTCCCCTTCCACCGGCAGCGGCACGCCGCGAGTGACCT
CTCCCACGCACCCGCTCTCCCAGATGAACCACCCTCCTCTTCCCGATCCT
TTGGGCCGGCCCGATGAAGATAGTTCCTCTTCGTCTTCCTCCTCCTGCAG
TTCGGCTTCGGACTCGGAGAGTGAGTCCGAGGAGATGAAATGCAGCAGTG
GCGGAGGAGCATCCGTGACCTCGAGCCACCATGGGCGCGGCGGTTTTGGT
GGCGCGGCCTCCTCCTCTCTGCTGAGCTGCGGCCATCAGAGCAGCGGCGG
GGCGAGCACCGGACCTCGCAAGAAGAAGAGCAAACGCATCTCCGAGTTGG
ACAACGAGAAGGTGCGCAATATCATGAAAGATAAGAACACCCCCTTCTGC
ACACCCAACGTGCAGACTCGGCGGGGTCGCGTCAAGATTGACGAGGTGAG
CCGCATGTTCCGTCACACCAATCGTTCTCTTGAGTACAAGAATCTGCCAT
TCATGATCCCTAGTATGCACCAAGTGTTAGAAGAGGCCATCAAAGTTTGC
AAAACCATGCAGGTGAACAACAAGGGCATTCAGATCATCTACACCCGCAA
TCATGAAGTGAAGAATGAGGTGGATCAGGTACGGTGTCGCCTGGGTAGCA
TGTGCAACCTGGCCCTCTCCACTCCCTTCCTCATGGAGCACACTATGCCT
GTGACACACCCTCCTGATGTGGCGCAGCGCACGGCCGATGCTTGTAACGA
CGGTGTCAAGGCCGTGTGGAACCTCAAAGAACTGCACACCCACCAATTGT
GCCCCCGCTCTTCTGATTACCGCAACATGATTATCCACGCTGCCACCCCC
GTGGACCTGTTGGGCGCTCTCAACCTGTGCCTGCCCCTGATGCAAAAGTT
TCCCAAACAGGTCATGGTGCGCATCTTCTCCACCAACCAGGGTGGGTTCA
TGCTGCCTATCTACGAGACGGCCGCGAAGGCCTACGCCGTGGGGCAGTTT
GAGAAGCCCACCGAGACCCCTCCCGAAGACCTGGACACCCTGAGCCTGGC
CATCGAGGCAGCCATCCAGGACCTGAGGAACAAATCTCAG
Amino acid sequence
(SEQ ID NO: 92)
MESSAKRKMDPDNPDEGPSSKVPRPETPVTKATTFLQTMLRKEVNSQLSL
GDPLFPELAEESLKTFEQVTEDCNENPEKDVLTELGDILAQAVNHAGIDS
SSTGPTLTTHSCSVSSAPLNKPTPTSVAVTNTPLPGASATPELSPRKKPR
KTTRPFKVIIKPPVPPAPIMLPLIKQEDIKPEPDFTIQYRNKIIDTAGCI
VISDSEEEQGEEVETRGATASSPSTGSGTPRVTSPTHPLSQMNHPPLPDP
LGRPDEDSSSSSSSSCSSASDSESESEEMKCSSGGGASVTSSHHGRGGFG
GAASSSLLSCGHQSSGGASTGPRKKKSKRISELDNEKVRNIMKDKNTPFC
TPNVQTRRGRVKIDEVSRMFRHTNRSLEYKNLPFMIPSMHQVLEEAIKVC
KTMQVNNKGIQIIYTRNHEVKNEVDQVRCRLGSMCNLALSTPFLMEHTMP
VTHPPDVAQRTADACNDGVKAVWNLKELHTHQLCPRSSDYRNMIIHAATP
VDLLGALNLCLPLMQKFPKQVMVRIFSTNQGGFMLPIYETAAKAYAVGQF
EKPTETPPEDLDTLSLAIEAAIQDLRNKSQ
Auxiliary sequences:
CMV promoter
(SEQ ID NO: 93)
TGATGCGGTTTTGGCAGTACACCAATGGGCGTGGATAGCGGTTTGACTCA
CGGGGATTTCCAAGTCTCCACCCCATTGACGTCAATGGGAGTTTGTTTTG
GCACCAAAATCAACGGGACTTTCCAAAATGTCGTAATAACCCCGCCCCGT
TGACGCAAATGGGCGGTAGGCGTGTACGGTGGGAGGTCTATATAAGCAGA
Self-cleaving 2A peptide from porcine
teschovirus-1
Nucleotide sequence
(SEQ ID NO: 37)
GGATCTGGCGCCACAAACTTCAGCCTGCTGAAGCAGGCCGGAGATGTGGA
AGAAAATCCTGGCCCC
Amino acid sequence
(SEQ ID NO: 38)
GSGATNFSLLKQAGDVEENPGP
Kozak
GCCACC
bGH polyA
(SEQ ID NO: 46)
CTGTGCCTTCTAGTTGCCAGCCATCTGTTGTTTGCCCCTCCCCCGTGCCT
TCCTTGACCCTGGAAGGTGCCACTCCCACTGTCCTTTCCTAATAAAATGA
GGAAATTGCATCGCATTGTCTGAGTAGGTGTCATTCTATTCTGGGGGGTG
GGGTGGGGCAGGACAGCAAGGGGGAGGATTGGGAAGAGAATAGCAGGCAG
GCTGGGGA
SV40 polyA
(SEQ ID NO: 47)
TAAGATACATTGATGAGTTTGGACAAACCACAACTAGAATGCAGTGAAAA
AAATGCTTTATTTGTGAAATTTGTGATGCTATTGCTTTATTTGTAACCAT
TATAAGCTGCAATAAACAAGTT

### Example 11

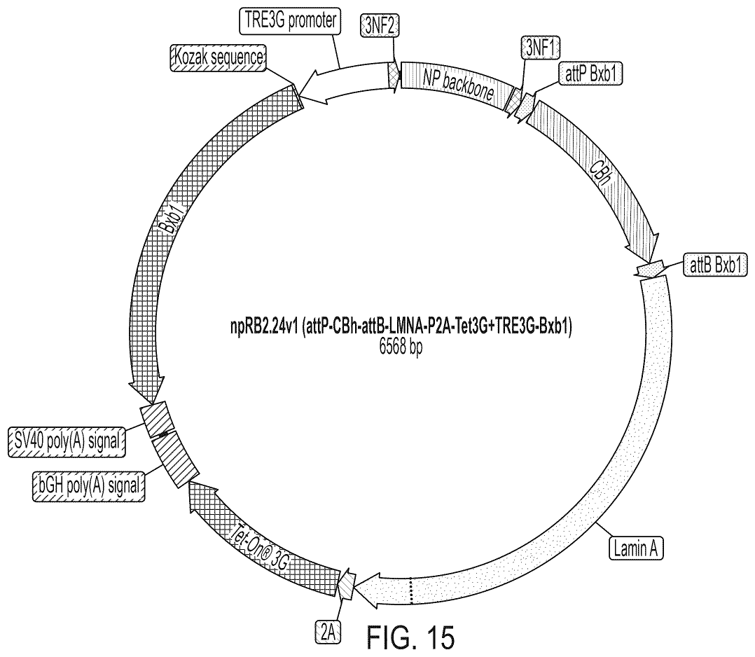

In the present example a single actuator-regulated effector element is intended to deliver biologically active lamin A for the treatment of progeria. Lamin A is expressed under the constitutive CBh promoter, and the gene is flanked on either side by one of two serine-recombinase recognition sequences (attP and attB). The actuator is tetracycline or a tetracycline derivative, which acts upon a tetracycline-inducible promoter and, in conjunction with the tet operator, activates expression of the serine integrase derived from Bxb1 bacteriophage. The integrase excises the genetic sequence of lamin A, turning gene expression off. FIG. 15 shows a plasmid map for the genetic construct described in this example.

Full sequence (except nanoplasmid backbone)
(SEQ ID NO: 94)
CTGGGGACTTTCCAGCCTGGGGACTTTCCAGCTGGGACTTTCCAGGGTCG
TGGTTTGTCTGGTCAACCACCGCGGTCTCAGTGGTGTACGGTACAAACCC
CGACCGTTACATAACTTACGGTAAATGGCCCGCCTGGCTGACCGCCCAAC
GACCCCCGCCCATTGACGTCAATAGTAACGCCAATAGGGACTTTCCATTG
ACGTCAATGGGTGGAGTATTTACGGTAAACTGCCCACTTGGCAGTACATC
AAGTGTATCATATGCCAAGTACGCCCCCTATTGACGTCAATGACGGTAAA
TGGCCCGCCTGGCATTGTGCCCAGTACATGACCTTATGGGACTTTCCTAC
TTGGCAGTACATCTACGTATTAGTCATCGCTATTACCATGGTCGAGGTGA
GCCCCACGTTCTGCTTCACTCTCCCCATCTCCCCCCCCTCCCCACCCCCA
ATTTTGTATTTATTTATTTTTTAATTATTTTGTGCAGCGATGGGGGGGGG
GGGGGGGGGGGGCGCGCGCCAGGCGGGGCGGGGCGGGGCGAGGGGCGGGG
CGGGGCGAGGCGGAGAGGTGCGGCGGCAGCCAATCAGAGCGGCGCGCTCC
GAAAGTTTCCTTTTATGGCGAGGCGGCGGCGGCGGCGGCCCTATAAAAAG
CGAAGCGCGCGGCGGGCGGGAGTCGCTGCGCGCTGCCTTCGCCCCGTGCC
CCGCTCCGCCGCCGCCTCGCGCCGCCCGCCCCGGCTCTGACTGACCGCGT
TACTCCCACAGGTGAGCGGGCGGGACGGCCCTTCTCCTCCGGGCTGTAAT
TAGCTGAGCAAGAGGTAAGGGTTTAAGGGATGGTTGGTTGGTGGGGTATT
AATGTTTAATTACCTGGAGCACCTGCCTGAAATCACTTTTTTTCAGGTTG
GTCGGCCGGCTTGTCGACGACGGCGGTCTCCGTCGTCAGGATCATCCGGG
CGCCACCATGGAGACCCCGTCCCAGCGGCGCGCCACCCGCAGCGGGGCGC
AGGCCAGCTCCACTCCGCTGTCGCCCACCCGCATCACCCGGCTGCAGGAG
AAGGAGGACCTGCAGGAGCTCAATGATCGCTTGGCGGTCTACATCGACCG
TGTGCGCTCGCTGGAAACGGAGAACGCAGGGCTGCGCCTTCGCATCACCG
AGTCTGAAGAGGTGGTCAGCCGCGAGGTGTCCGGCATCAAGGCCGCCTAC
GAGGCCGAGCTCGGGGATGCCCGCAAGACCCTTGACTCAGTAGCCAAGGA
GCGCGCCCGCCTGCAGCTGGAGCTGAGCAAAGTGCGTGAGGAGTTTAAGG
AGCTGAAAGCGCGCAATACCAAGAAGGAGGGTGACCTGATAGCTGCTCAG
GCTCGGCTGAAGGACCTGGAGGCTCTGCTGAACTCCAAGGAGGCCGCACT
GAGCACTGCTCTCAGTGAGAAGCGCACGCTGGAGGGCGAGCTGCATGATC
TGCGGGGCCAGGTGGCCAAGCTTGAGGCAGCCCTAGGTGAGGCCAAGAAG
CAACTTCAGGATGAGATGCTGCGGCGGGTGGATGCTGAGAACAGGCTGCA
GACCATGAAGGAGGAACTGGACTTCCAGAAGAACATCTACAGTGAGGAGC
TGCGTGAGACCAAGCGCCGTCATGAGACCCGACTGGTGGAGATTGACAAT
GGGAAGCAGCGTGAGTTTGAGAGCCGGCTGGCGGATGCGCTGCAGGAACT
GCGGGCCCAGCATGAGGACCAGGTGGAGCAGTATAAGAAGGAGCTGGAGA
AGACTTATTCTGCCAAGCTGGACAATGCCAGGCAGTCTGCTGAGAGGAAC
AGCAACCTGGTGGGGGCTGCCCACGAGGAGCTGCAGCAGTCGCGCATCCG
CATCGACAGCCTCTCTGCCCAGCTCAGCCAGCTCCAGAAGCAGCTGGCAG
CCAAGGAGGCGAAGCTTCGAGACCTGGAGGACTCACTGGCCCGTGAGCGG
GACACCAGCCGGCGGCTGCTGGCGGAAAAGGAGCGGGAGATGGCCGAGAT
GCGGGCAAGGATGCAGCAGCAGCTGGACGAGTACCAGGAGCTTCTGGACA
TCAAGCTGGCCCTGGACATGGAGATCCACGCCTACCGCAAGCTCTTGGAG
GGCGAGGAGGAGAGGCTACGCCTGTCCCCCAGCCCTACCTCGCAGCGCAG
CCGTGGCCGTGCTTCCTCTCACTCATCCCAGACACAGGGTGGGGGCAGCG
TCACCAAAAAGCGCAAACTGGAGTCCACTGAGAGCCGCAGCAGCTTCTCA
CAGCACGCACGCACTAGCGGGCGCGTGGCCGTGGAGGAGGTGGATGAGGA
GGGCAAGTTTGTCCGGCTGCGCAACAAGTCCAATGAGGACCAGTCCATGG
GCAATTGGCAGATCAAGCGCCAGAATGGAGATGATCCCTTGCTGACTTAC
CGGTTCCCACCAAAGTTCACCCTGAAGGCTGGGCAGGTGGTGACGATCTG
GGCTGCAGGAGCTGGGGCCACCCACAGCCCCCCTACCGACCTGGTGTGGA
AGGCACAGAACACCTGGGGCTGCGGGAACAGCCTGCGTACGGCTCTCATC
AACTCCACTGGGGAAGAAGTGGCCATGCGCAAGCTGGTGCGCTCAGTGAC
TGTGGTTGAGGACGACGAGGATGAGGATGGAGATGACCTGCTCCATCACC
ACCACGGCTCCCACTGCAGCAGCTCGGGGGACCCCGCTGAGTACAACCTG
CGCTCGCGCACCGTGCTGTGCGGGACCTGCGGGCAGCCTGCCGACAAGGC
ATCTGCCAGCGGCTCAGGAGCCCAGGTGGGCGGACCCATCTCCTCTGGCT
CTTCTGCCTCCAGTGTCACGGTCACTCGCAGCTACCGCAGTGTGGGGGGC
AGTGGGGGTGGCAGCTTCGGGGACAATCTGGTCACCCGCTCCTACCTCCT
GGGCAACTCCAGCCCCCGAACCCAGAGCCCCCAGAACTGCAGCATCATAC
AAGAGATGGGAATGAGGTGGGAGGTGGAAGAAGGGAGAAGAAAGGTGAGT
TTGAGCTGCCTTCCCGGCTCTGGAGCTACAAACTTCAGCCTGCTGAAGCA
GGCCGGAGATGTTGAAGAGAACCCCGGCCCTATGAGCAGACTGGATAAGT
CGAAGGTCATCAACTCCGCCCTCGAGCTGCTGAACGGCGTGGGAATCGAA
GGCCTGACAACCAGAAAGCTTGCCCAGAAACTGGGCGTGGAACAGCCTAC
CCTGTACTGGCACGTGAAGAACAAGCGGGCCCTGCTGGACGCCCTGCCTA
TCGAGATGCTGGACAGACACCACACGCACAGCTGTCCACTGGAAGGCGAA
AGCTGGCAGGACTTTCTGAGAAACAACGCCAAAAGCTACCGGTGTGCCCT
TCTTTCTCATAGAGATGGCGCCAAGGTGCACCTGGGCACCCGTCCAACCG
AGAAACAGTACGAGACACTGGAAAACCAGCTGGCTTTCCTGTGCCAGCAG
GGCTTCTCTCTGGAAAATGCCCTCTATGCCCTGTCTGCTGTGGGCCACTT
CACCCTGGGCTGCGTGTTGGAGGAGCAAGAGCACCAGGTGGCCAAGGAAG
AACGGGAAACACCCACCACCGACAGCATGCCACCTCTGCTGAAGCAAGCC
ATCGAGCTGTTCGACCGCCAGGGCGCCGAGCCTGCCTTTCTGTTTGGGCT
GGAGCTGATTATCTGCGGCCTGGAAAAGCAACTGAAGTGCGAGAGCGGCG
GACCTACCGATGCTCTGGACGATTTCGATCTGGATATGCTGCCCGCCGAC
GCACTGGACGACTTCGACCTGGACATGCTGCCCGCTGATGCCCTGGACGA
CTTCGACCTGGACATGCTGCCTGGATAACTGTGCCTTCTAGTTGCCAGCC
ATCTGTTGTTTGCCCCTCCCCCGTGCCTTCCTTGACCCTGGAAGGTGCCA
CTCCCACTGTCCTTTCCTAATAAAATGAGGAAATTGCATCGCATTGTCTG
AGTAGGTGTCATTCTATTCTGGGGGGTGGGGTGGGGCAGGACAGCAAGGG
GGAGGATTGGGAAGAGAATAGCAGGCAGGCTGGGGAGGGCCGCCGATCGA
ACTTGTTTATTGCAGCTTATAATGGTTACAAATAAAGCAATAGCATCACA
AATTTCACAAATAAAGCATTTTTTTCACTGCATTCTAGTTGTGGTTTGTC
CAAACTCATCAATGTATCTTACTAGCTCATGCCGGTGTGCAGTCTTTCCA
CCACGCTGCCCAGTCTCAGGTGTTGCTCGTACTCCTGTAGGTCGCCGAAG
TCGATGGTTCTTGTCAGGCCGCCCCGGACATCGAATGTCAACCGCACGTT
CATGGACCGCAGCCAGGTGTTCTTGGCGGCTGTGTCCTGTTCCCGCCACC
AGTCGCCGAACCGCTGTCCGGTTTCTCGCCACTCCCATCCGCTAGGCCGG
GCTTCCAGGCCCTCCAGTTCCTCCTGCCTAGCAGCCAGAGCGGCGATTCT
GGCGTCCAGGGCTTCTCTCTGTGGAGAACCGGCTCTATAGGCGGGGCTGC
CGATCAGGCTGGTCAGATCCACCAGCTCGGCGTTCACCTCGGCCAGTTCC
ACGGCGCTGTCGCTGCCGGCCACCCACACCTTCTCGAGTCTCTCAGCATC
GCCGAGCAGATCCAGCACTTGTTCCTCGCAGAAGGCATCCCACTCGGCCA
TGGCCACGGTGCCATTGCCGCAGTGTTTGGGGAAGCCCATGGATCTGCAC
CGGTATCTAGGGTGCTTTCTGCCTCCGCCGGCGAACTTGTAGGCGGGTTC
TCCGCACACGGCACAGAACAGGACCCTCAGCAGCAGGCTTGGGGTGGACA
CAGCAGGTTTAGCTCTAGAGGTCTTCACCAGCTCGGCCCGCAGGGCCTCC
AGCTGCTCTCTTGTCAGGATAGGTTCGGCCCGCACCAGAGGGGCGCCGTC
ATCATCTCGCACGGTCTTGCCGTTCAGGGTGGCGTATCCCAGCATGGCCT
CGCTGATCATGCTGCGCTTCAGGGCTGTGGCGCTCCACTCTCTGCCCTGA
GGCTCTCTTCCCTGCAGCTGGGCGAAGTAGTCCTTAGGAGACAGCACGCC
CCTTCTGTTCAAGTCGTGAGCCACCAGATGGAGTGGCTCGTGGTTGTCAA
CGACCCGGTGGTACACTTCCAGGATCCGCTCCCGCTGCACAGGATCAGGC
ACCAGTCTCCACTCGCCGTCCACTCTGGTAGGCAGGTAGCCCCAGGGGGG
CAGAGATCCTCTGTATTTGCCGGCTCTGATGTTGAAATGGGCGGCAGATC
TATTTCTTTCCTTGATAGCTTCCAGTTCCATCTGGGCCACGGTGCCCATC
AGGGCGATCACCACGGCGGCAAAAGGGGTGGTGGTGTCGAAGTGTGCTTC
TGTGGCGCTCACGACCAGCTTCTTGTGATCCTCGGCCCAGTGCACCAGCT
GTTGCAGGTGCCGGATGCTTCTGGTCAGTCTGTCCACCCGGTAGGCCACA
ATCACATCGAAAGGCTGCTCCTCAAAAGCCAGCCATCTAGCCAGGTTGGG
TCTCCGCTTTCTGTCAAAGGGGTCCACAGCGCCGGAAACGTCCAGGTCCT
CGGCGACGCCCACCACGTCCCAGCCCCGCTGGGCGCACAGCTGCTGACAG
CTCTCGAGCTGTCTCTCAGGGCTAGTTGTTGCGTCTGTGACTCTGCTCAG
CCGGATCACCACCAGAGCTCTCATGGTGGCTTTACGAGGGTAGGAAGTGG
TACGGAAAGTTGGTATAAGACAAAAGTGTTGTGGAATTGCTCCAGGCGAT
CTGACGGTTCACTAAACGAGCTCTGCTTTTATAGGCGCCCACCGTACACG
CCTAAAGCTTATACGTTCTCTATCACTGATAGGGAGTAAACTGGATATAC
GTTCTCTATCACTGATAGGGAGTAAACTGTAGATACGTTCTCTATCACTG
ATAGGGAGTAAACTGGTCATACGTTCTCTATCACTGATAGGGAGTAAACT
CCTTATACGTTCTCTATCACTGATAGGGAGTAAAGTCTGCATACGTTCTC
TATCACTGATAGGGAGTAAACTCTTCATACGTTCTCTATCACTGATAGGG
AGTAAACTCCTGGGGACTTTCCAGCTGGGGACTTTCCAGCTGGGACTTTC
CAGGAG
Transgene: lamin A
Nucleotide sequence
(SEQ ID NO: 95)
ATGGAGACCCCGTCCCAGCGGCGCGCCACCCGCAGCGGGGCGCAGGCCAG
CTCCACTCCGCTGTCGCCCACCCGCATCACCCGGCTGCAGGAGAAGGAGG
ACCTGCAGGAGCTCAATGATCGCTTGGCGGTCTACATCGACCGTGTGCGC
TCGCTGGAAACGGAGAACGCAGGGCTGCGCCTTCGCATCACCGAGTCTGA
AGAGGTGGTCAGCCGCGAGGTGTCCGGCATCAAGGCCGCCTACGAGGCCG
AGCTCGGGGATGCCCGCAAGACCCTTGACTCAGTAGCCAAGGAGCGCGCC
CGCCTGCAGCTGGAGCTGAGCAAAGTGCGTGAGGAGTTTAAGGAGCTGAA
AGCGCGCAATACCAAGAAGGAGGGTGACCTGATAGCTGCTCAGGCTCGGC
TGAAGGACCTGGAGGCTCTGCTGAACTCCAAGGAGGCCGCACTGAGCACT
GCTCTCAGTGAGAAGCGCACGCTGGAGGGCGAGCTGCATGATCTGCGGGG
CCAGGTGGCCAAGCTTGAGGCAGCCCTAGGTGAGGCCAAGAAGCAACTTC
AGGATGAGATGCTGCGGCGGGTGGATGCTGAGAACAGGCTGCAGACCATG
AAGGAGGAACTGGACTTCCAGAAGAACATCTACAGTGAGGAGCTGCGTGA
GACCAAGCGCCGTCATGAGACCCGACTGGTGGAGATTGACAATGGGAAGC
AGCGTGAGTTTGAGAGCCGGCTGGCGGATGCGCTGCAGGAACTGCGGGCC
CAGCATGAGGACCAGGTGGAGCAGTATAAGAAGGAGCTGGAGAAGACTTA
TTCTGCCAAGCTGGACAATGCCAGGCAGTCTGCTGAGAGGAACAGCAACC
TGGTGGGGGCTGCCCACGAGGAGCTGCAGCAGTCGCGCATCCGCATCGAC
AGCCTCTCTGCCCAGCTCAGCCAGCTCCAGAAGCAGCTGGCAGCCAAGGA
GGCGAAGCTTCGAGACCTGGAGGACTCACTGGCCCGTGAGCGGGACACCA
GCCGGCGGCTGCTGGCGGAAAAGGAGCGGGAGATGGCCGAGATGCGGGCA
AGGATGCAGCAGCAGCTGGACGAGTACCAGGAGCTTCTGGACATCAAGCT
GGCCCTGGACATGGAGATCCACGCCTACCGCAAGCTCTTGGAGGGCGAGG
AGGAGAGGCTACGCCTGTCCCCCAGCCCTACCTCGCAGCGCAGCCGTGGC
CGTGCTTCCTCTCACTCATCCCAGACACAGGGTGGGGGCAGCGTCACCAA
AAAGCGCAAACTGGAGTCCACTGAGAGCCGCAGCAGCTTCTCACAGCACG
CACGCACTAGCGGGCGCGTGGCCGTGGAGGAGGTGGATGAGGAGGGCAAG
TTTGTCCGGCTGCGCAACAAGTCCAATGAGGACCAGTCCATGGGCAATTG
GCAGATCAAGCGCCAGAATGGAGATGATCCCTTGCTGACTTACCGGTTCC
CACCAAAGTTCACCCTGAAGGCTGGGCAGGTGGTGACGATCTGGGCTGCA
GGAGCTGGGGCCACCCACAGCCCCCCTACCGACCTGGTGTGGAAGGCACA
GAACACCTGGGGCTGCGGGAACAGCCTGCGTACGGCTCTCATCAACTCCA
CTGGGGAAGAAGTGGCCATGCGCAAGCTGGTGCGCTCAGTGACTGTGGTT
GAGGACGACGAGGATGAGGATGGAGATGACCTGCTCCATCACCACCACGG
CTCCCACTGCAGCAGCTCGGGGGACCCCGCTGAGTACAACCTGCGCTCGC
GCACCGTGCTGTGCGGGACCTGCGGGCAGCCTGCCGACAAGGCATCTGCC
AGCGGCTCAGGAGCCCAGGTGGGCGGACCCATCTCCTCTGGCTCTTCTGC
CTCCAGTGTCACGGTCACTCGCAGCTACCGCAGTGTGGGGGGCAGTGGGG
GTGGCAGCTTCGGGGACAATCTGGTCACCCGCTCCTACCTCCTGGGCAAC
TCCAGCCCCCGAACCCAGAGCCCCCAGAACTGCAGCATCATACAAGAGAT
GGGAATGAGGTGGGAGGTGGAAGAAGGGAGAAGAAAGGTGAGTTTGAGCT
GCCTTCCC
Amino acid sequence
(SEQ ID NO: 96)
METPSQRRATRSGAQASSTPLSPTRITRLQEKEDLQELNDRLAVYIDRVR
SLETENAGLRLRITESEEVVSREVSGIKAAYEAELGDARKTLDSVAKERA
RLQLELSKVREEFKELKARNTKKEGDLIAAQARLKDLEALLNSKEAALST
ALSEKRTLEGELHDLRGQVAKLEAALGEAKKQLQDEMLRRVDAENRLQTM
KEELDFQKNIYSEELRETKRRHETRLVEIDNGKQREFESRLADALQELRA
QHEDQVEQYKKELEKTYSAKLDNARQSAERNSNLVGAAHEELQQSRIRID
SLSAQLSQLQKQLAAKEAKLRDLEDSLARERDTSRRLLAEKEREMAEMRA
RMQQQLDEYQELLDIKLALDMEIHAYRKLLEGEEERLRLSPSPTSQRSRG
RASSHSSQTQGGGSVTKKRKLESTESRSSFSQHARTSGRVAVEEVDEEGK
FVRLRNKSNEDQSMGNWQIKRQNGDDPLLTYRFPPKFTLKAGQVVTIWAA
GAGATHSPPTDLVWKAQNTWGCGNSLRTALINSTGEEVAMRKLVRSVTVV
EDDEDEDGDDLLHHHHGSHCSSSGDPAEYNLRSRTVLCGTCGQPADKASA
SGSGAQVGGPISSGSSASSVTVTRSYRSVGGSGGGSFGDNLVTRSYLLGN
SSPRTQSPQNCSIIQEMGMRWEVEEGRRKVSLSCLP
Actuator: Tetracycline or derivative
Actuator response element: TetRepressor/
Gen 3 TetOn, third generation rtTA/TRE3G
TRE3G
(SEQ ID NO: 45)
TTTACTCCCTATCAGTGATAGAGAACGTATGAAGAGTTTACTCCCTATCA
GTGATAGAGAACGTATGCAGACTTTACTCCCTATCAGTGATAGAGAACGT
ATAAGGAGTTTACTCCCTATCAGTGATAGAGAACGTATGACCAGTTTACT
CCCTATCAGTGATAGAGAACGTATCTACAGTTTACTCCCTATCAGTGATA
GAGAACGTATATCCAGTTTACTCCCTATCAGTGATAGAGAACGTATAAGC
TTTAGGCGTGTACGGTGGGCGCCTATAAAAGCAGAGCTCGTTTAGTGAAC
CGTCAGATCGCCTGGAGCAATTCCACAACACTTTTGTCTTATACCAACTT
TCCGTACCACTTCCTACCCTCGTAAA
TetOn3G
Nucleotide sequence
(SEQ ID NO: 43)
ATGAGCAGACTGGATAAGTCCAAGGTGATCAACAGCGCCCTGGAACTGCT
CAACGGCGTCGGCATCGAGGGACTCACCACCCGGAAGCTGGCCCAGAAAC
TGGGCGTGGAACAACCTACACTGTACTGGCACGTGAAGAACAAAAGAGCC
CTGCTGGATGCCCTGCCAATCGAGATGCTGGACCGGCACCATACCCACAG
CTGCCCCCTGGAGGGAGAAAGCTGGCAGGACTTCCTGAGAAACAACGCTA
AGAGCTACCGGTGCGCCCTGCTGAGCCACCGGGACGGCGCCAAGGTGCAC
TTGGGGACCAGACCCACGGAAAAGCAATACGAGACACTGGAGAACCAGCT
GGCCTTCCTGTGCCAGCAGGGCTTCAGCCTGGAGAATGCCCTCTACGCCC
TGTCTGCCGTGGGCCACTTTACCCTGGGATGTGTGCTGGAAGAGCAGGAG
CACCAGGTGGCCAAGGAAGAGAGGGAAACCCCTACCACCGACAGCATGCC
TCCTCTGCTGAAGCAAGCTATCGAGCTGTTCGACAGACAGGGCGCCGAGC
CTGCTTTCCTGTTTGGCCTGGAGCTGATCATCTGCGGCTTGGAGAAACAG
CTGAAGTGCGAGAGCGGCGGCCCTACCGACGCCCTGGACGATTTCGACCT
GGATATGCTGCCTGCCGACGCCCTGGACGACTTCGACCTGGATATGCTGC
CTGCCGATGCTCTGGACGACTTCGACCTGGACATGCTTCCAGGCTAA
Amino acid sequence
(SEQ ID NO: 44)
MSRLDKSKVINSALELLNGVGIEGLTTRKLAQKLGVEQPTLYWHVKNKRA
LLDALPIEMLDRHHTHSCPLEGESWQDFLRNNAKSYRCALLSHRDGAKVH
LGTRPTEKQYETLENQLAFLCQQGFSLENALYALSAVGHFTLGCVLEEQE
HQVAKEERETPTTDSMPPLLKQAIELFDRQGAEPAFLFGLELIICGLEKQ
LKCESGGPTDALDDFDLDMLPADALDDFDLDMLPADALDDFDLDMLPG
Effector element: Bxb1 serine integrase,
associated attB and attP sites
attP site
(SEQ ID NO: 97)
GTCGTGGTTTGTCTGGTCAACCACCGCGGTCTCAGTGGTGTACGGTACAA
ACCCCGAC
attB site
(SEQ ID NO: 98)
TCGGCCGGCTTGTCGACGACGGCGGTCTCCGTCGTCAGGATCATCCGGGC
Bxb1 serine integrase
Nucleotide sequence
(SEQ ID NO: 99)
ATGAGAGCTCTGGTGGTGATCCGGCTGAGCAGAGTCACAGACGCAACAAC
TAGCCCTGAGAGACAGCTCGAGAGCTGTCAGCAGCTGTGCGCCCAGCGGG
GCTGGGACGTGGTGGGCGTCGCCGAGGACCTGGACGTTTCCGGCGCTGTG
GACCCCTTTGACAGAAAGCGGAGACCCAACCTGGCTAGATGGCTGGCTTT
TGAGGAGCAGCCTTTCGATGTGATTGTGGCCTACCGGGTGGACAGACTGA
CCAGAAGCATCCGGCACCTGCAACAGCTGGTGCACTGGGCCGAGGATCAC
AAGAAGCTGGTCGTGAGCGCCACAGAAGCACACTTCGACACCACCACCCC
TTTTGCCGCCGTGGTGATCGCCCTGATGGGCACCGTGGCCCAGATGGAAC
TGGAAGCTATCAAGGAAAGAAATAGATCTGCCGCCCATTTCAACATCAGA
GCCGGCAAATACAGAGGATCTCTGCCCCCCTGGGGCTACCTGCCTACCAG
AGTGGACGGCGAGTGGAGACTGGTGCCTGATCCTGTGCAGCGGGAGCGGA
TCCTGGAAGTGTACCACCGGGTCGTTGACAACCACGAGCCACTCCATCTG
GTGGCTCACGACTTGAACAGAAGGGGCGTGCTGTCTCCTAAGGACTACTT
CGCCCAGCTGCAGGGAAGAGAGCCTCAGGGCAGAGAGTGGAGCGCCACAG
CCCTGAAGCGCAGCATGATCAGCGAGGCCATGCTGGGATACGCCACCCTG
AACGGCAAGACCGTGCGAGATGATGACGGCGCCCCTCTGGTGCGGGCCGA
ACCTATCCTGACAAGAGAGCAGCTGGAGGCCCTGCGGGCCGAGCTGGTGA
AGACCTCTAGAGCTAAACCTGCTGTGTCCACCCCAAGCCTGCTGCTGAGG
GTCCTGTTCTGTGCCGTGTGCGGAGAACCCGCCTACAAGTTCGCCGGCGG
AGGCAGAAAGCACCCTAGATACCGGTGCAGATCCATGGGCTTCCCCAAAC
ACTGCGGCAATGGCACCGTGGCCATGGCCGAGTGGGATGCCTTCTGCGAG
GAACAAGTGCTGGATCTGCTCGGCGATGCTGAGAGACTCGAGAAGGTGTG
GGTGGCCGGCAGCGACAGCGCCGTGGAACTGGCCGAGGTGAACGCCGAGC
TGGTGGATCTGACCAGCCTGATCGGCAGCCCCGCCTATAGAGCCGGTTCT
CCACAGAGAGAAGCCCTGGACGCCAGAATCGCCGCTCTGGCTGCTAGGCA
GGAGGAACTGGAGGGCCTGGAAGCCCGGCCTAGCGGATGGGAGTGGCGAG
AAACCGGACAGCGGTTCGGCGACTGGTGGCGGGAACAGGACACAGCCGCC
AAGAACACCTGGCTGCGGTCCATGAACGTGCGGTTGACATTCGATGTCCG
GGGCGGCCTGACAAGAACCATCGACTTCGGCGACCTACAGGAGTACGAGC
AACACCTGAGACTGGGCAGCGTGGTGGAAAGACTGCACACCGGCATGAGC
TAG
Amino acid sequence
(SEQ ID NO: 100)
MRALVVIRLSRVTDATTSPERQLESCQQLCAQRGWDVVGVAEDLDVSGAV
DPFDRKRRPNLARWLAFEEQPFDVIVAYRVDRLTRSIRHLQQLVHWAEDH
KKLVVSATEAHFDTTTPFAAVVIALMGTVAQMELEAIKERNRSAAHFNIR
AGKYRGSLPPWGYLPTRVDGEWRLVPDPVQRERILEVYHRVVDNHEPLHL
VAHDLNRRGVLSPKDYFAQLQGREPQGREWSATALKRSMISEAMLGYATL
NGKTVRDDDGAPLVRAEPILTREQLEALRAELVKTSRAKPAVSTPSLLLR
VLFCAVCGEPAYKFAGGGRKHPRYRCRSMGFPKHCGNGTVAMAEWDAFCE
EQVLDLLGDAERLEKVWVAGSDSAVELAEVNAELVDLTSLIGSPAYRAGS
PQREALDARIAALAARQEELEGLEARPSGWEWRETGQRFGDWWREQDTAA
KNTWLRSMNVRLTFDVRGGLTRTIDFGDLQEYEQHLRLGSVVERLHTGMS
Auxiliary sequences:
CBh promoter
(SEQ ID NO: 32)
CGTTACATAACTTACGGTAAATGGCCCGCCTGGCTGACCGCCCAACGACC
CCCGCCCATTGACGTCAATAGTAACGCCAATAGGGACTTTCCATTGACGT
CAATGGGTGGAGTATTTACGGTAAACTGCCCACTTGGCAGTACATCAAGT
GTATCATATGCCAAGTACGCCCCCTATTGACGTCAATGACGGTAAATGGC
CCGCCTGGCATTGTGCCCAGTACATGACCTTATGGGACTTTCCTACTTGG
CAGTACATCTACGTATTAGTCATCGCTATTACCATGGTCGAGGTGAGCCC
CACGTTCTGCTTCACTCTCCCCATCTCCCCCCCCTCCCCACCCCCAATTT
TGTATTTATTTATTTTTTAATTATTTTGTGCAGCGATGGGGGCGGGGGGG
GGGGGGGGGCGCGCGCCAGGCGGGGCGGGGCGGGGCGAGGGGCGGGGCGG
GGCGAGGCGGAGAGGTGCGGCGGCAGCCAATCAGAGCGGCGCGCTCCGAA
AGTTTCCTTTTATGGCGAGGCGGCGGCGGCGGCGGCCCTATAAAAAGCGA
AGCGCGCGGCGGGCGGGAGTCGCTGCGCGCTGCCTTCGCCCCGTGCCCCG
CTCCGCCGCCGCCTCGCGCCGCCCGCCCCGGCTCTGACTGACCGCGTTAC
TCCCACAGGTGAGCGGGCGGGACGGCCCTTCTCCTCCGGGCTGTAATTAG
CTGAGCAAGAGGTAAGGGTTTAAGGGATGGTTGGTTGGTGGGGTATTAAT
GTTTAATTACCTGGAGCACCTGCCTGAAATCACTTTTTTTCAGGTTGG
Self-cleaving 2A peptide from porcine
teschovirus-1
Nucleotide sequence
(SEQ ID NO: 37)
GGATCTGGCGCCACAAACTTCAGCCTGCTGAAGCAGGCCGGAGATGTGGA
AGAAAATCCTGGCCCC
Amino acid sequence
(SEQ ID NO: 38)
GSGATNFSLLKQAGDVEENPGP
Kozak
GCCACC
bGH polyA
(SEQ ID NO: 46)
CTGTGCCTTCTAGTTGCCAGCCATCTGTTGTTTGCCCCTCCCCCGTGCCT
TCCTTGACCCTGGAAGGTGCCACTCCCACTGTCCTTTCCTAATAAAATGA
GGAAATTGCATCGCATTGTCTGAGTAGGTGTCATTCTATTCTGGGGGGTG
GGGTGGGGCAGGACAGCAAGGGGGAGGATTGGGAAGAGAATAGCAGGCAG
GCTGGGGA
SV40 polyA
(SEQ ID NO: 47)
TAAGATACATTGATGAGTTTGGACAAACCACAACTAGAATGCAGTGAAAA
AAATGCTTTATTTGTGAAATTTGTGATGCTATTGCTTTATTTGTAACCAT
TATAAGCTGCAATAAACAAGTT

### Example 12

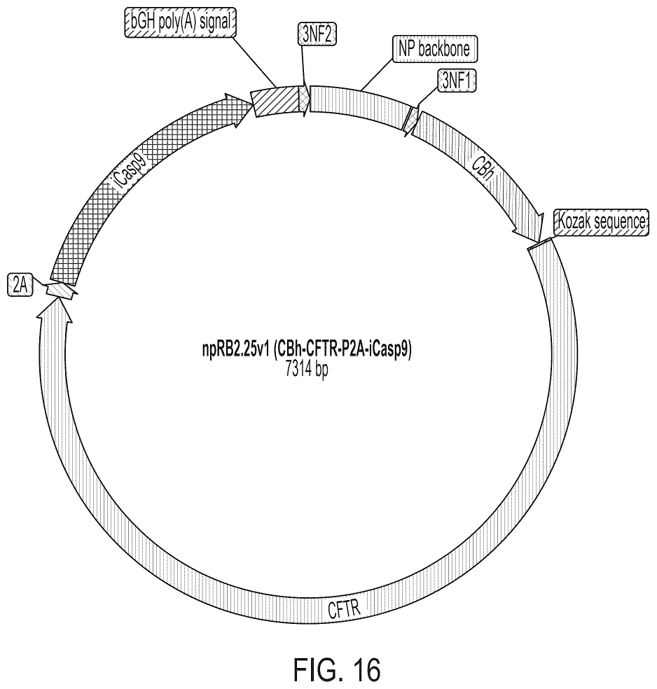

In the present example a single actuator-regulated is intended to deliver a functional copy of cystic fibrosis transmembrane conductance regulator (CFTR) for treatment of cystic fibrosis. A CBh promoter controls expression of CFTR and inducible Caspase 9 (iCasp9), which are connected by a self-cleaving peptide (P2A). The actuator is AP20187, a ligand that causes dimerization of caspase 9 and induction of apoptosis. FIG. 16 shows a plasmid map for the genetic construct described in this example.

Full sequence (except nanoplasmid backbone)
(SEQ ID NO: 101)
CTGGGGACTTTCCAGCCTGGGGACTTTCCAGCTGGGACTTTCCAGGCGTTACATA
ACTTACGGTAAATGGCCCGCCTGGCTGACCGCCCAACGACCCCCGCCCATTGACG
TCAATAGTAACGCCAATAGGGACTTTCCATTGACGTCAATGGGTGGAGTATTTACG
GTAAACTGCCCACTTGGCAGTACATCAAGTGTATCATATGCCAAGTACGCCCCCTA
TTGACGTCAATGACGGTAAATGGCCCGCCTGGCATTGTGCCCAGTACATGACCTTA
TGGGACTTTCCTACTTGGCAGTACATCTACGTATTAGTCATCGCTATTACCATGGTC
GAGGTGAGCCCCACGTTCTGCTTCACTCTCCCCATCTCCCCCCCCTCCCCACCCCC
AATTTTGTATTTATTTATTTTTTAATTATTTTGTGCAGCGATGGGGGCGGGGGGGGG
GGGGGGGGCGCGCCAGGCGGGGCGGGGCGGGGCGAGGGGCGGGGCGGGGCG
AGGCGGAGAGGTGCGGCGGCAGCCAATCAGAGCGGCGCGCTCCGAAAGTTTCCT
TTTATGGCGAGGCGGCGGCGGCGGCGGCCCTATAAAAAGCGAAGCGCGCGGCGG
GCGGGAGTCGCTGCGCGCTGCCTTCGCCCCGTGCCCCGCTCCGCCGCCGCCTCGC
GCCGCCCGCCCCGGCTCTGACTGACCGCGTTACTCCCACAGGTGAGCGGGCGGG
ACGGCCCTTCTCCTCCGGGCTGTAATTAGCTGAGCAAGAGGTAAGGGTTTAAGGG
ATGGTTGGTTGGTGGGGTATTAATGTTTAATTACCTGGAGCACCTGCCTGAAATCA
CTTTTTTTCAGGTTGGGCCACCATGCAGAGGTCGCCTCTGGAAAAGGCCAGCGTT
GTCTCCAAACTTTTTTTCAGCTGGACCAGACCAATTTTGAGGAAAGGATACAGAC
AGCGCCTGGAATTGTCAGACATATACCAAATCCCTTCTGTTGATTCTGCTGACAAT
CTATCTGAAAAATTGGAAAGAGAATGGGATAGAGAGCTGGCTTCAAAGAAAAATC
CTAAACTCATTAATGCCCTTCGGCGATGTTTTTTCTGGAGATTTATGTTCTATGGAAT
CTTTTTATATTTAGGGGAAGTCACCAAAGCAGTACAGCCTCTCTTACTGGGAAGAA
TCATAGCTTCCTATGACCCGGATAACAAGGAGGAACGCTCTATCGCGATTTATCTAG
GCATAGGCTTATGCCTTCTCTTTATTGTGAGGACACTGCTCCTACACCCAGCCATTT
TTGGCCTTCATCACATTGGAATGCAGATGAGAATAGCTATGTTTAGTTTGATTTATA
AGAAGACTTTAAAGCTGTCAAGCCGTGTTCTAGATAAAATAAGTATTGGACAACTT
GTTAGTCTCCTTTCCAACAACCTGAACAAATTTGATGAAGGACTTGCATTGGCACA
TTTCGTGTGGATCGCTCCTTTGCAAGTGGCACTCCTCATGGGGCTAATCTGGGAGT
TGTTACAGGCGTCTGCCTTCTGTGGACTTGGTTTCCTGATAGTCCTTGCCCTTTTTC
AGGCTGGGCTAGGGAGAATGATGATGAAGTACAGAGATCAGAGAGCTGGGAAGA
TCAGTGAAAGACTTGTGATTACCTCAGAAATGATTGAAAATATCCAATCTGTTAAG
GCATACTGCTGGGAAGAAGCAATGGAAAAAATGATTGAAAACTTAAGACAAACA
GAACTGAAACTGACTCGGAAGGCAGCCTATGTGAGATACTTCAATAGCTCAGCCT
TCTTCTTCTCAGGGTTCTTTGTGGTGTTTTTATCTGTGCTTCCCTATGCACTAATCAA
AGGAATCATCCTCCGGAAAATATTCACCACCATCTCATTCTGCATTGTTCTGCGCAT
GGCGGTCACTCGGCAATTTCCCTGGGCTGTACAAACATGGTATGACTCTCTTGGAG
CAATAAACAAAATACAGGATTTCTTACAAAAGCAAGAATATAAGACATTGGAATAT
AACTTAACGACTACAGAAGTAGTGATGGAGAATGTAACAGCCTTCTGGGAGGAGG
GATTTGGGGAATTATTTGAGAAAGCAAAACAAAACAATAACAATAGAAAAACTTC
TAATGGTGATGACAGCCTCTTCTTCAGTAATTTCTCACTTCTTGGTACTCCTGTCCT
GAAAGATATTAATTTCAAGATAGAAAGAGGACAGTTGTTGGCGGTTGCTGGATCC
ACTGGAGCAGGCAAGACTTCACTTCTAATGGTGATTATGGGAGAACTGGAGCCTT
CAGAGGGTAAAATTAAGCACAGTGGAAGAATTTCATTCTGTTCTCAGTTTTCCTGG
ATTATGCCTGGCACCATTAAAGAAAATATCATCTTTGGTGTTTCCTATGATGAATATA
GATACAGAAGCGTCATCAAAGCATGCCAACTAGAAGAGGACATCTCCAAGTTTGC
AGAGAAAGACAATATAGTTCTTGGAGAAGGTGGAATCACACTGAGTGGAGGTCA
ACGAGCAAGAATTTCTTTAGCAAGAGCAGTATACAAAGATGCTGATTTGTATTTATT
AGACTCTCCTTTTGGATACCTAGATGTTTTAACAGAAAAAGAAATATTTGAAAGCT
GTGTCTGTAAACTGATGGCTAACAAAACTAGGATTTTGGTCACTTCTAAAATGGAA
CATTTAAAGAAAGCTGACAAAATATTAATTTTGCATGAAGGTAGCAGCTATTTTTAT
GGGACATTTTCAGAACTCCAAAATCTACAGCCAGACTTTAGCTCAAAACTCATGG
GATGTGATTCTTTCGACCAATTTAGTGCAGAAAGAAGAAATTCAATCCTAACTGAG
ACCTTACACCGTTTCTCATTAGAAGGAGATGCTCCTGTCTCCTGGACAGAAACAA
AAAAACAATCTTTTAAACAGACTGGAGAGTTTGGGGAAAAAAGGAAGAATTCTAT
TCTCAATCCAATCAACTCTATACGAAAATTTTCCATTGTGCAAAAGACTCCCTTACA
AATGAATGGCATCGAAGAGGATTCTGATGAGCCTTTAGAGAGAAGGCTGTCCTTA
GTACCAGATTCTGAGCAGGGAGAGGCGATACTGCCTCGCATCAGCGTGATCAGCA
CTGGCCCCACGCTTCAGGCACGAAGGAGGCAGTCTGTCCTGAACCTGATGACAC
ACTCAGTTAACCAAGGTCAGAACATTCACCGAAAGACAACAGCATCCACACGAA
AAGTGTCACTGGCCCCTCAGGCAAACTTGACTGAACTGGATATATATTCAAGAAG
GTTATCTCAAGAAACTGGCTTGGAAATAAGTGAAGAAATTAACGAAGAAGACTTA
AAGGAGTGCTTTTTTGATGATATGGAGAGCATACCAGCAGTGACTACATGGAACAC
ATACCTTCGATATATTACTGTCCACAAGAGCTTAATTTTTGTGCTAATTTGGTGCTTA
GTAATTTTTCTGGCAGAGGTGGCTGCTTCTTTGGTTGTGCTGTGGCTCCTTGGAAA
CACTCCTCTTCAAGACAAAGGGAATAGTACTCATAGTAGAAATAACAGCTATGCAG
TGATTATCACCAGCACCAGTTCGTATTATGTGTTTTACATTTACGTGGGAGTAGCCG
ACACTTTGCTTGCTATGGGATTCTTCAGAGGTCTACCACTGGTGCATACTCTAATCA
CAGTGTCGAAAATTTTACACCACAAAATGTTACATTCTGTTCTTCAAGCACCTATG
TCAACCCTCAACACGTTGAAAGCAGGTGGGATTCTTAATAGATTCTCCAAAGATAT
AGCAATTTTGGATGACCTTCTGCCTCTTACCATATTTGACTTCATCCAGTTGTTATTA
ATTGTGATTGGAGCTATAGCAGTTGTCGCAGTTTTACAACCCTACATCTTTGTTGCA
ACAGTGCCAGTGATAGTGGCTTTTATTATGTTGAGAGCATATTTCCTCCAAACCTCA
CAGCAACTCAAACAACTGGAATCTGAAGGCAGGAGTCCAATTTTCACTCATCTTG
TTACAAGCTTAAAAGGACTATGGACACTTCGTGCCTTCGGACGGCAGCCTTACTTT
GAAACTCTGTTCCACAAAGCTCTGAATTTACATACTGCCAACTGGTTCTTGTACCT
GTCAACACTGCGCTGGTTCCAAATGAGAATAGAAATGATTTTTGTCATCTTCTTCAT
TGCTGTTACCTTCATTTCCATTTTAACAACAGGAGAAGGAGAAGGAAGAGTTGGT
ATTATCCTGACTTTAGCCATGAATATCATGAGTACATTGCAGTGGGCTGTAAACTCC
AGCATAGATGTGGATAGCTTGATGCGATCTGTGAGCCGAGTCTTTAAGTTCATTGA
CATGCCAACAGAAGGTAAACCTACCAAGTCAACCAAACCATACAAGAATGGCCAA
CTCTCGAAAGTTATGATTATTGAGAATTCACACGTGAAGAAAGATGACATCTGGCC
CTCAGGGGGCCAAATGACTGTCAAAGATCTCACAGCAAAATACACAGAAGGTGG
AAATGCCATATTAGAGAACATTTCCTTCTCAATAAGTCCTGGCCAGAGGGTGGGCC
TCTTGGGAAGAACTGGATCAGGGAAGAGTACTTTGTTATCAGCTTTTTTGAGACTA
CTGAACACTGAAGGAGAAATCCAGATCGATGGTGTGTCTTGGGATTCAATAACTTT
GCAACAGTGGAGGAAAGCCTTTGGAGTGATACCACAGAAAGTATTTATTTTTTCTG
GAACATTTAGAAAAAACTTGGATCCCTATGAACAGTGGAGTGATCAAGAAATATG
GAAAGTTGCAGATGAGGTTGGGCTCAGATCTGTGATAGAACAGTTTCCTGGGAAG
CTTGACTTTGTCCTTGTGGATGGGGGCTGTGTCCTAAGCCATGGCCACAAGCAGTT
GATGTGCTTGGCTAGATCTGTTCTCAGTAAGGCGAAGATCTTGCTGCTTGATGAAC
CCAGTGCTCATTTGGATCCAGTAACATACCAAATAATTAGAAGAACTCTAAAACAA
GCATTTGCTGATTGCACAGTAATTCTCTGTGAACACAGGATAGAAGCAATGCTGGA
ATGCCAACAATTTTTGGTCATAGAAGAGAACAAAGTGCGGCAGTACGATTCCATC
CAGAAACTGCTGAACGAGAGGAGCCTCTTCCGGCAAGCCATCAGCCCCTCCGAC
AGGGTGAAGCTCTTTCCCCACCGGAACTCAAGCAAGTGCAAGTCTAAGCCCCAG
ATTGCTGCTCTGAAAGAGGAGACAGAAGAAGAGGTGCAAGATACAAGGCTTGGA
TCTGGCGCTACTAACTTCAGTCTGCTGAAGCAGGCCGGGGACGTGGAAGAGAAC
CCCGGTCCTATGCTGGAAGGCGTGCAGGTGGAAACCATCAGCCCCGGCGACGGTA
GAACCTTTCCCAAGCGCGGCCAGACCTGCGTGGTCCACTACACCGGCATGCTGGA
AGATGGCAAGAAGGTGGACAGCAGCAGAGATAGAAACAAGCCCTTCAAGTTCAT
GCTGGGCAAACAGGAGGTGATTAGGGGCTGGGAGGAAGGCGTGGCCCAGATGAG
CGTTGGACAGCGGGCCAAACTGACAATCTCTCCTGACTACGCCTACGGAGCTACC
GGCCACCCCGGGATCATCCCTCCTCACGCCACCCTGGTCTTTGATGTGGAGCTGCT
GAAACTGGAAAGCGGCGGCGGAAGCGGCGTGGACGGCTTCGGCGATGTGGGCGC
CCTGGAATCTCTGAGAGGAAATGCCGACCTGGCCTACATCCTGAGCATGGAACCG
TGCGGCCACTGTCTGATCATCAACAACGTGAACTTCTGCAGAGAGAGCGGCCTGA
GAACAAGAACAGGCTCCAACATCGATTGCGAGAAACTGCGGAGACGGTTCAGCA
GCCTCCATTTCATGGTCGAAGTGAAGGGCGACCTGACCGCCAAGAAAATGGTGCT
GGCCCTGCTGGAACTGGCCAGACAGGACCATGGCGCTCTGGACTGCTGCGTGGT
GGTGATCCTGTCCCACGGCTGTCAGGCTAGCCACCTGCAATTTCCTGGAGCCGTGT
ACGGCACCGACGGATGCCCCGTGTCTGTTGAGAAGATCGTCAACATCTTTAACGG
CACCAGCTGCCCTTCTTTGGGCGGGAAGCCTAAGTTGTTCTTCATCCAGGCCTGTG
GCGGAGAACAGAAGGACCACGGCTTTGAGGTGGCTTCCACAAGCCCTGAGGATG
AGAGCCCTGGCTCTAATCCAGAGCCTGACGCCACACCTTTCCAAGAGGGCCTGCG
GACCTTCGATCAGCTGGACGCCATCAGCTCTCTGCCTACCCCTAGCGACATCTTCG
TGTCCTACAGCACCTTCCCTGGATTTGTGTCCTGGCGGGACCCCAAGAGCGGAAG
CTGGTACGTGGAGACACTGGACGATATTTTTGAGCAGTGGGCCCACAGCGAGGAC
CTGCAGAGCCTGCTGCTCAGAGTGGCCAACGCCGTAAGCGTGAAAGGCATCTACA
AGCAGATGCCTGGCTGCTTCAACTTCCTGAGAAAGAAGCTGTTCTTCAAGACCAG
CGTGGATTATCCATACGACGTGCCCGACTACGCCCTGGACTAACTGTGCCTTCTAG
TTGCCAGCCATCTGTTGTTTGCCCCTCCCCCGTGCCTTCCTTGACCCTGGAAGGTG
CCACTCCCACTGTCCTTTCCTAATAAAATGAGGAAATTGCATCGCATTGTCTGAGT
AGGTGTCATTCTATTCTGGGGGGTGGGGTGGGGCAGGACAGCAAGGGGGAGGAT
TGGGAAGAGAATAGCAGGCAGGCTGGGGACTGGGGACTTTCCAGCTGGGGACTT
TCCAGCTGGGACTTTCCAGGAG
Transgene: cystic fibrosis conductance regulator
Nucleotide sequence
(SEQ ID NO: 102)
ATGCAGAGGTCGCCTCTGGAAAAGGCCAGCGTTGTCTCCAAACTTTTTTTCAGCT
GGACCAGACCAATTTTGAGGAAAGGATACAGACAGCGCCTGGAATTGTCAGACAT
ATACCAAATCCCTTCTGTTGATTCTGCTGACAATCTATCTGAAAAATTGGAAAGAG
AATGGGATAGAGAGCTGGCTTCAAAGAAAAATCCTAAACTCATTAATGCCCTTCGG
CGATGTTTTTTCTGGAGATTTATGTTCTATGGAATCTTTTTATATTTAGGGGAAGTCA
CCAAAGCAGTACAGCCTCTCTTACTGGGAAGAATCATAGCTTCCTATGACCCGGAT
AACAAGGAGGAACGCTCTATCGCGATTTATCTAGGCATAGGCTTATGCCTTCTCTTT
ATTGTGAGGACACTGCTCCTACACCCAGCCATTTTTGGCCTTCATCACATTGGAAT
GCAGATGAGAATAGCTATGTTTAGTTTGATTTATAAGAAGACTTTAAAGCTGTCAA
GCCGTGTTCTAGATAAAATAAGTATTGGACAACTTGTTAGTCTCCTTTCCAACAAC
CTGAACAAATTTGATGAAGGACTTGCATTGGCACATTTCGTGTGGATCGCTCCTTT
GCAAGTGGCACTCCTCATGGGGCTAATCTGGGAGTTGTTACAGGCGTCTGCCTTCT
GTGGACTTGGTTTCCTGATAGTCCTTGCCCTTTTTCAGGCTGGGCTAGGGAGAATG
ATGATGAAGTACAGAGATCAGAGAGCTGGGAAGATCAGTGAAAGACTTGTGATTA
CCTCAGAAATGATTGAAAATATCCAATCTGTTAAGGCATACTGCTGGGAAGAAGCA
ATGGAAAAAATGATTGAAAACTTAAGACAAACAGAACTGAAACTGACTCGGAAG
GCAGCCTATGTGAGATACTTCAATAGCTCAGCCTTCTTCTTCTCAGGGTTCTTTGTG
GTGTTTTTATCTGTGCTTCCCTATGCACTAATCAAAGGAATCATCCTCCGGAAAATA
TTCACCACCATCTCATTCTGCATTGTTCTGCGCATGGCGGTCACTCGGCAATTTCCC
TGGGCTGTACAAACATGGTATGACTCTCTTGGAGCAATAAACAAAATACAGGATTT
CTTACAAAAGCAAGAATATAAGACATTGGAATATAACTTAACGACTACAGAAGTAG
TGATGGAGAATGTAACAGCCTTCTGGGAGGAGGGATTTGGGGAATTATTTGAGAA
AGCAAAACAAAACAATAACAATAGAAAAACTTCTAATGGTGATGACAGCCTCTTC
TTCAGTAATTTCTCACTTCTTGGTACTCCTGTCCTGAAAGATATTAATTTCAAGATA
GAAAGAGGACAGTTGTTGGCGGTTGCTGGATCCACTGGAGCAGGCAAGACTTCA
CTTCTAATGGTGATTATGGGAGAACTGGAGCCTTCAGAGGGTAAAATTAAGCACA
GTGGAAGAATTTCATTCTGTTCTCAGTTTTCCTGGATTATGCCTGGCACCATTAAAG
AAAATATCATCTTTGGTGTTTCCTATGATGAATATAGATACAGAAGCGTCATCAAAG
CATGCCAACTAGAAGAGGACATCTCCAAGTTTGCAGAGAAAGACAATATAGTTCT
TGGAGAAGGTGGAATCACACTGAGTGGAGGTCAACGAGCAAGAATTTCTTTAGC
AAGAGCAGTATACAAAGATGCTGATTTGTATTTATTAGACTCTCCTTTTGGATACCT
AGATGTTTTAACAGAAAAAGAAATATTTGAAAGCTGTGTCTGTAAACTGATGGCTA
ACAAAACTAGGATTTTGGTCACTTCTAAAATGGAACATTTAAAGAAAGCTGACAA
AATATTAATTTTGCATGAAGGTAGCAGCTATTTTTATGGGACATTTTCAGAACTCCA
AAATCTACAGCCAGACTTTAGCTCAAAACTCATGGGATGTGATTCTTTCGACCAAT
TTAGTGCAGAAAGAAGAAATTCAATCCTAACTGAGACCTTACACCGTTTCTCATTA
GAAGGAGATGCTCCTGTCTCCTGGACAGAAACAAAAAAACAATCTTTTAAACAGA
CTGGAGAGTTTGGGGAAAAAAGGAAGAATTCTATTCTCAATCCAATCAACTCTATA
CGAAAATTTTCCATTGTGCAAAAGACTCCCTTACAAATGAATGGCATCGAAGAGG
ATTCTGATGAGCCTTTAGAGAGAAGGCTGTCCTTAGTACCAGATTCTGAGCAGGG
AGAGGCGATACTGCCTCGCATCAGCGTGATCAGCACTGGCCCCACGCTTCAGGCA
CGAAGGAGGCAGTCTGTCCTGAACCTGATGACACACTCAGTTAACCAAGGTCAG
AACATTCACCGAAAGACAACAGCATCCACACGAAAAGTGTCACTGGCCCCTCAG
GCAAACTTGACTGAACTGGATATATATTCAAGAAGGTTATCTCAAGAAACTGGCTT
GGAAATAAGTGAAGAAATTAACGAAGAAGACTTAAAGGAGTGCTTTTTTGATGAT
ATGGAGAGCATACCAGCAGTGACTACATGGAACACATACCTTCGATATATTACTGT
CCACAAGAGCTTAATTTTTGTGCTAATTTGGTGCTTAGTAATTTTTCTGGCAGAGGT
GGCTGCTTCTTTGGTTGTGCTGTGGCTCCTTGGAAACACTCCTCTTCAAGACAAA
GGGAATAGTACTCATAGTAGAAATAACAGCTATGCAGTGATTATCACCAGCACCAG
TTCGTATTATGTGTTTTACATTTACGTGGGAGTAGCCGACACTTTGCTTGCTATGGG
ATTCTTCAGAGGTCTACCACTGGTGCATACTCTAATCACAGTGTCGAAAATTTTAC
ACCACAAAATGTTACATTCTGTTCTTCAAGCACCTATGTCAACCCTCAACACGTTG
AAAGCAGGTGGGATTCTTAATAGATTCTCCAAAGATATAGCAATTTTGGATGACCT
TCTGCCTCTTACCATATTTGACTTCATCCAGTTGTTATTAATTGTGATTGGAGCTATA
GCAGTTGTCGCAGTTTTACAACCCTACATCTTTGTTGCAACAGTGCCAGTGATAGT
GGCTTTTATTATGTTGAGAGCATATTTCCTCCAAACCTCACAGCAACTCAAACAAC
TGGAATCTGAAGGCAGGAGTCCAATTTTCACTCATCTTGTTACAAGCTTAAAAGG
ACTATGGACACTTCGTGCCTTCGGACGGCAGCCTTACTTTGAAACTCTGTTCCACA
AAGCTCTGAATTTACATACTGCCAACTGGTTCTTGTACCTGTCAACACTGCGCTGG
TTCCAAATGAGAATAGAAATGATTTTTGTCATCTTCTTCATTGCTGTTACCTTCATTT
CCATTTTAACAACAGGAGAAGGAGAAGGAAGAGTTGGTATTATCCTGACTTTAGC
CATGAATATCATGAGTACATTGCAGTGGGCTGTAAACTCCAGCATAGATGTGGATA
GCTTGATGCGATCTGTGAGCCGAGTCTTTAAGTTCATTGACATGCCAACAGAAGGT
AAACCTACCAAGTCAACCAAACCATACAAGAATGGCCAACTCTCGAAAGTTATGA
TTATTGAGAATTCACACGTGAAGAAAGATGACATCTGGCCCTCAGGGGGCCAAAT
GACTGTCAAAGATCTCACAGCAAAATACACAGAAGGTGGAAATGCCATATTAGAG
AACATTTCCTTCTCAATAAGTCCTGGCCAGAGGGTGGGCCTCTTGGGAAGAACTG
GATCAGGGAAGAGTACTTTGTTATCAGCTTTTTTGAGACTACTGAACACTGAAGG
AGAAATCCAGATCGATGGTGTGTCTTGGGATTCAATAACTTTGCAACAGTGGAGG
AAAGCCTTTGGAGTGATACCACAGAAAGTATTTATTTTTTCTGGAACATTTAGAAA
AAACTTGGATCCCTATGAACAGTGGAGTGATCAAGAAATATGGAAAGTTGCAGAT
GAGGTTGGGCTCAGATCTGTGATAGAACAGTTTCCTGGGAAGCTTGACTTTGTCCT
TGTGGATGGGGGCTGTGTCCTAAGCCATGGCCACAAGCAGTTGATGTGCTTGGCT
AGATCTGTTCTCAGTAAGGCGAAGATCTTGCTGCTTGATGAACCCAGTGCTCATTT
GGATCCAGTAACATACCAAATAATTAGAAGAACTCTAAAACAAGCATTTGCTGATT
GCACAGTAATTCTCTGTGAACACAGGATAGAAGCAATGCTGGAATGCCAACAATT
TTTGGTCATAGAAGAGAACAAAGTGCGGCAGTACGATTCCATCCAGAAACTGCTG
AACGAGAGGAGCCTCTTCCGGCAAGCCATCAGCCCCTCCGACAGGGTGAAGCTC
TTTCCCCACCGGAACTCAAGCAAGTGCAAGTCTAAGCCCCAGATTGCTGCTCTGA
AAGAGGAGACAGAAGAAGAGGTGCAAGATACAAGGCTT
Amino acid sequence
(SEQ ID NO: 103)
MQRSPLEKASVVSKLFFSWTRPILRKGYRQRLELSDIYQIPSVDSADNLSEKLEREWD
RELASKKNPKLINALRRCFFWRFMFYGIFLYLGEVTKAVQPLLLGRIIASYDPDNKEE
RSIAIYLGIGLCLLFIVRTLLLHPAIFGLHHIGMQMRIAMFSLIYKKTLKLSSRVLDKISI
GQLVSLLSNNLNKFDEGLALAHFVWIAPLQVALLMGLIWELLQASAFCGLGFLIVLA
LFQAGLGRMMMKYRDQRAGKISERLVITSEMIENIQSVKAYCWEEAMEKMIENLRQ
TELKLTRKAAYVRYFNSSAFFFSGFFVVFLSVLPYALIKGIILRKIFTTISFCIVLRMAVT
RQFPWAVQTWYDSLGAINKIQDFLQKQEYKTLEYNLTTTEVVMENVTAFWEEGFGE
LFEKAKQNNNNRKTSNGDDSLFFSNFSLLGTPVLKDINFKIERGQLLAVAGSTGAGKT
SLLMVIMGELEPSEGKIKHSGRISFCSQFSWIMPGTIKENIIFGVSYDEYRYRSVIKACQ
LEEDISKFAEKDNIVLGEGGITLSGGQRARISLARAVYKDADLYLLDSPFGYLDVLTEK
EIFESCVCKLMANKTRILVTSKMEHLKKADKILILHEGSSYFYGTFSELQNLQPDFSSK
LMGCDSFDQFSAERRNSILTETLHRFSLEGDAPVSWTETKKQSFKQTGEFGEKRKNSI
LNPINSIRKFSIVQKTPLQMNGIEEDSDEPLERRLSLVPDSEQGEAILPRISVISTGPTLQ
ARRRQSVLNLMTHSVNQGQNIHRKTTASTRKVSLAPQANLTELDIYSRRLSQETGLEI
SEEINEEDLKECFFDDMESIPAVTTWNTYLRYITVHKSLIFVLIWCLVIFLAEVAASLVV
LWLLGNTPLQDKGNSTHSRNNSYAVIITSTSSYYVFYIYVGVADTLLAMGFFRGLPLV
HTLITVSKILHHKMLHSVLQAPMSTLNTLKAGGILNRFSKDIAILDDLLPLTIFDFIQLL
LIVIGAIAVVAVLQPYIFVATVPVIVAFIMLRAYFLQTSQQLKQLESEGRSPIFTHLVTSL
KGLWTLRAFGRQPYFETLFHKALNLHTANWFLYLSTLRWFQMRIEMIFVIFFIAVTFIS
ILTTGEGEGRVGIILTLAMNIMSTLQWAVNSSIDVDSLMRSVSRVFKFIDMPTEGKPTK
STKPYKNGQLSKVMIIENSHVKKDDIWPSGGQMTVKDLTAKYTEGGNAILENISFSIS
PGQRVGLLGRTGSGKSTLLSAFLRLLNTEGEIQIDGVSWDSITLQQWRKAFGVIPQKV
FIFSGTFRKNLDPYEQWSDQEIWKVADEVGLRSVIEQFPGKLDFVLVDGGCVLSHGH
KQLMCLARSVLSKAKILLLDEPSAHLDPVTYQIIRRTLKQAFADCTVILCEHRIEAMLE
CQQFLVIEENKVRQYDSIQKLLNERSLFRQAISPSDRVKLFPHRNSSKCKSKPQIAALK
EETEEEVQDTRL
Actuator: AP20187 or other dimerizer capable of dimerizing iCasp9
Effector element: iCasp9
Nucleotide sequence
(SEQ ID NO: 104)
ATGCTCGAGGGAGTGCAGGTGGAGACTATCTCCCCAGGAGACGGGCGCACCTTCC
CCAAGCGCGGCCAGACCTGCGTGGTGCACTACACCGGGATGCTTGAAGATGGAA
AGAAAGTTGATTCCTCCCGGGACAGAAACAAGCCCTTTAAGTTTATGCTAGGCAA
GCAGGAGGTGATCCGAGGCTGGGAAGAAGGGGTTGCCCAGATGAGTGTGGGTCA
GAGAGCCAAACTGACTATATCTCCAGATTATGCCTATGGTGCCACTGGGCACCCAG
GCATCATCCCACCACATGCCACTCTCGTCTTCGATGTGGAGCTTCTAAAACTGGAA
TCTGGCGGTGGATCCGGAGTCGACGGATTTGGTGATGTCGGTGCTCTTGAGAGTT
TGAGGGGAAATGCAGATTTGGCTTACATCCTGAGCATGGAGCCCTGTGGCCACTG
CCTCATTATCAACAATGTGAACTTCTGCCGTGAGTCCGGGCTCCGCACCCGCACTG
GCTCCAACATCGACTGTGAGAAGTTGCGGCGTCGCTTCTCCTCGCTGCATTTCATG
GTGGAGGTGAAGGGCGACCTGACTGCCAAGAAAATGGTGCTGGCTTTGCTGGAG
CTGGCGCGGCAGGACCACGGTGCTCTGGACTGCTGCGTGGTGGTCATTCTCTCTC
ACGGCTGTCAGGCCAGCCACCTGCAGTTCCCAGGGGCTGTCTACGGCACAGATGG
ATGCCCTGTGTCGGTCGAGAAGATTGTGAACATCTTCAATGGGACCAGCTGCCCC
AGCCTGGGAGGGAAGCCCAAGCTCTTTTTCATCCAGGCCTGTGGTGGGGAGCAG
AAAGACCATGGGTTTGAGGTGGCCTCCACTTCCCCTGAAGACGAGTCCCCTGGCA
GTAACCCCGAGCCAGATGCCACCCCGTTCCAGGAAGGTTTGAGGACCTTCGACCA
GCTGGACGCCATATCTAGTTTGCCCACACCCAGTGACATCTTTGTGTCCTACTCTAC
TTTCCCAGGTTTTGTTTCCTGGAGGGACCCCAAGAGTGGCTCCTGGTACGTTGAG
ACCCTGGACGACATCTTTGAGCAGTGGGCTCACTCTGAAGACCTGCAGTCCCTCC
TGCTTAGGGTCGCTAATGCTGTTTCGGTGAAAGGGATTTATAAACAGATGCCTGGT
TGCTTTAATTTCCTCCGGAAAAAACTTTTCTTTAAAACATCAGTCGACTATCCGTAC
GACGTACCAGACTACGCACTCGACTAA
Amino acid sequence
(SEQ ID NO: 103)
MQRSPLEKASVVSKLFFSWTRPILRKGYRQRLELSDIYQIPSVDSADNLSEKLEREWD
RELASKKNPKLINALRRCFFWRFMFYGIFLYLGEVTKAVQPLLLGRIIASYDPDNKEE
RSIAIYLGIGLCLLFIVRTLLLHPAIFGLHHIGMQMRIAMFSLIYKKTLKLSSRVLDKISI
GQLVSLLSNNLNKFDEGLALAHFVWIAPLQVALLMGLIWELLQASAFCGLGFLIVLA
LFQAGLGRMMMKYRDQRAGKISERLVITSEMIENIQSVKAYCWEEAMEKMIENLRQ
TELKLTRKAAYVRYFNSSAFFFSGFFVVFLSVLPYALIKGIILRKIFTTISFCIVLRMAVT
RQFPWAVQTWYDSLGAINKIQDFLQKQEYKTLEYNLTTTEVVMENVTAFWEEGFGE
LFEKAKQNNNNRKTSNGDDSLFFSNFSLLGTPVLKDINFKIERGQLLAVAGSTGAGKT
SLLMVIMGELEPSEGKIKHSGRISFCSQFSWIMPGTIKENIIFGVSYDEYRYRSVIKACQ
LEEDISKFAEKDNIVLGEGGITLSGGQRARISLARAVYKDADLYLLDSPFGYLDVLTEK
EIFESCVCKLMANKTRILVTSKMEHLKKADKILILHEGSSYFYGTFSELQNLQPDFSSK
LMGCDSFDQFSAERRNSILTETLHRFSLEGDAPVSWTETKKQSFKQTGEFGEKRKNSI
LNPINSIRKESIVQKTPLQMNGIEEDSDEPLERRLSLVPDSEQGEAILPRISVISTGPTLQ
ARRRQSVLNLMTHSVNQGQNIHRKTTASTRKVSLAPQANLTELDIYSRRLSQETGLEI
SEEINEEDLKECFFDDMESIPAVTTWNTYLRYITVHKSLIFVLIWCLVIFLAEVAASLVV
LWLLGNTPLQDKGNSTHSRNNSYAVIITSTSSYYVFYIYVGVADTLLAMGFFRGLPLV
HTLITVSKILHHKMLHSVLQAPMSTLNTLKAGGILNRFSKDIAILDDLLPLTIFDFIQLL
LIVIGAIAVVAVLQPYIFVATVPVIVAFIMLRAYFLQTSQQLKQLESEGRSPIFTHLVTSL
KGLWTLRAFGRQPYFETLFHKALNLHTANWFLYLSTLRWFQMRIEMIFVIFFIAVTFIS
ILTTGEGEGRVGIILTLAMNIMSTLQWAVNSSIDVDSLMRSVSRVFKFIDMPTEGKPTK
STKPYKNGQLSKVMIIENSHVKKDDIWPSGGQMTVKDLTAKYTEGGNAILENISFSIS
PGQRVGLLGRTGSGKSTLLSAFLRLLNTEGEIQIDGVSWDSITLQQWRKAFGVIPQKV
FIFSGTFRKNLDPYEQWSDQEIWKVADEVGLRSVIEQFPGKLDFVLVDGGCVLSHGH
KQLMCLARSVLSKAKILLLDEPSAHLDPVTYQIIRRTLKQAFADCTVILCEHRIEAMLE
CQQFLVIEENKVRQYDSIQKLLNERSLFRQAISPSDRVKLFPHRNSSKCKSKPQIAALK
EETEEEVQDTRL
Auxiliary sequences:
CBh promoter
(SEQ ID NO: 32)
CGTTACATAACTTACGGTAAATGGCCCGCCTGGCTGACCGCCCAACGACCCCCGC
CCATTGACGTCAATAGTAACGCCAATAGGGACTTTCCATTGACGTCAATGGGTGGA
GTATTTACGGTAAACTGCCCACTTGGCAGTACATCAAGTGTATCATATGCCAAGTAC
GCCCCCTATTGACGTCAATGACGGTAAATGGCCCGCCTGGCATTGTGCCCAGTACA
TGACCTTATGGGACTTTCCTACTTGGCAGTACATCTACGTATTAGTCATCGCTATTAC
CATGGTCGAGGTGAGCCCCACGTTCTGCTTCACTCTCCCCATCTCCCCCCCCTCCC
CACCCCCAATTTTGTATTTATTTATTTTTTAATTATTTTGTGCAGCGATGGGGGCGGG
GGGGGGGGGGGGGCGCGCGCCAGGCGGGGCGGGGCGGGGCGAGGGGCGGGGC
GGGGCGAGGCGGAGAGGTGCGGCGGCAGCCAATCAGAGCGGCGCGCTCCGAAA
GTTTCCTTTTATGGCGAGGCGGCGGCGGCGGCGGCCCTATAAAAAGCGAAGCGCG
CGGCGGGCGGGAGTCGCTGCGCGCTGCCTTCGCCCCGTGCCCCGCTCCGCCGCCG
CCTCGCGCCGCCCGCCCCGGCTCTGACTGACCGCGTTACTCCCACAGGTGAGCGG
GCGGGACGGCCCTTCTCCTCCGGGCTGTAATTAGCTGAGCAAGAGGTAAGGGTTT
AAGGGATGGTTGGTTGGTGGGGTATTAATGTTTAATTACCTGGAGCACCTGCCTGA
AATCACTTTTTTTCAGGTTGG
Self-cleaving 2A peptide from porcine teschovirus-1
Nucleotide sequence
(SEQ ID NO: 37)
GGATCTGGCGCCACAAACTTCAGCCTGCTGAAGCAGGCCGGAGATGTGGAAGAA
AATCCTGGCCCC
Amino acid sequence
(SEQ ID NO: 38)
GSGATNFSLLKQAGDVEENPGP
Kozak
GCCACC
bGH polyA
(SEQ ID NO: 46)
CTGTGCCTTCTAGTTGCCAGCCATCTGTTGTTTGCCCCTCCCCCGTGCCTTCCTTGA
CCCTGGAAGGTGCCACTCCCACTGTCCTTTCCTAATAAAATGAGGAAATTGCATCG
CATTGTCTGAGTAGGTGTCATTCTATTCTGGGGGGTGGGGTGGGGCAGGACAGCA
AGGGGGAGGATTGGGAAGAGAATAGCAGGCAGGCTGGGGA

### Example 13

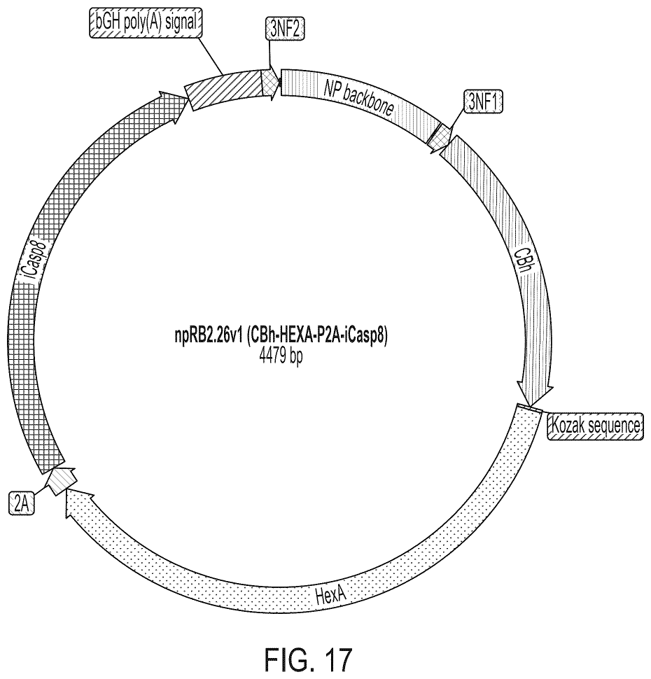

In the present example a single actuator-regulated is intended to deliver a functional copy of hexosaminidase subunit alpha (HexA) for the treatment of Tay-Sachs Disease. A CBh promoter controls expression of HexA and inducible Caspase 8 (iCasp8), which are connected by a self-cleaving peptide (P2A). The actuator is AP20187, a ligand that causes dimerization of caspase 8 and induction of apoptosis. FIG. 17 shows a plasmid map for the genetic construct described in this example.

Full sequence (except nanoplasmid backbone)
(SEQ ID NO: 105)
CTGGGGACTTTCCAGCCTGGGGACTTTCCAGCTGGGACTTTCCAGGCGTTACATA
ACTTACGGTAAATGGCCCGCCTGGCTGACCGCCCAACGACCCCCGCCCATTGACG
TCAATAGTAACGCCAATAGGGACTTTCCATTGACGTCAATGGGTGGAGTATTTACG
GTAAACTGCCCACTTGGCAGTACATCAAGTGTATCATATGCCAAGTACGCCCCCTA
TTGACGTCAATGACGGTAAATGGCCCGCCTGGCATTGTGCCCAGTACATGACCTTA
TGGGACTTTCCTACTTGGCAGTACATCTACGTATTAGTCATCGCTATTACCATGGTC
GAGGTGAGCCCCACGTTCTGCTTCACTCTCCCCATCTCCCCCCCCTCCCCACCCCC
AATTTTGTATTTATTTATTTTTTAATTATTTTGTGCAGCGATGGGGGCGGGGGGGGG
GGGGGGGCGCGCGCCAGGCGGGGCGGGGCGGGGCGAGGGGGGGGCGGGGCG
AGGCGGAGAGGTGCGGCGGCAGCCAATCAGAGCGGCGCGCTCCGAAAGTTTCCT
TTTATGGCGAGGCGGCGGCGGCGGCGGCCCTATAAAAAGCGAAGCGCGCGGCGG
GCGGGAGTCGCTGCGCGCTGCCTTCGCCCCGTGCCCCGCTCCGCCGCCGCCTCGC
GCCGCCCGCCCCGGCTCTGACTGACCGCGTTACTCCCACAGGTGAGCGGGCGGG
ACGGCCCTTCTCCTCCGGGCTGTAATTAGCTGAGCAAGAGGTAAGGGTTTAAGGG
ATGGTTGGTTGGTGGGGTATTAATGTTTAATTACCTGGAGCACCTGCCTGAAATCA
CTTTTTTTCAGGTTGGGCCACCATGACAAGCTCCAGGCTTTGGTTTTCGCTGCTGC
TGGCGGCAGCGTTCGCAGGACGGGCGACGGCCCTCTGGCCCTGGCCTCAGAACT
TCCAAACCTCCGACCAGCGCTACGTCCTTTACCCGAACAACTTTCAATTCCAGTAC
GATGTCAGCTCGGCCGCGCAGCCCGGCTGCTCAGTCCTCGACGAGGCCTTCCAGC
GCTATCGTGACCTGCTTTTCGGTTCCGGGTCTTGGCCCCGTCCTTACCTCACAGGT
TGGCCGCACCAAGCTTACCCTGTCTTCCTAGGGAAACGGCATACACTGGAGAAGA
ATGTGTTGGTTGTCTCTGTAGTCACACCTGGATGTAACCAGCTTCCTACTTTGGAG
TCAGTGGAGAATTATACCCTGACCATAAATGATGACCAGTGTTTACTCCTCTCTGA
GACTGTCTGGGGAGCTCTCCGAGGTCTGGAGACTTTTAGCCAGCTTGTTTGGAAA
TCTGCTGAGGGCACATTCTTTATCAACAAGACTGAGATTGAGGACTTTCCCCGCTT
TCCTCACCGGGGCTTGCTGTTGGATACATCTCGCCATTACCTGCCACTCTCTAGCAT
CCTGGACACTCTGGATGTCATGGCGTACAATAAATTGAACGTGTTCCACTGGCATC
TGGTAGATGATCCTTCCTTCCCATATGAGAGCTTCACTTTTCCAGAGCTCATGAGA
AAGGGGTCCTACAACCCTGTCACCCACATCTACACAGCACAGGATGTGAAGGAGG
TCATTGAATACGCACGGCTCCGGGGTATCCGTGTGCTTGCAGAGTTTGACACTCCT
GGCCACACTTTGTCCTGGGGACCAGGTATCCCTGGATTACTGACTCCTTGCTACTC
TGGGTCTGAGCCCTCTGGCACCTTTGGACCAGTGAATCCCAGTCTCAATAATACCT
ATGAGTTCATGAGCACATTCTTCTTAGAAGTCAGCTCTGTCTTCCCAGATTTTTATC
TTCATCTTGGAGGAGATGAGGTTGATTTCACCTGCTGGAAGTCCAACCCAGAGAT
CCAGGACTTTATGAGGAAGAAAGGCTTCGGTGAGGACTTCAAGCAGCTGGAGTC
CTTCTACATCCAGACGCTGCTGGACATCGTCTCTTCTTATGGCAAGGGCTATGTGG
TGTGGCAGGAGGTGTTTGATAATAAAGTAAAGATTCAGCCAGACACAATCATACA
GGTGTGGCGAGAGGATATTCCAGTGAACTATATGAAGGAGCTGGAACTGGTCACC
AAGGCCGGCTTCCGGGCCCTTCTCTCTGCCCCCTGGTACCTGAACCGTATATCCTA
TGGCCCTGACTGGAAGGATTTCTACATAGTGGAACCCCTGGCATTTGAAGGTACCC
CTGAGCAGAAGGCTCTGGTGATTGGTGGAGAGGCTTGTATGTGGGGAGAATATGT
GGACAACACAAACCTGGTCCCCAGGCTCTGGCCCAGAGCAGGGGCTGTTGCCGA
AAGGCTGTGGAGCAACAAGTTGACATCTGACCTGACATTTGCCTATGAACGTTTG
TCACACTTCCGCTGTGAATTGCTGAGGCGAGGTGTCCAGGCCCAACCCCTCAATG
TAGGCTTCTGTGAGCAGGAGTTTGAACAGACCGGATCTGGCGCTACTAACTTCAG
TCTGCTGAAGCAGGCCGGGGACGTGGAAGAGAACCCCGGTCCTATGGGGAGTAG
CAAGAGCAAGCCTAAGGACCCCAGCCAGCGCTCTAGAGGAGTGCAGGTGGAGAC
TATCTCCCCAGGAGACGGGCGCACCTTCCCCAAGCGCGGCCAGACCTGCGTGGTG
CACTACACCGGGATGCTTGAAGATGGAAAGAAAGTTGATTCCTCCCGGGACAGAA
ACAAGCCCTTTAAGTTTATGCTAGGCAAGCAGGAGGTGATCCGAGGCTGGGAAGA
AGGGGTTGCCCAGATGAGTGTGGGTCAGAGAGCCAAACTGACTATATCTCCAGAT
TATGCCTATGGTGCCACTGGGCACCCAGGCATCATCCCACCACATGCCACTCTCGT
CTTCGATGTGGAGCTTCTAAAACTGGAAACTAGTGGAGGTGGAGGTTCTAGAGGA
TCCCATATGACAATCTCGGACTCTCCAAGAGAACAGGATAGTGAATCACAGACTTT
GGACAAAGTTTACCAAATGAAAAGCAAACCTCGGGGATACTGTCTGATCATCAAC
AATCACAATTTTGCAAAAGCACGGGAGAAAGTGCCCAAACTTCACAGCATTAGGG
ACAGGAATGGAACACACTTGGATGCAGGGGCTTTGACCACGACCTTTGAAGAGC
TTCATTTTGAGATCAAGCCCCACGATGACTGCACAGTAGAGCAAATCTATGAGATT
TTGAAAATCTACCAACTCATGGACCACAGTAACATGGACTGCTTCATCTGCTGTAT
CCTCTCCCATGGAGACAAGGGCATCATCTATGGCACTGATGGACAGGAGGCCCCC
ATCTATGAGCTGACATCTCAGTTCACTGGTTTGAAGTGCCCTTCCCTTGCTGGAAA
ACCCAAAGTGTTTTTTATTCAGGCTTGTCAGGGGGATAACTACCAGAAAGGTATAC
CTGTTGAGACTGATTCAGAGGAGCAACCCTATTTAGAAATGGATTTATCATCACCT
CAAACGAGATATATCCCGGATGAGGCTGACTTTCTGCTGGGGATGGCCACTGTGA
ATAACTGTGTTTCCTACCGAAACCCTGCAGAGGGAACCTGGTACATCCAGTCACTT
TGCCAGAGCCTGAGAGAGCGATGTCCTCGAGGCGATGATATTCTCACCATCCTGAC
TGAAGTGAACTATGAAGTAAGCAACAAGGATGACAAGAAAAACATGGGGAAACA
GATGCCTCAGCCTACTTTCACACTAAGAAAAAAACTTGTCTTCCCTTCTGATTGAC
TGTGCCTTCTAGTTGCCAGCCATCTGTTGTTTGCCCCTCCCCCGTGCCTTCCTTGAC
CCTGGAAGGTGCCACTCCCACTGTCCTTTCCTAATAAAATGAGGAAATTGCATCGC
ATTGTCTGAGTAGGTGTCATTCTATTCTGGGGGGTGGGGTGGGGCAGGACAGCAA
GGGGGAGGATTGGGAAGAGAATAGCAGGCAGGCTGGGGACTGGGGACTTTCCAG
CTGGGGACTTTCCAGCTGGGACTTTCCAGGAG
Transgene: hexosaminidase A
Nucleotide sequence
(SEQ ID NO: 106)
ATGACAAGCTCCAGGCTTTGGTTTTCGCTGCTGCTGGCGGCAGCGTTCGCAGGAC
GGGCGACGGCCCTCTGGCCCTGGCCTCAGAACTTCCAAACCTCCGACCAGCGCTA
CGTCCTTTACCCGAACAACTTTCAATTCCAGTACGATGTCAGCTCGGCCGCGCAGC
CCGGCTGCTCAGTCCTCGACGAGGCCTTCCAGCGCTATCGTGACCTGCTTTTCGGT
TCCGGGTCTTGGCCCCGTCCTTACCTCACAGGTTGGCCGCACCAAGCTTACCCTGT
CTTCCTAGGGAAACGGCATACACTGGAGAAGAATGTGTTGGTTGTCTCTGTAGTC
ACACCTGGATGTAACCAGCTTCCTACTTTGGAGTCAGTGGAGAATTATACCCTGAC
CATAAATGATGACCAGTGTTTACTCCTCTCTGAGACTGTCTGGGGAGCTCTCCGAG
GTCTGGAGACTTTTAGCCAGCTTGTTTGGAAATCTGCTGAGGGCACATTCTTTATC
AACAAGACTGAGATTGAGGACTTTCCCCGCTTTCCTCACCGGGGCTTGCTGTTGG
ATACATCTCGCCATTACCTGCCACTCTCTAGCATCCTGGACACTCTGGATGTCATGG
CGTACAATAAATTGAACGTGTTCCACTGGCATCTGGTAGATGATCCTTCCTTCCCAT
ATGAGAGCTTCACTTTTCCAGAGCTCATGAGAAAGGGGTCCTACAACCCTGTCAC
CCACATCTACACAGCACAGGATGTGAAGGAGGTCATTGAATACGCACGGCTCCGG
GGTATCCGTGTGCTTGCAGAGTTTGACACTCCTGGCCACACTTTGTCCTGGGGACC
AGGTATCCCTGGATTACTGACTCCTTGCTACTCTGGGTCTGAGCCCTCTGGCACCT
TTGGACCAGTGAATCCCAGTCTCAATAATACCTATGAGTTCATGAGCACATTCTTCT
TAGAAGTCAGCTCTGTCTTCCCAGATTTTTATCTTCATCTTGGAGGAGATGAGGTT
GATTTCACCTGCTGGAAGTCCAACCCAGAGATCCAGGACTTTATGAGGAAGAAAG
GCTTCGGTGAGGACTTCAAGCAGCTGGAGTCCTTCTACATCCAGACGCTGCTGGA
CATCGTCTCTTCTTATGGCAAGGGCTATGTGGTGTGGCAGGAGGTGTTTGATAATA
AAGTAAAGATTCAGCCAGACACAATCATACAGGTGTGGCGAGAGGATATTCCAGT
GAACTATATGAAGGAGCTGGAACTGGTCACCAAGGCCGGCTTCCGGGCCCTTCTC
TCTGCCCCCTGGTACCTGAACCGTATATCCTATGGCCCTGACTGGAAGGATTTCTAC
ATAGTGGAACCCCTGGCATTTGAAGGTACCCCTGAGCAGAAGGCTCTGGTGATTG
GTGGAGAGGCTTGTATGTGGGGAGAATATGTGGACAACACAAACCTGGTCCCCAG
GCTCTGGCCCAGAGCAGGGGCTGTTGCCGAAAGGCTGTGGAGCAACAAGTTGAC
ATCTGACCTGACATTTGCCTATGAACGTTTGTCACACTTCCGCTGTGAATTGCTGA
GGCGAGGTGTCCAGGCCCAACCCCTCAATGTAGGCTTCTGTGAGCAGGAGTTTGA
ACAGACC
Amino acid sequence
(SEQ ID NO: 107)
MTSSRLWFSLLLAAAFAGRATALWPWPQNFQTSDQRYVLYPNNFQFQYDVSSAAQP
GCSVLDEAFQRYRDLLFGSGSWPRPYLTGWPHQAYPVFLGKRHTLEKNVLVVSVVT
PGCNQLPTLESVENYTLTINDDQCLLLSETVWGALRGLETFSQLVWKSAEGTFFINKT
EIEDFPRFPHRGLLLDTSRHYLPLSSILDTLDVMAYNKLNVFHWHLVDDPSFPYESFTF
PELMRKGSYNPVTHIYTAQDVKEVIEYARLRGIRVLAEFDTPGHTLSWGPGIPGLLTP
CYSGSEPSGTFGPVNPSLNNTYEFMSTFFLEVSSVFPDFYLHLGGDEVDFTCWKSNPE
IQDFMRKKGFGEDFKQLESFYIQTLLDIVSSYGKGYVVWQEVFDNKVKIQPDTIIQV
WREDIPVNYMKELELVTKAGFRALLSAPWYLNRISYGPDWKDFYIVEPLAFEGTPEQ
KALVIGGEACMWGEYVDNTNLVPRLWPRAGAVAERLWSNKLTSDLTFAYERLSHFRC
ELLRRGVQAQPLNVGFCEQEFEQT
Actuator: AP20187 or other dimerizer capable of dimerizing iCasp8
Effector element: iCasp8
Nucleotide sequence
(SEQ ID NO: 108)
ATGGGGAGTAGCAAGAGCAAGCCTAAGGACCCCAGCCAGCGCTCTAGAGGAGTG
CAGGTGGAGACTATCTCCCCAGGAGACGGGCGCACCTTCCCCAAGCGCGGCCAG
ACCTGCGTGGTGCACTACACCGGGATGCTTGAAGATGGAAAGAAAGTTGATTCCT
CCCGGGACAGAAACAAGCCCTTTAAGTTTATGCTAGGCAAGCAGGAGGTGATCCG
AGGCTGGGAAGAAGGGGTTGCCCAGATGAGTGTGGGTCAGAGAGCCAAACTGAC
TATATCTCCAGATTATGCCTATGGTGCCACTGGGCACCCAGGCATCATCCCACCACA
TGCCACTCTCGTCTTCGATGTGGAGCTTCTAAAACTGGAAACTAGTGGAGGTGGA
GGTTCTAGAGGATCCCATATGACAATCTCGGACTCTCCAAGAGAACAGGATAGTG
AATCACAGACTTTGGACAAAGTTTACCAAATGAAAAGCAAACCTCGGGGATACTG
TCTGATCATCAACAATCACAATTTTGCAAAAGCACGGGAGAAAGTGCCCAAACTT
CACAGCATTAGGGACAGGAATGGAACACACTTGGATGCAGGGGCTTTGACCACG
ACCTTTGAAGAGCTTCATTTTGAGATCAAGCCCCACGATGACTGCACAGTAGAGC
AAATCTATGAGATTTTGAAAATCTACCAACTCATGGACCACAGTAACATGGACTGC
TTCATCTGCTGTATCCTCTCCCATGGAGACAAGGGCATCATCTATGGCACTGATGG
ACAGGAGGCCCCCATCTATGAGCTGACATCTCAGTTCACTGGTTTGAAGTGCCCTT
CCCTTGCTGGAAAACCCAAAGTGTTTTTTATTCAGGCTTGTCAGGGGGATAACTAC
CAGAAAGGTATACCTGTTGAGACTGATTCAGAGGAGCAACCCTATTTAGAAATGG
ATTTATCATCACCTCAAACGAGATATATCCCGGATGAGGCTGACTTTCTGCTGGGG
ATGGCCACTGTGAATAACTGTGTTTCCTACCGAAACCCTGCAGAGGGAACCTGGT
ACATCCAGTCACTTTGCCAGAGCCTGAGAGAGCGATGTCCTCGAGGCGATGATATT
CTCACCATCCTGACTGAAGTGAACTATGAAGTAAGCAACAAGGATGACAAGAAA
AACATGGGGAAACAGATGCCTCAGCCTACTTTCACACTAAGAAAAAAACTTGTCT
TCCCTTCTGATTGA
Amino acid sequence
(SEQ ID NO: 107)
MTSSRLWFSLLLAAAFAGRATALWPWPQNFQTSDQRYVLYPNNFQFQYDVSSAAQP
GCSVLDEAFQRYRDLLFGSGSWPRPYLTGWPHQAYPVFLGKRHTLEKNVLVVSVVT
PGCNQLPTLESVENYTLTINDDQCLLLSETVWGALRGLETFSQLVWKSAEGTFFINKT
EIEDFPRFPHRGLLLDTSRHYLPLSSILDTLDVMAYNKLNVFHWHLVDDPSFPYESFTF
PELMRKGSYNPVTHIYTAQDVKEVIEYARLRGIRVLAEFDTPGHTLSWGPGIPGLLTP
CYSGSEPSGTFGPVNPSLNNTYEFMSTFFLEVSSVFPDFYLHLGGDEVDFTCWKSNPE
IQDFMRKKGFGEDFKQLESFYIQTLLDIVSSYGKGYVVWQEVFDNKVKIQPDTIIQV
WREDIPVNYMKELELVTKAGFRALLSAPWYLNRISYGPDWKDFYIVEPLAFEGTPEQ
KALVIGGEACMWGEYVDNTNLVPRLWPRAGAVAERLWSNKLTSDLTFAYERLSHFRC
ELLRRGVQAQPLNVGFCEQEFEQT
Auxiliary sequences:
CBh promoter
(SEQ ID NO: 32)
CGTTACATAACTTACGGTAAATGGCCCGCCTGGCTGACCGCCCAACGACCCCCGC
CCATTGACGTCAATAGTAACGCCAATAGGGACTTTCCATTGACGTCAATGGGTGGA
GTATTTACGGTAAACTGCCCACTTGGCAGTACATCAAGTGTATCATATGCCAAGTAC
GCCCCCTATTGACGTCAATGACGGTAAATGGCCCGCCTGGCATTGTGCCCAGTACA
TGACCTTATGGGACTTTCCTACTTGGCAGTACATCTACGTATTAGTCATCGCTATTAC
CATGGTCGAGGTGAGCCCCACGTTCTGCTTCACTCTCCCCATCTCCCCCCCCTCCC
CACCCCCAATTTTGTATTTATTTATTTTTTAATTATTTTGTGCAGCGATGGGGGCGGG
GGGGGGGGGGGGGCGCGCGCCAGGCGGGGCGGGGCGGGGCGAGGGGCGGGGC
GGGGCGAGGCGGAGAGGTGCGGCGGCAGCCAATCAGAGCGGCGCGCTCCGAAA
GTTTCCTTTTATGGCGAGGCGGCGGCGGCGGCGGCCCTATAAAAAGCGAAGCGCG
CGGCGGGCGGGAGTCGCTGCGCGCTGCCTTCGCCCCGTGCCCCGCTCCGCCGCCG
CCTCGCGCCGCCCGCCCCGGCTCTGACTGACCGCGTTACTCCCACAGGTGAGCGG
GCGGGACGGCCCTTCTCCTCCGGGCTGTAATTAGCTGAGCAAGAGGTAAGGGTTT
AAGGGATGGTTGGTTGGTGGGGTATTAATGTTTAATTACCTGGAGCACCTGCCTGA
AATCACTTTTTTTCAGGTTGG
Self-cleaving 2A peptide from porcine teschovirus-1
Nucleotide sequence
(SEQ ID NO: 37)
GGATCTGGCGCCACAAACTTCAGCCTGCTGAAGCAGGCCGGAGATGTGGAAGAA
AATCCTGGCCCC
Amino acid sequence
(SEQ ID NO: 38)
GSGATNFSLLKQAGDVEENPGP
Kozak
GCCACC
bGH polyA
(SEQ ID NO: 46)
CTGTGCCTTCTAGTTGCCAGCCATCTGTTGTTTGCCCCTCCCCCGTGCCTTCCTTGA
CCCTGGAAGGTGCCACTCCCACTGTCCTTTCCTAATAAAATGAGGAAATTGCATCG
CATTGTCTGAGTAGGTGTCATTCTATTCTGGGGGGTGGGGTGGGGCAGGACAGCA
AGGGGGAGGATTGGGAAGAGAATAGCAGGCAGGCTGGGGA

### Example 14

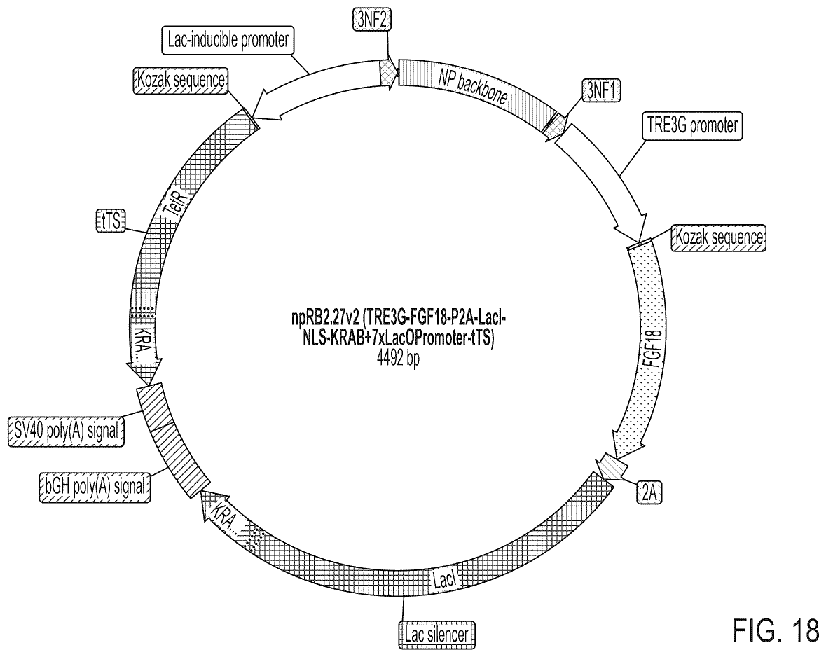

In the present example, a system containing a toggle-regulated effector element is intended to deliver biologically active fibroblast growth factor 18 for the treatment of osteoarthritis. The system is constructed of a constitutive CMV promoter followed by several TetO sequences driving the expression of both fibroblast growth factor 18 and the lac repressor protein fused to KRAB. A second promoter followed by LacO or CuO sequences drives the expression of the Tet repressor protein fused to KRAB (tet transcriptional silencer/tTS). When tetracycline is added, fibroblast growth factor 18 and the lac repressor/cumate repressor protein are expressed, allowing the switch to reach a stable equilibrium in which the lac/cumate-responsive second promoter is continuously repressed, and fibroblast growth factor 18 is continuously produced. When IPTG or cumate is added, the lac/cumate repressor protein can no longer bind to the LacO/CuO sequences, allowing the TetR-KRAB fusion protein to be expressed. The switch reaches a new stable equilibrium in which the TetR-KRAB fusion protein is continuously expressed, repressing the expression of fibroblast growth factor 18. FIG. 18 shows a plasmid map for the genetic construct described in this example.

Full sequence (except nanoplasmid backbone)
(SEQ ID NO: 109)
CTGGGGACTTTCCAGCCTGGGGACTTTCCAGCTGGGACTTTCCAGGGAGTTTACT
CCCTATCAGTGATAGAGAACGTATGAAGAGTTTACTCCCTATCAGTGATAGAGAAC
GTATGCAGACTTTACTCCCTATCAGTGATAGAGAACGTATAAGGAGTTTACTCCCTA
TCAGTGATAGAGAACGTATGACCAGTTTACTCCCTATCAGTGATAGAGAACGTATC
TACAGTTTACTCCCTATCAGTGATAGAGAACGTATATCCAGTTTACTCCCTATCAGT
GATAGAGAACGTATAAGCTTTAGGCGTGTACGGTGGGCGCCTATAAAAGCAGAGC
TCGTTTAGTGAACCGTCAGATCGCCTGGAGCAATTCCACAACACTTTTGTCTTATA
CCAACTTTCCGTACCACTTCCTACCCTCGTAAAGCCACCATGTATTCAGCGCCCTC
CGCCTGCACTTGCCTGTGTTTACACTTCCTGCTGCTGTGCTTCCAGGTACAGGTGC
TGGTTGCCGAGGAGAACGTGGACTTCCGCATCCACGTGGAGAACCAGACGCGGG
CTCGGGACGATGTGAGCCGTAAGCAGCTGCGGCTGTACCAGCTCTACAGCCGGAC
CAGTGGGAAACACATCCAGGTCCTGGGCCGCAGGATCAGTGCCCGCGGCGAGGA
TGGGGACAAGTATGCCCAGCTCCTAGTGGAGACAGACACCTTCGGTAGTCAAGTC
CGGATCAAGGGCAAGGAGACGGAATTCTACCTGTGCATGAACCGCAAAGGCAAG
CTCGTGGGGAAGCCCGATGGCACCAGCAAGGAGTGTGTGTTCATCGAGAAGGTT
CTGGAGAACAACTACACGGCCCTGATGTCGGCTAAGTACTCCGGCTGGTACGTGG
GCTTCACCAAGAAGGGGCGGCCGCGGAAGGGCCCCAAGACCCGGGAGAACCAG
CAGGACGTGCATTTCATGAAGCGCTACCCCAAGGGGCAGCCGGAGCTTCAGAAG
CCCTTCAAGTACACGACGGTGACCAAGAGGTCCCGTCGGATCCGGCCCACACACC
CTGCCGGATCTGGCGCCACAAACTTCAGCCTGCTGAAGCAGGCCGGAGATGTGG
AAGAAAATCCTGGCCCCGTGAAACCAGTAACGTTATACGATGTCGCAGAGTATGC
CGGTGTCTCTTATCAGACCGTTTCCCGCGTGGTGAACCAGGCCAGCCACGTTTCTG
CGAAAACGCGGGAAAAAGTGGAAGCGGCGATGGCGGAGCTGAATTACATTCCCA
ACCGCGTGGCACAACAACTGGCGGGCAAACAGTCGTTGCTGATTGGCGTTGCCA
CCTCCAGTCTGGCCCTGCACGCGCCGTCGCAAATTGTCGCGGCGATTAAATCTCGC
GCCGATCAACTGGGTGCCAGCGTGGTGGTGTCGATGGTAGAACGAAGCGGCGTC
GAAGCCTGTAAAGCGGCGGTGCACAATCTTCTCGCGCAACGCGTCAGTGGGCTGA
TCATTAACTATCCGCTGGATGACCAGGATGCCATTGCTGTGGAAGCTGCCTGCACT
AATGTTCCGGCGTTATTTCTTGATGTCTCTGACCAGACACCCATCAACAGTATTATT
TTCTCCCATGAAGACGGTACGCGACTGGGCGTGGAGCATCTGGTCGCATTGGGTC
ACCAGCAAATCGCGCTGTTAGCGGGCCCATTAAGTTCTGTCTCGGCGCGTCTGCGT
CTGGCTGGCTGGCATAAATATCTCACTCGCAATCAAATTCAGCCGATAGCGGAACG
GGAAGGCGACTGGAGTGCCATGTCCGGTTTTCAACAAACCATGCAAATGCTGAAT
GAGGGCATCGTTCCCACTGCGATGCTGGTTGCCAACGATCAGATGGCGCTGGGCG
CAATGCGCGCCATTACCGAGTCCGGGCTGCGCGTTGGTGCGGATATTTCGGTAGTG
GGATACGACGATACCGAAGACAGCTCATGTTATATCCCGCCGTTAACCACCATCAA
ACAGGATTTTCGCCTGCTGGGGCAAACCAGCGTGGACCGCTTGCTGCAACTCTCT
CAGGGCCAGGCGGTGAAGGGCAATCAGCTGTTGCCCGTCTCACTGGTGAAAAGA
AAAACCACCCTGGCGCCCAATACGCAAACCGCCTCTCCCCGCGCGTTGGCCGATT
CATTAATGCAGCTGGCACGACAGGTTTCCCGACTGGAAAGCGGGCAGCCAAAAA
AGAAGAGAAAGCTAGCAGTGTCAGTGACATTTGAAGATGTGGCTGTGCTCTTTAC
TCGGGACGAGTGGAAGAAGCTGGATCTGTCTCAGAGAAGCCTGTACCGTGAGGT
GATGCTGGAGAATTACAGCAACCTGGCCTCCATGGCAGGATTCCTGTTTACCAAAC
CAAAGGTGATCTCCCTGTTGCAGCAAGGAGAGGATCCCTGGTAACTGTGCCTTCT
AGTTGCCAGCCATCTGTTGTTTGCCCCTCCCCCGTGCCTTCCTTGACCCTGGAAGG
TGCCACTCCCACTGTCCTTTCCTAATAAAATGAGGAAATTGCATCGCATTGTCTGA
GTAGGTGTCATTCTATTCTGGGGGGTGGGGTGGGGCAGGACAGCAAGGGGGAGG
ATTGGGAAGAGAATAGCAGGCAGGCTGGGGAAACTTGTTTATTGCAGCTTATAATG
GTTACAAATAAAGCAATAGCATCACAAATTTCACAAATAAAGCATTTTTTTCACTG
CATTCTAGTTGTGGTTTGTCCAAACTCATCAATGTATCTTATTACCAGGGATCCTCT
CCTTGCTGCAACAGGGAGATCACCTTTGGTTTGGTAAACAGGAATCCTGCCATGG
AGGCCAGGTTGCTGTAATTCTCCAGCATCACCTCACGGTACAGGCTTCTCTGAGAC
AGATCCAGCTTCTTCCACTCGTCCCGAGTAAAGAGCACAGCCACATCTTCAAATGT
CACTGACACTGCTAGCTTTCTCTTCTTTTTTGGTTTATTACCATCCTCAATGGGTGT
ATGCTTTTTTTCATCTAACTGTGCAGAAAATCCAAATATTAATGATTTCAGCCCGAA
ATGGAAGGCCATTTCAGCAGAGGTTCTCGCCTGAATATTAAATGCCTCTTGTAATA
ATGGTGGTGCAGCGTCTATCACATGATTATTTTCTATCTGGTTTGTTGCTTGCTCCTC
TAATACTGCACCCAACGTAAAATGGCTGATAGATTGCAGAATGAAAAGAGCCTCCT
CGACCGAAAACCCTGCATCGCATAGACAGCGTAGTTGCGCCTCTGCTTGTTCAAA
CTGGGGGGGCGTAGGAGAGGTCCCTATATGCAATCGGGCTCCATCACGATGGACC
AGTAATGCTTTACGGAAACTCAGAGCATTTTCCTGGAGAAACTGCTGCCAACTCT
CAGTCGGTAACGGTGCTGAACGGGTGTGATGCTTCGCCAGTATTGCCTCTGAAAG
CATGTTCATAAGAGTCTGCTTGTTGCGCACGTGCCAATACAATGTAGGCTGCTCTA
CACCTAGCTTCTGGGCGAGTTTACGGGTTGTTAAACCTTCGATTCCGACCTCATTA
AGCAGCTCTAATGCGCTGTTAATCACTTTACTTTTATCTAATCTAGACATGGTGGCT
TTACGAGGGTAGGAAGTGGTACGGAAAGTTGGTATAAGACAAAAGTGTTGTGGAA
TTGCTCCAGGCGATCTGACGGTTCACTAAACGAGCTCTGCTTTTATAGGCGCCCAC
CGTACACGCCTAAAGCTTATACGTAATTGTGAGCGCTCACAATTGTAAACTGGATAT
ACGTAATTGTGAGCGCTCACAATTGTAAACTGTAGATACGTAATTGTGAGCGCTCA
CAATTGTAAACTGGTCATACGTAATTGTGAGCGCTCACAATTGTAAACTCCTTATAC
GTAATTGTGAGCGCTCACAATTGTAAAGTCTGCATACGTAATTGTGAGCGCTCACA
ATTGTAAACTCTTCATACGTAATTGTGAGCGCTCACAATTGTAAACTCCTGGGGAC
TTTCCAGCTGGGGACTTTCCAGCTGGGACTTTCCAGGAG (SEQ ID NO: 109)
Transgene: fibroblast growth factor 18
Nucleotide sequence
(SEQ ID NO: 110)
ATGTATTCAGCGCCCTCCGCCTGCACTTGCCTGTGTTTACACTTCCTGCTGCTGTGC
TTCCAGGTACAGGTGCTGGTTGCCGAGGAGAACGTGGACTTCCGCATCCACGTGG
AGAACCAGACGCGGGCTCGGGACGATGTGAGCCGTAAGCAGCTGCGGCTGTACC
AGCTCTACAGCCGGACCAGTGGGAAACACATCCAGGTCCTGGGCCGCAGGATCA
GTGCCCGCGGCGAGGATGGGGACAAGTATGCCCAGCTCCTAGTGGAGACAGACA
CCTTCGGTAGTCAAGTCCGGATCAAGGGCAAGGAGACGGAATTCTACCTGTGCAT
GAACCGCAAAGGCAAGCTCGTGGGGAAGCCCGATGGCACCAGCAAGGAGTGTGT
GTTCATCGAGAAGGTTCTGGAGAACAACTACACGGCCCTGATGTCGGCTAAGTAC
TCCGGCTGGTACGTGGGCTTCACCAAGAAGGGGCGGCCGCGGAAGGGCCCCAAG
ACCCGGGAGAACCAGCAGGACGTGCATTTCATGAAGCGCTACCCCAAGGGGCAG
CCGGAGCTTCAGAAGCCCTTCAAGTACACGACGGTGACCAAGAGGTCCCGTCGG
ATCCGGCCCACACACCCTGCC
Amino acid sequence
(SEQ ID NO: 111)
MYSAPSACTCLCLHFLLLCFQVQVLVAEENVDFRIHVENQTRARDDVSRKQLRLYQL
YSRTSGKHIQVLGRRISARGEDGDKYAQLLVETDTFGSQVRIKGKETEFYLCMNRKG
KLVGKPDGTSKECVFIEKVLENNYTALMSAKYSGWYVGFTKKGRPRKGPKTRENQQ
DVHFMKRYPKGQPELQKPFKYTTVTKRSRRIRPTHPA
Actuator: Tetracycline or derivative, IPTG or cumate
Actuator response elements: Tet Repressor-KRAB (tTS)/TRE3G, Lac Repressor-KRAB/
CMV-7x LacO or cumate repressor/CuOn promoter
tTS
Nucleotide sequence
(SEQ ID NO: 112)
ATGTCTAGATTAGATAAAAGTAAAGTGATTAACAGCGCATTAGAGCTGCTTAATGA
GGTCGGAATCGAAGGTTTAACAACCCGTAAACTCGCCCAGAAGCTAGGTGTAGAG
CAGCCTACATTGTATTGGCACGTGCGCAACAAGCAGACTCTTATGAACATGCTTTC
AGAGGCAATACTGGCGAAGCATCACACCCGTTCAGCACCGTTACCGACTGAGAGT
TGGCAGCAGTTTCTCCAGGAAAATGCTCTGAGTTTCCGTAAAGCATTACTGGTCCA
TCGTGATGGAGCCCGATTGCATATAGGGACCTCTCCTACGCCCCCCCAGTTTGAAC
AAGCAGAGGCGCAACTACGCTGTCTATGCGATGCAGGGTTTTCGGTCGAGGAGGC
TCTTTTCATTCTGCAATCTATCAGCCATTTTACGTTGGGTGCAGTATTAGAGGAGCA
AGCAACAAACCAGATAGAAAATAATCATGTGATAGACGCTGCACCACCATTATTAC
AAGAGGCATTTAATATTCAGGCGAGAACCTCTGCTGAAATGGCCTTCCATTTCGGG
CTGAAATCATTAATATTTGGATTTTCTGCACAGTTAGATGAAAAAAAGCATACACC
CATTGAGGATGGTAATAAACCAAAAAAGAAGAGAAAGCTAGCAGTGTCAGTGAC
ATTTGAAGATGTGGCTGTGCTCTTTACTCGGGACGAGTGGAAGAAGCTGGATCTG
TCTCAGAGAAGCCTGTACCGTGAGGTGATGCTGGAGAATTACAGCAACCTGGCCT
CCATGGCAGGATTCCTGTTTACCAAACCAAAGGTGATCTCCCTGTTGCAGCAAGG
AGAGGATCCCTGGTAA
Amino acid sequence
(SEQ ID NO: 20)
MSRLDKSKVINSALELLNEVGIEGLTTRKLAQKLGVEQPTLYWHVRNKQTLMNMLS
EAILAKHHTRSAPLPTESWQQFLQENALSFRKALLVHRDGARLHIGTSPTPPQFEQAE
AQLRCLCDAGFSVEEALFILQSISHFTLGAVLEEQATNQIENNHVIDAAPPLLQEAFNI
QARTSAEMAFHFGLKSLIFGFSAQLDEKKHTPIEDGNKPKKKRKLAVSVTFEDVAVLF
TRDEWKKLDLSQRSLYREVMLENYSNLASMAGFLFTKPKVISLLQQGEDPW
7x LacO Promoter
(SEQ ID NO: 113)
GAGTTTACAATTGTGAGCGCTCACAATTACGTATGAAGAGTTTACAATTGTGAGCG
CTCACAATTACGTATGCAGACTTTACAATTGTGAGCGCTCACAATTACGTATAAGG
AGTTTACAATTGTGAGCGCTCACAATTACGTATGACCAGTTTACAATTGTGAGCGC
TCACAATTACGTATCTACAGTTTACAATTGTGAGCGCTCACAATTACGTATATCCAG
TTTACAATTGTGAGCGCTCACAATTACGTATAAGCTTTAGGCGTGTACGGTGGGCG
CCTATAAAAGCAGAGCTCGTTTAGTGAACCGTCAGATCGCCTGGAGCAATTCCAC
AACACTTTTGTCTTATACCAACTTTCCGTACCACTTCCTACCCTCGTAAA
Lac repressor-KRAB
Nucleotide sequence
(SEQ ID NO: 114)
GTGAAACCAGTAACGTTATACGATGTCGCAGAGTATGCCGGTGTCTCTTATCAGAC
CGTTTCCCGCGTGGTGAACCAGGCCAGCCACGTTTCTGCGAAAACGCGGGAAAA
AGTGGAAGCGGCGATGGCGGAGCTGAATTACATTCCCAACCGCGTGGCACAACA
ACTGGCGGGCAAACAGTCGTTGCTGATTGGCGTTGCCACCTCCAGTCTGGCCCTG
CACGCGCCGTCGCAAATTGTCGCGGCGATTAAATCTCGCGCCGATCAACTGGGTG
CCAGCGTGGTGGTGTCGATGGTAGAACGAAGCGGCGTCGAAGCCTGTAAAGCGG
CGGTGCACAATCTTCTCGCGCAACGCGTCAGTGGGCTGATCATTAACTATCCGCTG
GATGACCAGGATGCCATTGCTGTGGAAGCTGCCTGCACTAATGTTCCGGCGTTATT
TCTTGATGTCTCTGACCAGACACCCATCAACAGTATTATTTTCTCCCATGAAGACG
GTACGCGACTGGGCGTGGAGCATCTGGTCGCATTGGGTCACCAGCAAATCGCGCT
GTTAGCGGGCCCATTAAGTTCTGTCTCGGCGCGTCTGCGTCTGGCTGGCTGGCATA
AATATCTCACTCGCAATCAAATTCAGCCGATAGCGGAACGGGAAGGCGACTGGAG
TGCCATGTCCGGTTTTCAACAAACCATGCAAATGCTGAATGAGGGCATCGTTCCCA
CTGCGATGCTGGTTGCCAACGATCAGATGGCGCTGGGCGCAATGCGCGCCATTAC
CGAGTCCGGGCTGCGCGTTGGTGCGGATATTTCGGTAGTGGGATACGACGATACCG
AAGACAGCTCATGTTATATCCCGCCGTTAACCACCATCAAACAGGATTTTCGCCTG
CTGGGGCAAACCAGCGTGGACCGCTTGCTGCAACTCTCTCAGGGCCAGGCGGTG
AAGGGCAATCAGCTGTTGCCCGTCTCACTGGTGAAAAGAAAAACCACCCTGGCG
CCCAATACGCAAACCGCCTCTCCCCGCGCGTTGGCCGATTCATTAATGCAGCTGGC
ACGACAGGTTTCCCGACTGGAAAGCGGGCAGCCAAAAAAGAAGAGAAAGCTAG
CAGTGTCAGTGACATTTGAAGATGTGGCTGTGCTCTTTACTCGGGACGAGTGGAA
GAAGCTGGATCTGTCTCAGAGAAGCCTGTACCGTGAGGTGATGCTGGAGAATTAC
AGCAACCTGGCCTCCATGGCAGGATTCCTGTTTACCAAACCAAAGGTGATCTCCCT
GTTGCAGCAAGGAGAGGATCCCTGGTAA
Amino acid sequence
(SEQ ID NO: 115)
VKPVTLYDVAEYAGVSYQTVSRVVNQASHVSAKTREKVEAAMAELNYIPNRVAQQL
AGKQSLLIGVATSSLALHAPSQIVAAIKSRADQLGASVVVSMVERSGVEACKAAVHN
LLAQRVSGLIINYPLDDQDAIAVEAACTNVPALFLDVSDQTPINSIIFSHEDGTRLGVEH
LVALGHQQIALLAGPLSSVSARLRLAGWHKYLTRNQIQPIAEREGDWSAMSGFQQTM
QMLNEGIVPTAMLVANDQMALGAMRAITESGLRVGADISVVGYDDTEDSSCYIPPLT
TIKQDFRLLGQTSVDRLLQLSQGQAVKGNQLLPVSLVKRKTTLAPNTQTASPRALAD
SLMQLARQVSRLESGQPKKKRKLAVSVTFEDVAVLFTRDEWKKLDLSQRSLYREVM
LENYSNLASMAGFLFTKPKVISLLQQGEDPW
7x CuO Promoter
(SEQ ID NO: 116)
GAGTTTACAACAAACAGACAATCTGGTCTGTTTGTAACGTATGAAGAGTTTACAA
CAAACAGACAATCTGGTCTGTTTGTAACGTATGCAGACTTTACAACAAACAGACA
ATCTGGTCTGTTTGTAACGTATAAGGAGTTTACAACAAACAGACAATCTGGTCTGT
TTGTAACGTATGACCAGTTTACAACAAACAGACAATCTGGTCTGTTTGTAACGTAT
CTACAGTTTACAACAAACAGACAATCTGGTCTGTTTGTAACGTATATCCAGTTTAC
AACAAACAGACAATCTGGTCTGTTTGTAACGTATAAGCTTTAGGCGTGTACGGTGG
GCGCCTATAAAAGCAGAGCTCGTTTAGTGAACCGTCAGATCGCCTGGAGCAATTC
CACAACACTTTTGTCTTATACCAACTTTCCGTACCACTTCCTACCCTCGTAAA
Cumate repressor
Nucleotide sequence
(SEQ ID NO: 117)
ATGAGTCCAAAGAGAAGAACACAGGCAGAGCGCGCAATGGAGACCCAGGGCAA
GTTGATTGCAGCGGCCCTGGGGGTTTTACGGGAAAAAGGTTACGCGGGATTCCGG
ATCGCAGATGTGCCCGGTGCTGCAGGTGTCTCGAGAGGAGCGCAGAGCCATCATT
TCCCGACAAAGCTTGAGCTTCTGCTTGCCACTTTTGAATGGCTTTACGAACAGATC
ACCGAACGCAGTCGGGCTCGATTAGCGAAATTGAAGCCAGAGGATGACGTCATCC
AGCAAATGCTGGACGACGCCGCCGAATTTTTCCTCGACGATGACTTCTCTATCAGC
CTTGATTTGATTGTGGCTGCCGACCGGGATCCAGCGTTACGCGAGGGTATTCAGCG
CACGGTAGAGAGGAATCGGTTTGTCGTCGAGGATATGTGGCTTGGTGTTCTGGTG
AGCCGTGGTCTTTCGCGTGATGATGCAGAAGATATCCTTTGGTTGATATTCAATTCG
GTGCGTGGGCTTGCTGTTCGTAGCCTATGGCAGAAGGACAAAGAACGCTTTGAGC
GTGTCAGGAACTCGACACTCGAAATTGCGCGAGAGCGGTACGCGAAATTCAAGC
GCTAG
Amino acid sequence
(SEQ ID NO: 118)
MSPKRRTQAERAMETQGKLIAAALGVLREKGYAGFRIADVPGAAGVSRGAQSHHFP
TKLELLLATFEWLYEQITERSRARLAKLKPEDDVIQQMLDDAAEFFLDDDFSISLDLIV
AADRDPALREGIQRTVERNRFVVEDMWLGVLVSRGLSRDDAEDILWLIFNSVRGLAV
RSLWQKDKERFERVRNSTLEIARERYAKFKR
Auxiliary sequences:
Self-cleaving 2A peptide from porcine teschovirus-1
Nucleotide sequence
(SEQ ID NO: 37)
GGATCTGGCGCCACAAACTTCAGCCTGCTGAAGCAGGCCGGAGATGTGGAAGAA
AATCCTGGCCCC
Amino acid sequence
(SEQ ID NO: 38)
GSGATNFSLLKQAGDVEENPGP
Kozak
GCCACC
bGH polyA
(SEQ ID NO: 46)
CTGTGCCTTCTAGTTGCCAGCCATCTGTTGTTTGCCCCTCCCCCGTGCCTTCCTTGA
CCCTGGAAGGTGCCACTCCCACTGTCCTTTCCTAATAAAATGAGGAAATTGCATCG
CATTGTCTGAGTAGGTGTCATTCTATTCTGGGGGGGGGGTGGGGCAGGACAGCA
AGGGGGAGGATTGGGAAGAGAATAGCAGGCAGGCTGGGGA
SV40 polyA
(SEQ ID NO: 47)
TAAGATACATTGATGAGTTTGGACAAACCACAACTAGAATGCAGTGAAAAAAATG
CTTTATTTGTGAAATTTGTGATGCTATTGCTTTATTTGTAACCATTATAAGCTGCAAT
AAACAAGTT

### EQUIVALENTS

Those skilled in the art will recognize or be able to ascertain using no more than routine experimentation, many equivalents to the specific embodiments of the invention described in the present application. Such equivalents are intended to be encompassed by the following claims.

All references, including patent documents, are incorporated by reference in their entirety.

1. A system for permanently reducing gene expression from a therapeutic construct or a plethora of constructs within one or more cells comprised of at least:
(a) a therapeutic transgene containing nucleic acid construct delivered in a formulation capable of entry into cells and enablement of stable therapeutic transgene expression for a period of at least 4 months;
(b) an actuator comprised of one or more chemical or biological molecules;
(c) an actuator response element and/or actuator response sequence that enables expression, activation, or stabilizes an effector element or an effector sequence;
(d) an effector sequence delivered in cis with the therapeutic transgene; and
(e) an effector element expressed from said effector sequence capable of permanently reducing expression of said therapeutic transgene in a manner that elicits minimal bystander effect and is therefore safe to surrounding tissues, organs, and organism in its entirety.

2. The system of claim 1, wherein the system is intended for delivery to subcutaneous tissues.

3-19. (canceled)

20. The system of claim 1, wherein the system is intended to be used in conjunction with therapeutic gene therapy or delivered in conjunction with a therapeutic transgene for the treatment of human, plant, or veterinary diseases or for the augmentation of human, veterinary, or plant function.

21-35. (canceled)

36. The system of claim 1, wherein the system is delivered as part of a viral or non-viral formulation, in vivo or ex vivo.

37. The system of claim 1, wherein the system is delivered by physical methods such as electroporation, nanoparticle bombardment, ultrasound mediated transfection, or chemically mediated transfection.

38-58. (canceled)

59. The system of claim 1, wherein the actuator is a small molecule, peptide, protein or nucleic acid that is capable of increasing, decreasing, or modulating local or systemic inflammation, or a part of the inflammatory response.

60-70. (canceled)

71. The system of claim 1, wherein the actuator is comprised at least in part from an exogenously administered small molecule, peptide, protein, RNA, DNA, or pharmacological formulation.

72-173. (canceled)

174. The system of claim 1, wherein the effector sequence is comprised at least in part from GTGTAGTTCGACCATTCGTG (SEQ ID NO: 1).

175. The system of claim 1, wherein the effector sequence is comprised at least in part from GTTCAGGATCACGTTACCGC (SEQ ID NO: 2).

176. The system of claim 1, wherein the effector sequence is comprised at least in part from GGGCGAGGAGCTGTTCACCG (SEQ ID NO: 3).

177. The system of claim 1, wherein the effector sequence is comprised at least in part from GAAGTTCGAGGGCGACACCC (SEQ ID NO: 4).

178. The system of claim 1, wherein the effector sequence is comprised at least in part from GCAGATGTAGTGTTTCCACA (SEQ ID NO: 5).

179. The system of claim 1, wherein the effector sequence is comprised at least in part from CCTAAGGTTAAGTCGCCCTCG (SEQ ID NO: 6).

180. The system of claim 1, wherein the effector sequence is comprised at least in part from CAACAAGATGAAGAGCACCAA (SEQ ID NO: 7).

181-189. (canceled)

190. The system of claim 1, wherein the actuator response element is a nucleic acid sequence or a protein that interacts with an actuator to enable activity of the effector element or effector sequence.

191-209. (canceled)


SEQUENCE LISTING (118 sequences)

SEQ ID NO: 1 - DNA, 20 bp, synthetic construct
  source 1..20: mol_type = other DNA
gtgtagttcgaccattcgtg

SEQ ID NO: 2 - DNA, 20 bp, synthetic construct
  source 1..20: mol_type = other DNA
gttcaggatcacgttaccgc

SEQ ID NO: 3 - DNA, 20 bp, synthetic construct
  source 1..20: mol_type = other DNA
gggcgaggagctgttcaccg

SEQ ID NO: 4 - DNA, 20 bp, synthetic construct
  source 1..20: mol_type = other DNA
gaagttcgagggcgacaccc

SEQ ID NO: 5 - DNA, 20 bp, synthetic construct
  source 1..20: mol_type = other DNA
gcagatgtagtgtttccaca

SEQ ID NO: 6 - DNA, 21 bp, synthetic construct
  source 1..21: mol_type = other DNA
cctaaggttaagtcgccctcg

SEQ ID NO: 7 - DNA, 21 bp, synthetic construct
  source 1..21: mol_type = other DNA
caacaagatgaagagcaccaa

SEQ ID NO: 8 - DNA, 459 bp, synthetic construct
  source 1..459: mol_type = other DNA
ctagctggcttgttgtccacaaccattaaaccttaaaagctttaaaagccttatatattcttttttttcttataaaacttaaaaccttagaggctatttaagttgctgatttatattaattttattgttcaaacatgagagcttagtacgtgaaacatgagagcttagtacattagccatgagagcttagtacattagccatgagggtttagttcattaaacatgagagcttagtacattaaacatgagagcttagtacattaaacatgagagcttagtacatactatcaacaggttgaactgctgatctgtacagtagaattggtaaagagagttgtgtaaaatattgagttcgcacatcttgttgtctgattattgatttttggcgaaaccatttgatcatatgacaagatgtgtatctaccttaacttaatgattttgataaaaatcattaggtac

SEQ ID NO: 9 - DNA, 7848 bp, synthetic construct
  source 1..7848: mol_type = other DNA
ctggggactttccagcctggggactttccagctgggactttccaggggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagggttcaggatcacgttaccgcggggccaccatggccctgtggatgagactgctgcctctgctggccctcctggccctgtggggccccgaccccgccgccgcttttgtgaaccagcacctgtgcggaagcgacctggtcgaggccctgtacctggtgtgcggcgagcggggcttcttctacacccctagaaccaagcgggaagccgaggatctgcaggtgggccaggtggagctgggcggcggaccaggcgccggctccctgcaacctctggctctggaaggatctagacagaagagaggcatcgtggagcagtgttgtacaagcatctgcagcctgtatcagctggaaaactactgcaatggatctggcgccacaaacttcagcctgctgaagcaggccggagatgtggaagaaaatcctggccccatgtctagattagataaaagtaaagtgattaacagcgcattagagctgcttaatgaggtcggaatcgaaggtttaacaacccgtaaactcgcccagaagctaggtgtagagcagcctacattgtattggcacgtgcgcaacaagcagactcttatgaacatgctttcagaggcaatactggcgaagcatcacacccgttcagcaccgttaccgactgagagttggcagcagtttctccaggaaaatgctctgagtttccgtaaagcattactggtccatcgtgatggagcccgattgcatatagggacctctcctacgcccccccagtttgaacaagcagaggcgcaactacgctgtctatgcgatgcagggttttcggtcgaggaggctcttttcattctgcaatctatcagccattttacgttgggtgcagtattagaggagcaagcaacaaaccagatagaaaataatcatgtgatagacgctgcaccaccattattacaagaggcatttaatattcaggcgagaacctctgctgaaatggccttccatttcgggctgaaatcattaatatttggattttctgcacagttagatgaaaaaaagcatacacccattgaggatggtaataaaccaaaaaagaagagaaagctagcagtgtcagtgacatttgaagatgtggctgtgctctttactcgggacgagtggaagaagctggatctgtctcagagaagcctgtaccgtgaggtgatgctggagaattacagcaacctggcctccatggcaggattcctgtttaccaaaccaaaggtgatctccctgttgcagcaaggagaggatccctggggctccggagagggcaggggaagtcttctaacatgcggggacgtggaggaaaatcccggccccatgtctagactggacaagagcaaagtcataaacggcgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagcaattgagctgttcgaccggcagggagccgaacctgccttccttttcggcctggaactaatcatatgtggcctggagaaacagctaaagtgcgaaagcggcgggccggccgacgcccttgacgattttgacttagacatgctcccagccgatgcccttgacgactttgaccttgatatgctgcctgctgacgctcttgacgattttgaccttgacatgctccccgggtaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcaggctggggaaaaaaagcaccgactcggtgccactttttcaagttgataacggactagccttattttaacttgctatttctagctctaaaacgcggtaacgtgatcctgaaccgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcaacttgtttattgcagcttataatggttacaaataaagcaatagcatcacaaatttcacaaataaagcatttttttcactgcattctagttgtggtttgtccaaactcatcaatgtatcttatcattattaggcgtagtcgggcacgtcgtaggggtatacttttctctttttctttggaggtccggattactttttcttttttgcctggccggcctttttcgtggccgccggccttttgtcgcctcccagctgagacaggtcgatccgtgtctcgtacaggccggtgatgctctggtggatcagggtggcgtccagcacctctttggtgctggtgtacctcttccggtcgatggtggtgtcaaagtacttgaaggcggcaggggctcccagattggtcagggtaaacaggtggatgatattctcggcctgctctctgatgggcttatcccggtgcttgttgtaggcggacagcactttgtccagattagcgtcggccaggatcactctcttggagaactcgctgatctgctcgatgatctcgtccaggtagtgcttgtgctgttccacaaacagctgtttctgctcattatcctcgggggagcccttcagcttctcatagtggctggccaggtacaggaagttcacatatttggagggcagggccagttcgtttcccttctgcagttcgccggcagaggccagcattctcttccggccgttttccagctcgaacagggagtacttaggcagcttgatgatcaggtcctttttcacttctttgtagcccttggcttccagaaagtcgatgggattcttctcgaagctgcttctttccatgatggtgatccccagcagctctttcacactcttcagtttcttggacttgcccttttccactttggccaccaccagcacagaataggccacggtggggctgtcgaagccgccgtacttcttagggtcccagtccttctttctggcgatcagcttatcgctgttcctcttgggcaggatagactctttgctgaagccgcctgtctgcacctcggtctttttcacgatattcacttggggcatgctcagcactttccgcacggtggcaaaatcccggcccttatcccacacgatctccccggtttcgccgtttgtctcgatcagaggccgcttccggatctcgccgttggccagggtaatctcggtcttgaaaaagttcatgatgttgctgtagaagaagtacttggcggtagccttgccgatttcctgctcgctcttggcgatcatcttccgcacgtcgtacaccttgtagtcgccgtacacgaactcgctttccagcttagggtactttttgatcagggcggttcccacgacggcgttcaggtaggcgtcgtgggcgtggtggtagttgttgatctcgcgcactttgtaaaactggaaatccttccggaaatcggacaccagcttggacttcagggtgatcactttcacttcccggatcagcttgtcattctcgtcgtacttagtgttcatccgggagtccaggatctgtgccacgtgctttgtgatctgccgggtttccaccagctgtctcttgatgaagccggccttatccagttcgctcaggccgcctctctcggccttggtcagattgtcgaactttctctgggtaatcagcttggcgttcagcagctgccgccagtagttcttcatcttcttcacgacctcttcggagggcacgttgtcgctcttgccccggttcttgtcgcttctggtcagcaccttgttgtcgatggagtcgtccttcagaaagctctgaggcacgatatggtccacatcgtagtcggacagccggttgatgtccagttcctggtccacgtacatatcccgcccattctgcaggtagtacaggtacagcttctcgttctgcagctgggtgttttccacggggtgttctttcaggatctggctgcccagctctttgatgccctcttcgatccgcttcattctctcgcggctgttcttctgtcccttctgggtggtctggttctctctggccatttcgatcacgatgttctcgggcttgtgccggcccatcactttcacgagctcgtccaccaccttcactgtctgcaggatgcccttcttaatggcggggctgccggccagattggcaatgtgctcgtgcaggctatcgccctggccggacacctgggctttctggatgtcctctttaaaggtcaggctgtcgtcgtggatcagctgcatgaagtttctgttggcgaagccgtcggacttcaggaaatccaggattgtcttgccggactgcttgtcccggatgccgttgatcagcttccggctcagcctgccccagccggtgtatctccgccgcttcagctgcttcatcactttgtcgtcgaacaggtgggcataggttttcagccgttcctcgatcatctctctgtcctcaaacagtgtcagggtcagcacgatatcttccagaatgtcctcgttttcctcattgtccaggaagtccttgtccttgataattttcagcagatcgtggtatgtgcccagggaggcgttgaaccgatcttccacgccggagatttccacggagtcgaagcactcgattttcttgaagtagtcctctttcagctgcttcacggtcactttccggttggtcttgaacagcaggtccacgatggcctttttctgctcgccgctcaggaaggcgggctttctcattccctcggtcacgtatttcactttggtcagctcgttatacacggtgaagtactcgtacagcaggctgtgcttgggcagcaccttctcgttgggcaggttcttatcgaagttggtcatccgctcgatgaagctctgggcggaagcgcccttgtccaccacttcctcgaagttccagggggtgatggtttcctcgctctttctggtcatccaggcgaatctgctgtttcccctggccagagggcccacgtagtaggggatgcggaaggtcaggatcttctcgatcttttcccggttgtccttcaggaatgggtaaaaatcttcctgccgccgcagaatggcgtgcagctctcccaggtggatctggtgggggatgctgccgttgtcgaaggtccgctgcttccgcagcaggtcctctctgttcagcttcacgagcagttcctcggtgccgtccatcttttccaggatgggcttgatgaacttgtagaactcttcctggctggctccgccgtcaatgtagccggcgtagccgttcttgctctggtcgaagaaaatctctttgtacttctcaggcagctgctgccgcacgagagctttcagcagggtcaggtcctggtggtgctcgtcgtatctcttgatcatagaggcgctcaggggggccttggtgatctcggtgttcactctcaggatgtcgctcagcaggatggcgtcggacaggttcttggcggccagaaacaggtcggcgtactggtcgccgatctgggccagcaggttgtccaggtcgtcgtcgtaggtgtccttgctcagctgcagtttggcatcctcggccaggtcgaagttgctcttgaagttgggggtcaggcccaggctcagggcaatcaggtttccgaacaggccattcttcttctcgccgggcagctgggcgatcagattttccagccgtctgctcttgctcagtctggcagacaggatggccttggcgtccacgccgctggcgttgatggggttttcctcgaacagctggttgtaggtctgcaccagctggatgaacagcttgtccacgtcgctgttgtcggggttcaggtcgccctcgatcaggaagtggccccggaacttgatcatgtgggccagggccagatagatcagccgcaggtcggccttgtcggtgctgtccaccagtttctttctcaggtggtagatggtggggtacttctcgtggtaggccacctcgtccacgatgttgccgaagatggggtgccgctcgtgcttcttatcctcttccaccaggaaggactcttccagtctgtggaagaagctgtcgtccaccttggccatctcgttgctgaagatctcttgcagatagcagatccggttcttccgtctggtgtatcttcttctggcggttctcttcagccgggtggcctcggctgtttcgccgctgtcgaacagcagggctccgatcaggttcttcttgatgctgtgccggtcggtgttgcccagcaccttgaatttcttgctgggcaccttgtactcgtcggtgatcacggcccagcccacagagttggtgccgatgtccaggccgatgctgtacttcttgtcggctgctgggactccgtggataccgaccttccgcttcttctttggggccatcttatcgtcatcgtctttgtaatcaatatcatgatccttgtagtctccgtcgtggtccttatagtccatggtggccaattcggggccgcggaggctggatcggtcccggtgtcttctatggaggtcaaaacagcgtggatggcgtctccaggcgatctgacggttcactaaacgagctctgcttatataggtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagctcggtaccctggggactttccagctggggactttccagctgggactttccaggag

SEQ ID NO: 10 - DNA, 46 bp, synthetic construct
  source 1..46: mol_type = other DNA
ctggggactttccagcctggggactttccagctgggactttccagg

SEQ ID NO: 11 - DNA, 47 bp, synthetic construct
  source 1..47: mol_type = other DNA
ctggggactttccagctggggactttccagctgggactttccaggag

SEQ ID NO: 12 - DNA, 4272 bp, synthetic construct
  source 1..4272: mol_type = other DNA
atggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagtaa

SEQ ID NO: 13 - protein, 1423 aa, synthetic construct
MDYKDHDGDYKDHDIDYKDDDDKMAPKKKRKVGIHGVPAADKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKK

SEQ ID NO: 14 - DNA, 96 bp, synthetic construct
  source 1..96: mol_type = other DNA
gttcaggatcacgttaccgcgttttagagctagaaatagcaagttaaaataaggctagtccgttatcaacttgaaaaagtggcaccgagtcggtgc

SEQ ID NO: 15 - DNA, 241 bp, synthetic construct
  source 1..241: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggac

SEQ ID NO: 16 - DNA, 20 bp, synthetic construct
  source 1..20: mol_type = other DNA
caagtccatgtgcaatggcg

SEQ ID NO: 17 - DNA, 744 bp, synthetic construct
  source 1..744: mol_type = other DNA
atgtctagactggacaagagcaaagtcataaacggcgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagcaattgagctgttcgaccggcagggagccgaacctgccttccttttcggcctggaactaatcatatgtggcctggagaaacagctaaagtgcgaaagcggcgggccggccgacgcccttgacgattttgacttagacatgctcccagccgatgcccttgacgactttgaccttgatatgctgcctgctgacgctcttgacgattttgaccttgacatgctccccggg

SEQ ID NO: 18 - protein, 248 aa, synthetic construct
MSRLDKSKVINGALELLNGVGIEGLTTRKLAQKLGVEQPTLYWHVKNKRALLDALPIEMLDRHHTHFCPLEGESWQDFLRNNAKSFRCALLSHRDGAKVHLGTRPTEKQYETLENQLAFLCQQGFSLENALYALSAVGHFTLGCVLEEQEHQVAKEERETPTTDSMPPLLRQAIELFDRQGAEPAFLFGLELIICGLEKQLKCESGGPADALDDFDLDMLPADALDDFDLDMLPADALDDFDLDMLPG

SEQ ID NO: 19 - DNA, 846 bp, synthetic construct
  source 1..846: mol_type = other DNA
atgtctagattagataaaagtaaagtgattaacagcgcattagagctgcttaatgaggtcggaatcgaaggtttaacaacccgtaaactcgcccagaagctaggtgtagagcagcctacattgtattggcacgtgcgcaacaagcagactcttatgaacatgctttcagaggcaatactggcgaagcatcacacccgttcagcaccgttaccgactgagagttggcagcagtttctccaggaaaatgctctgagtttccgtaaagcattactggtccatcgtgatggagcccgattgcatatagggacctctcctacgcccccccagtttgaacaagcagaggcgcaactacgctgtctatgcgatgcagggttttcggtcgaggaggctcttttcattctgcaatctatcagccattttacgttgggtgcagtattagaggagcaagcaacaaaccagatagaaaataatcatgtgatagacgctgcaccaccattattacaagaggcatttaatattcaggcgagaacctctgctgaaatggccttccatttcgggctgaaatcattaatatttggattttctgcacagttagatgaaaaaaagcatacacccattgaggatggtaataaaccaaaaaagaagagaaagctagcagtgtcagtgacatttgaagatgtggctgtgctctttactcgggacgagtggaagaagctggatctgtctcagagaagcctgtaccgtgaggtgatgctggagaattacagcaacctggcctccatggcaggattcctgtttaccaaaccaaaggtgatctccctgttgcagcaaggagaggatccctgg

SEQ ID NO: 20 - protein, 282 aa, synthetic construct
MSRLDKSKVINSALELLNEVGIEGLTTRKLAQKLGVEQPTLYWHVRNKQTLMNMLSEAILAKHHTRSAPLPTESWQQFLQENALSFRKALLVHRDGARLHIGTSPTPPQFEQAEAQLRCLCDAGFSVEEALFILQSISHFTLGAVLEEQATNQIENNHVIDAAPPLLQEAFNIQARTSAEMAFHFGLKSLIFGFSAQLDEKKHTPIEDGNKPKKKRKLAVSVTFEDVAVLFTRDEWKKLDLSQRSLYREVMLENYSNLASMAGFLFTKPKVISLLQQGEDPW

SEQ ID NO: 21 - DNA, 330 bp, synthetic construct
  source 1..330: mol_type = other DNA
atggccctgtggatgagactgctgcctctgctggccctcctggccctgtggggccccgaccccgccgccgcttttgtgaaccagcacctgtgcggaagcgacctggtcgaggccctgtacctggtgtgcggcgagcggggcttcttctacacccctagaaccaagcgggaagccgaggatctgcaggtgggccaggtggagctgggcggcggaccaggcgccggctccctgcaacctctggctctggaaggatctagacagaagagaggcatcgtggagcagtgttgtacaagcatctgcagcctgtatcagctggaaaactactgcaat

SEQ ID NO: 22 - protein, 110 aa, synthetic construct
MALWMRLLPLLALLALWGPDPAAAFVNQHLCGSDLVEALYLVCGERGFFYTPRTKREAEDLQVGQVELGGGPGAGSLQPLALEGSRQKRGIVEQCCTSICSLYQLENYCN

SEQ ID NO: 23 - DNA, 424 bp, synthetic construct
  source 1..424: mol_type = other DNA
ggtaccgagctcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgacctatataagcagagctcgtttagtgaaccgtcagatcgcctggagacgccatccacgctgttttgacctccatagaagacaccgggaccgatccagcctccgcggccccgaattg

SEQ ID NO: 24 - DNA, 225 bp, synthetic construct
  source 1..225: mol_type = other DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 25 - DNA, 222 bp, synthetic construct
  source 1..222: mol_type = other DNA
cagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggta

SEQ ID NO: 26 - DNA, 54 bp, Thosea asigna virus
  source 1..54: mol_type = genomic DNA
gagggcaggggaagtcttctaacatgcggggacgtggaggaaaatcccggcccc

SEQ ID NO: 27 - protein, 18 aa, Thosea asigna virus
EGRGSLLTCGDVEENPGP

SEQ ID NO: 28 - DNA, 57 bp, synthetic construct
  source 1..57: mol_type = other DNA
gccacaaacttcagcctgctgaagcaggccggagatgtggaagaaaatcctggcccc

SEQ ID NO: 29 - protein, 19 aa, synthetic construct
ATNFSLLKQAGDVEENPGP

SEQ ID NO: 30 - DNA, 232 bp, synthetic construct
  source 1..232: mol_type = other DNA
ggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagg

SEQ ID NO: 31 - DNA, 6969 bp, synthetic construct
  source 1..6969: mol_type = other DNA
ctggggactttccagcctggggactttccagctgggactttccaggcgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggtcgcaacgggtttgccgccagagtgaacacgccaccatggactggacatggcgggtgttctgcctgctggccgtggcccctggtgctcacagcgaagtgcagctggtggaaagcggcggcggcctggtgcagcccggcggaagcctccgcctgagttgcgctgccagcggcttcaccttcagcgactactggatgtactgggtgcggcaggcccctggcaaaggcctggaatgggtctctgaaatcaacaccaacggccttatcaccaagtaccccgactccgtgaagggccggttcaccatctccagagataatgccaaaaacaccctgtacctgcagatgaacagcctgcggcccgaggacacagccgtgtactactgcgctagaagcccttctggctttaaccggggccagggcacccttgttacagtgtcaagcggcggaggtggctctggcggtggatctgaggtgcagctggttgagagcggaggcggactggtgcaacctggcaatagcctgagactgtcttgtgccgcctctggtttcaccttttctagcttcggcatgagctgggtcagacaggctcctggcaagggcctggaatgggtgtccagcatcagcggaagcggctccgacaccctgtacgccgacagcgtgaaagggagattcacaatcagcagggacaatgccaagaccaccctgtacctgcaaatgaacagcctgagacctgaggacaccgccgtgtactattgcaccatcggcggaagcctgagcagatccagccagggcaccctggtgacagtgtcttctggtggtggcggatcaggaggcgggagcgaggtgcagctggtcgagagcggcggcggactggtgcagccaggcggaagcctgagactgagctgtgccgctagcggcttcacattcagcgattactggatgtactgggtgcggcaggcccctggaaagggcctcgagtgggtctccgagatcaacacaaacggcctgatcacaaagtacccagatagcgtgaagggcagatttacaatttctagagacaacgccaagaacaccctgtatctgcagatgaacagcctgcggcctgaggataccgccgtgtactactgcgccagaagccccagcggattcaacagaggccagggcaccctggtgaccgtgtcctccagaaagaggcggggatctggcgccacaaacttcagcctgctgaagcaggccggagatgtggaagaaaatcctggccccatgagcagactggataagtccaaggtgatcaacagcgccctggaactgctcaacggcgtcggcatcgagggactcaccacccggaagctggcccagaaactgggcgtggaacaacctacactgtactggcacgtgaagaacaaaagagccctgctggatgccctgccaatcgagatgctggaccggcaccatacccacagctgccccctggagggagaaagctggcaggacttcctgagaaacaacgctaagagctaccggtgcgccctgctgagccaccgggacggcgccaaggtgcacttggggaccagacccacggaaaagcaatacgagacactggagaaccagctggccttcctgtgccagcagggcttcagcctggagaatgccctctacgccctgtctgccgtgggccactttaccctgggatgtgtgctggaagagcaggagcaccaggtggccaaggaagagagggaaacccctaccaccgacagcatgcctcctctgctgaagcaagctatcgagctgttcgacagacagggcgccgagcctgctttcctgtttggcctggagctgatcatctgcggcttggagaaacagctgaagtgcgagagcggcggccctaccgacgccctggacgatttcgacctggatatgctgcctgccgacgccctggacgacttcgacctggatatgctgcctgccgatgctctggacgacttcgacctggacatgcttccaggctaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcaggctggggaaaaaaagcggttttaggggattgtaaccccgcagagtcccgcaaactctttattttagtcccttcagggactaaaactctggcggcaaacccgttgcgacgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcaacttgtttattgcagcttataatggttacaaataaagcaatagcatcacaaatttcacaaataaagcatttttttcactgcattctagttgtggtttgtccaaactcatcaatgtatcttatcattattaggcgtagtcgggcacgtcgtaggggtatacttttctctttttctttggaggtccggactttttgaagtcctctctctgccggaactcggcctttgtcacttctcccagggcggacacgatgtacttctcgaacaccttcagattctggatgccgatgctcttggcgatcacttctttctcgttggcgttcttgaacaggatcttctggttcttgctcagtgtctcgaacttgttgtcgtgcttagacacgatcagggacacggtgctgctggtgaaggcgttgtagtagacgaactcgggttcctgcatgtccttggtctggatcaggatcagggagtccttgtacaggctaaagcagaactcgtagttctcgtccatcagaatccagtccttgatctcgcccttcttggaccgggccacggccttgttgggcagcaccttcagggcgaagtccatggtgtagatgggcacggcgtagaacttgttggtctttttgtgcttgaagatgtccacccggaacatgtccccgttcttcacgatcttgccgttcacttttctgatcttgcccagctcgagggccttcagcacgccttctttgccgccgtaggactggtagaactcttcctcttttctgaaggtttcctcgtgcagggcgccagagggcttcttccgctcgggcttggacacgaagatctcgtcgatcttatccagcaccttctgccggaagccgctgaagggctcgaagaacttccgcttgttcttgtagtccagctcagagatcttcttggcgtacagctcggcgctgttgctttcctgttctttcttgaagtcggagaaggccttcacgatgctgttgttggcgtaggcaatgatcacggcgtcgatggcgtggtgcagatggttgttccgatccttggcgctgaagccccaggtgtgtctcagggcgctggtcagcatgccgctcttggcttccacgtgcaccttggagcctttctgggtatcgttcagcttggtgttctcgtcgtcggacaggggcaggaaatccaggtagtcctttgtgtagttcagcaccagccgggcgatataccgggtgtcgttcaggttccggtccttgaagttcttctgctctttgtccttgtaattcttgtccaggattctcttctgtttcttggtgggcaggttcttggccagcacctcgattttctgccacttggcgctgtcgttgccgaaggcctcgaagggggtctggttcagtttttcctggttctgtttggtgaacaccagcaccttgttcatgtagctgtcgtcgaaggaccggctgtaggggtagatgtggtcgatttccagcatcttctcgtcctgcagatcgctgatcttgatcttctcgccgctgtaggcgcagaactctttctgttctttgaacagccgcagcttcaggatgttcttgctgttgatcttcagtcccagcttctcgcattccagctcagcgtccttcttggccttgtagttttcgttctgctctttctcgatcttggcccgctggctgtggttcttgcccacttcccgggccagctcgatgttgatcttgtgcaccttgccgtatttcttgagcagggcattcagcaccttccggtattctttgatggcccgcagcaccacggggttggtcacttcgtccttgtagtaggtttcgttgaaggcgggcaggaagtccttcttgtcctcgttgatggccaccttcaggttcagctcgttgcaggcctcgtcgtacttcttgccttccagcatcaggggggtgaccagcttcagagccttgaagctgatgttcaggtgatccttgaattccagcttgctcaggctgtcgatctggttctgattcaggtcgtatttggccagggccttcttcagcttaatctcgtccttgatcagggtgatgtccttggcgatctcattcagatcgtcctggctcaggttgtgctcgcccagggccttgatgaactctttgtacttcttgaactcgatgaagtaggtgcccttctcgcccttaaactcgtagtcgtcgctcaggcccagcagcttcttggtctgcttgtaggtcagggtgccgttcttcagcacctcattcagcagggcgttcaggtcgtccttggtgtacaggatgccctcggtgttcttcagattgttcagcaggttgatgatccgggtcagggccacgaacatgaaggccagggggctgttcttgggggctctcttctcgtcggtgaagaagctgcagttgcccacgaggtggctgaagtccttcagggctctcttgtagaaggcgacgctcagcacctcttcctcgaatttcttgctgaagctgaagccgaactctctctgcttcttgaagatcagcttcagctcatccttcaggaagctctgggcgatacaccgctcgtagctttctttcttgttccgcacgttggtgaattctttgctgttctctttgaacttctggaagtactctttgtacaggtactcgcccacggactggtagttggccagcttttcctcgttctgcttgatggccttcaggatggcgcccttctctttgtcgtcgctgttcttgatgtcgtcgtagcctctccgcttggcaatgtgcaggatcactctggcgaagtcctgcttgctcagcagctcgttcagggcccggaaccgcagctcgtaggggctgatcaggctgcccttgtaggccttggccagggactcgtcgaagctctggtagtcctcgtagttcagcttgaactcgttggcgatcaggtgcttcaggtggttcagccgggccttccgtctggccagtctcttccgggcgcttctggccagccttctgggcagggccaggctctcgccggtcttggggttttccaccttggtgaagatccgcacgccgcagtccttcagctcgtcgttctcgctgaaggcccagccgatgctgctgatgccgatgtcgaaggccaggattctggccatggtggctttacgagggtaggaagtggtacggaaagttggtataagacaaaagtgttgtggaattgctccaggcgatctgacggttcactaaacgagctctgcttttataggcgcccaccgtacacgcctaaagcttatacgttctctatcactgatagggagtaaactggatatacgttctctatcactgatagggagtaaactgtagatacgttctctatcactgatagggagtaaactggtcatacgttctctatcactgatagggagtaaactccttatacgttctctatcactgatagggagtaaagtctgcatacgttctctatcactgatagggagtaaactcttcatacgttctctatcactgatagggagtaaactggggactttccagctggggactttccagctgggactttccaggag

SEQ ID NO: 32 - DNA, 798 bp, synthetic construct
  source 1..798: mol_type = other DNA
cgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttgg

SEQ ID NO: 33 - DNA, 1146 bp, synthetic construct
  source 1..1146: mol_type = other DNA
atggactggacatggcgggtgttctgcctgctggccgtggcccctggtgctcacagcgaagtgcagctggtggaaagcggcggcggcctggtgcagcccggcggaagcctccgcctgagttgcgctgccagcggcttcaccttcagcgactactggatgtactgggtgcggcaggcccctggcaaaggcctggaatgggtctctgaaatcaacaccaacggccttatcaccaagtaccccgactccgtgaagggccggttcaccatctccagagataatgccaaaaacaccctgtacctgcagatgaacagcctgcggcccgaggacacagccgtgtactactgcgctagaagcccttctggctttaaccggggccagggcacccttgttacagtgtcaagcggcggaggtggctctggcggtggatctgaggtgcagctggttgagagcggaggcggactggtgcaacctggcaatagcctgagactgtcttgtgccgcctctggtttcaccttttctagcttcggcatgagctgggtcagacaggctcctggcaagggcctggaatgggtgtccagcatcagcggaagcggctccgacaccctgtacgccgacagcgtgaaagggagattcacaatcagcagggacaatgccaagaccaccctgtacctgcaaatgaacagcctgagacctgaggacaccgccgtgtactattgcaccatcggcggaagcctgagcagatccagccagggcaccctggtgacagtgtcttctggtggtggcggatcaggaggcgggagcgaggtgcagctggtcgagagcggcggcggactggtgcagccaggcggaagcctgagactgagctgtgccgctagcggcttcacattcagcgattactggatgtactgggtgcggcaggcccctggaaagggcctcgagtgggtctccgagatcaacacaaacggcctgatcacaaagtacccagatagcgtgaagggcagatttacaatttctagagacaacgccaagaacaccctgtatctgcagatgaacagcctgcggcctgaggataccgccgtgtactactgcgccagaagccccagcggattcaacagaggccagggcaccctggtgaccgtgtcctcc

SEQ ID NO: 34 - protein, 382 aa, synthetic construct
MDWTWRVFCLLAVAPGAHSEVQLVESGGGLVQPGGSLRLSCAASGFTFSDYWMYWVRQAPGKGLEWVSEINTNGLITKYPDSVKGRFTISRDNAKNTLYLQMNSLRPEDTAVYYCARSPSGFNRGQGTLVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLVESGGGLVQPGGSLRLSCAASGFTFSDYWMYWVRQAPGKGLEWVSEINTNGLITKYPDSVKGRFTISRDNAKNTLYLQMNSLRPEDTAVYYCARSPSGFNRGQGTLVTVSS

SEQ ID NO: 35 - DNA, 12 bp, synthetic construct
  source 1..12: mol_type = other DNA
agaaagaggcgg

SEQ ID NO: 36 - protein, 4 aa, synthetic construct
RKRR

SEQ ID NO: 37 - DNA, 66 bp, unidentified
  source 1..66: mol_type = genomic DNA; note = porcine teschovirus-1
ggatctggcgccacaaacttcagcctgctgaagcaggccggagatgtggaagaaaatcctggcccc

SEQ ID NO: 38 - protein, 22 aa, unidentified
  source 1..22: note = porcine teschovirus-1
GSGATNFSLLKQAGDVEENPGP

SEQ ID NO: 39 - DNA, 3012 bp, synthetic construct
  source 1..3012: mol_type = other DNA
atggccagaatcctggccttcgacatcggcatcagcagcatcggctgggccttcagcgagaacgacgagctgaaggactgcggcgtgcggatcttcaccaaggtggaaaaccccaagaccggcgagagcctggccctgcccagaaggctggccagaagcgcccggaagagactggccagacggaaggcccggctgaaccacctgaagcacctgatcgccaacgagttcaagctgaactacgaggactaccagagcttcgacgagtccctggccaaggcctacaagggcagcctgatcagcccctacgagctgcggttccgggccctgaacgagctgctgagcaagcaggacttcgccagagtgatcctgcacattgccaagcggagaggctacgacgacatcaagaacagcgacgacaaagagaagggcgccatcctgaaggccatcaagcagaacgaggaaaagctggccaactaccagtccgtgggcgagtacctgtacaaagagtacttccagaagttcaaagagaacagcaaagaattcaccaacgtgcggaacaagaaagaaagctacgagcggtgtatcgcccagagcttcctgaaggatgagctgaagctgatcttcaagaagcagagagagttcggcttcagcttcagcaagaaattcgaggaagaggtgctgagcgtcgccttctacaagagagccctgaaggacttcagccacctcgtgggcaactgcagcttcttcaccgacgagaagagagcccccaagaacagccccctggccttcatgttcgtggccctgacccggatcatcaacctgctgaacaatctgaagaacaccgagggcatcctgtacaccaaggacgacctgaacgccctgctgaatgaggtgctgaagaacggcaccctgacctacaagcagaccaagaagctgctgggcctgagcgacgactacgagtttaagggcgagaagggcacctacttcatcgagttcaagaagtacaaagagttcatcaaggccctgggcgagcacaacctgagccaggacgatctgaatgagatcgccaaggacatcaccctgatcaaggacgagattaagctgaagaaggccctggccaaatacgacctgaatcagaaccagatcgacagcctgagcaagctggaattcaaggatcacctgaacatcagcttcaaggctctgaagctggtcacccccctgatgctggaaggcaagaagtacgacgaggcctgcaacgagctgaacctgaaggtggccatcaacgaggacaagaaggacttcctgcccgccttcaacgaaacctactacaaggacgaagtgaccaaccccgtggtgctgcgggccatcaaagaataccggaaggtgctgaatgccctgctcaagaaatacggcaaggtgcacaagatcaacatcgagctggcccgggaagtgggcaagaaccacagccagcgggccaagatcgagaaagagcagaacgaaaactacaaggccaagaaggacgctgagctggaatgcgagaagctgggactgaagatcaacagcaagaacatcctgaagctgcggctgttcaaagaacagaaagagttctgcgcctacagcggcgagaagatcaagatcagcgatctgcaggacgagaagatgctggaaatcgaccacatctacccctacagccggtccttcgacgacagctacatgaacaaggtgctggtgttcaccaaacagaaccaggaaaaactgaaccagacccccttcgaggccttcggcaacgacagcgccaagtggcagaaaatcgaggtgctggccaagaacctgcccaccaagaaacagaagagaatcctggacaagaattacaaggacaaagagcagaagaacttcaaggaccggaacctgaacgacacccggtatatcgcccggctggtgctgaactacacaaaggactacctggatttcctgcccctgtccgacgacgagaacaccaagctgaacgatacccagaaaggctccaaggtgcacgtggaagccaagagcggcatgctgaccagcgccctgagacacacctggggcttcagcgccaaggatcggaacaaccatctgcaccacgccatcgacgccgtgatcattgcctacgccaacaacagcatcgtgaaggccttctccgacttcaagaaagaacaggaaagcaacagcgccgagctgtacgccaagaagatctctgagctggactacaagaacaagcggaagttcttcgagcccttcagcggcttccggcagaaggtgctggataagatcgacgagatcttcgtgtccaagcccgagcggaagaagccctctggcgccctgcacgaggaaaccttcagaaaagaggaagagttctaccagtcctacggcggcaaagaaggcgtgctgaaggccctcgagctgggcaagatcagaaaagtgaacggcaagatcgtgaagaacggggacatgttccgggtggacatcttcaagcacaaaaagaccaacaagttctacgccgtgcccatctacaccatggacttcgccctgaaggtgctgcccaacaaggccgtggcccggtccaagaagggcgagatcaaggactggattctgatggacgagaactacgagttctgctttagcctgtacaaggactccctgatcctgatccagaccaaggacatgcaggaacccgagttcgtctactacaacgccttcaccagcagcaccgtgtccctgatcgtgtctaagcacgacaacaagttcgagacactgagcaagaaccagaagatcctgttcaagaacgccaacgagaaagaagtgatcgccaagagcatcggcatccagaatctgaaggtgttcgagaagtacatcgtgtccgccctgggagaagtgacaaaggccgagttccggcagagagaggacttcaaaaagtccggacctccaaagaaaaagagaaaagtatacccctacgacgtgcccgactacgcctaa

SEQ ID NO: 40 - protein, 1003 aa, synthetic construct
MARILAFDIGISSIGWAFSENDELKDCGVRIFTKVENPKTGESLALPRRLARSARKRLARRKARLNHLKHLIANEFKLNYEDYQSFDESLAKAYKGSLISPYELRFRALNELLSKQDFARVILHIAKRRGYDDIKNSDDKEKGAILKAIKQNEEKLANYQSVGEYLYKEYFQKFKENSKEFTNVRNKKESYERCIAQSFLKDELKLIFKKQREFGFSFSKKFEEEVLSVAFYKRALKDFSHLVGNCSFFTDEKRAPKNSPLAFMFVALTRIINLLNNLKNTEGILYTKDDLNALLNEVLKNGTLTYKQTKKLLGLSDDYEFKGEKGTYFIEFKKYKEFIKALGEHNLSQDDLNEIAKDITLIKDEIKLKKALAKYDLNQNQIDSLSKLEFKDHLNISFKALKLVTPLMLEGKKYDEACNELNLKVAINEDKKDFLPAFNETYYKDEVTNPVVLRAIKEYRKVLNALLKKYGKVHKINIELAREVGKNHSQRAKIEKEQNENYKAKKDAELECEKLGLKINSKNILKLRLFKEQKEFCAYSGEKIKISDLQDEKMLEIDHIYPYSRSFDDSYMNKVLVFTKQNQEKLNQTPFEAFGNDSAKWQKIEVLAKNLPTKKQKRILDKNYKDKEQKNFKDRNLNDTRYIARLVLNYTKDYLDFLPLSDDENTKLNDTQKGSKVHVEAKSGMLTSALRHTWGFSAKDRNNHLHHAIDAVIIAYANNSIVKAFSDFKKEQESNSAELYAKKISELDYKNKRKFFEPFSGFRQKVLDKIDEIFVSKPERKKPSGALHEETFRKEEEFYQSYGGKEGVLKALELGKIRKVNGKIVKNGDMFRVDIFKHKKTNKFYAVPIYTMDFALKVLPNKAVARSKKGEIKDWILMDENYEFCFSLYKDSLILIQTKDMQEPEFVYYNAFTSSTVSLIVSKHDNKFETLSKNQKILFKNANEKEVIAKSIGIQNLKVFEKYIVSALGEVTKAEFRQREDFKKSGPPKKKRKVYPYDVPDYA

SEQ ID NO: 41 - DNA, 22 bp, synthetic construct
  source 1..22: mol_type = other DNA
tcgcaacgggtttgccgccaga

SEQ ID NO: 42 - DNA, 99 bp, synthetic construct
  source 1..99: mol_type = other DNA
tcgcaacgggtttgccgccagagttttagtccctgaagggactaaaataaagagtttgcgggactctgcggggttacaatcccctaaaaccgctttttt

SEQ ID NO: 43 - DNA, 747 bp, synthetic construct
  source 1..747: mol_type = other DNA
atgagcagactggataagtccaaggtgatcaacagcgccctggaactgctcaacggcgtcggcatcgagggactcaccacccggaagctggcccagaaactgggcgtggaacaacctacactgtactggcacgtgaagaacaaaagagccctgctggatgccctgccaatcgagatgctggaccggcaccatacccacagctgccccctggagggagaaagctggcaggacttcctgagaaacaacgctaagagctaccggtgcgccctgctgagccaccgggacggcgccaaggtgcacttggggaccagacccacggaaaagcaatacgagacactggagaaccagctggccttcctgtgccagcagggcttcagcctggagaatgccctctacgccctgtctgccgtgggccactttaccctgggatgtgtgctggaagagcaggagcaccaggtggccaaggaagagagggaaacccctaccaccgacagcatgcctcctctgctgaagcaagctatcgagctgttcgacagacagggcgccgagcctgctttcctgtttggcctggagctgatcatctgcggcttggagaaacagctgaagtgcgagagcggcggccctaccgacgccctggacgatttcgacctggatatgctgcctgccgacgccctggacgacttcgacctggatatgctgcctgccgatgctctggacgacttcgacctggacatgcttccaggctaa

SEQ ID NO: 44 - protein, 248 aa, synthetic construct
MSRLDKSKVINSALELLNGVGIEGLTTRKLAQKLGVEQPTLYWHVKNKRALLDALPIEMLDRHHTHSCPLEGESWQDFLRNNAKSYRCALLSHRDGAKVHLGTRPTEKQYETLENQLAFLCQQGFSLENALYALSAVGHFTLGCVLEEQEHQVAKEERETPTTDSMPPLLKQAIELFDRQGAEPAFLFGLELIICGLEKQLKCESGGPTDALDDFDLDMLPADALDDFDLDMLPADALDDFDLDMLPG

SEQ ID NO: 45 - DNA, 376 bp, synthetic construct
  source 1..376: mol_type = other DNA
tttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttaggcgtgtacggtgggcgcctataaaagcagagctcgtttagtgaaccgtcagatcgcctggagcaattccacaacacttttgtcttataccaactttccgtaccacttcctaccctcgtaaa

SEQ ID NO: 46 - DNA, 208 bp, synthetic construct
  source 1..208: mol_type = other DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcaggctgggga

SEQ ID NO: 47 - DNA, 122 bp, synthetic construct
  source 1..122: mol_type = other DNA
taagatacattgatgagtttggacaaaccacaactagaatgcagtgaaaaaaatgctttatttgtgaaatttgtgatgctattgctttatttgtaaccattataagctgcaataaacaagtt

SEQ ID NO: 48 - DNA, 5816 bp, synthetic construct
  source 1..5816: mol_type = other DNA
ctggggactttccagcctggggactttccagctgggactttccaggtagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgccaccatggccttcttgtggctgctcagctgctgggccctgctgggcaccacattcggcctgcttgtgcctagagagctgagcggctctagcccagtgctggaggaaacccacccagcccaccagcagggagctagccggcctggacccagagacgcccaggcccaccccggaagacccagagccgtgcctacccagtgcgacgtgcctcccaacagcagattcgactgcgcccctgataaggccattacccaggagcagtgtgaagctagaggctgctgctacatccctgccaagcagggcctgcaaggcgcccagatgggccaaccttggtgcttcttcccaccctcttaccctagctacaagctggaaaacctgagctcttctgagatgggctacaccgccaccctgacaagaaccaccccaacattcttcccaaaagacatcctgaccctgagactggacgtgatgatggaaaccgagaaccggctgcatttcaccattaaggaccccgccaacagacggtacgaggtccccctggaaacaccccacgtgcacagccgggccccttcccctctgtacagtgtggaattcagcgaggaacccttcggcgtgatcgtgagaaggcagctggatggcagagtgctgctcaacaccacagtggctccattattcttcgccgaccagttcctgcagctgagcaccagcctgccctcacagtacatcacaggcctcgccgaacacctgtctcctctcatgctgagcaccagctggacaagaatcacactgtggaaccgggacctggcccccacacctggggctaatctgtacggctcccaccccttctacctggcgctggaagatggcggcagcgcccacggcgttttcctgctgaacagcaatgccatggacgtggtgctgcagcctagccctgccctgagctggcggagcaccggcggaatcctggacgtgtacatcttcctgggacccgagcctaagagcgtggtgcagcaatatctggatgtggtgggctacccctttatgcctccttactggggcctgggttttcacctgtgtagatggggctactcatctaccgcgatcaccagacaggtggttgagaatatgacccgggcccacttccctctcgacgtgcagtggaacgacctggactacatggacagcagaagagatttcacgttcaacaaggacggcttcagggatttccctgctatggtgcaggagctgcaccagggaggcagacggtacatgatgatcgtggatcctgctatcagcagcagcggccccgccggcagctatagaccttatgacgagggactgagacggggcgttttcatcacaaacgagacaggacagcctcttatcggcaaggtgtggcccggctctaccgcctttcctgacttcacaaatcctaccgccctggcctggtgggaagatatggtcgccgagttccacgaccaggtccctttcgacgggatgtggatcgacatgaacgagcccagtaacttcattagaggctctgaagatggatgtcctaacaacgagctggagaaccctccttacgtgcctggcgtggtcggcggaactctgcaggccgctacaatctgtgccagctcccaccagttcctgagcacccactacaacctgcacaatctgtacggactgacagaggctatcgccagtcatagagccctggtcaaggccagaggcaccagacccttcgtgatctccagaagcaccttcgccggacacggcagatatgccggccactggacaggcgacgtgtggtcctcttgggagcagctcgcaagctctgtgccggaaatcctgcaatttaacttgctgggcgtgcctctggtgggcgctgatgtgtgcggctttctgggcaacacatctgaagaattgtgcgtgcggtggacccagctgggcgctttttacccttttatgagaaatcacaactccctgctgagcctgccacaggagccttacagcttcagcgaacctgcccaacaggccatgcggaaggccttaacactgcgatacgcactgctgcctcacctgtacaccctgttccaccaggctcatgtggccggcgaaacagtggccagaccactgttcctggagttccccaaggatagctccacatggaccgtggaccaccagctgctgtggggcgaggccctgctgatcacccctgtgctgcaggccggcaaagctgaggtgaccggctacttccctctgggcacctggtacgacctgcagaccgtgcctgttgaggctctgggatctctgccccccccacctgccgcccctcgcgagcctgctatccactctgaaggccagtgggtgacactgcctgccccactcgacaccatcaacgtgcacctgagagccggttacatcatccccctgcagggccccggcctgaccaccaccgaaagccggcagcaacctatggccctcgccgtggccctgaccaagggcggcgaggccaggggtgagctattttgggacgacggcgagagcctggaggtgctggagagaggcgcctacacacaagttatcttcctggctcggaacaacaccatcgtgaacgagctggtgcgggtgacaagcgagggcgccggcctccagctgcagaaagtgacggtgctgggcgtggccacagcccctcagcaggtgctgagcaacggcgtgccagtcagcaatttcacctacagccctgataccaaggttctggacatctgcgtgagcctgctcatgggcgaacagttcctggtgtcctggtgcggatctggcgccacaaacttcagcctgctgaagcaggccggagatgtggaagaaaatcctggccccatgagcagactggataagtccaaggtgatcaacagcgccctggaactgctcaacggcgtcggcatcgagggactcaccacccggaagctggcccagaaactgggcgtggaacaacctacactgtactggcacgtgaagaacaaaagagccctgctggatgccctgccaatcgagatgctggaccggcaccatacccacagctgccccctggagggagaaagctggcaggacttcctgagaaacaacgctaagagctaccggtgcgccctgctgagccaccgggacggcgccaaggtgcacttggggaccagacccacggaaaagcaatacgagacactggagaaccagctggccttcctgtgccagcagggcttcagcctggagaatgccctctacgccctgtctgccgtgggccactttaccctgggatgtgtgctggaagagcaggagcaccaggtggccaaggaagagagggaaacccctaccaccgacagcatgcctcctctgctgaagcaagctatcgagctgttcgacagacagggcgccgagcctgctttcctgtttggcctggagctgatcatctgcggcttggagaaacagctgaagtgcgagagcggcggccctaccgacgccctggacgatttcgacctggatatgctgcctgccgacgccctggacgacttcgacctggatatgctgcctgccgatgctctggacgacttcgacctggacatgcttccaggctaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcaggctggggacaacttgtttattgcagcttataatggttacaaataaagcaatagcatcacaaatttcacaaataaagcatttttttcactgcattctagttgtggtttgtccaaactcatcaatgtatcttatttgtttgtttgagcgcgtttcgtcctatttgggactcatcagctggatgtagtaggtggtcgtattcttcacctctccagatcgaaatctggtatgttttaccacgaatccaggacgcgcctttgtttgtttgtcattattagagctttaaatctctgtaggtagtttgtccaattatgtcacaccacagaagtaaggttccttcacaaagagatcgcctgacacgatttcctgcacaggcttgagccatatactcatacatcgcatcttggccacgttttccacgggtttcaaaattaatctcaagttctacgcttaacgctttcgcctgttcccagttattaatatattcaacgctagaactcccctcagcgaagggaaggctgagcactacacgcgaagcaccatcaccgaaccttttgataaactcttccgttccgacttgctccatcaacggttcagtgagacttaaacctaactctttcttaatagtttcggcattatccacttttagtgcgagaaccttcgtcagtcctggatacgtcactttgaccacgcctccagcttttccagagagcgggttttcattatctacagagtatcccgcagcgtcgtatttattgtcggtactataaaaccctttccaatcatcgtcataatttccttgtgtaccagattttggcttttgtatacctttttgaatggaatctacataaccaggtttagtcccgtggtacgaagaaaagttttccatcacaaaagatttagaagaatcaacaacatcatcaggatccatggtggctttacgagggtaggaagtggtacggaaagttggtataagacaaaagtgttgtggaattgctccaggcgatctgacggttcactaaacgagctctgcttttataggcgcccaccgtacacgcctaaagcttatacgttctctatcactgatagggagtaaactggatatacgttctctatcactgatagggagtaaactgtagatacgttctctatcactgatagggagtaaactggtcatacgttctctatcactgatagggagtaaactccttatacgttctctatcactgatagggagtaaagtctgcatacgttctctatcactgatagggagtaaactcttcatacgttctctatcactgatagggagtaaactggggactttccagctggggactttccagctgggactttccaggag

SEQ ID NO: 49 - DNA, 589 bp, synthetic construct
  source 1..589: mol_type = other DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatc

SEQ ID NO: 50 - DNA, 2808 bp, synthetic construct
  source 1..2808: mol_type = other DNA
atggccttcttgtggctgctcagctgctgggccctgctgggcaccacattcggcctgcttgtgcctagagagctgagcggctctagcccagtgctggaggaaacccacccagcccaccagcagggagctagccggcctggacccagagacgcccaggcccaccccggaagacccagagccgtgcctacccagtgcgacgtgcctcccaacagcagattcgactgcgcccctgataaggccattacccaggagcagtgtgaagctagaggctgctgctacatccctgccaagcagggcctgcaaggcgcccagatgggccaaccttggtgcttcttcccaccctcttaccctagctacaagctggaaaacctgagctcttctgagatgggctacaccgccaccctgacaagaaccaccccaacattcttcccaaaagacatcctgaccctgagactggacgtgatgatggaaaccgagaaccggctgcatttcaccattaaggaccccgccaacagacggtacgaggtccccctggaaacaccccacgtgcacagccgggccccttcccctctgtacagtgtggaattcagcgaggaacccttcggcgtgatcgtgagaaggcagctggatggcagagtgctgctcaacaccacagtggctccattattcttcgccgaccagttcctgcagctgagcaccagcctgccctcacagtacatcacaggcctcgccgaacacctgtctcctctcatgctgagcaccagctggacaagaatcacactgtggaaccgggacctggcccccacacctggggctaatctgtacggctcccaccccttctacctggcgctggaagatggcggcagcgcccacggcgttttcctgctgaacagcaatgccatggacgtggtgctgcagcctagccctgccctgagctggcggagcaccggcggaatcctggacgtgtacatcttcctgggacccgagcctaagagcgtggtgcagcaatatctggatgtggtgggctacccctttatgcctccttactggggcctgggttttcacctgtgtagatggggctactcatctaccgcgatcaccagacaggtggttgagaatatgacccgggcccacttccctctcgacgtgcagtggaacgacctggactacatggacagcagaagagatttcacgttcaacaaggacggcttcagggatttccctgctatggtgcaggagctgcaccagggaggcagacggtacatgatgatcgtggatcctgctatcagcagcagcggccccgccggcagctatagaccttatgacgagggactgagacggggcgttttcatcacaaacgagacaggacagcctcttatcggcaaggtgtggcccggctctaccgcctttcctgacttcacaaatcctaccgccctggcctggtgggaagatatggtcgccgagttccacgaccaggtccctttcgacgggatgtggatcgacatgaacgagcccagtaacttcattagaggctctgaagatggatgtcctaacaacgagctggagaaccctccttacgtgcctggcgtggtcggcggaactctgcaggccgctacaatctgtgccagctcccaccagttcctgagcacccactacaacctgcacaatctgtacggactgacagaggctatcgccagtcatagagccctggtcaaggccagaggcaccagacccttcgtgatctccagaagcaccttcgccggacacggcagatatgccggccactggacaggcgacgtgtggtcctcttgggagcagctcgcaagctctgtgccggaaatcctgcaatttaacttgctgggcgtgcctctggtgggcgctgatgtgtgcggctttctgggcaacacatctgaagaattgtgcgtgcggtggacccagctgggcgctttttacccttttatgagaaatcacaactccctgctgagcctgccacaggagccttacagcttcagcgaacctgcccaacaggccatgcggaaggccttaacactgcgatacgcactgctgcctcacctgtacaccctgttccaccaggctcatgtggccggcgaaacagtggccagaccactgttcctggagttccccaaggatagctccacatggaccgtggaccaccagctgctgtggggcgaggccctgctgatcacccctgtgctgcaggccggcaaagctgaggtgaccggctacttccctctgggcacctggtacgacctgcagaccgtgcctgttgaggctctgggatctctgccccccccacctgccgcccctcgcgagcctgctatccactctgaaggccagtgggtgacactgcctgccccactcgacaccatcaacgtgcacctgagagccggttacatcatccccctgcagggccccggcctgaccaccaccgaaagccggcagcaacctatggccctcgccgtggccctgaccaagggcggcgaggccaggggtgagctattttgggacgacggcgagagcctggaggtgctggagagaggcgcctacacacaagttatcttcctggctcggaacaacaccatcgtgaacgagctggtgcgggtgacaagcgagggcgccggcctccagctgcagaaagtgacggtgctgggcgtggccacagcccctcagcaggtgctgagcaacggcgtgccagtcagcaatttcacctacagccctgataccaaggttctggacatctgcgtgagcctgctcatgggcgaacagttcctggtgtcctggtgctaa

SEQ ID NO: 51 - protein, 935 aa, synthetic construct
MAFLWLLSCWALLGTTFGLLVPRELSGSSPVLEETHPAHQQGASRPGPRDAQAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 52 - DNA, 657 bp, unidentified
  source 1..657: mol_type = genomic DNA; note = Diptheria toxin subunit A
atggatcctgatgatgttgttgattcttctaaatcttttgtgatggaaaacttttcttcgtaccacgggactaaacctggttatgtagattccattcaaaaaggtatacaaaagccaaaatctggtacacaaggaaattatgacgatgattggaaagggttttatagtaccgacaataaatacgacgctgcgggatactctgtagataatgaaaacccgctctctggaaaagctggaggcgtggtcaaagtgacgtatccaggactgacgaaggttctcgcactaaaagtggataatgccgaaactattaagaaagagttaggtttaagtctcactgaaccgttgatggagcaagtcggaacggaagagtttatcaaaaggttcggtgatggtgcttcgcgtgtagtgctcagccttcccttcgctgaggggagttctagcgttgaatatattaataactgggaacaggcgaaagcgttaagcgtagaacttgagattaattttgaaacccgtggaaaacgtggccaagatgcgatgtatgagtatatggctcaagcctgtgcaggaaatcgtgtcaggcgatctctttgtgaaggaaccttacttctgtggtgtgacataattggacaaactacctacagagatttaaagctctaa

SEQ ID NO: 53 - protein, 218 aa, unidentified
  source 1..218: note = Diptheria toxin subunit A
MDPDDVVDSSKSFVMENFSSYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSLCEGTLLLWCDIIGQTTYRDLKL

SEQ ID NO: 54 - DNA, 110 bp, synthetic construct
  source 1..110: mol_type = other DNA
ggcgcgtcctggattcgtggtaaaacataccagatttcgatctggagaggtgaagaatacgaccacctactacatccagctgatgagtcccaaataggacgaaacgcgct

SEQ ID NO: 55 - DNA, 12 bp, synthetic construct
  source 1..12: mol_type = other DNA
caaacaaacaaa

SEQ ID NO: 56 - DNA, 3538 bp, synthetic construct
  source 1..3538: mol_type = other DNA
ctggggactttccagcctggggactttccagctgggactttccaggcgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggataacttcgtatagcatacattatacgaagttatgttggacgcgtcgctagcgccaccatggccacagacagcagaacaagctggctgctcaccgtgtccctgctgtgcctgctgtggccccaggaggccagcgccttccccgctatgcctctgagcagcctgttcagcaacgccgtgctgagagccagaggcaagcggcacggcgagggcaccttcacctctgatctgtccaagcagatggaagaggaagctgtgcggctgtttatcgagtggctgaaaaacggcggaccttctagcggcgcccctccacctagctaatgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcaggctggggagggccgccgatcgaacttgtttattgcagcttataatggttacaaataaagcaatagcatcacaaatttcacaaataaagcatttttttcactgcattctagttgtggtttgtccaaactcatcaatgtatcttatttgtttgtttgagcgcgtttcgtcctatttgggactcatcagctggatgtagtaggtggtcgtattcttcacctctccagatcgaaatctggtatgttttaccacgaatccaggacgcgcctttgtttgtttggactagaagcggccgcctactaatcgccatcttccagcaggcgcaccattgcccctgtttcactatccaggttacggatatagttcatgacaatatttacattggtccagccaccagcttgcatgatctccggtattgaaactccagcgcgggccatatctcgcgcggctccgacacgggcactgtgtccagaccaggccaggtatctctgaccagagtcatccttagcgccgtaaatcaatcgatgagttgcttcaaaaatcccttccagggcgcgagttgatagctggctggtggcagatggcgcggcaacaccattttttctgacccggcaaaacaggtagttattcggatcatcagctacaccagagacggaaatccatcgctcgaccagtttagttacccccaggctaagtgccttctctacacctgcggtgctaaccagcgttttcgttctgccaatatggattaacattctcccaccgtcagtacgtgagatatctttaaccctgatcctggcaatttcggctatacgtaacagggtgttataagcaatccccagaaatgccagattacgtatatcctggcagcgatcgctattttccatgagtgaacgaacctggtcgaaatcagtgcgttcgaacgctagagcctgttttgcacgttcaccggcatcaacgttttcttttcggatccgccgcataaccagtgaaacagcattgctgtcacttggtcgtggcagcccggaccgacgatgaagcatgtttagctggcccaaatgttgctggatagtttttactgccagaccgcgcgcctgaagatatagaagataatcgcgaacatcttcaggttctgcgggaaaccatttccggttattcaacttgcaccatgccgcccacgaccggcaaacggacagaagcattttccaggtatgctcagaaaacgcctggcgatccctgaacatgtccatcaggttcttgcgaacctcatcactcgttgcatcgaccggtaatgcaggcaaattttggtgtacggtcagtaaattggacaccttcctcttcttcttaggcgcgcctctcctttccaggatctcaaagcaataactatgagaattctgagcatcggcgtcgtgaaactcggagaacacgctttcccaatcgtctggctcataatcaggaaaatgggtgtccccttccacctcagcgtcaatgtgagtcaggtacagtttctgggccttaggcaggaactgctcgatcactctccctccgccgatgaccatgatctcgggcacatctccgcaagcggcaatggcttcgtccacacttttcacccatgtcaccctgtcgtcggtagagggctgggagctcagaatgatgttcttgcgtccaggcaggggccggccaatggactcccaagtgtgtctgcccatgatcactggcttgttcagggtgttcctcttgaaccaagccagatcggcgggcagattccatggcatagcgttttccatcccgatcacgtagtccacggccagagcggcaatcagagagatcatggtggcagcctgcttttttgtacaaacttgataacttcgtatagcatacattatacgaagttatcctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcaccggatcaattgccgacccctccccccaacttctcggggactgtgggcgatgtgcgctctgcccactgacgggcaccggagccctggggactttccagctggggactttccagctgggactttccaggag

SEQ ID NO: 57 - DNA, 34 bp, synthetic construct
  source 1..34: mol_type = other DNA
ataacttcgtatagcatacattatacgaagttat

SEQ ID NO: 58 - DNA, 261 bp, synthetic construct
  source 1..261: mol_type = other DNA
atggccacagacagcagaacaagctggctgctcaccgtgtccctgctgtgcctgctgtggccccaggaggccagcgccttccccgctatgcctctgagcagcctgttcagcaacgccgtgctgagagccagaggcaagcggcacggcgagggcaccttcacctctgatctgtccaagcagatggaagaggaagctgtgcggctgtttatcgagtggctgaaaaacggcggaccttctagcggcgcccctccacctagctaa

SEQ ID NO: 59 - protein, 86 aa, synthetic construct
MATDSRTSWLLTVSLLCLLWPQEASAFPAMPLSSLFSNAVLRARGKRHGEGTFTSDLSKQMEEEAVRLFIEWLKNGGPSSGAPPPS

SEQ ID NO: 60 - DNA, 1533 bp, synthetic construct
  source 1..1533: mol_type = other DNA
atgatctctctgattgccgctctggccgtggactacgtgatcgggatggaaaacgctatgccatggaatctgcccgccgatctggcttggttcaagaggaacaccctgaacaagccagtgatcatgggcagacacacttgggagtccattggccggcccctgcctggacgcaagaacatcattctgagctcccagccctctaccgacgacagggtgacatgggtgaaaagtgtggacgaagccattgccgcttgcggagatgtgcccgagatcatggtcatcggcggagggagagtgatcgagcagttcctgcctaaggcccagaaactgtacctgactcacattgacgctgaggtggaaggggacacccattttcctgattatgagccagacgattgggaaagcgtgttctccgagtttcacgacgccgatgctcagaattctcatagttattgctttgagatcctggaaaggagaggcgcgcctaagaagaagaggaaggtgtccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattag

SEQ ID NO: 61 - protein, 510 aa, synthetic construct
MISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERRGAPKKKRKVSNLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGD

SEQ ID NO: 62 - DNA, 6127 bp, synthetic construct
  source 1..6127: mol_type = other DNA
ctggggactttccagcctggggactttccagctgggactttccaggcgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttgggccaccatggccttcttgtggctgctcagctgctgggccctgctgggcaccacattcggcctgcttgtgcctagagagctgagcggctctagcccagtgctggaggaaacccacccagcccaccagcagggagctagccggcctggacccagagacgcccaggcccaccccggaagacccagagccgtgcctacccagtgcgacgtgcctcccaacagcagattcgactgcgcccctgataaggccattacccaggagcagtgtgaagctagaggctgctgctacatccctgccaagcagggcctgcaaggcgcccagatgggccaaccttggtgcttcttcccaccctcttaccctagctacaagctggaaaacctgagctcttctgagatgggctacaccgccaccctgacaagaaccaccccaacattcttcccaaaagacatcctgaccctgagactggacgtgatgatggaaaccgagaaccggctgcatttcaccattaaggaccccgccaacagacggtacgaggtccccctggaaacaccccacgtgcacagccgggccccttcccctctgtacagtgtggaattcagcgaggaacccttcggcgtgatcgtgagaaggcagctggatggcagagtgctgctcaacaccacagtggctccattattcttcgccgaccagttcctgcagctgagcaccagcctgccctcacagtacatcacaggcctcgccgaacacctgtctcctctcatgctgagcaccagctggacaagaatcacactgtggaaccgggacctggcccccacacctggggctaatctgtacggctcccaccccttctacctggcgctggaagatggcggcagcgcccacggcgttttcctgctgaacagcaatgccatggacgtggtgctgcagcctagccctgccctgagctggcggagcaccggcggaatcctggacgtgtacatcttcctgggacccgagcctaagagcgtggtgcagcaatatctggatgtggtgggctacccctttatgcctccttactggggcctgggttttcacctgtgtagatggggctactcatctaccgcgatcaccagacaggtggttgagaatatgacccgggcccacttccctctcgacgtgcagtggaacgacctggactacatggacagcagaagagatttcacgttcaacaaggacggcttcagggatttccctgctatggtgcaggagctgcaccagggaggcagacggtacatgatgatcgtggatcctgctatcagcagcagcggccccgccggcagctatagaccttatgacgagggactgagacggggcgttttcatcacaaacgagacaggacagcctcttatcggcaaggtgtggcccggctctaccgcctttcctgacttcacaaatcctaccgccctggcctggtgggaagatatggtcgccgagttccacgaccaggtccctttcgacgggatgtggatcgacatgaacgagcccagtaacttcattagaggctctgaagatggatgtcctaacaacgagctggagaaccctccttacgtgcctggcgtggtcggcggaactctgcaggccgctacaatctgtgccagctcccaccagttcctgagcacccactacaacctgcacaatctgtacggactgacagaggctatcgccagtcatagagccctggtcaaggccagaggcaccagacccttcgtgatctccagaagcaccttcgccggacacggcagatatgccggccactggacaggcgacgtgtggtcctcttgggagcagctcgcaagctctgtgccggaaatcctgcaatttaacttgctgggcgtgcctctggtgggcgctgatgtgtgcggctttctgggcaacacatctgaagaattgtgcgtgcggtggacccagctgggcgctttttacccttttatgagaaatcacaactccctgctgagcctgccacaggagccttacagcttcagcgaacctgcccaacaggccatgcggaaggccttaacactgcgatacgcactgctgcctcacctgtacaccctgttccaccaggctcatgtggccggcgaaacagtggccagaccactgttcctggagttccccaaggatagctccacatggaccgtggaccaccagctgctgtggggcgaggccctgctgatcacccctgtgctgcaggccggcaaagctgaggtgaccggctacttccctctgggcacctggtacgacctgcagaccgtgcctgttgaggctctgggatctctgccccccccacctgccgcccctcgcgagcctgctatccactctgaaggccagtgggtgacactgcctgccccactcgacaccatcaacgtgcacctgagagccggttacatcatccccctgcagggccccggcctgaccaccaccgaaagccggcagcaacctatggccctcgccgtggccctgaccaagggcggcgaggccaggggtgagctattttgggacgacggcgagagcctggaggtgctggagagaggcgcctacacacaagttatcttcctggctcggaacaacaccatcgtgaacgagctggtgcgggtgacaagcgagggcgccggcctccagctgcagaaagtgacggtgctgggcgtggccacagcccctcagcaggtgctgagcaacggcgtgccagtcagcaatttcacctacagccctgataccaaggttctggacatctgcgtgagcctgctcatgggcgaacagttcctggtgtcctggtgcggatctggcgccacaaacttcagcctgctgaagcaggccggagatgtggaagaaaatcctggccccatgagcagactggataagtccaaggtgatcaacagcgccctggaactgctcaacggcgtcggcatcgagggactcaccacccggaagctggcccagaaactgggcgtggaacaacctacactgtactggcacgtgaagaacaaaagagccctgctggatgccctgccaatcgagatgctggaccggcaccatacccacagctgccccctggagggagaaagctggcaggacttcctgagaaacaacgctaagagctaccggtgcgccctgctgagccaccgggacggcgccaaggtgcacttggggaccagacccacggaaaagcaatacgagacactggagaaccagctggccttcctgtgccagcagggcttcagcctggagaatgccctctacgccctgtctgccgtgggccactttaccctgggatgtgtgctggaagagcaggagcaccaggtggccaaggaagagagggaaacccctaccaccgacagcatgcctcctctgctgaagcaagctatcgagctgttcgacagacagggcgccgagcctgctttcctgtttggcctggagctgatcatctgcggcttggagaaacagctgaagtgcgagagcggcggccctaccgacgccctggacgatttcgacctggatatgctgcctgccgacgccctggacgacttcgacctggatatgctgcctgccgatgctctggacgacttcgacctggacatgcttccaggctaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcaggctggggacaacttgtttattgcagcttataatggttacaaataaagcaatagcatcacaaatttcacaaataaagcatttttttcactgcattctagttgtggtttgtccaaactcatcaatgtatcttatttgtttgtttgagcgcgtttcgtcctatttgggactcatcagctggatgtagtaggtggtcgtattcttcacctctccagatcgaaatctggtatgttttaccacgaatccaggacgcgcctttgtttgtttgtcattattaattgggggggttgcacacaaacagcatcagggccagcacggccacggtaggatggctcaggctgtcgacgataacaggctcgttgcggatgttggtcagggtcacctggtttgggaatgtatcttgcacgctctcctgcacgcctctggacaggttgtcccagttgttttccaggctgatcatagcagcgtcaggctgaaaggcggtgccggtctggatggacacccgcactctattagagatgtaccggaatctggcggcttcagccaccatttggataatcacgatcagtgttctggccttttcctcgttgtcatttccgccgctccggaagaaggagattccgtgtgtaagagcttgcaggcccagggggatctgctgtctgctctggtgggcccatctttccaggtcgccgtatgtgccgtagaagggcagagagtgctgatcggtgccggtgaacaggtagtcagaggcgctgctaggggcgtcccgcaggaagtaagactgggtgccggctctgtaggccaccacataggcgtttgtcacgtcaatgcccacctcgatgctctctgtatcgctgttgctcagctcgacggtgatgtatctgtttctctcctgcagggttgttggatcaggcagcacagggatgtcgtggatcagtccacctctgagccgttccctcagggcctcgatgaactgcttgtagctctggctggtggcgccctcggtgctgaatttgataggtctatcttccatggtggctttacgagggtaggaagtggtacggaaagttggtataagacaaaagtgttgtggaattgctccaggcgatctgacggttcactaaacgagctctgcttttataggcgcccaccgtacacgcctaaagcttatacgttctctatcactgatagggagtaaactggatatacgttctctatcactgatagggagtaaactgtagatacgttctctatcactgatagggagtaaactggtcatacgttctctatcactgatagggagtaaactccttatacgttctctatcactgatagggagtaaagtctgcatacgttctctatcactgatagggagtaaactcttcatacgttctctatcactgatagggagtaaactggggactttccagctggggactttccagctgggactttccaggag

SEQ ID NO: 63 - DNA, 759 bp, synthetic construct
  source 1..759: mol_type = other DNA
atggaagatagacctatcaaattcagcaccgagggcgccaccagccagagctacaagcagttcatcgaggccctgagggaacggctcagaggtggactgatccacgacatccctgtgctgcctgatccaacaaccctgcaggagagaaacagatacatcaccgtcgagctgagcaacagcgatacagagagcatcgaggtgggcattgacgtgacaaacgcctatgtggtggcctacagagccggcacccagtcttacttcctgcgggacgcccctagcagcgcctctgactacctgttcaccggcaccgatcagcactctctgcccttctacggcacatacggcgacctggaaagatgggcccaccagagcagacagcagatccccctgggcctgcaagctcttacacacggaatctccttcttccggagcggcggaaatgacaacgaggaaaaggccagaacactgatcgtgattatccaaatggtggctgaagccgccagattccggtacatctctaatagagtgcgggtgtccatccagaccggcaccgcctttcagcctgacgctgctatgatcagcctggaaaacaactgggacaacctgtccagaggcgtgcaggagagcgtgcaagatacattcccaaaccaggtgaccctgaccaacatccgcaacgagcctgttatcgtcgacagcctgagccatcctaccgtggccgtgctggccctgatgctgtttgtgtgcaacccccccaattaa

SEQ ID NO: 64 - protein, 252 aa, synthetic construct
MEDRPIKFSTEGATSQSYKQFIEALRERLRGGLIHDIPVLPDPTTLQERNRYITVELSNSDTESIEVGIDVTNAYVVAYRAGTQSYFLRDAPSSASDYLFTGTDQHSLPFYGTYGDLERWAHQSRQQIPLGLQALTHGISFFRSGGNDNEEKARTLIVIIQMVAEAARFRYISNRVRVSIQTGTAFQPDAAMISLENNWDNLSRGVQESVQDTFPNQVTLTNIRNEPVIVDSLSHPTVAVLALMLFVCNPPN

SEQ ID NO: 65 - DNA, 4424 bp, synthetic construct
  source 1..4424: mol_type = other DNA
ctggggactttccagcctggggactttccagctgggactttccaggcgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttgggttggacgcgtctggcgacagacagccggcgctagcgccaccatgaaaatcatcctgtggctgtgcgtgttcggcctctttctggccaccctgttccccatcagctggcagatgcctgtggaatctggcctgagctctgaggatagcgccagcagcgagagcttcgccaagagaatcaagcggcacggcgagggaacattcaccagcgacgtgtcctcctacctggaaggccaggccgctcaggagttcatcgcctggctggtggacggcagaggctaatgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcaggctggggagggccgccgatcgaacttgtttattgcagcttataatggttacaaataaagcaatagcatcacaaatttcacaaataaagcatttttttcactgcattctagttgtggtttgtccaaactcatcaatgtatcttatttgtttgtttgagcgcgtttcgtcctatttgggactcatcagctggatgtagtaggtggtcgtattcttcacctctccagatcgaaatctggtatgttttaccacgaatccaggacgcgcctttgtttgtttggactagaagcggccgcctattattcctcgccctggcctccctccttggactcgcacagtctctctgggatgtggctgatgttccgtctcgggatcttgaaaggatcctcgatgttctgatcctcccactgtttgccgacgctagccaggtttctgaaggtttctctcatcacgtctctgtacaggttcttttggctagggtcgagcagagcccactcttccagagtgaaattcacagccacgtcctcgaaggccacgctgtcgcttcctccgccgccagagccgccgccaccgctgctggtcttcttgcctgtgtgtgtgcgctggtgtgtggtgaggtggtcgcttcttgaaaagctttttccacactcaggacacttatagggcttctctccggtgtgtgttctctggtgctctgtcagatgaccgctctgggaaaagctcttgccgcattctgggcacttataaggcttttcgccggtgtgggtcctttggtgctcggtcaggttatcggctctggagaagctcttgccgcattcgggacacttataaggtttctcgcctgtgtgggtccgctggtgtcttgtcaggtcggcaggagagctgaaggattttccgcactcagggcacttgtaaggtttttcgcctgtatgggttctctggtgccgggccagatctctgcaatcggagaagctcttgccacattcaggacatttgtagggtttctcaccggtatgggtccgctggtgccgcaccaggtggccagggtcgctaaaagatttgccgcactctgggcacttgtagggcttctcgccaggctccaggctgccgcctccgccgctcccgcctccgccgctcaggggcaggctattttgggagaagtatttgaagtactctctaagtggcagcaggcagtttttcaccagcaggtcgaccttaggggcatccagcttggatctgcttctcacctgagcttgcaggtactcttcttccttaggtgtcagaggggcgtgcttggacttcaggccagggatattgctccacacccgcatggcattctggtagtcgcggccccggacatcctgcagggtcacggcctcggtctgcaggaatcttgtggttgtctcctggtcgtcctcggtcagcagcaggttgtccatgaagatccagaagaaaggtctctggctttcctgccgaggcagggcgtactgcaggatacggtggaattgaaacatgtaccagccggggcaccggtcgcagctgctgcccaggggctgggtgctgccgtacaccagatcgaaaggtccccatttctcgacatctctccgcaccacgtttgtcacatcttccacgtacttcagggtgccgccgccagacccgcttccagattccagaaatcccaggctcttcagcaccttgtcgatgtttctgaacaggctcagcactctcacaggctgcctcttccaagccgacactgtcttgtatatttccataggtcccatgtggctgcctctcaggctgagaggcaccagtccgctgctgaaggaggggccgcgggagttggcgttagaatttccggagctcacgcaggcaaagtactccttcaggggggcgaacaggtgtctgatcacaggcacgctccaggatctgcccagcagccgctgtctggccagtctgctcatgttagaaacgtcggtgtagtgcacggggaagccaaaaactctttccatctcggtgcaccacaggatgtcctccttctcgttcataaacacagggaagtgctgatctttgccctgcttgatgctgttgctcctggtggtgatggttctcaccttactgaacttggcgattctgccgtgttccagacactcctgcagttccagcttgtcattcacggtagaggcgagaggtctgttcatgcctggcaggttgccccagaagtaccgggcccggtgggcggcagacacttccttagcatcaatcatcacagggttagactccaggaatctgctgatgtcccgtttgtcgctcacgcccatggccaccacgttctcgaacagccagaaaaaaggtctgtcatcgccttccttgggacgggcatcgtgcagcagccggtagaactcgaagaacagtctgcctgtgccctcgtacaggccctttctagcggggttcacgatgctcaggtcgttgcaggggcttccgccgataaccaggtcgaaggggccccactcctggatatgcttctgggtcacagaccggacgtcgcccacgtacatgatcttgccttggtgccgcaccatgcccacggtgatagaatcttcgcacacttcactggcgatgtatctgtccacctggatgcccaggtccttcagcaccagcaggccggtggcgatgccgtcaaagaggctcagcacccggattggcttccgcttctcggctggcacaggagggtacaccttaggtgggtcgaactcctggtcgtggttgacctttctcttttttttaggggccatggtggcagcctgcttttttgtacaaacttgcctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcaccggatcaattgccgacccctccccccaacttctcggggactgtgggcgatgtgcgctctgcccactgacgggcaccggagccctggggactttccagctggggactttccagctgggactttccaggag

SEQ ID NO: 66 - DNA, 18 bp, synthetic construct
  source 1..18: mol_type = other DNA
tggcgacagacagccggc

SEQ ID NO: 67 - DNA, 237 bp, synthetic construct
  source 1..237: mol_type = other DNA
atgaaaatcatcctgtggctgtgcgtgttcggcctctttctggccaccctgttccccatcagctggcagatgcctgtggaatctggcctgagctctgaggatagcgccagcagcgagagcttcgccaagagaatcaagcggcacggcgagggaacattcaccagcgacgtgtcctcctacctggaaggccaggccgctcaggagttcatcgcctggctggtggacggcagaggctaa

SEQ ID NO: 68 - protein, 78 aa, synthetic construct
MKIILWLCVFGLFLATLFPISWQMPVESGLSSEDSASSESFAKRIKRHGEGTFTSDVSSYLEGQAAQEFIAWLVDGRG

SEQ ID NO: 69 - DNA, 2493 bp, synthetic construct
  source 1..2493: mol_type = other DNA
atggcccctaaaaaaaagagaaaggtcaaccacgaccaggagttcgacccacctaaggtgtaccctcctgtgccagccgagaagcggaagccaatccgggtgctgagcctctttgacggcatcgccaccggcctgctggtgctgaaggacctgggcatccaggtggacagatacatcgccagtgaagtgtgcgaagattctatcaccgtgggcatggtgcggcaccaaggcaagatcatgtacgtgggcgacgtccggtctgtgacccagaagcatatccaggagtggggccccttcgacctggttatcggcggaagcccctgcaacgacctgagcatcgtgaaccccgctagaaagggcctgtacgagggcacaggcagactgttcttcgagttctaccggctgctgcacgatgcccgtcccaaggaaggcgatgacagaccttttttctggctgttcgagaacgtggtggccatgggcgtgagcgacaaacgggacatcagcagattcctggagtctaaccctgtgatgattgatgctaaggaagtgtctgccgcccaccgggcccggtacttctggggcaacctgccaggcatgaacagacctctcgcctctaccgtgaatgacaagctggaactgcaggagtgtctggaacacggcagaatcgccaagttcagtaaggtgagaaccatcaccaccaggagcaacagcatcaagcagggcaaagatcagcacttccctgtgtttatgaacgagaaggaggacatcctgtggtgcaccgagatggaaagagtttttggcttccccgtgcactacaccgacgtttctaacatgagcagactggccagacagcggctgctgggcagatcctggagcgtgcctgtgatcagacacctgttcgcccccctgaaggagtactttgcctgcgtgagctccggaaattctaacgccaactcccgcggcccctccttcagcagcggactggtgcctctcagcctgagaggcagccacatgggacctatggaaatatacaagacagtgtcggcttggaagaggcagcctgtgagagtgctgagcctgttcagaaacatcgacaaggtgctgaagagcctgggatttctggaatctggaagcgggtctggcggcggcaccctgaagtacgtggaagatgtgacaaacgtggtgcggagagatgtcgagaaatggggacctttcgatctggtgtacggcagcacccagcccctgggcagcagctgcgaccggtgccccggctggtacatgtttcaattccaccgtatcctgcagtacgccctgcctcggcaggaaagccagagacctttcttctggatcttcatggacaacctgctgctgaccgaggacgaccaggagacaaccacaagattcctgcagaccgaggccgtgaccctgcaggatgtccggggccgcgactaccagaatgccatgcgggtgtggagcaatatccctggcctgaagtccaagcacgcccctctgacacctaaggaagaagagtacctgcaagctcaggtgagaagcagatccaagctggatgcccctaaggtcgacctgctggtgaaaaactgcctgctgccacttagagagtacttcaaatacttctcccaaaatagcctgcccctgagcggcggaggcgggagcggcggaggcggcagcctggagcctggcgagaagccctacaagtgcccagagtgcggcaaatcttttagcgaccctggccacctggtgcggcaccagcggacccataccggtgagaaaccctacaaatgtcctgaatgtggcaagagcttctccgattgcagagatctggcccggcaccagagaacccatacaggcgaaaaaccttacaagtgccctgagtgcggaaaatccttcagctctcctgccgacctgacaagacaccagcggacccacacaggcgagaaaccttataagtgtcccgaatgcggcaagagcttctccagagccgataacctgaccgagcaccaaaggacccacaccggcgaaaagccttataagtgcccagaatgcggcaagagcttttcccagagcggtcatctgacagagcaccagagaacacacaccggagagaagccctataagtgtcctgagtgtggaaaaagcttttcaagaagcgaccacctcaccacacaccagcgcacacacacaggcaagaagaccagcagcggtggcggcggctctggcggcggaggaagcgacagcgtggccttcgaggacgtggctgtgaatttcactctggaagagtgggctctgctcgaccctagccaaaagaacctgtacagagacgtgatgagagaaaccttcagaaacctggctagcgtcggcaaacagtgggaggatcagaacatcgaggatcctttcaagatcccgagacggaacatcagccacatcccagagagactgtgcgagtccaaggagggaggccagggcgaggaataa

SEQ ID NO: 70 - protein, 830 aa, synthetic construct
MAPKKKRKVNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLSGGGGSGGGGSLEPGEKPYKCPECGKSFSDPGHLVRHQRTHTGEKPYKCPECGKSFSDCRDLARHQRTHTGEKPYKCPECGKSFSSPADLTRHQRTHTGEKPYKCPECGKSFSRADNLTEHQRTHTGEKPYKCPECGKSFSQSGHLTEHQRTHTGEKPYKCPECGKSFSRSDHLTTHQRTHTGKKTSSGGGGSGGGGSDSVAFEDVAVNFTLEEWALLDPSQKNLYRDVMRETFRNLASVGKQWEDQNIEDPFKIPRRNISHIPERLCESKEGGQGEE

SEQ ID NO: 71 - DNA, 5509 bp, synthetic construct
  source 1..5509: mol_type = other DNA
ctggggactttccagcctggggactttccagctgggactttccaggggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagggttggacgcgtctggcgacagacagccggcgctagcgccaccatgcactggggcaccctgtgcggcttcctgtggctgtggccctacctgttctacgtgcaggcttatggcgagggcacattcaccagcgactacagcaagtacctggataagatcgcccagaaagcctttgtgcagtggctgatcgccggaggaccttccagcggcgcccctcctccatctggatctggcgccacaaacttcagcctgctgaagcaggccggagatgtggaagaaaatcctggccccatggcttacccatacgatgttccagattacgctagtttggtgaggcagcagagaccgatgacatccactttgcctttctctccacaggtgtccactcccaggtccaagtttggaagatctaccatgccaaagagacccagacccgtgatcatgagtccaaagagaagaacacaggcagagcgcgcaatggagacccagggcaagttgattgcagcggccctgggggttttacgggaaaaaggttacgcgggattccggatcgcagatgtgcccggtgctgcaggtgtctcgagaggagcgcagagccatcatttcccgacaaagcttgagcttctgcttgccacttttgaatggctttacgaacagatcaccgaacgcagtcgggctcgattagcgaaattgaagccagaggatgacgtcatccagcaaatgctggacgacgccgccgaatttttcctcgacgatgacttctctatcagccttgatttgattgtggctgccgaccgggatccagcgttacgcgagggtattcagcgcacggtagagaggaatcggtttgtcgtcgaggatatgtggcttggtgttctggtgagccgtggtctttcgcgtgatgatgcagaagatatcctttggttgatattcaattcggtgcgtgggcttgctgttcgtagcctatggcagaaggacaaagaacgctttgagcgtgtcaggaactcgacactcgaaattgcgcgagagcggtacgcgaaattcaagcgcgcggccaaaccctctggccggtctctctctgttcgataataactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcaggctggggagggccgccgatcgaacttgtttattgcagcttataatggttacaaataaagcaatagcatcacaaatttcacaaataaagcatttttttcactgcattctagttgtggtttgtccaaactcatcaatgtatcttagactagaagcggccgcctattattcctcgccctggcctccctccttggactcgcacagtctctctgggatgtggctgatgttccgtctcgggatcttgaaaggatcctcgatgttctgatcctcccactgtttgccgacgctagccaggtttctgaaggtttctctcatcacgtctctgtacaggttcttttggctagggtcgagcagagcccactcttccagagtgaaattcacagccacgtcctcgaaggccacgctgtcgcttcctccgccgccagagccgccgccaccgctgctggtcttcttgcctgtgtgtgtgcgctggtgtgtggtgaggtggtcgcttcttgaaaagctttttccacactcaggacacttatagggcttctctccggtgtgtgttctctggtgctctgtcagatgaccgctctgggaaaagctcttgccgcattctgggcacttataaggcttttcgccggtgtgggtcctttggtgctcggtcaggttatcggctctggagaagctcttgccgcattcgggacacttataaggtttctcgcctgtgtgggtccgctggtgtcttgtcaggtcggcaggagagctgaaggattttccgcactcagggcacttgtaaggtttttcgcctgtatgggttctctggtgccgggccagatctctgcaatcggagaagctcttgccacattcaggacatttgtagggtttctcaccggtatgggtccgctggtgccgcaccaggtggccagggtcgctaaaagatttgccgcactctgggcacttgtagggcttctcgccaggctccaggctgccgcctccgccgctcccgcctccgccgctcaggggcaggctattttgggagaagtatttgaagtactctctaagtggcagcaggcagtttttcaccagcaggtcgaccttaggggcatccagcttggatctgcttctcacctgagcttgcaggtactcttcttccttaggtgtcagaggggcgtgcttggacttcaggccagggatattgctccacacccgcatggcattctggtagtcgcggccccggacatcctgcagggtcacggcctcggtctgcaggaatcttgtggttgtctcctggtcgtcctcggtcagcagcaggttgtccatgaagatccagaagaaaggtctctggctttcctgccgaggcagggcgtactgcaggatacggtggaattgaaacatgtaccagccggggcaccggtcgcagctgctgcccaggggctgggtgctgccgtacaccagatcgaaaggtccccatttctcgacatctctccgcaccacgtttgtcacatcttccacgtacttcagggtgccgccgccagacccgcttccagattccagaaatcccaggctcttcagcaccttgtcgatgtttctgaacaggctcagcactctcacaggctgcctcttccaagccgacactgtcttgtatatttccataggtcccatgtggctgcctctcaggctgagaggcaccagtccgctgctgaaggaggggccgcgggagttggcgttagaatttccggagctcacgcaggcaaagtactccttcaggggggcgaacaggtgtctgatcacaggcacgctccaggatctgcccagcagccgctgtctggccagtctgctcatgttagaaacgtcggtgtagtgcacggggaagccaaaaactctttccatctcggtgcaccacaggatgtcctccttctcgttcataaacacagggaagtgctgatctttgccctgcttgatgctgttgctcctggtggtgatggttctcaccttactgaacttggcgattctgccgtgttccagacactcctgcagttccagcttgtcattcacggtagaggcgagaggtctgttcatgcctggcaggttgccccagaagtaccgggcccggtgggcggcagacacttccttagcatcaatcatcacagggttagactccaggaatctgctgatgtcccgtttgtcgctcacgcccatggccaccacgttctcgaacagccagaaaaaaggtctgtcatcgccttccttgggacgggcatcgtgcagcagccggtagaactcgaagaacagtctgcctgtgccctcgtacaggccctttctagcggggttcacgatgctcaggtcgttgcaggggcttccgccgataaccaggtcgaaggggccccactcctggatatgcttctgggtcacagaccggacgtcgcccacgtacatgatcttgccttggtgccgcaccatgcccacggtgatagaatcttcgcacacttcactggcgatgtatctgtccacctggatgcccaggtccttcagcaccagcaggccggtggcgatgccgtcaaagaggctcagcacccggattggcttccgcttctcggctggcacaggagggtacaccttaggtgggtcgaactcctggtcgtggttgacctttctcttttttttaggggccatggtggcagcctgcttttttgtacaaacttggaattcggtaccggatccagtcgactagaccaaacttggacctgggagtggacacctgtggagagaaaggcaaagtggatgtcattgtcactcaagtgtatggccagatcgggccaggtgaatatcaaatcctcctcgtttttggaaactgacaatcttagcgcagaagtaatgcccgcttttgagagggagtactcaccccaacagctggatctcaagcctgccacacctcacctcgaccatccgccgtctcaagaccgcctactttaattacatcatcagcagcacctccgccagaaacaaccccgaccgccacccgctgccgcccgccacggtgctcagcctaccttgcgactgtgactggttagacgcctttctcgagaggttttccgatccggtcgatgcggactcgctcaggtccctcggtggcggagtaccgttcggaggccgacgggtttccgatccaagagtactggaaagaccgcgaagagtttgtcctcaaccgcgagcccaacagctggccctcgcagacagcgatgcggaagagagtgaccgcggaggctggatcggtcccggtgtcttctatggaggtcaaaacagcgtggatggcgtctccaggcgatatgaggcgcgcccggttcactaaacaccggtaacaaacagacaatctggtctgtttgattataccggttgcttatataggcctcccaccgtacacgcctacctcgacccggcaagcttgcccatttgcgtcaacggggcgggggtattacgacattttggaaagtcccgttgattttggtgccaaaacaaactcccattgacgtcaatggggtggagacttggaaatccccgtgagtcaaaccgctatccacgcccattggtgtactgccaaaaccgcatcaccatggtaatagcgatgactaatacgtagatgtactgccaagtaggaaagtcccgtaaggtcatgtactgggcataatgccaggcgggccatttaccgtcattgacgtcaatagggggcggacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgctggggactttccagctggggactttccagctgggactttccaggag

SEQ ID NO: 72 - DNA, 1210 bp, synthetic construct
  source 1..1210: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaatacccccgccccgttgacgcaaatgggcaagcttgccgggtcgaggtaggcgtgtacggtgggaggcctatataagcaaccggtataatcaaacagaccagattgtctgtttgttaccggtgtttagtgaaccgggcgcgcctcatatcgcctggagacgccatccacgctgttttgacctccatagaagacaccgggaccgatccagcctccgcggtcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagacggcggatggtcgaggtgaggtgtggcaggcttgagatccagctgttggggtgagtactccctctcaaaagcgggcattacttctgcgctaagattgtcagtttccaaaaacgaggaggatttgatattcacctggcccgatctggccatacacttgagtgacaatgacatccactttgcctttctctccacaggtgtccactcccaggtccaagtttggtctagtcgactggatccggtaccgaattc

SEQ ID NO: 73 - DNA, 27 bp, synthetic construct
  source 1..27: mol_type = other DNA
atcaaacagaccagattgtctgtttgt

SEQ ID NO: 74 - DNA, 798 bp, synthetic construct
  source 1..798: mol_type = other DNA
atggcttacccatacgatgttccagattacgctagtttggtgaggcagcagagaccgatgacatccactttgcctttctctccacaggtgtccactcccaggtccaagtttggaagatctaccatgccaaagagacccagacccgtgatcatgagtccaaagagaagaacacaggcagagcgcgcaatggagacccagggcaagttgattgcagcggccctgggggttttacgggaaaaaggttacgcgggattccggatcgcagatgtgcccggtgctgcaggtgtctcgagaggagcgcagagccatcatttcccgacaaagcttgagcttctgcttgccacttttgaatggctttacgaacagatcaccgaacgcagtcgggctcgattagcgaaattgaagccagaggatgacgtcatccagcaaatgctggacgacgccgccgaatttttcctcgacgatgacttctctatcagccttgatttgattgtggctgccgaccgggatccagcgttacgcgagggtattcagcgcacggtagagaggaatcggtttgtcgtcgaggatatgtggcttggtgttctggtgagccgtggtctttcgcgtgatgatgcagaagatatcctttggttgatattcaattcggtgcgtgggcttgctgttcgtagcctatggcagaaggacaaagaacgctttgagcgtgtcaggaactcgacactcgaaattgcgcgagagcggtacgcgaaattcaagcgcgcggccaaaccctctggccggtctctctctgttcgataa

SEQ ID NO: 75 - protein, 265 aa, synthetic construct
MAYPYDVPDYASLVRQQRPMTSTLPFSPQVSTPRSKFGRSTMPKRPRPVIMSPKRRTQAERAMETQGKLIAAALGVLREKGYAGFRIADVPGAAGVSRGAQSHHFPTKLELLLATFEWLYEQITERSRARLAKLKPEDDVIQQMLDDAAEFFLDDDFSISLDLIVAADRDPALREGIQRTVERNRFVVEDMWLGVLVSRGLSRDDAEDILWLIFNSVRGLAVRSLWQKDKERFERVRNSTLEIARERYAKFKRAAKPSGRSLSVR

SEQ ID NO: 76 - DNA, 180 bp, synthetic construct
  source 1..180: mol_type = other DNA
atgcactggggcaccctgtgcggcttcctgtggctgtggccctacctgttctacgtgcaggcttatggcgagggcacattcaccagcgactacagcaagtacctggataagatcgcccagaaagcctttgtgcagtggctgatcgccggaggaccttccagcggcgcccctcctccatct

SEQ ID NO: 77 - protein, 60 aa, synthetic construct
MHWGTLCGFLWLWPYLFYVQAYGEGTFTSDYSKYLDKIAQKAFVQWLIAGGPSSGAPPPS

SEQ ID NO: 78 - DNA, 7744 bp, synthetic construct
  source 1..7744: mol_type = other DNA
ctggggactttccagcctggggactttccagctgggactttccaggcgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttgggccaccatggccttcttgtggctgctcagctgctgggccctgctgggcaccacattcggcctgcttgtgcctagagagctgagcggctctagcccagtgctggaggaaacccacccagcccaccagcagggagctagccggcctggacccagagacgcccaggcccaccccggaagacccagagccgtgcctacccagtgcgacgtgcctcccaacagcagattcgactgcgcccctgataaggccattacccaggagcagtgtgaagctagaggctgctgctacatccctgccaagcagggcctgcaaggcgcccagatgggccaaccttggtgcttcttcccaccctcttaccctagctacaagctggaaaacctgagctcttctgagatgggctacaccgccaccctgacaagaaccaccccaacattcttcccaaaagacatcctgaccctgagactggacgtgatgatggaaaccgagaaccggctgcatttcaccattaaggaccccgccaacagacggtacgaggtccccctggaaacaccccacgtgcacagccgggccccttcccctctgtacagtgtggaattcagcgaggaacccttcggcgtgatcgtgagaaggcagctggatggcagagtgctgctcaacaccacagtggctccattattcttcgccgaccagttcctgcagctgagcaccagcctgccctcacagtacatcacaggcctcgccgaacacctgtctcctctcatgctgagcaccagctggacaagaatcacactgtggaaccgggacctggcccccacacctggggctaatctgtacggctcccaccccttctacctggcgctggaagatggcggcagcgcccacggcgttttcctgctgaacagcaatgccatggacgtggtgctgcagcctagccctgccctgagctggcggagcaccggcggaatcctggacgtgtacatcttcctgggacccgagcctaagagcgtggtgcagcaatatctggatgtggtgggctacccctttatgcctccttactggggcctgggttttcacctgtgtagatggggctactcatctaccgcgatcaccagacaggtggttgagaatatgacccgggcccacttccctctcgacgtgcagtggaacgacctggactacatggacagcagaagagatttcacgttcaacaaggacggcttcagggatttccctgctatggtgcaggagctgcaccagggaggcagacggtacatgatgatcgtggatcctgctatcagcagcagcggccccgccggcagctatagaccttatgacgagggactgagacggggcgttttcatcacaaacgagacaggacagcctcttatcggcaaggtgtggcccggctctaccgcctttcctgacttcacaaatcctaccgccctggcctggtgggaagatatggtcgccgagttccacgaccaggtccctttcgacgggatgtggatcgacatgaacgagcccagtaacttcattagaggctctgaagatggatgtcctaacaacgagctggagaaccctccttacgtgcctggcgtggtcggcggaactctgcaggccgctacaatctgtgccagctcccaccagttcctgagcacccactacaacctgcacaatctgtacggactgacagaggctatcgccagtcatagagccctggtcaaggccagaggcaccagacccttcgtgatctccagaagcaccttcgccggacacggcagatatgccggccactggacaggcgacgtgtggtcctcttgggagcagctcgcaagctctgtgccggaaatcctgcaatttaacttgctgggcgtgcctctggtgggcgctgatgtgtgcggctttctgggcaacacatctgaagaattgtgcgtgcggtggacccagctgggcgctttttacccttttatgagaaatcacaactccctgctgagcctgccacaggagccttacagcttcagcgaacctgcccaacaggccatgcggaaggccttaacactgcgatacgcactgctgcctcacctgtacaccctgttccaccaggctcatgtggccggcgaaacagtggccagaccactgttcctggagttccccaaggatagctccacatggaccgtggaccaccagctgctgtggggcgaggccctgctgatcacccctgtgctgcaggccggcaaagctgaggtgaccggctacttccctctgggcacctggtacgacctgcagaccgtgcctgttgaggctctgggatctctgccccccccacctgccgcccctcgcgagcctgctatccactctgaaggccagtgggtgacactgcctgccccactcgacaccatcaacgtgcacctgagagccggttacatcatccccctgcagggccccggcctgaccaccaccgaaagccggcagcaacctatggccctcgccgtggccctgaccaagggcggcgaggccaggggtgagctattttgggacgacggcgagagcctggaggtgctggagagaggcgcctacacacaagttatcttcctggctcggaacaacaccatcgtgaacgagctggtgcgggtgacaagcgagggcgccggcctccagctgcagaaagtgacggtgctgggcgtggccacagcccctcagcaggtgctgagcaacggcgtgccagtcagcaatttcacctacagccctgataccaaggttctggacatctgcgtgagcctgctcatgggcgaacagttcctggtgtcctggtgcggatctggcgccacaaacttcagcctgctgaagcaggccggagatgtggaagaaaatcctggccccatgtctagattagataaaagtaaagtgattaacagcgcattagagctgcttaatgaggtcggaatcgaaggtttaacaacccgtaaactcgcccagaagctaggtgtagagcagcctacattgtattggcacgtgcgcaacaagcagactcttatgaacatgctttcagaggcaatactggcgaagcatcacacccgttcagcaccgttaccgactgagagttggcagcagtttctccaggaaaatgctctgagtttccgtaaagcattactggtccatcgtgatggagcccgattgcatatagggacctctcctacgcccccccagtttgaacaagcagaggcgcaactacgctgtctatgcgatgcagggttttcggtcgaggaggctcttttcattctgcaatctatcagccattttacgttgggtgcagtattagaggagcaagcaacaaaccagatagaaaataatcatgtgatagacgctgcaccaccattattacaagaggcatttaatattcaggcgagaacctctgctgaaatggccttccatttcgggctgaaatcattaatatttggattttctgcacagttagatgaaaaaaagcatacacccattgaggatggtaataaaccaaaaaagaagagaaagctagcagtgtcagtgacatttgaagatgtggctgtgctctttactcgggacgagtggaagaagctggatctgtctcagagaagcctgtaccgtgaggtgatgctggagaattacagcaacctggcctccatggcaggattcctgtttaccaaaccaaaggtgatctccctgttgcagcaaggagaggatccctggggctccggagagggcaggggaagtcttctaacatgcggggacgtggaggaaaatcccggccccatgtctagactggacaagagcaaagtcataaacggcgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagcaattgagctgttcgaccggcagggagccgaacctgccttccttttcggcctggaactaatcatatgtggcctggagaaacagctaaagtgcgaaagcggcgggccggccgacgcccttgacgattttgacttagacatgctcccagccgatgcccttgacgactttgaccttgatatgctgcctgctgacgctcttgacgattttgaccttgacatgctccccgggtaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcaggctggggaaacttgtttattgcagcttataatggttacaaataaagcaatagcatcacaaatttcacaaataaagcatttttttcactgcattctagttgtggtttgtccaaactcatcaatgtatcttatcattattagctggcgctggtcttgaaaaacagcttctttctcaagaagttgaagcagccaggcatctgcttgtagatgcccttaacggacacggcattagccacccgcagcagcagagattgaaggtcctcgctgtgggcccactgctcaaagatatcgtccagtgtctccacgtaccaggagccgctcttagggtcccgccagctcacgaatcctggaaaggtgctatatgacacgaagatatcgctaggtgtaggcaggctggagatggcatccagctggtcaaaagttctcaggccctcctgaaaaggggtggcgtctggctcaggattagagcctggagactcgtcctcggggcttgtgctggccacctcgaagccgtggtccttctgttcgccgccacaagcctggatgaagaacagcttgggcttgccgcccaggctggggcaggatgtgccgttgaagatgttcacgatcttttccacggacacagggcaaccgtcggtgccgtacactgctccagggaactgcaggtggctagcctgacagccatggctcaggatgaccaccacgcagcagtccagggcgccgtggtcctgctgggccagttcgagcagggccagcaccatcttcttggcggtgagatctcctttcacctcgaccatgaagtgcagagagctgaatcttctccgcagcttctcgcagtcgatgttgctgccggttctggtgcgcagtccgctctctctgcagaagttcacgttgttgatgatcaggcagtggccacaaggctccatgctcagaatgtaagccagatcggcgttgcctctgaggctttccagggcgcccacgtcgccgaagccgtccacgccggaaccgccgccgccgctgccaccgcctccagagccgccgcctccgctctccagcttcagcagctccacatcaaagaccagggtggcgtggggggggatgatgccagggtgtccggtagcgccgtaggcgtagtcaggagaaattgtcagtttggcccgctggcccacgctcatttgggccacgccttcctcccagcctctgatcacctcctgtttgcccagcatgaacttgaaaggcttgttccgatctctgctgctgtcgaatttctttccatcttccagcattccggtgtagtgcaccacacaggtctgtcctcttttggggaatgttctgccatcgccaggagagatggtttccacctgaactccttccaggcttcctcctccgctgtattccagcttgctgatccgtctgaacacgtggtagtacaggtcccaggcttgcagcaggtccttcacattcccgcttttcatgtacttccggcaccactcctgggcttccatcagatcgcggccataggcctggttgaagcttgtttccttcagggtctgagggcctctttccatcatggcgtgcaggggttccaggacctcgaacatgcccttcacgttccgctcgccgaagtacagtctactagcctcttccaggccctcgtgccacatctcatgccacaggattctagaggccatggtggccaattcggggccgcggaggctggatcggtcccggtgtcttctatggaggtcaaaacagcgtggatggcgtctccaggcgatctgacggttcactaaacgagctctgcttatataggtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagctcggtaccctggggactttccagctggggactttccagctgggactttccaggag

SEQ ID NO: 79 - DNA, 1554 bp, synthetic construct
  source 1..1554: mol_type = other DNA
atggcctctagaatcctgtggcatgagatgtggcacgagggcctggaagaggctagtagactgtacttcggcgagcggaacgtgaagggcatgttcgaggtcctggaacccctgcacgccatgatggaaagaggccctcagaccctgaaggaaacaagcttcaaccaggcctatggccgcgatctgatggaagcccaggagtggtgccggaagtacatgaaaagcgggaatgtgaaggacctgctgcaagcctgggacctgtactaccacgtgttcagacggatcagcaagctggaatacagcggaggaggaagcctggaaggagttcaggtggaaaccatctctcctggcgatggcagaacattccccaaaagaggacagacctgtgtggtgcactacaccggaatgctggaagatggaaagaaattcgacagcagcagagatcggaacaagcctttcaagttcatgctgggcaaacaggaggtgatcagaggctgggaggaaggcgtggcccaaatgagcgtgggccagcgggccaaactgacaatttctcctgactacgcctacggcgctaccggacaccctggcatcatccccccccacgccaccctggtctttgatgtggagctgctgaagctggagagcggaggcggcggctctggaggcggtggcagcggcggcggcggttccggcgtggacggcttcggcgacgtgggcgccctggaaagcctcagaggcaacgccgatctggcttacattctgagcatggagccttgtggccactgcctgatcatcaacaacgtgaacttctgcagagagagcggactgcgcaccagaaccggcagcaacatcgactgcgagaagctgcggagaagattcagctctctgcacttcatggtcgaggtgaaaggagatctcaccgccaagaagatggtgctggccctgctcgaactggcccagcaggaccacggcgccctggactgctgcgtggtggtcatcctgagccatggctgtcaggctagccacctgcagttccctggagcagtgtacggcaccgacggttgccctgtgtccgtggaaaagatcgtgaacatcttcaacggcacatcctgccccagcctgggcggcaagcccaagctgttcttcatccaggcttgtggcggcgaacagaaggaccacggcttcgaggtggccagcacaagccccgaggacgagtctccaggctctaatcctgagccagacgccaccccttttcaggagggcctgagaacttttgaccagctggatgccatctccagcctgcctacacctagcgatatcttcgtgtcatatagcacctttccaggattcgtgagctggcgggaccctaagagcggctcctggtacgtggagacactggacgatatctttgagcagtgggcccacagcgaggaccttcaatctctgctgctgcgggtggctaatgccgtgtccgttaagggcatctacaagcagatgcctggctgcttcaacttcttgagaaagaagctgtttttcaagaccagcgccagctaa

SEQ ID NO: 80 - protein, 517 aa, synthetic construct
MASRILWHEMWHEGLEEASRLYFGERNVKGMFEVLEPLHAMMERGPQTLKETSFNQAYGRDLMEAQEWCRKYMKSGNVKDLLQAWDLYYHVFRRISKLEYSGGGSLEGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLESGGGGSGGGGSGGGGSGVDGFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSAS

SEQ ID NO: 81 - DNA, 3961 bp, synthetic construct
  source 1..3961: mol_type = other DNA
ctggggactttccagcctggggactttccagctgggactttccaggcgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttgggccaccatggtttccccagacccccaaattatcgtggtggcccccgagaccgaactcgcgtctatgcaagtccaacgcactgaggacggggtaaccattatccagatattttgggtgggccgcaaaggcgagctacttagacgcaccccggtgagctcggccatgcagacaccaatgggaatcccaatggggaagtcgatgctggtgcttctcaccttcttggccttcgcctcgtgctgcattgctgcttaccgccccagtgagaccctgtgcggcggggagctggtggacaccctccagttcgtctgtggggaccgcggcttctacttcagcaggcccgcaagccgtgtgagccgtcgcagccgtggcatcgttgaggagtgctgtttccgcagctgtgacctggccctcctggagacgtactgtgctacccccgccaagtccgagagggacgtgtcgacccctccgaccgtgcttccggacaacttccccagataccccgtgggcaagttcttccaatatgacacctggaagcagtccacccagcgcctgcgcaggggcctgcctgccctcctgcgtgcccgccggggtcacgtgctcgccaaggagctcgaggcgttcagggaggccaaacgtcaccgtcccctgattgctctacccacccaagaccccgcccacgggggcgcccccccagagatggccagcaatcggaagggatctggcgccacaaacttcagcctgctgaagcaggccggagatgtggaagaaaatcctggccccatgagcagactggataagtccaaggtgatcaacagcgccctggaactgctcaacggcgtcggcatcgagggactcaccacccggaagctggcccagaaactgggcgtggaacaacctacactgtactggcacgtgaagaacaaaagagccctgctggatgccctgccaatcgagatgctggaccggcaccatacccacagctgccccctggagggagaaagctggcaggacttcctgagaaacaacgctaagagctaccggtgcgccctgctgagccaccgggacggcgccaaggtgcacttggggaccagacccacggaaaagcaatacgagacactggagaaccagctggccttcctgtgccagcagggcttcagcctggagaatgccctctacgccctgtctgccgtgggccactttaccctgggatgtgtgctggaagagcaggagcaccaggtggccaaggaagagagggaaacccctaccaccgacagcatgcctcctctgctgaagcaagctatcgagctgttcgacagacagggcgccgagcctgctttcctgtttggcctggagctgatcatctgcggcttggagaaacagctgaagtgcgagagcggcggccctaccgacgccctggacgatttcgacctggatatgctgcctgccgacgccctggacgacttcgacctggatatgctgcctgccgatgctctggacgacttcgacctggacatgcttccaggctaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcaggctggggaaacttgtttattgcagcttataatggttacaaataaagcaatagcatcacaaatttcacaaataaagcatttttttcactgcattctagttgtggtttgtccaaactcatcaatgtatcttatcattaatctgtcaggaagttgtggaggcacctcatcatggagaggccagagggcagctgtggccaggcgccttcgctcgtggaacgcttgtggcctgtcgcgggtggctggaaggtcctctgcttcttatccgggaagagaaggacgtccaagtcagagtcaataaccagctgggccacccggaatgcgagggtgctgcctgaggggatggtgaccgtcttcttctggctcagatggccctggccctcaccctgcaagcacgtggctccgggcagggaaaaccggcccgagccctcccgcttgtgggtgcgcgtgacttccacctccttctgtgtctgcagcacctcagtcaccacgtacacgttgtccccgcggctgcgcagctgctgcaggactttgtgttctggctgccgcaggtgcctctcatggagcagagtctgccaggtgttagggtccacactcagcgagtacacattcattgaggtgctggagctgtcagacaccgcggccccgcctgcgatctttgcctgtcctggggctgccagctccacgctgccctgtatctgcccatccatggcatcgtagaagtggaagctcctgccacgctgcacgtctggttccgcggcatccggctccaggatgtccttgatagacaggttgacacacttataacggggtttccagaaccatgagcttgagggcttcctaaccaccaggcagtagggctggaagccagtggagctctgcaggctggtcacagggatgaactccccaccatggtccagctcctggaccactctccggactacccgctcaaaggccgaccccatggtggctttacgagggtaggaagtggtacggaaagttggtataagacaaaagtgttgtggaattgctccaggcgatctgacggttcactaaacgagctctgcttttataggcgcccaccgtacacgcctaaagcttatacgttctctatcactgatagggagtaaactggatatacgttctctatcactgatagggagtaaactgtagatacgttctctatcactgatagggagtaaactggtcatacgttctctatcactgatagggagtaaactccttatacgttctctatcactgatagggagtaaagtctgcatacgttctctatcactgatagggagtaaactcttcatacgttctctatcactgatagggagtaaactggggactttccagctggggactttccagctgggactttccaggag

SEQ ID NO: 82 - DNA, 708 bp, synthetic construct
  source 1..708: mol_type = other DNA
atggtttccccagacccccaaattatcgtggtggcccccgagaccgaactcgcgtctatgcaagtccaacgcactgaggacggggtaaccattatccagatattttgggtgggccgcaaaggcgagctacttagacgcaccccggtgagctcggccatgcagacaccaatgggaatcccaatggggaagtcgatgctggtgcttctcaccttcttggccttcgcctcgtgctgcattgctgcttaccgccccagtgagaccctgtgcggcggggagctggtggacaccctccagttcgtctgtggggaccgcggcttctacttcagcaggcccgcaagccgtgtgagccgtcgcagccgtggcatcgttgaggagtgctgtttccgcagctgtgacctggccctcctggagacgtactgtgctacccccgccaagtccgagagggacgtgtcgacccctccgaccgtgcttccggacaacttccccagataccccgtgggcaagttcttccaatatgacacctggaagcagtccacccagcgcctgcgcaggggcctgcctgccctcctgcgtgcccgccggggtcacgtgctcgccaaggagctcgaggcgttcagggaggccaaacgtcaccgtcccctgattgctctacccacccaagaccccgcccacgggggcgcccccccagagatggccagcaatcggaag

SEQ ID NO: 83 - protein, 236 aa, synthetic construct
MVSPDPQIIVVAPETELASMQVQRTEDGVTIIQIFWVGRKGELLRRTPVSSAMQTPMGIPMGKSMLVLLTFLAFASCCIAAYRPSETLCGGELVDTLQFVCGDRGFYFSRPASRVSRRSRGIVEECCFRSCDLALLETYCATPAKSERDVSTPPTVLPDNFPRYPVGKFFQYDTWKQSTQRLRRGLPALLRARRGHVLAKELEAFREAKRHRPLIALPTQDPAHGGAPPEMASNRK

SEQ ID NO: 84 - DNA, 825 bp, synthetic construct
  source 1..825: mol_type = other DNA
atggggtcggcctttgagcgggtagtccggagagtggtccaggagctggaccatggtggggagttcatccctgtgaccagcctgcagagctccactggcttccagccctactgcctggtggttaggaagccctcaagctcatggttctggaaaccccgttataagtgtgtcaacctgtctatcaaggacatcctggagccggatgccgcggaaccagacgtgcagcgtggcaggagcttccacttctacgatgccatggatgggcagatacagggcagcgtggagctggcagccccaggacaggcaaagatcgcaggcggggccgcggtgtctgacagctccagcacctcaatgaatgtgtactcgctgagtgtggaccctaacacctggcagactctgctccatgagaggcacctgcggcagccagaacacaaagtcctgcagcagctgcgcagccgcggggacaacgtgtacgtggtgactgaggtgctgcagacacagaaggaggtggaagtcacgcgcacccacaagcgggagggctcgggccggttttccctgcccggagccacgtgcttgcagggtgagggccagggccatctgagccagaagaagacggtcaccatcccctcaggcagcaccctcgcattccgggtggcccagctggttattgactctgacttggacgtccttctcttcccggataagaagcagaggaccttccagccacccgcgacaggccacaagcgttccacgagcgaaggcgcctggccacagctgccctctggcctctccatgatgaggtgcctccacaacttcctgacagat

SEQ ID NO: 85 - protein, 275 aa, synthetic construct
MGSAFERVVRRVVQELDHGGEFIPVTSLQSSTGFQPYCLVVRKPSSSWFWKPRYKCVNLSIKDILEPDAAEPDVQRGRSFHFYDAMDGQIQGSVELAAPGQAKIAGGAAVSDSSSTSMNVYSLSVDPNTWQTLLHERHLRQPEHKVLQQLRSRGDNVYVVTEVLQTQKEVEVTRTHKREGSGRFSLPGATCLQGEGQGHLSQKKTVTIPSGSTLAFRVAQLVIDSDLDVLLFPDKKQRTFQPPATGHKRSTSEGAWPQLPSGLSMMRCLHNFLTD

SEQ ID NO: 86 - DNA, 5986 bp, synthetic construct
  source 1..5986: mol_type = other DNA
ctggggactttccagcctggggactttccagctgggactttccaggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagccaccatggagttttcaagtccttccagagaggaatgtcccaagcctttgagtagggtaagcatcatggctggcagcctcacaggattgcttctacttcaggcagtgtcgtgggcatcaggtgcccgcccctgcatccctaaaagcttcggctacagctcggtggtgtgtgtctgcaatgccacatactgtgactcctttgaccccccgacctttcctgcccttggtaccttcagccgctatgagagtacacgcagtgggcgacggatggagctgagtatggggcccatccaggctaatcacacgggcacaggcctgctactgaccctgcagccagaacagaagttccagaaagtgaagggatttggaggggccatgacagatgctgctgctctcaacatccttgccctgtcaccccctgcccaaaatttgctacttaaatcgtacttctctgaagaaggaatcggatataacatcatccgggtacccatggccagctgtgacttctccatccgcacctacacctatgcagacacccctgatgatttccagttgcacaacttcagcctcccagaggaagataccaagctcaagatacccctgattcaccgagccctgcagttggcccagcgtcccgtttcactccttgccagcccctggacatcacccacttggctcaagaccaatggagcggtgaatgggaaggggtcactcaagggacagcccggagacatctaccaccagacctgggccagatactttgtgaagttcctggatgcctatgctgagcacaagttacagttctgggcagtgacagctgaaaatgagccttctgctgggctgttgagtggataccccttccagtgcctgggcttcacccctgaacatcagcgagacttcattgcccgtgacctaggtcctaccctcgccaacagtactcaccacaatgtccgcctactcatgctggatgaccaacgcttgctgctgccccactgggcaaaggtggtactgacagacccagaagcagctaaatatgttcatggcattgctgtacattggtacctggactttctggctccagccaaagccaccctaggggagacacaccgcctgttccccaacaccatgctctttgcctcagaggcctgtgtgggctccaagttctgggagcagagtgtgcggctaggctcctgggatcgagggatgcagtacagccacagcatcatcacgaacctcctgtaccatgtggtcggctggaccgactggaaccttgccctgaaccccgaaggaggacccaattgggtgcgtaactttgtcgacagtcccatcattgtagacatcaccaaggacacgttttacaaacagcccatgttctaccaccttggccacttcagcaagttcattcctgagggctcccagagagtggggctggttgccagtcagaagaacgacctggacgcagtggcactgatgcatcccgatggctctgctgttgtggtcgtgctaaaccgctcctctaaggatgtgcctcttaccatcaaggatcctgctgtgggcttcctggagacaatctcacctggctactccattcacacctacctgtggcgtcgccagggctctggagccacaaacttcagcctgctgaagcaggccggagatgtggaagaaaatcctggccccatgagcagactggataagtccaaggtgatcaacagcgccctggaactgctcaacggcgtcggcatcgagggactcaccacccggaagctggcccagaaactgggcgtggaacaacctacactgtactggcacgtgaagaacaaaagagccctgctggatgccctgccaatcgagatgctggaccggcaccatacccacagctgccccctggagggagaaagctggcaggacttcctgagaaacaacgctaagagctaccggtgcgccctgctgagccaccgggacggcgccaaggtgcacttggggaccagacccacggaaaagcaatacgagacactggagaaccagctggccttcctgtgccagcagggcttcagcctggagaatgccctctacgccctgtctgccgtgggccactttaccctgggatgtgtgctggaagagcaggagcaccaggtggccaaggaagagagggaaacccctaccaccgacagcatgcctcctctgctgaagcaagctatcgagctgttcgacagacagggcgccgagcctgctttcctgtttggcctggagctgatcatctgcggcttggagaaacagctgaagtgcgagagcggcggccctaccgacgccctggacgatttcgacctggatatgctgcctgccgacgccctggacgacttcgacctggatatgctgcctgccgatgctctggacgacttcgacctggacatgcttccaggctaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcaggctggggaaacttgtttattgcagcttataatggttacaaataaagcaatagcatcacaaatttcacaaataaagcatttttttcactgcattctagttgtggtttgtccaaactcatcaatgtatcttattacccggggagcatgtcaaggtccaaatcgtcaagagcgtcagcaggcagcatatcaaggtcaaagtcgtcaagggcatcggctgggagcatgtctaagtcaaaatcgtcaagggcgtcggccggcccgccgctttcgcactttagctgtttctccaggccacatatgattagttccaggccgaaaaggaaggcaggttcggctccctgatggtcgaacagctcaattgcttgtctcagaagtgggggcatagaatcggtggtaggtgtctctctttcctcttttgctacttgatgctcctgatcctccaatacgcagcccagtgtaaagtggcccacggcggacagagcgtacagtgcgttctccagggagaagccttgctgacacaggaacgcgagctgattttccagggtttcgtactgtttctctgttgggcgggtgccgagatgcactttagccccgtcgcgatgtgagaggagagcacagcggaatgacttggcgttgttccgcagaaagtcttgccatgactcgccttccagggggcagaagtgggtatgatgcctgtccagcatctcgattgccagggcatcgagcagggcccgcttgttcttcacgtgccagtacagggtaggctgctcaactcccagcttttgagcgagtttccttgtcgtcaggccttcgataccgacttcattgagtaattccagagcagagtttatgactttgctcttgtccagtctagacatggggccaggattttcttccacatctccggcctgcttcagcaggctgaagtttgtggctccagagccctgagatttgttcctcaggtcctggatggctgcctcgatggccaggctcagggtgtccaggtcttcgggaggggtctcggtgggcttctcaaactgccccacggcgtaggccttcgcggccgtctcgtagataggcagcatgaacccaccctggttggtggagaagatgcgcaccatgacctgtttgggaaacttttgcatcaggggcaggcacaggttgagagcgcccaacaggtccacgggggtggcagcgtggataatcatgttgcggtaatcagaagagcgggggcacaattggtgggtgtgcagttctttgaggttccacacggccttgacaccgtcgttacaagcatcggccgtgcgctgcgccacatcaggagggtgtgtcacaggcatagtgtgctccatgaggaagggagtggagagggccaggttgcacatgctacccaggcgacaccgtacctgatccacctcattcttcacttcatgattgcgggtgtagatgatctgaatgcccttgttgttcacctgcatggttttgcaaactttgatggcctcttctaacacttggtgcatactagggatcatgaatggcagattcttgtactcaagagaacgattggtgtgacggaacatgcggctcacctcgtcaatcttgacgcgaccccgccgagtctgcacgttgggtgtgcagaagggggtgttcttatctttcatgatattgcgcaccttctcgttgtccaactcggagatgcgtttgctcttcttcttgcgaggtccggtgctcgccccgccgctgctctgatggccgcagctcagcagagaggaggaggccgcgccaccaaaaccgccgcgcccatggtggctcgaggtcacggatgctcctccgccactgctgcatttcatctcctcggactcactctccgagtccgaagccgaactgcaggaggaggaagacgaagaggaactatcttcatcgggccggcccaaaggatcgggaagaggagggtggttcatctgggagagcgggtgcgtgggagaggtcactcgcggcgtgccgctgccggtggaaggggaagacgcggtagcaccgcgggtctcgacttcttcaccctgttcttcctcgctatcagagatcacgatacagccggcggtatcgataatcttgttgcggtactggatggtaaagtcgggctcgggcttgatgtcttcctgtttgatgaggggcagcatgataggcgcgggaggcacgggcggtttaataatcaccttgaaaggacgcgtggttttgcgcggtttcttacgcgggctgagctcgggagtagcggatgccccggggagaggagtgttagtaaccgcgacgctggtgggggtcggcttgttaagaggggcgctgctaacgctgcaagagtgggttgtcagcgtggggccggtgctactggaatcgataccggcatgattgacagcctgggcgaggatgtcaccgagttctgtcaggacatctttttcggggttctcgttgcaatcctcggtcacttgttcaaaggttttgagggattcttcggccaattctgggaacagcgggtctcccaggctcagctgactgttaacctccttccttaacatagtctgcaggaacgtcgtggccttggtcacgggtgtctcgggccgtggcaccttggaggaagggccctcgtcaggattatcagggtccatctttctcttggcagaggactccatggtggctttacgagggtaggaagtggtacggaaagttggtataagacaaaagtgttgtggaattgctccaggcgatctgacggttcactaaacgagctctgcttttataggcgcccaccgtacacgcctaaagcttatacgttctctatcactgatagggagtaaactggatatacgttctctatcactgatagggagtaaactgtagatacgttctctatcactgatagggagtaaactggtcatacgttctctatcactgatagggagtaaactccttatacgttctctatcactgatagggagtaaagtctgcatacgttctctatcactgatagggagtaaactcttcatacgttctctatcactgatagggagtaaactggggactttccagctggggactttccagctgggactttccaggaga

SEQ ID NO: 87 - DNA, 1608 bp, synthetic construct
  source 1..1608: mol_type = other DNA
atggagttttcaagtccttccagagaggaatgtcccaagcctttgagtagggtaagcatcatggctggcagcctcacaggattgcttctacttcaggcagtgtcgtgggcatcaggtgcccgcccctgcatccctaaaagcttcggctacagctcggtggtgtgtgtctgcaatgccacatactgtgactcctttgaccccccgacctttcctgcccttggtaccttcagccgctatgagagtacacgcagtgggcgacggatggagctgagtatggggcccatccaggctaatcacacgggcacaggcctgctactgaccctgcagccagaacagaagttccagaaagtgaagggatttggaggggccatgacagatgctgctgctctcaacatccttgccctgtcaccccctgcccaaaatttgctacttaaatcgtacttctctgaagaaggaatcggatataacatcatccgggtacccatggccagctgtgacttctccatccgcacctacacctatgcagacacccctgatgatttccagttgcacaacttcagcctcccagaggaagataccaagctcaagatacccctgattcaccgagccctgcagttggcccagcgtcccgtttcactccttgccagcccctggacatcacccacttggctcaagaccaatggagcggtgaatgggaaggggtcactcaagggacagcccggagacatctaccaccagacctgggccagatactttgtgaagttcctggatgcctatgctgagcacaagttacagttctgggcagtgacagctgaaaatgagccttctgctgggctgttgagtggataccccttccagtgcctgggcttcacccctgaacatcagcgagacttcattgcccgtgacctaggtcctaccctcgccaacagtactcaccacaatgtccgcctactcatgctggatgaccaacgcttgctgctgccccactgggcaaaggtggtactgacagacccagaagcagctaaatatgttcatggcattgctgtacattggtacctggactttctggctccagccaaagccaccctaggggagacacaccgcctgttccccaacaccatgctctttgcctcagaggcctgtgtgggctccaagttctgggagcagagtgtgcggctaggctcctgggatcgagggatgcagtacagccacagcatcatcacgaacctcctgtaccatgtggtcggctggaccgactggaaccttgccctgaaccccgaaggaggacccaattgggtgcgtaactttgtcgacagtcccatcattgtagacatcaccaaggacacgttttacaaacagcccatgttctaccaccttggccacttcagcaagttcattcctgagggctcccagagagtggggctggttgccagtcagaagaacgacctggacgcagtggcactgatgcatcccgatggctctgctgttgtggtcgtgctaaaccgctcctctaaggatgtgcctcttaccatcaaggatcctgctgtgggcttcctggagacaatctcacctggctactccattcacacctacctgtggcgtcgccag

SEQ ID NO: 88 - protein, 536 aa, synthetic construct
MEFSSPSREECPKPLSRVSIMAGSLTGLLLLQAVSWASGARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 89 - DNA, 747 bp, synthetic construct
  source 1..747: mol_type = other DNA
atgtctagactggacaagagcaaagtcataaactctgctctggaattactcaatgaagtcggtatcgaaggcctgacgacaaggaaactcgctcaaaagctgggagttgagcagcctaccctgtactggcacgtgaagaacaagcgggccctgctcgatgccctggcaatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggatcaggagcatcaagtagcaaaagaggaaagagagacacctaccaccgattctatgcccccacttctgagacaagcaattgagctgttcgaccatcagggagccgaacctgccttccttttcggcctggaactaatcatatgtggcctggagaaacagctaaagtgcgaaagcggcgggccggccgacgcccttgacgattttgacttagacatgctcccagccgatgcccttgacgactttgaccttgatatgctgcctgctgacgctcttgacgatttggaccttgacatgctccccgggtaa

SEQ ID NO: 90 - protein, 248 aa, synthetic construct
MSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGGPADALDDFDLDMLPADALDDFDLDMLPADALDDLDLDMLPG

SEQ ID NO: 91 - DNA, 1740 bp, synthetic construct
  source 1..1740: mol_type = other DNA
atggagtcctctgccaagagaaagatggaccctgataatcctgacgagggcccttcctccaaggtgccacggcccgagacacccgtgaccaaggccacgacgttcctgcagactatgttaaggaaggaggttaacagtcagctgagcctgggagacccgctgttcccagaattggccgaagaatccctcaaaacctttgaacaagtgaccgaggattgcaacgagaaccccgaaaaagatgtcctgacagaactcggtgacatcctcgcccaggctgtcaatcatgccggtatcgattccagtagcaccggccccacgctgacaacccactcttgcagcgttagcagcgcccctcttaacaagccgacccccaccagcgtcgcggttactaacactcctctccccggggcatccgctactcccgagctcagcccgcgtaagaaaccgcgcaaaaccacgcgtcctttcaaggtgattattaaaccgcccgtgcctcccgcgcctatcatgctgcccctcatcaaacaggaagacatcaagcccgagcccgactttaccatccagtaccgcaacaagattatcgataccgccggctgtatcgtgatctctgatagcgaggaagaacagggtgaagaagtcgagacccgcggtgctaccgcgtcttccccttccaccggcagcggcacgccgcgagtgacctctcccacgcacccgctctcccagatgaaccaccctcctcttcccgatcctttgggccggcccgatgaagatagttcctcttcgtcttcctcctcctgcagttcggcttcggactcggagagtgagtccgaggagatgaaatgcagcagtggcggaggagcatccgtgacctcgagccaccatgggcgcggcggttttggtggcgcggcctcctcctctctgctgagctgcggccatcagagcagcggcggggcgagcaccggacctcgcaagaagaagagcaaacgcatctccgagttggacaacgagaaggtgcgcaatatcatgaaagataagaacacccccttctgcacacccaacgtgcagactcggcggggtcgcgtcaagattgacgaggtgagccgcatgttccgtcacaccaatcgttctcttgagtacaagaatctgccattcatgatccctagtatgcaccaagtgttagaagaggccatcaaagtttgcaaaaccatgcaggtgaacaacaagggcattcagatcatctacacccgcaatcatgaagtgaagaatgaggtggatcaggtacggtgtcgcctgggtagcatgtgcaacctggccctctccactcccttcctcatggagcacactatgcctgtgacacaccctcctgatgtggcgcagcgcacggccgatgcttgtaacgacggtgtcaaggccgtgtggaacctcaaagaactgcacacccaccaattgtgcccccgctcttctgattaccgcaacatgattatccacgctgccacccccgtggacctgttgggcgctctcaacctgtgcctgcccctgatgcaaaagtttcccaaacaggtcatggtgcgcatcttctccaccaaccagggtgggttcatgctgcctatctacgagacggccgcgaaggcctacgccgtggggcagtttgagaagcccaccgagacccctcccgaagacctggacaccctgagcctggccatcgaggcagccatccaggacctgaggaacaaatctcag

SEQ ID NO: 92 - protein, 580 aa, synthetic construct
MESSAKRKMDPDNPDEGPSSKVPRPETPVTKATTFLQTMLRKEVNSQLSLGDPLFPELAEESLKTFEQVTEDCNENPEKDVLTELGDILAQAVNHAGIDSSSTGPTLTTHSCSVSSAPLNKPTPTSVAVTNTPLPGASATPELSPRKKPRKTTRPFKVIIKPPVPPAPIMLPLIKQEDIKPEPDFTIQYRNKIIDTAGCIVISDSEEEQGEEVETRGATASSPSTGSGTPRVTSPTHPLSQMNHPPLPDPLGRPDEDSSSSSSSSCSSASDSESESEEMKCSSGGGASVTSSHHGRGGFGGAASSSLLSCGHQSSGGASTGPRKKKSKRISELDNEKVRNIMKDKNTPFCTPNVQTRRGRVKIDEVSRMFRHTNRSLEYKNLPFMIPSMHQVLEEAIKVCKTMQVNNKGIQIIYTRNHEVKNEVDQVRCRLGSMCNLALSTPFLMEHTMPVTHPPDVAQRTADACNDGVKAVWNLKELHTHQLCPRSSDYRNMIIHAATPVDLLGALNLCLPLMQKFPKQVMVRIFSTNQGGFMLPIYETAAKAYAVGQFEKPTETPPEDLDTLSLAIEAAIQDLRNKSQ

SEQ ID NO: 93 - DNA, 200 bp, synthetic construct
  source 1..200: mol_type = other DNA
tgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcaga

SEQ ID NO: 94 - DNA, 6107 bp, synthetic construct
  source 1..6107: mol_type = other DNA
ctggggactttccagcctggggactttccagctgggactttccagggtcgtggtttgtctggtcaaccaccgcggtctcagtggtgtacggtacaaaccccgaccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggtcggccggcttgtcgacgacggcggtctccgtcgtcaggatcatccgggcgccaccatggagaccccgtcccagcggcgcgccacccgcagcggggcgcaggccagctccactccgctgtcgcccacccgcatcacccggctgcaggagaaggaggacctgcaggagctcaatgatcgcttggcggtctacatcgaccgtgtgcgctcgctggaaacggagaacgcagggctgcgccttcgcatcaccgagtctgaagaggtggtcagccgcgaggtgtccggcatcaaggccgcctacgaggccgagctcggggatgcccgcaagacccttgactcagtagccaaggagcgcgcccgcctgcagctggagctgagcaaagtgcgtgaggagtttaaggagctgaaagcgcgcaataccaagaaggagggtgacctgatagctgctcaggctcggctgaaggacctggaggctctgctgaactccaaggaggccgcactgagcactgctctcagtgagaagcgcacgctggagggcgagctgcatgatctgcggggccaggtggccaagcttgaggcagccctaggtgaggccaagaagcaacttcaggatgagatgctgcggcgggtggatgctgagaacaggctgcagaccatgaaggaggaactggacttccagaagaacatctacagtgaggagctgcgtgagaccaagcgccgtcatgagacccgactggtggagattgacaatgggaagcagcgtgagtttgagagccggctggcggatgcgctgcaggaactgcgggcccagcatgaggaccaggtggagcagtataagaaggagctggagaagacttattctgccaagctggacaatgccaggcagtctgctgagaggaacagcaacctggtgggggctgcccacgaggagctgcagcagtcgcgcatccgcatcgacagcctctctgcccagctcagccagctccagaagcagctggcagccaaggaggcgaagcttcgagacctggaggactcactggcccgtgagcgggacaccagccggcggctgctggcggaaaaggagcgggagatggccgagatgcgggcaaggatgcagcagcagctggacgagtaccaggagcttctggacatcaagctggccctggacatggagatccacgcctaccgcaagctcttggagggcgaggaggagaggctacgcctgtcccccagccctacctcgcagcgcagccgtggccgtgcttcctctcactcatcccagacacagggtgggggcagcgtcaccaaaaagcgcaaactggagtccactgagagccgcagcagcttctcacagcacgcacgcactagcgggcgcgtggccgtggaggaggtggatgaggagggcaagtttgtccggctgcgcaacaagtccaatgaggaccagtccatgggcaattggcagatcaagcgccagaatggagatgatcccttgctgacttaccggttcccaccaaagttcaccctgaaggctgggcaggtggtgacgatctgggctgcaggagctggggccacccacagcccccctaccgacctggtgtggaaggcacagaacacctggggctgcgggaacagcctgcgtacggctctcatcaactccactggggaagaagtggccatgcgcaagctggtgcgctcagtgactgtggttgaggacgacgaggatgaggatggagatgacctgctccatcaccaccacggctcccactgcagcagctcgggggaccccgctgagtacaacctgcgctcgcgcaccgtgctgtgcgggacctgcgggcagcctgccgacaaggcatctgccagcggctcaggagcccaggtgggcggacccatctcctctggctcttctgcctccagtgtcacggtcactcgcagctaccgcagtgtggggggcagtgggggtggcagcttcggggacaatctggtcacccgctcctacctcctgggcaactccagcccccgaacccagagcccccagaactgcagcatcatacaagagatgggaatgaggtgggaggtggaagaagggagaagaaaggtgagtttgagctgccttcccggctctggagctacaaacttcagcctgctgaagcaggccggagatgttgaagagaaccccggccctatgagcagactggataagtcgaaggtcatcaactccgccctcgagctgctgaacggcgtgggaatcgaaggcctgacaaccagaaagcttgcccagaaactgggcgtggaacagcctaccctgtactggcacgtgaagaacaagcgggccctgctggacgccctgcctatcgagatgctggacagacaccacacgcacagctgtccactggaaggcgaaagctggcaggactttctgagaaacaacgccaaaagctaccggtgtgcccttctttctcatagagatggcgccaaggtgcacctgggcacccgtccaaccgagaaacagtacgagacactggaaaaccagctggctttcctgtgccagcagggcttctctctggaaaatgccctctatgccctgtctgctgtgggccacttcaccctgggctgcgtgttggaggagcaagagcaccaggtggccaaggaagaacgggaaacacccaccaccgacagcatgccacctctgctgaagcaagccatcgagctgttcgaccgccagggcgccgagcctgcctttctgtttgggctggagctgattatctgcggcctggaaaagcaactgaagtgcgagagcggcggacctaccgatgctctggacgatttcgatctggatatgctgcccgccgacgcactggacgacttcgacctggacatgctgcccgctgatgccctggacgacttcgacctggacatgctgcctggataactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcaggctggggagggccgccgatcgaacttgtttattgcagcttataatggttacaaataaagcaatagcatcacaaatttcacaaataaagcatttttttcactgcattctagttgtggtttgtccaaactcatcaatgtatcttactagctcatgccggtgtgcagtctttccaccacgctgcccagtctcaggtgttgctcgtactcctgtaggtcgccgaagtcgatggttcttgtcaggccgccccggacatcgaatgtcaaccgcacgttcatggaccgcagccaggtgttcttggcggctgtgtcctgttcccgccaccagtcgccgaaccgctgtccggtttctcgccactcccatccgctaggccgggcttccaggccctccagttcctcctgcctagcagccagagcggcgattctggcgtccagggcttctctctgtggagaaccggctctataggcggggctgccgatcaggctggtcagatccaccagctcggcgttcacctcggccagttccacggcgctgtcgctgccggccacccacaccttctcgagtctctcagcatcgccgagcagatccagcacttgttcctcgcagaaggcatcccactcggccatggccacggtgccattgccgcagtgtttggggaagcccatggatctgcaccggtatctagggtgctttctgcctccgccggcgaacttgtaggcgggttctccgcacacggcacagaacaggaccctcagcagcaggcttggggtggacacagcaggtttagctctagaggtcttcaccagctcggcccgcagggcctccagctgctctcttgtcaggataggttcggcccgcaccagaggggcgccgtcatcatctcgcacggtcttgccgttcagggtggcgtatcccagcatggcctcgctgatcatgctgcgcttcagggctgtggcgctccactctctgccctgaggctctcttccctgcagctgggcgaagtagtccttaggagacagcacgccccttctgttcaagtcgtgagccaccagatggagtggctcgtggttgtcaacgacccggtggtacacttccaggatccgctcccgctgcacaggatcaggcaccagtctccactcgccgtccactctggtaggcaggtagccccaggggggcagagatcctctgtatttgccggctctgatgttgaaatgggcggcagatctatttctttccttgatagcttccagttccatctgggccacggtgcccatcagggcgatcaccacggcggcaaaaggggtggtggtgtcgaagtgtgcttctgtggcgctcacgaccagcttcttgtgatcctcggcccagtgcaccagctgttgcaggtgccggatgcttctggtcagtctgtccacccggtaggccacaatcacatcgaaaggctgctcctcaaaagccagccatctagccaggttgggtctccgctttctgtcaaaggggtccacagcgccggaaacgtccaggtcctcggcgacgcccaccacgtcccagccccgctgggcgcacagctgctgacagctctcgagctgtctctcagggctagttgttgcgtctgtgactctgctcagccggatcaccaccagagctctcatggtggctttacgagggtaggaagtggtacggaaagttggtataagacaaaagtgttgtggaattgctccaggcgatctgacggttcactaaacgagctctgcttttataggcgcccaccgtacacgcctaaagcttatacgttctctatcactgatagggagtaaactggatatacgttctctatcactgatagggagtaaactgtagatacgttctctatcactgatagggagtaaactggtcatacgttctctatcactgatagggagtaaactccttatacgttctctatcactgatagggagtaaagtctgcatacgttctctatcactgatagggagtaaactcttcatacgttctctatcactgatagggagtaaactcctggggactttccagctggggactttccagctgggactttccaggag

SEQ ID NO: 95 - DNA, 2058 bp, synthetic construct
  source 1..2058: mol_type = other DNA
atggagaccccgtcccagcggcgcgccacccgcagcggggcgcaggccagctccactccgctgtcgcccacccgcatcacccggctgcaggagaaggaggacctgcaggagctcaatgatcgcttggcggtctacatcgaccgtgtgcgctcgctggaaacggagaacgcagggctgcgccttcgcatcaccgagtctgaagaggtggtcagccgcgaggtgtccggcatcaaggccgcctacgaggccgagctcggggatgcccgcaagacccttgactcagtagccaaggagcgcgcccgcctgcagctggagctgagcaaagtgcgtgaggagtttaaggagctgaaagcgcgcaataccaagaaggagggtgacctgatagctgctcaggctcggctgaaggacctggaggctctgctgaactccaaggaggccgcactgagcactgctctcagtgagaagcgcacgctggagggcgagctgcatgatctgcggggccaggtggccaagcttgaggcagccctaggtgaggccaagaagcaacttcaggatgagatgctgcggcgggtggatgctgagaacaggctgcagaccatgaaggaggaactggacttccagaagaacatctacagtgaggagctgcgtgagaccaagcgccgtcatgagacccgactggtggagattgacaatgggaagcagcgtgagtttgagagccggctggcggatgcgctgcaggaactgcgggcccagcatgaggaccaggtggagcagtataagaaggagctggagaagacttattctgccaagctggacaatgccaggcagtctgctgagaggaacagcaacctggtgggggctgcccacgaggagctgcagcagtcgcgcatccgcatcgacagcctctctgcccagctcagccagctccagaagcagctggcagccaaggaggcgaagcttcgagacctggaggactcactggcccgtgagcgggacaccagccggcggctgctggcggaaaaggagcgggagatggccgagatgcgggcaaggatgcagcagcagctggacgagtaccaggagcttctggacatcaagctggccctggacatggagatccacgcctaccgcaagctcttggagggcgaggaggagaggctacgcctgtcccccagccctacctcgcagcgcagccgtggccgtgcttcctctcactcatcccagacacagggtgggggcagcgtcaccaaaaagcgcaaactggagtccactgagagccgcagcagcttctcacagcacgcacgcactagcgggcgcgtggccgtggaggaggtggatgaggagggcaagtttgtccggctgcgcaacaagtccaatgaggaccagtccatgggcaattggcagatcaagcgccagaatggagatgatcccttgctgacttaccggttcccaccaaagttcaccctgaaggctgggcaggtggtgacgatctgggctgcaggagctggggccacccacagcccccctaccgacctggtgtggaaggcacagaacacctggggctgcgggaacagcctgcgtacggctctcatcaactccactggggaagaagtggccatgcgcaagctggtgcgctcagtgactgtggttgaggacgacgaggatgaggatggagatgacctgctccatcaccaccacggctcccactgcagcagctcgggggaccccgctgagtacaacctgcgctcgcgcaccgtgctgtgcgggacctgcgggcagcctgccgacaaggcatctgccagcggctcaggagcccaggtgggcggacccatctcctctggctcttctgcctccagtgtcacggtcactcgcagctaccgcagtgtggggggcagtgggggtggcagcttcggggacaatctggtcacccgctcctacctcctgggcaactccagcccccgaacccagagcccccagaactgcagcatcatacaagagatgggaatgaggtgggaggtggaagaagggagaagaaaggtgagtttgagctgccttccc

SEQ ID NO: 96 - protein, 686 aa, synthetic construct
METPSQRRATRSGAQASSTPLSPTRITRLQEKEDLQELNDRLAVYIDRVRSLETENAGLRLRITESEEVVSREVSGIKAAYEAELGDARKTLDSVAKERARLQLELSKVREEFKELKARNTKKEGDLIAAQARLKDLEALLNSKEAALSTALSEKRTLEGELHDLRGQVAKLEAALGEAKKQLQDEMLRRVDAENRLQTMKEELDFQKNIYSEELRETKRRHETRLVEIDNGKQREFESRLADALQELRAQHEDQVEQYKKELEKTYSAKLDNARQSAERNSNLVGAAHEELQQSRIRIDSLSAQLSQLQKQLAAKEAKLRDLEDSLARERDTSRRLLAEKEREMAEMRARMQQQLDEYQELLDIKLALDMEIHAYRKLLEGEEERLRLSPSPTSQRSRGRASSHSSQTQGGGSVTKKRKLESTESRSSFSQHARTSGRVAVEEVDEEGKFVRLRNKSNEDQSMGNWQIKRQNGDDPLLTYRFPPKFTLKAGQVVTIWAAGAGATHSPPTDLVWKAQNTWGCGNSLRTALINSTGEEVAMRKLVRSVTVVEDDEDEDGDDLLHHHHGSHCSSSGDPAEYNLRSRTVLCGTCGQPADKASASGSGAQVGGPISSGSSASSVTVTRSYRSVGGSGGGSFGDNLVTRSYLLGNSSPRTQSPQNCSIIQEMGMRWEVEEGRRKVSLSCLP

SEQ ID NO: 97 - DNA, 58 bp, synthetic construct
  source 1..58: mol_type = other DNA
gtcgtggtttgtctggtcaaccaccgcggtctcagtggtgtacggtacaaaccccgac

SEQ ID NO: 98 - DNA, 50 bp, synthetic construct
  source 1..50: mol_type = other DNA
tcggccggcttgtcgacgacggcggtctccgtcgtcaggatcatccgggc

SEQ ID NO: 99 - DNA, 1503 bp, synthetic construct
  source 1..1503: mol_type = other DNA
atgagagctctggtggtgatccggctgagcagagtcacagacgcaacaactagccctgagagacagctcgagagctgtcagcagctgtgcgcccagcggggctgggacgtggtgggcgtcgccgaggacctggacgtttccggcgctgtggacccctttgacagaaagcggagacccaacctggctagatggctggcttttgaggagcagcctttcgatgtgattgtggcctaccgggtggacagactgaccagaagcatccggcacctgcaacagctggtgcactgggccgaggatcacaagaagctggtcgtgagcgccacagaagcacacttcgacaccaccaccccttttgccgccgtggtgatcgccctgatgggcaccgtggcccagatggaactggaagctatcaaggaaagaaatagatctgccgcccatttcaacatcagagccggcaaatacagaggatctctgcccccctggggctacctgcctaccagagtggacggcgagtggagactggtgcctgatcctgtgcagcgggagcggatcctggaagtgtaccaccgggtcgttgacaaccacgagccactccatctggtggctcacgacttgaacagaaggggcgtgctgtctcctaaggactacttcgcccagctgcagggaagagagcctcagggcagagagtggagcgccacagccctgaagcgcagcatgatcagcgaggccatgctgggatacgccaccctgaacggcaagaccgtgcgagatgatgacggcgcccctctggtgcgggccgaacctatcctgacaagagagcagctggaggccctgcgggccgagctggtgaagacctctagagctaaacctgctgtgtccaccccaagcctgctgctgagggtcctgttctgtgccgtgtgcggagaacccgcctacaagttcgccggcggaggcagaaagcaccctagataccggtgcagatccatgggcttccccaaacactgcggcaatggcaccgtggccatggccgagtgggatgccttctgcgaggaacaagtgctggatctgctcggcgatgctgagagactcgagaaggtgtgggtggccggcagcgacagcgccgtggaactggccgaggtgaacgccgagctggtggatctgaccagcctgatcggcagccccgcctatagagccggttctccacagagagaagccctggacgccagaatcgccgctctggctgctaggcaggaggaactggagggcctggaagcccggcctagcggatgggagtggcgagaaaccggacagcggttcggcgactggtggcgggaacaggacacagccgccaagaacacctggctgcggtccatgaacgtgcggttgacattcgatgtccggggcggcctgacaagaaccatcgacttcggcgacctacaggagtacgagcaacacctgagactgggcagcgtggtggaaagactgcacaccggcatgagctag

SEQ ID NO: 100 - protein, 500 aa, synthetic construct [sequence omitted]

SEQ ID NO: 101 - DNA, 6853 bp, synthetic construct
  source 1..6853: mol_type = other DNA
ctggggactttccagcctggggactttccagctgggactttccaggcgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttgggccaccatgcagaggtcgcctctggaaaaggccagcgttgtctccaaactttttttcagctggaccagaccaattttgaggaaaggatacagacagcgcctggaattgtcagacatataccaaatcccttctgttgattctgctgacaatctatctgaaaaattggaaagagaatgggatagagagctggcttcaaagaaaaatcctaaactcattaatgcccttcggcgatgttttttctggagatttatgttctatggaatctttttatatttaggggaagtcaccaaagcagtacagcctctcttactgggaagaatcatagcttcctatgacccggataacaaggaggaacgctctatcgcgatttatctaggcataggcttatgccttctctttattgtgaggacactgctcctacacccagccatttttggccttcatcacattggaatgcagatgagaatagctatgtttagtttgatttataagaagactttaaagctgtcaagccgtgttctagataaaataagtattggacaacttgttagtctcctttccaacaacctgaacaaatttgatgaaggacttgcattggcacatttcgtgtggatcgctcctttgcaagtggcactcctcatggggctaatctgggagttgttacaggcgtctgccttctgtggacttggtttcctgatagtccttgccctttttcaggctgggctagggagaatgatgatgaagtacagagatcagagagctgggaagatcagtgaaagacttgtgattacctcagaaatgattgaaaatatccaatctgttaaggcatactgctgggaagaagcaatggaaaaaatgattgaaaacttaagacaaacagaactgaaactgactcggaaggcagcctatgtgagatacttcaatagctcagccttcttcttctcagggttctttgtggtgtttttatctgtgcttccctatgcactaatcaaaggaatcatcctccggaaaatattcaccaccatctcattctgcattgttctgcgcatggcggtcactcggcaatttccctgggctgtacaaacatggtatgactctcttggagcaataaacaaaatacaggatttcttacaaaagcaagaatataagacattggaatataacttaacgactacagaagtagtgatggagaatgtaacagccttctgggaggagggatttggggaattatttgagaaagcaaaacaaaacaataacaatagaaaaacttctaatggtgatgacagcctcttcttcagtaatttctcacttcttggtactcctgtcctgaaagatattaatttcaagatagaaagaggacagttgttggcggttgctggatccactggagcaggcaagacttcacttctaatggtgattatgggagaactggagccttcagagggtaaaattaagcacagtggaagaatttcattctgttctcagttttcctggattatgcctggcaccattaaagaaaatatcatctttggtgtttcctatgatgaatatagatacagaagcgtcatcaaagcatgccaactagaagaggacatctccaagtttgcagagaaagacaatatagttcttggagaaggtggaatcacactgagtggaggtcaacgagcaagaatttctttagcaagagcagtatacaaagatgctgatttgtatttattagactctccttttggatacctagatgttttaacagaaaaagaaatatttgaaagctgtgtctgtaaactgatggctaacaaaactaggattttggtcacttctaaaatggaacatttaaagaaagctgacaaaatattaattttgcatgaaggtagcagctatttttatgggacattttcagaactccaaaatctacagccagactttagctcaaaactcatgggatgtgattctttcgaccaatttagtgcagaaagaagaaattcaatcctaactgagaccttacaccgtttctcattagaaggagatgctcctgtctcctggacagaaacaaaaaaacaatcttttaaacagactggagagtttggggaaaaaaggaagaattctattctcaatccaatcaactctatacgaaaattttccattgtgcaaaagactcccttacaaatgaatggcatcgaagaggattctgatgagcctttagagagaaggctgtccttagtaccagattctgagcagggagaggcgatactgcctcgcatcagcgtgatcagcactggccccacgcttcaggcacgaaggaggcagtctgtcctgaacctgatgacacactcagttaaccaaggtcagaacattcaccgaaagacaacagcatccacacgaaaagtgtcactggcccctcaggcaaacttgactgaactggatatatattcaagaaggttatctcaagaaactggcttggaaataagtgaagaaattaacgaagaagacttaaaggagtgcttttttgatgatatggagagcataccagcagtgactacatggaacacataccttcgatatattactgtccacaagagcttaatttttgtgctaatttggtgcttagtaatttttctggcagaggtggctgcttctttggttgtgctgtggctccttggaaacactcctcttcaagacaaagggaatagtactcatagtagaaataacagctatgcagtgattatcaccagcaccagttcgtattatgtgttttacatttacgtgggagtagccgacactttgcttgctatgggattcttcagaggtctaccactggtgcatactctaatcacagtgtcgaaaattttacaccacaaaatgttacattctgttcttcaagcacctatgtcaaccctcaacacgttgaaagcaggtgggattcttaatagattctccaaagatatagcaattttggatgaccttctgcctcttaccatatttgacttcatccagttgttattaattgtgattggagctatagcagttgtcgcagttttacaaccctacatctttgttgcaacagtgccagtgatagtggcttttattatgttgagagcatatttcctccaaacctcacagcaactcaaacaactggaatctgaaggcaggagtccaattttcactcatcttgttacaagcttaaaaggactatggacacttcgtgccttcggacggcagccttactttgaaactctgttccacaaagctctgaatttacatactgccaactggttcttgtacctgtcaacactgcgctggttccaaatgagaatagaaatgatttttgtcatcttcttcattgctgttaccttcatttccattttaacaacaggagaaggagaaggaagagttggtattatcctgactttagccatgaatatcatgagtacattgcagtgggctgtaaactccagcatagatgtggatagcttgatgcgatctgtgagccgagtctttaagttcattgacatgccaacagaaggtaaacctaccaagtcaaccaaaccatacaagaatggccaactctcgaaagttatgattattgagaattcacacgtgaagaaagatgacatctggccctcagggggccaaatgactgtcaaagatctcacagcaaaatacacagaaggtggaaatgccatattagagaacatttccttctcaataagtcctggccagagggtgggcctcttgggaagaactggatcagggaagagtactttgttatcagcttttttgagactactgaacactgaaggagaaatccagatcgatggtgtgtcttgggattcaataactttgcaacagtggaggaaagcctttggagtgataccacagaaagtatttattttttctggaacatttagaaaaaacttggatccctatgaacagtggagtgatcaagaaatatggaaagttgcagatgaggttgggctcagatctgtgatagaacagtttcctgggaagcttgactttgtccttgtggatgggggctgtgtcctaagccatggccacaagcagttgatgtgcttggctagatctgttctcagtaaggcgaagatcttgctgcttgatgaacccagtgctcatttggatccagtaacataccaaataattagaagaactctaaaacaagcatttgctgattgcacagtaattctctgtgaacacaggatagaagcaatgctggaatgccaacaatttttggtcatagaagagaacaaagtgcggcagtacgattccatccagaaactgctgaacgagaggagcctcttccggcaagccatcagcccctccgacagggtgaagctctttccccaccggaactcaagcaagtgcaagtctaagccccagattgctgctctgaaagaggagacagaagaagaggtgcaagatacaaggcttggatctggcgctactaacttcagtctgctgaagcaggccggggacgtggaagagaaccccggtcctatgctggaaggcgtgcaggtggaaaccatcagccccggcgacggtagaacctttcccaagcgcggccagacctgcgtggtccactacaccggcatgctggaagatggcaagaaggtggacagcagcagagatagaaacaagcccttcaagttcatgctgggcaaacaggaggtgattaggggctgggaggaaggcgtggcccagatgagcgttggacagcgggccaaactgacaatctctcctgactacgcctacggagctaccggccaccccgggatcatccctcctcacgccaccctggtctttgatgtggagctgctgaaactggaaagcggcggcggaagcggcgtggacggcttcggcgatgtgggcgccctggaatctctgagaggaaatgccgacctggcctacatcctgagcatggaaccgtgcggccactgtctgatcatcaacaacgtgaacttctgcagagagagcggcctgagaacaagaacaggctccaacatcgattgcgagaaactgcggagacggttcagcagcctccatttcatggtcgaagtgaagggcgacctgaccgccaagaaaatggtgctggccctgctggaactggccagacaggaccatggcgctctggactgctgcgtggtggtgatcctgtcccacggctgtcaggctagccacctgcaatttcctggagccgtgtacggcaccgacggatgccccgtgtctgttgagaagatcgtcaacatctttaacggcaccagctgcccttctttgggcgggaagcctaagttgttcttcatccaggcctgtggcggagaacagaaggaccacggctttgaggtggcttccacaagccctgaggatgagagccctggctctaatccagagcctgacgccacacctttccaagagggcctgcggaccttcgatcagctggacgccatcagctctctgcctacccctagcgacatcttcgtgtcctacagcaccttccctggatttgtgtcctggcgggaccccaagagcggaagctggtacgtggagacactggacgatatttttgagcagtgggcccacagcgaggacctgcagagcctgctgctcagagtggccaacgccgtaagcgtgaaaggcatctacaagcagatgcctggctgcttcaacttcctgagaaagaagctgttcttcaagaccagcgtggattatccatacgacgtgcccgactacgccctggactaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcaggctggggactggggactttccagctggggactttccagctgggactttccaggag

SEQ ID NO: 102 - DNA, 4440 bp, synthetic construct
  source 1..4440: mol_type = other DNA
atgcagaggtcgcctctggaaaaggccagcgttgtctccaaactttttttcagctggaccagaccaattttgaggaaaggatacagacagcgcctggaattgtcagacatataccaaatcccttctgttgattctgctgacaatctatctgaaaaattggaaagagaatgggatagagagctggcttcaaagaaaaatcctaaactcattaatgcccttcggcgatgttttttctggagatttatgttctatggaatctttttatatttaggggaagtcaccaaagcagtacagcctctcttactgggaagaatcatagcttcctatgacccggataacaaggaggaacgctctatcgcgatttatctaggcataggcttatgccttctctttattgtgaggacactgctcctacacccagccatttttggccttcatcacattggaatgcagatgagaatagctatgtttagtttgatttataagaagactttaaagctgtcaagccgtgttctagataaaataagtattggacaacttgttagtctcctttccaacaacctgaacaaatttgatgaaggacttgcattggcacatttcgtgtggatcgctcctttgcaagtggcactcctcatggggctaatctgggagttgttacaggcgtctgccttctgtggacttggtttcctgatagtccttgccctttttcaggctgggctagggagaatgatgatgaagtacagagatcagagagctgggaagatcagtgaaagacttgtgattacctcagaaatgattgaaaatatccaatctgttaaggcatactgctgggaagaagcaatggaaaaaatgattgaaaacttaagacaaacagaactgaaactgactcggaaggcagcctatgtgagatacttcaatagctcagccttcttcttctcagggttctttgtggtgtttttatctgtgcttccctatgcactaatcaaaggaatcatcctccggaaaatattcaccaccatctcattctgcattgttctgcgcatggcggtcactcggcaatttccctgggctgtacaaacatggtatgactctcttggagcaataaacaaaatacaggatttcttacaaaagcaagaatataagacattggaatataacttaacgactacagaagtagtgatggagaatgtaacagccttctgggaggagggatttggggaattatttgagaaagcaaaacaaaacaataacaatagaaaaacttctaatggtgatgacagcctcttcttcagtaatttctcacttcttggtactcctgtcctgaaagatattaatttcaagatagaaagaggacagttgttggcggttgctggatccactggagcaggcaagacttcacttctaatggtgattatgggagaactggagccttcagagggtaaaattaagcacagtggaagaatttcattctgttctcagttttcctggattatgcctggcaccattaaagaaaatatcatctttggtgtttcctatgatgaatatagatacagaagcgtcatcaaagcatgccaactagaagaggacatctccaagtttgcagagaaagacaatatagttcttggagaaggtggaatcacactgagtggaggtcaacgagcaagaatttctttagcaagagcagtatacaaagatgctgatttgtatttattagactctccttttggatacctagatgttttaacagaaaaagaaatatttgaaagctgtgtctgtaaactgatggctaacaaaactaggattttggtcacttctaaaatggaacatttaaagaaagctgacaaaatattaattttgcatgaaggtagcagctatttttatgggacattttcagaactccaaaatctacagccagactttagctcaaaactcatgggatgtgattctttcgaccaatttagtgcagaaagaagaaattcaatcctaactgagaccttacaccgtttctcattagaaggagatgctcctgtctcctggacagaaacaaaaaaacaatcttttaaacagactggagagtttggggaaaaaaggaagaattctattctcaatccaatcaactctatacgaaaattttccattgtgcaaaagactcccttacaaatgaatggcatcgaagaggattctgatgagcctttagagagaaggctgtccttagtaccagattctgagcagggagaggcgatactgcctcgcatcagcgtgatcagcactggccccacgcttcaggcacgaaggaggcagtctgtcctgaacctgatgacacactcagttaaccaaggtcagaacattcaccgaaagacaacagcatccacacgaaaagtgtcactggcccctcaggcaaacttgactgaactggatatatattcaagaaggttatctcaagaaactggcttggaaataagtgaagaaattaacgaagaagacttaaaggagtgcttttttgatgatatggagagcataccagcagtgactacatggaacacataccttcgatatattactgtccacaagagcttaatttttgtgctaatttggtgcttagtaatttttctggcagaggtggctgcttctttggttgtgctgtggctccttggaaacactcctcttcaagacaaagggaatagtactcatagtagaaataacagctatgcagtgattatcaccagcaccagttcgtattatgtgttttacatttacgtgggagtagccgacactttgcttgctatgggattcttcagaggtctaccactggtgcatactctaatcacagtgtcgaaaattttacaccacaaaatgttacattctgttcttcaagcacctatgtcaaccctcaacacgttgaaagcaggtgggattcttaatagattctccaaagatatagcaattttggatgaccttctgcctcttaccatatttgacttcatccagttgttattaattgtgattggagctatagcagttgtcgcagttttacaaccctacatctttgttgcaacagtgccagtgatagtggcttttattatgttgagagcatatttcctccaaacctcacagcaactcaaacaactggaatctgaaggcaggagtccaattttcactcatcttgttacaagcttaaaaggactatggacacttcgtgccttcggacggcagccttactttgaaactctgttccacaaagctctgaatttacatactgccaactggttcttgtacctgtcaacactgcgctggttccaaatgagaatagaaatgatttttgtcatcttcttcattgctgttaccttcatttccattttaacaacaggagaaggagaaggaagagttggtattatcctgactttagccatgaatatcatgagtacattgcagtgggctgtaaactccagcatagatgtggatagcttgatgcgatctgtgagccgagtctttaagttcattgacatgccaacagaaggtaaacctaccaagtcaaccaaaccatacaagaatggccaactctcgaaagttatgattattgagaattcacacgtgaagaaagatgacatctggccctcagggggccaaatgactgtcaaagatctcacagcaaaatacacagaaggtggaaatgccatattagagaacatttccttctcaataagtcctggccagagggtgggcctcttgggaagaactggatcagggaagagtactttgttatcagcttttttgagactactgaacactgaaggagaaatccagatcgatggtgtgtcttgggattcaataactttgcaacagtggaggaaagcctttggagtgataccacagaaagtatttattttttctggaacatttagaaaaaacttggatccctatgaacagtggagtgatcaagaaatatggaaagttgcagatgaggttgggctcagatctgtgatagaacagtttcctgggaagcttgactttgtccttgtggatgggggctgtgtcctaagccatggccacaagcagttgatgtgcttggctagatctgttctcagtaaggcgaagatcttgctgcttgatgaacccagtgctcatttggatccagtaacataccaaataattagaagaactctaaaacaagcatttgctgattgcacagtaattctctgtgaacacaggatagaagcaatgctggaatgccaacaatttttggtcatagaagagaacaaagtgcggcagtacgattccatccagaaactgctgaacgagaggagcctcttccggcaagccatcagcccctccgacagggtgaagctctttccccaccggaactcaagcaagtgcaagtctaagccccagattgctgctctgaaagaggagacagaagaagaggtgcaagatacaaggctt

SEQ ID NO: 103 - protein, 1480 aa, synthetic construct
MQRSPLEKASVVSKLFFSWTRPILRKGYRQRLELSDIYQIPSVDSADNLSEKLEREWDRELASKKNPKLINALRRCFFWRFMFYGIFLYLGEVTKAVQPLLLGRIIASYDPDNKEERSIAIYLGIGLCLLFIVRTLLLHPAIFGLHHIGMQMRIAMFSLIYKKTLKLSSRVLDKISIGQLVSLLSNNLNKFDEGLALAHFVWIAPLQVALLMGLIWELLQASAFCGLGFLIVLALFQAGLGRMMMKYRDQRAGKISERLVITSEMIENIQSVKAYCWEEAMEKMIENLRQTELKLTRKAAYVRYFNSSAFFFSGFFVVFLSVLPYALIKGIILRKIFTTISFCIVLRMAVTRQFPWAVQTWYDSLGAINKIQDFLQKQEYKTLEYNLTTTEVVMENVTAFWEEGFGELFEKAKQNNNNRKTSNGDDSLFFSNFSLLGTPVLKDINFKIERGQLLAVAGSTGAGKTSLLMVIMGELEPSEGKIKHSGRISFCSQFSWIMPGTIKENIIFGVSYDEYRYRSVIKACQLEEDISKFAEKDNIVLGEGGITLSGGQRARISLARAVYKDADLYLLDSPFGYLDVLTEKEIFESCVCKLMANKTRILVTSKMEHLKKADKILILHEGSSYFYGTFSELQNLQPDFSSKLMGCDSFDQFSAERRNSILTETLHRFSLEGDAPVSWTETKKQSFKQTGEFGEKRKNSILNPINSIRKFSIVQKTPLQMNGIEEDSDEPLERRLSLVPDSEQGEAILPRISVISTGPTLQARRRQSVLNLMTHSVNQGQNIHRKTTASTRKVSLAPQANLTELDIYSRRLSQETGLEISEEINEEDLKECFFDDMESIPAVTTWNTYLRYITVHKSLIFVLIWCLVIFLAEVAASLVVLWLLGNTPLQDKGNSTHSRNNSYAVIITSTSSYYVFYIYVGVADTLLAMGFFRGLPLVHTLITVSKILHHKMLHSVLQAPMSTLNTLKAGGILNRFSKDIAILDDLLPLTIFDFIQLLLIVIGAIAVVAVLQPYIFVATVPVIVAFIMLRAYFLQTSQQLKQLESEGRSPIFTHLVTSLKGLWTLRAFGRQPYFETLFHKALNLHTANWFLYLSTLRWFQMRIEMIFVIFFIAVTFISILTTGEGEGRVGIILTLAMNIMSTLQWAVNSSIDVDSLMRSVSRVFKFIDMPTEGKPTKSTKPYKNGQLSKVMIIENSHVKKDDIWPSGGQMTVKDLTAKYTEGGNAILENISFSISPGQRVGLLGRTGSGKSTLLSAFLRLLNTEGEIQIDGVSWDSITLQQWRKAFGVIPQKVFIFSGTFRKNLDPYEQWSDQEIWKVADEVGLRSVIEQFPGKLDFVLVDGGCVLSHGHKQLMCLARSVLSKAKILLLDEPSAHLDPVTYQIIRRTLKQAFADCTVILCEHRIEAMLECQQFLVIEENKVRQYDSIQKLLNERSLFRQAISPSDRVKLFPHRNSSKCKSKPQIAALKEETEEEVQDTRL

SEQ ID NO: 104 - DNA, 1242 bp, synthetic construct
  source 1..1242: mol_type = other DNA
atgctcgagggagtgcaggtggagactatctccccaggagacgggcgcaccttccccaagcgcggccagacctgcgtggtgcactacaccgggatgcttgaagatggaaagaaagttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaatctggcggtggatccggagtcgacggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcagtcgactatccgtacgacgtaccagactacgcactcgactaa

SEQ ID NO: 105 - DNA, 4018 bp, synthetic construct
  source 1..4018: mol_type = other DNA
ctggggactttccagcctggggactttccagctgggactttccaggcgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttgggccaccatgacaagctccaggctttggttttcgctgctgctggcggcagcgttcgcaggacgggcgacggccctctggccctggcctcagaacttccaaacctccgaccagcgctacgtcctttacccgaacaactttcaattccagtacgatgtcagctcggccgcgcagcccggctgctcagtcctcgacgaggccttccagcgctatcgtgacctgcttttcggttccgggtcttggccccgtccttacctcacaggttggccgcaccaagcttaccctgtcttcctagggaaacggcatacactggagaagaatgtgttggttgtctctgtagtcacacctggatgtaaccagcttcctactttggagtcagtggagaattataccctgaccataaatgatgaccagtgtttactcctctctgagactgtctggggagctctccgaggtctggagacttttagccagcttgtttggaaatctgctgagggcacattctttatcaacaagactgagattgaggactttccccgctttcctcaccggggcttgctgttggatacatctcgccattacctgccactctctagcatcctggacactctggatgtcatggcgtacaataaattgaacgtgttccactggcatctggtagatgatccttccttcccatatgagagcttcacttttccagagctcatgagaaaggggtcctacaaccctgtcacccacatctacacagcacaggatgtgaaggaggtcattgaatacgcacggctccggggtatccgtgtgcttgcagagtttgacactcctggccacactttgtcctggggaccaggtatccctggattactgactccttgctactctgggtctgagccctctggcacctttggaccagtgaatcccagtctcaataatacctatgagttcatgagcacattcttcttagaagtcagctctgtcttcccagatttttatcttcatcttggaggagatgaggttgatttcacctgctggaagtccaacccagagatccaggactttatgaggaagaaaggcttcggtgaggacttcaagcagctggagtccttctacatccagacgctgctggacatcgtctcttcttatggcaagggctatgtggtgtggcaggaggtgtttgataataaagtaaagattcagccagacacaatcatacaggtgtggcgagaggatattccagtgaactatatgaaggagctggaactggtcaccaaggccggcttccgggcccttctctctgccccctggtacctgaaccgtatatcctatggccctgactggaaggatttctacatagtggaacccctggcatttgaaggtacccctgagcagaaggctctggtgattggtggagaggcttgtatgtggggagaatatgtggacaacacaaacctggtccccaggctctggcccagagcaggggctgttgccgaaaggctgtggagcaacaagttgacatctgacctgacatttgcctatgaacgtttgtcacacttccgctgtgaattgctgaggcgaggtgtccaggcccaacccctcaatgtaggcttctgtgagcaggagtttgaacagaccggatctggcgctactaacttcagtctgctgaagcaggccggggacgtggaagagaaccccggtcctatggggagtagcaagagcaagcctaaggaccccagccagcgctctagaggagtgcaggtggagactatctccccaggagacgggcgcaccttccccaagcgcggccagacctgcgtggtgcactacaccgggatgcttgaagatggaaagaaagttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaaactagtggaggtggaggttctagaggatcccatatgacaatctcggactctccaagagaacaggatagtgaatcacagactttggacaaagtttaccaaatgaaaagcaaacctcggggatactgtctgatcatcaacaatcacaattttgcaaaagcacgggagaaagtgcccaaacttcacagcattagggacaggaatggaacacacttggatgcaggggctttgaccacgacctttgaagagcttcattttgagatcaagccccacgatgactgcacagtagagcaaatctatgagattttgaaaatctaccaactcatggaccacagtaacatggactgcttcatctgctgtatcctctcccatggagacaagggcatcatctatggcactgatggacaggaggcccccatctatgagctgacatctcagttcactggtttgaagtgcccttcccttgctggaaaacccaaagtgttttttattcaggcttgtcagggggataactaccagaaaggtatacctgttgagactgattcagaggagcaaccctatttagaaatggatttatcatcacctcaaacgagatatatcccggatgaggctgactttctgctggggatggccactgtgaataactgtgtttcctaccgaaaccctgcagagggaacctggtacatccagtcactttgccagagcctgagagagcgatgtcctcgaggcgatgatattctcaccatcctgactgaagtgaactatgaagtaagcaacaaggatgacaagaaaaacatggggaaacagatgcctcagcctactttcacactaagaaaaaaacttgtcttcccttctgattgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcaggctggggactggggactttccagctggggactttccagctgggactttccaggag

SEQ ID NO: 106 - DNA, 1620 bp, synthetic construct
  source 1..1620: mol_type = other DNA
atgacaagctccaggctttggttttcgctgctgctggcggcagcgttcgcaggacgggcgacggccctctggccctggcctcagaacttccaaacctccgaccagcgctacgtcctttacccgaacaactttcaattccagtacgatgtcagctcggccgcgcagcccggctgctcagtcctcgacgaggccttccagcgctatcgtgacctgcttttcggttccgggtcttggccccgtccttacctcacaggttggccgcaccaagcttaccctgtcttcctagggaaacggcatacactggagaagaatgtgttggttgtctctgtagtcacacctggatgtaaccagcttcctactttggagtcagtggagaattataccctgaccataaatgatgaccagtgtttactcctctctgagactgtctggggagctctccgaggtctggagacttttagccagcttgtttggaaatctgctgagggcacattctttatcaacaagactgagattgaggactttccccgctttcctcaccggggcttgctgttggatacatctcgccattacctgccactctctagcatcctggacactctggatgtcatggcgtacaataaattgaacgtgttccactggcatctggtagatgatccttccttcccatatgagagcttcacttttccagagctcatgagaaaggggtcctacaaccctgtcacccacatctacacagcacaggatgtgaaggaggtcattgaatacgcacggctccggggtatccgtgtgcttgcagagtttgacactcctggccacactttgtcctggggaccaggtatccctggattactgactccttgctactctgggtctgagccctctggcacctttggaccagtgaatcccagtctcaataatacctatgagttcatgagcacattcttcttagaagtcagctctgtcttcccagatttttatcttcatcttggaggagatgaggttgatttcacctgctggaagtccaacccagagatccaggactttatgaggaagaaaggcttcggtgaggacttcaagcagctggagtccttctacatccagacgctgctggacatcgtctcttcttatggcaagggctatgtggtgtggcaggaggtgtttgataataaagtaaagattcagccagacacaatcatacaggtgtggcgagaggatattccagtgaactatatgaaggagctggaactggtcaccaaggccggcttccgggcccttctctctgccccctggtacctgaaccgtatatcctatggccctgactggaaggatttctacatagtggaacccctggcatttgaaggtacccctgagcagaaggctctggtgattggtggagaggcttgtatgtggggagaatatgtggacaacacaaacctggtccccaggctctggcccagagcaggggctgttgccgaaaggctgtggagcaacaagttgacatctgacctgacatttgcctatgaacgtttgtcacacttccgctgtgaattgctgaggcgaggtgtccaggcccaacccctcaatgtaggcttctgtgagcaggagtttgaacagacc

SEQ ID NO: 107 - protein, 540 aa, synthetic construct
MTSSRLWFSLLLAAAFAGRATALWPWPQNFQTSDQRYVLYPNNFQFQYDVSSAAQPGCSVLDEAFQRYRDLLFGSGSWPRPYLTGWPHQAYPVFLGKRHTLEKNVLVVSVVTPGCNQLPTLESVENYTLTINDDQCLLLSETVWGALRGLETFSQLVWKSAEGTFFINKTEIEDFPRFPHRGLLLDTSRHYLPLSSILDTLDVMAYNKLNVFHWHLVDDPSFPYESFTFPELMRKGSYNPVTHIYTAQDVKEVIEYARLRGIRVLAEFDTPGHTLSWGPGIPGLLTPCYSGSEPSGTFGPVNPSLNNTYEFMSTFFLEVSSVFPDFYLHLGGDEVDFTCWKSNPEIQDFMRKKGFGEDFKQLESFYIQTLLDIVSSYGKGYVVWQEVFDNKVKIQPDTIIQVWREDIPVNYMKELELVTKAGFRALLSAPWYLNRISYGPDWKDFYIVEPLAFEGTPEQKALVIGGEACMWGEYVDNTNLVPRLWPRAGAVAERLWSNKLTSDLTFAYERLSHFRCELLRRGVQAQPLNVGFCEQEFEQT

SEQ ID NO: 108 - DNA, 1227 bp, synthetic construct
  source 1..1227: mol_type = other DNA
atggggagtagcaagagcaagcctaaggaccccagccagcgctctagaggagtgcaggtggagactatctccccaggagacgggcgcaccttccccaagcgcggccagacctgcgtggtgcactacaccgggatgcttgaagatggaaagaaagttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaaactagtggaggtggaggttctagaggatcccatatgacaatctcggactctccaagagaacaggatagtgaatcacagactttggacaaagtttaccaaatgaaaagcaaacctcggggatactgtctgatcatcaacaatcacaattttgcaaaagcacgggagaaagtgcccaaacttcacagcattagggacaggaatggaacacacttggatgcaggggctttgaccacgacctttgaagagcttcattttgagatcaagccccacgatgactgcacagtagagcaaatctatgagattttgaaaatctaccaactcatggaccacagtaacatggactgcttcatctgctgtatcctctcccatggagacaagggcatcatctatggcactgatggacaggaggcccccatctatgagctgacatctcagttcactggtttgaagtgcccttcccttgctggaaaacccaaagtgttttttattcaggcttgtcagggggataactaccagaaaggtatacctgttgagactgattcagaggagcaaccctatttagaaatggatttatcatcacctcaaacgagatatatcccggatgaggctgactttctgctggggatggccactgtgaataactgtgtttcctaccgaaaccctgcagagggaacctggtacatccagtcactttgccagagcctgagagagcgatgtcctcgaggcgatgatattctcaccatcctgactgaagtgaactatgaagtaagcaacaaggatgacaagaaaaacatggggaaacagatgcctcagcctactttcacactaagaaaaaaacttgtcttcccttctgattga

SEQ ID NO: 109 - DNA, 4032 bp, synthetic construct
  source 1..4032: mol_type = other DNA
ctggggactttccagcctggggactttccagctgggactttccagggagtttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttaggcgtgtacggtgggcgcctataaaagcagagctcgtttagtgaaccgtcagatcgcctggagcaattccacaacacttttgtcttataccaactttccgtaccacttcctaccctcgtaaagccaccatgtattcagcgccctccgcctgcacttgcctgtgtttacacttcctgctgctgtgcttccaggtacaggtgctggttgccgaggagaacgtggacttccgcatccacgtggagaaccagacgcgggctcgggacgatgtgagccgtaagcagctgcggctgtaccagctctacagccggaccagtgggaaacacatccaggtcctgggccgcaggatcagtgcccgcggcgaggatggggacaagtatgcccagctcctagtggagacagacaccttcggtagtcaagtccggatcaagggcaaggagacggaattctacctgtgcatgaaccgcaaaggcaagctcgtggggaagcccgatggcaccagcaaggagtgtgtgttcatcgagaaggttctggagaacaactacacggccctgatgtcggctaagtactccggctggtacgtgggcttcaccaagaaggggcggccgcggaagggccccaagacccgggagaaccagcaggacgtgcatttcatgaagcgctaccccaaggggcagccggagcttcagaagcccttcaagtacacgacggtgaccaagaggtcccgtcggatccggcccacacaccctgccggatctggcgccacaaacttcagcctgctgaagcaggccggagatgtggaagaaaatcctggccccgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatttcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagccaaaaaagaagagaaagctagcagtgtcagtgacatttgaagatgtggctgtgctctttactcgggacgagtggaagaagctggatctgtctcagagaagcctgtaccgtgaggtgatgctggagaattacagcaacctggcctccatggcaggattcctgtttaccaaaccaaaggtgatctccctgttgcagcaaggagaggatccctggtaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcaggctggggaaacttgtttattgcagcttataatggttacaaataaagcaatagcatcacaaatttcacaaataaagcatttttttcactgcattctagttgtggtttgtccaaactcatcaatgtatcttattaccagggatcctctccttgctgcaacagggagatcacctttggtttggtaaacaggaatcctgccatggaggccaggttgctgtaattctccagcatcacctcacggtacaggcttctctgagacagatccagcttcttccactcgtcccgagtaaagagcacagccacatcttcaaatgtcactgacactgctagctttctcttcttttttggtttattaccatcctcaatgggtgtatgctttttttcatctaactgtgcagaaaatccaaatattaatgatttcagcccgaaatggaaggccatttcagcagaggttctcgcctgaatattaaatgcctcttgtaataatggtggtgcagcgtctatcacatgattattttctatctggtttgttgcttgctcctctaatactgcacccaacgtaaaatggctgatagattgcagaatgaaaagagcctcctcgaccgaaaaccctgcatcgcatagacagcgtagttgcgcctctgcttgttcaaactgggggggcgtaggagaggtccctatatgcaatcgggctccatcacgatggaccagtaatgctttacggaaactcagagcattttcctggagaaactgctgccaactctcagtcggtaacggtgctgaacgggtgtgatgcttcgccagtattgcctctgaaagcatgttcataagagtctgcttgttgcgcacgtgccaatacaatgtaggctgctctacacctagcttctgggcgagtttacgggttgttaaaccttcgattccgacctcattaagcagctctaatgcgctgttaatcactttacttttatctaatctagacatggtggctttacgagggtaggaagtggtacggaaagttggtataagacaaaagtgttgtggaattgctccaggcgatctgacggttcactaaacgagctctgcttttataggcgcccaccgtacacgcctaaagcttatacgtaattgtgagcgctcacaattgtaaactggatatacgtaattgtgagcgctcacaattgtaaactgtagatacgtaattgtgagcgctcacaattgtaaactggtcatacgtaattgtgagcgctcacaattgtaaactccttatacgtaattgtgagcgctcacaattgtaaagtctgcatacgtaattgtgagcgctcacaattgtaaactcttcatacgtaattgtgagcgctcacaattgtaaactcctggggactttccagctggggactttccagctgggactttccaggag

SEQ ID NO: 110 - DNA, 621 bp, synthetic construct
  source 1..621: mol_type = other DNA
atgtattcagcgccctccgcctgcacttgcctgtgtttacacttcctgctgctgtgcttccaggtacaggtgctggttgccgaggagaacgtggacttccgcatccacgtggagaaccagacgcgggctcgggacgatgtgagccgtaagcagctgcggctgtaccagctctacagccggaccagtgggaaacacatccaggtcctgggccgcaggatcagtgcccgcggcgaggatggggacaagtatgcccagctcctagtggagacagacaccttcggtagtcaagtccggatcaagggcaaggagacggaattctacctgtgcatgaaccgcaaaggcaagctcgtggggaagcccgatggcaccagcaaggagtgtgtgttcatcgagaaggttctggagaacaactacacggccctgatgtcggctaagtactccggctggtacgtgggcttcaccaagaaggggcggccgcggaagggccccaagacccgggagaaccagcaggacgtgcatttcatgaagcgctaccccaaggggcagccggagcttcagaagcccttcaagtacacgacggtgaccaagaggtcccgtcggatccggcccacacaccctgcc

SEQ ID NO: 111 - protein, 207 aa, synthetic construct
MYSAPSACTCLCLHFLLLCFQVQVLVAEENVDFRIHVENQTRARDDVSRKQLRLYQLYSRTSGKHIQVLGRRISARGEDGDKYAQLLVETDTFGSQVRIKGKETEFYLCMNRKGKLVGKPDGTSKECVFIEKVLENNYTALMSAKYSGWYVGFTKKGRPRKGPKTRENQQDVHFMKRYPKGQPELQKPFKYTTVTKRSRRIRPTHPA

SEQ ID NO: 112 - DNA, 849 bp, synthetic construct
  source 1..849: mol_type = other DNA
atgtctagattagataaaagtaaagtgattaacagcgcattagagctgcttaatgaggtcggaatcgaaggtttaacaacccgtaaactcgcccagaagctaggtgtagagcagcctacattgtattggcacgtgcgcaacaagcagactcttatgaacatgctttcagaggcaatactggcgaagcatcacacccgttcagcaccgttaccgactgagagttggcagcagtttctccaggaaaatgctctgagtttccgtaaagcattactggtccatcgtgatggagcccgattgcatatagggacctctcctacgcccccccagtttgaacaagcagaggcgcaactacgctgtctatgcgatgcagggttttcggtcgaggaggctcttttcattctgcaatctatcagccattttacgttgggtgcagtattagaggagcaagcaacaaaccagatagaaaataatcatgtgatagacgctgcaccaccattattacaagaggcatttaatattcaggcgagaacctctgctgaaatggccttccatttcgggctgaaatcattaatatttggattttctgcacagttagatgaaaaaaagcatacacccattgaggatggtaataaaccaaaaaagaagagaaagctagcagtgtcagtgacatttgaagatgtggctgtgctctttactcgggacgagtggaagaagctggatctgtctcagagaagcctgtaccgtgaggtgatgctggagaattacagcaacctggcctccatggcaggattcctgtttaccaaaccaaaggtgatctccctgttgcagcaaggagaggatccctggtaa

SEQ ID NO: 113 - DNA, 386 bp, synthetic construct
  source 1..386: mol_type = other DNA
gagtttacaattgtgagcgctcacaattacgtatgaagagtttacaattgtgagcgctcacaattacgtatgcagactttacaattgtgagcgctcacaattacgtataaggagtttacaattgtgagcgctcacaattacgtatgaccagtttacaattgtgagcgctcacaattacgtatctacagtttacaattgtgagcgctcacaattacgtatatccagtttacaattgtgagcgctcacaattacgtataagctttaggcgtgtacggtgggcgcctataaaagcagagctcgtttagtgaaccgtcagatcgcctggagcaattccacaacacttttgtcttataccaactttccgtaccacttcctaccctcgtaaa

SEQ ID NO: 114 - DNA, 1296 bp, synthetic construct
  source 1..1296: mol_type = other DNA
gtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatttcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagccaaaaaagaagagaaagctagcagtgtcagtgacatttgaagatgtggctgtgctctttactcgggacgagtggaagaagctggatctgtctcagagaagcctgtaccgtgaggtgatgctggagaattacagcaacctggcctccatggcaggattcctgtttaccaaaccaaaggtgatctccctgttgcagcaaggagaggatccctggtaa

SEQ ID NO: 115 - protein, 431 aa, synthetic construct
VKPVTLYDVAEYAGVSYQTVSRVVNQASHVSAKTREKVEAAMAELNYIPNRVAQQLAGKQSLLIGVATSSLALHAPSQIVAAIKSRADQLGASVVVSMVERSGVEACKAAVHNLLAQRVSGLIINYPLDDQDAIAVEAACTNVPALFLDVSDQTPINSIIFSHEDGTRLGVEHLVALGHQQIALLAGPLSSVSARLRLAGWHKYLTRNQIQPIAEREGDWSAMSGFQQTMQMLNEGIVPTAMLVANDQMALGAMRAITESGLRVGADISVVGYDDTEDSSCYIPPLTTIKQDFRLLGQTSVDRLLQLSQGQAVKGNQLLPVSLVKRKTTLAPNTQTASPRALADSLMQLARQVSRLESGQPKKKRKLAVSVTFEDVAVLFTRDEWKKLDLSQRSLYREVMLENYSNLASMAGFLFTKPKVISLLQQGEDPW

SEQ ID NO: 116 - DNA, 442 bp, synthetic construct
  source 1..442: mol_type = other DNA
gagtttacaacaaacagacaatctggtctgtttgtaacgtatgaagagtttacaacaaacagacaatctggtctgtttgtaacgtatgcagactttacaacaaacagacaatctggtctgtttgtaacgtataaggagtttacaacaaacagacaatctggtctgtttgtaacgtatgaccagtttacaacaaacagacaatctggtctgtttgtaacgtatctacagtttacaacaaacagacaatctggtctgtttgtaacgtatatccagtttacaacaaacagacaatctggtctgtttgtaacgtataagctttaggcgtgtacggtgggcgcctataaaagcagagctcgtttagtgaaccgtcagatcgcctggagcaattccacaacacttttgtcttataccaactttccgtaccacttcctaccctcgtaaa

SEQ ID NO: 117 - DNA, 612 bp, synthetic construct
  source 1..612: mol_type = other DNA
atgagtccaaagagaagaacacaggcagagcgcgcaatggagacccagggcaagttgattgcagcggccctgggggttttacgggaaaaaggttacgcgggattccggatcgcagatgtgcccggtgctgcaggtgtctcgagaggagcgcagagccatcatttcccgacaaagcttgagcttctgcttgccacttttgaatggctttacgaacagatcaccgaacgcagtcgggctcgattagcgaaattgaagccagaggatgacgtcatccagcaaatgctggacgacgccgccgaatttttcctcgacgatgacttctctatcagccttgatttgattgtggctgccgaccgggatccagcgttacgcgagggtattcagcgcacggtagagaggaatcggtttgtcgtcgaggatatgtggcttggtgttctggtgagccgtggtctttcgcgtgatgatgcagaagatatcctttggttgatattcaattcggtgcgtgggcttgctgttcgtagcctatggcagaaggacaaagaacgctttgagcgtgtcaggaactcgacactcgaaattgcgcgagagcggtacgcgaaattcaagcgctag

SEQ ID NO: 118 - protein, 203 aa, synthetic construct
MSPKRRTQAERAMETQGKLIAAALGVLREKGYAGFRIADVPGAAGVSRGAQSHHFPTKLELLLATFEWLYEQITERSRARLAKLKPEDDVIQQMLDDAAEFFLDDDFSISLDLIVAADRDPALREGIQRTVERNRFVVEDMWLGVLVSRGLSRDDAEDILWLIFNSVRGLAVRSLWQKDKERFERVRNSTLEIARERYAKFKR